(19) United States
(12) Patent Application Publication
(10) Pub. No.: US 2026/0151493 A1
(43) Pub. Date: Jun. 4, 2026

(54) TRI-AGONISTS OF THE GLP-1, GIP, AND AMYLIN RECEPTORS AND USES THEREOF

(71) Applicant: Novo Nordisk A/S, Bagsvaerd (DK)

(72) Inventors: Thomas Kruse, Herlev (DK); Jesper F. Lau, Farum (DK); Charlotte Stahl Madsen, Maaloev (DK); Christina Sophie Haxvig, Hilleroed (DK)

(21) Appl. No.: 19/404,431

(22) Filed: Dec. 1, 2025

Related U.S. Application Data
(63) Continuation of application No. PCT/EP2025/084534, filed on Nov. 27, 2025.

(30) Foreign Application Priority Data
Nov. 29, 2024 (WO) PCT/EP2024/084031
May 27, 2025 (EP) 25179080.4

(51) Int. Cl. A61K 47/64; A61K 47/65; A61P 3/04

(52) U.S. Cl. CPC A61K 47/64; A61K 47/65; A61P 3/04

(57) ABSTRACT

The present invention relates to a GLP-1-/GlP-/amylin-receptor tri-agonist having a balanced potency ratio across all three receptors and/or having improved pharmacokinetic properties. The invention also relates to pharmaceutical compositions comprising such a GLP-1-/GlP-/amylin-receptor tri-agonist as well as their use as a medicament for the treatment of subjects with overweight, obesity and associated co-morbidity.

### CROSS-REFERENCE TO RELATED APPLICATIONS

This application is a continuation of International Application PCT/EP2025/084534, filed Nov. 27, 2025, which claims priority to International Application PCT/EP2024/084031, filed Nov. 29, 2024, and European Patent Application 25179080.4 filed May 27, 2025, the contents of which are incorporated by reference in their entirety.

### TECHNICAL FIELD

GLP-1-/GlP-/amylin-receptor tri-agonists and compositions comprising such compounds for use in medicine.

### INCORPORATION-BY-REFERENCE OF THE SEQUENCE LISTING

The instant application contains a Sequence Listing which has been submitted in XML format via USPTO patent electronic filing system and is hereby incorporated by reference in its entirety. Said XML file, created on Dec. 1, 2025, is named “240066US01.xml”, and is 298,806 bytes in size.

### BACKGROUND

Overweight and obesity are the abnormal or excessive accumulation of body fat that present a risk to an individual's overall health. The WHO considers body mass index (BMI) to be the most convenient population-level measure of overweight and obesity. In adults, a body mass index (BMI) greater than or equal to 25 is considered overweight, and a BMI of greater than or equal to 30 is considered obese. Obesity is further subclassified into class I (BMI 30-34.9), class II (BMI 35-39.9) and class III (BMI>40).

Obesity is a leading risk factor in a large number of serious conditions, including type 2 diabetes and its associated co-morbidities, and cardiovascular diseases such as heart disease and stroke, which are the leading causes of death worldwide. Obesity is now recognised by the World Health Organization (WHO) as an issue that has grown to epidemic proportion, even in children: in 2016, 1.9 billion adults worldwide were reported to have obesity; in 2019, 38.3 million children under the age of 5 worldwide were reported to have obesity. According to the WHO, 422 million people worldwide have diabetes, and 1.6 million deaths are directly attributed to diabetes each year. There is, therefore, a huge incentive for the individual, as well as society, to try to prevent and/or treat obesity.

When life-style modification such as diet and exercise alone do not suffice in reducing the body mass index (BMI) of an individual living with obesity to an acceptable level, treatment with pharmaceutical drugs such as liraglutide, orlistat and naltrexone-bupropion have been shown to cause some weight loss. Nonetheless, these weight losses are often not sustained and are too small for individuals with class II and class III obesity. In these cases, bariatric surgery has proven necessary. While bariatric surgery is currently the most effective treatment in terms of obtaining long-term weight loss, it is an invasive procedure associated with high risk to the patient and high cost. Therefore, an efficacious and minimally invasive treatment would be a significant improvement in the treatment of obesity.

GLP-1 is a 30 or 31-amino acid polypeptide that is synthesised and secreted from enteroendocrine L-cells. GLP-1 is an incretin hormone, decreasing blood sugar levels in a glucose-dependent manner by enhancing the secretion of insulin. Endogenous GLP-1 is rapidly degraded, primarily by dipeptidyl peptidase-4 (DPP-4), resulting in a half-life of <2 minutes.

Several marketed products containing a long-acting GLP-1 receptor agonist as the active pharmaceutical ingredient are approved for treatment of type 2 diabetes. These include dulaglutide (Trulicity®), exenatide (Byetta®, Bydureon®), liraglutide (Victoza®), lixisenatide (Lyxumia®), and semaglutide (Ozempic®).

Two marketed products containing a GLP-1 receptor agonist as the active pharmaceutical ingredient are approved for use in individuals living with overweight and have at least one weight-related co-morbidity or in individuals living with obesity: liraglutide (Saxenda®) and semaglutide (Wegovy®). The maximum efficacy that can be achieved with a GLP-1 receptor agonist is limited by tolerability. At increasing doses, side-effects such as nausea and vomiting become increasingly pronounced.

Native human GlP is a 42-amino acid polypeptide synthesized in and secreted by specialized enteroendocrine K-cells. These cells are concentrated primarily in the duodenum and proximal jejunum, although they also can be found throughout the intestine. The main stimulant for GlP secretion is ingestion of carbohydrate- and lipid-rich meals. Following ingestion, circulating plasma GlP levels increase 10- to 20-fold. Like GLP-1, GlP is an incretin hormone and in healthy humans it appears to be a more powerful incretin than GLP-1. However, in individuals living with type-2-diabetes GlP has lost its incretin effect. The half-life of intact GlP is estimated to be approximately 7 minutes in healthy subjects and approximately 5 minutes in people living with type-2 diabetes.

Long acting (or protracted) GlP analogues have been shown to lower body weight and improve glycaemic control. With regards to body weight reduction, this effect is comparatively less than long-acting GLP-1 analogues in rodent models (Mroz et al, Mol Metab, 2019, 20: 51-62). Moreover, GlP analogues induce body weight loss by an additive/synergistic action with long-acting GLP-1 analogues in dual administration (Finan et al, Sci Transl Med, 2013, 5 (209): 209ra151; Nørregaard et al, Diabetes Obes Metab, 2018, 20 (1): 60-68), and as such represent suitable candidates for amplification of GLP-1-based pharmacology. GlPR agonism can also be included as a partner to GLP-1 receptor agonism as a single molecule co-agonist to amplify GLP-1 driven body weight loss and improvement in glycaemic control, as has been shown in preclinical animal models (Finan et al, Sci Transl Med, 2013, 5 (209): 209ra151; Coskun et al, Mol Metab, 2018, 18: 3-14). Two different peptides (MAR709 and LY3298176, the latter known as tirzepatide) with high potency on both GLP-1R and GlPR have been tested in multi-dose clinical trial studies. The clinical results have demonstrated improvements in glycaemic control and body weight that exceed that achieved with comparable dosing of benchmark GLP-1 specific agonists (Frias et al, Cell Metab, 2017, 26 (2): 343-352; Frias et al, Lancet, 2018, 392 (10160): 2180-2193), demonstrating the translational aspects and therapeutic benefits of co-targeting GLP-1 and GlP receptors.

Lately this concept of co-targeting GLP-1 and GlP receptors with a GLP-1/GlP co-agonist has been proven, as the compound tirzepatide has been approved in 2022 for the treatment of diabetes. Furthermore, tirzepatide is also useful for the treatment of obesity, as it has shown in a phase 3 clinical trial that a high dose of tirzepatide (15 mg) helped patients to lose (mean) 20.9% of their body weight after 72 weeks of treatment, including a 20-week dose-escalation period (A M Jastreboff, L J Aronne, N N Ahmad, et al., N Engl J Med 2022; 387:205-216). Tirzepatide was recently approved for weight management in people with a BMI>30 or a BMI>27 and at least one weight related co-mobility (tradename: Zepbound®).

Beside tirzepatide, which is described in WO 2016/111971 A1, GLP-1/GlP co-agonists and their potential medical uses are described in several patent applications such as WO 2006/086769, WO 2010/011439, WO 2013/164483, WO 2014/192284, WO 2015/067715, WO 2015/022420, WO 2015/086728, WO 2015/086729, WO 2016/111971, WO 2020/023386, US 2014/162945, US 2014/357552, and WO 2022/018186.

Amylin is a 37-amino acid long polypeptide hormone produced in pancreatic beta (β)-cell from where it is co-secreted with insulin. Amylin has a half-life of 15-20 minutes. It produces its effects in several different organ systems, primarily acting via amylin receptors 1-3 (AMYR1-3). Amylin is an important regulator of energy metabolism in health and disease, inhibiting glucagon secretion, delaying gastric emptying, signalling satiety, and suppressing appetite. Other amylin actions have also been reported, such as on the cardiovascular system and on bone.

Clinical studies have shown that amylin receptor agonists may be useful for the treatment of overweight, obesity, type 1 diabetes and/or type 2 diabetes. Currently, there is one product on the market (Symlin®) which contains an amylin receptor agonist (pramlintide acetate) as the active pharmaceutical ingredient. Symlin®, a liquid pharmaceutical composition for subcutaneous administration, is approved for use in patients with type 1 or type 2 diabetes who use basal and mealtime insulin and have failed to achieve desired glycaemic control, despite optimal insulin therapy. Pramlintide for use in people living with overweight and obesity was also investigated. Pramlintide has a short half-life (less than 1 hour), necessitating administration thrice daily. Consequently, there is a large diurnal difference in the pramlintide plasma concentrations.

There has been a similar desire to be able to prolong the action of amylin and the co-targeting of the amylin receptor and the GLP-1 receptor has been described. Amylin receptor agonists and their potential medical uses are described in several patent applications such as WO 2012/168432, WO 2016/034604, WO 2022/129254, WO 2022/063925, or US 2022/0288168

A fixed-dose combination of an amylin receptor agonist, cagrilintide, and a GLP-1 receptor agonist, semaglutide, is currently under investigation for the treatment of overweight and obesity (Lancet 2021; 397: 1736-48). The drug products being investigated are separate liquid pharmaceutical compositions for subcutaneous use. A clinical trial has demonstrated that a combination of cagrilintide and semaglutide induced greater weight loss in people living with obesity than the maximal approved dose of semaglutide monotherapy. Worsening of the side-effect profile was not observed. Co-agonists of GLP-1 and amylin receptors, and their potential medical uses are described in several patent applications such as WO 2022/129526 A1. Therein peptide co-agonists of the human GLP-1R and amylin receptor are disclosed, which are potent and are balanced, i.e., having a similar level of activation of both receptor systems, and which are showing oral bioavailability. Another example is WO 2007/022123, which describes hybrid polypeptides including exendin covalently linked to amylin. However, there is no co-agonist of GLP-1 and amylin receptors which has so far obtained market approval.

Finally, WO 2023/288313 and WO 2024/015922 disclose multi-agonist peptides useful as agents for the treatment and prevention of metabolic diseases and disorders, in particular diabetes and obesity. WO 2023/288313 discloses peptides including two or more component peptides including amylin, GlP, GLP-1, and/or calcitonin. The specifically disclosed peptides are tri-agonists of the GLP-1, GlP, and amylin receptors, showing activity on all three receptors and reduction of food intake and body weight in animals.

Whilst current therapy options and investigatory drugs provide promise, individuals living with overweight, obesity and/or associated co-morbidities can, at best, at current time hope to be treated with injectable pharmaceutical formulations or medications with some efficacy. There still remains a need in the art for a more efficacious medicament, which is potent in vitro and potent on weight loss, and which does not simultaneously result in a proportionally increased level of side-effects, and which has improved pharmacokinetic properties, has improved chemical stability, is suitable for once weekly dosing in humans, and/or is suitable for oral administration.

### SUMMARY

The present invention relates to a GLP-1-/GlP-/amylin-receptor tri-agonist comprising a peptide according to Formula I:

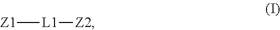

wherein Z1 is a peptide GLP-1-/GlP-receptor co-agonist, L1 is a peptide linker and Z2 is a peptide amylin-receptor agonist and wherein the peptide according to formula (I) comprises one lysine (Lys, K).

In one aspect the present invention relates to a GLP-1-/GlP-/amylin-receptor tri-agonist comprising a peptide according to Formula I:

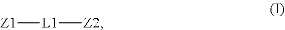

comprising one lysine (Lys, K) residue; wherein:
Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).

In some embodiments of the peptide Z1 X₂ is Aib, X₁₂ is Ile (I) or Lys (K), X₂₀ is Arg (R), X₂₄ is Glu (E), X₂₇ is Leu (L) or I (Ile), X₂₈ is Ala (A), X₃₀ is Gly (G) or Ala (A), X₃₁ is Gly (G), Ala (A) or Pro (P), X₃₃ is Ser (S) and X₃₄ is Gly (G).

In some embodiments of the peptide Z2 X₅₂ is Ser (S), X₅₃ is Glu (E) or His (H), X₅₈ is Ala (A) or Gln (Q), X₅₉ is Leu (L) or Thr (T), X₆₀ is Gly (G) or Gln (Q), X₆₈ is Gln (Q), Glu (E), or Lys (K), and X₇₂ is Leu (L).

In some embodiments, the GLP-1-/GlP-/amylin-receptor tri-agonist has an amino acid sequence which comprises or consists of
YX₂EGTFTSDYSKLLEEIAAREFIEWLLAGGPSSGAGASELSTAQLGRLSAELHQLATLPRTE TGSGSP (SEQ ID NO: 230), or
YX₂EGTFTSDYSILLEEIAAREFIEWLLAGGASSGAGEAPGEAPGASHLSTAQTQRLSAELHK LATLPRTETGSGSP (SEQ ID NO: 243), or
YX₂EGTFTSDYSKLLEEIAAREFIEWLLAGGPSSGAGASELSTAALGRLSAELHQLATLPRTE TGSGSP (SEQ ID NO: 244), or
YX₂EGTFTSDYSKLLEEIAAREFIEWLIAGAPSSGAGASELSTAALGRLSAELHQLATLPRTET GSGSP (SEQ ID NO: 246), or
YX₂EGTFTSDYSKLLEEIAAREFIEWLIAGAPSSGAGASELSTAALGRLSAELHELATLPRTET GSGSP (SEQ ID NO: 250), or
YX₂EGTFTSDYSKLLEEIAAREFIEWLLAGGGSSGAGASELSTAALGRLSAELHELATLPRTE TGSGSP (SEQ ID NO: 251), or
YX₂EGTFTSDYSKLLEEIAAREFIEWLLAGGPSSGAGASELSTAALGRLSAELHELATLPRTE TGSGSP (SEQ ID NO: 252), wherein in each amino acid X₂ is Aib.

In some embodiments, the GLP-1-/GlP-/amylin-receptor tri-agonist comprises a protraction moiety that increases half-life of the peptide Z1-L1-Z2, e.g. attached to via a lysine residue at position 12 of Z1 (X₁₂) or at position 18 of Z2 (X₆₈).

In a second aspect, the invention further relates to pharmaceutical compositions comprising such GLP-1-/GlP-/amylin-receptor tri-agonist and one or more pharmaceutically acceptable excipients, as well as the medical use of said tri-agonists, in particularly for use in the treatment of subjects with an initial body mass index (BMI) of 27 or more, such as 30 or more, optionally in the presence of at least one weight-related co-morbidity.

In a third aspect the invention relates to a balanced GLP-1-/GlP-/amylin-receptor tri-agonist that is capable of selectively activating or “agonising” all three of the GLP-1 receptor, the GlP receptor, and the amylin receptor to a similar level.

Also or alternatively, in a fourth aspect, the invention relates to a GLP-1-/GlP-/amylin-receptor tri-agonist with improved pharmacokinetic properties.

Also or alternatively, in a fifth aspect, the invention relates to a GLP-1-/GlP-/amylin-receptor tri-agonist which is suitable for once weekly administration.

Also or alternatively, in a sixth aspect, the invention relates to a GLP-1-/GlP-/amylin-receptor tri-agonist which is suitable for oral administration.

Also or alternatively, in a seventh aspect, the invention relates to a GLP-1-/GlP-/amylin-receptor tri-agonist that reduces food intake.

Also or alternatively, in a seventh aspect, the invention relates to a GLP-1-/GlP-/amylin-receptor tri-agonist with improved chemical stability.

The invention may also solve further problems that will be apparent from the disclosure of the exemplary embodiments and aspects.

### DESCRIPTION

The present invention relates to a GLP-1-/GlP-/amylin-receptor tri-agonist comprising a peptide according to Formula I:

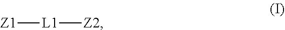

wherein Z1 is a peptide GLP-1-/GlP-receptor co-agonist, L1 is a peptide linker and Z2 is a peptide amylin-receptor agonist and wherein the peptide according to formula (I) comprises one lysine (Lys, K).

The compounds disclosed herein are referred to as “GLP-1-/GlP-/amylin-receptor tri-agonist” or “GLP-1 receptor-GlP receptor-amylin receptor tri-agonists” or “GLP-1-/GlP-/amylin-receptor triple agonist” or “GLP-1 receptor-GlP receptor-amylin receptor triple agonists”.

The GLP-1-/GlP-/amylin-receptor tri-agonist comprises a peptide Z1, which is a GLP-1-/GlP receptor co-agonist, a peptide linker L1, and a peptide Z2, which is an amylin receptor agonist. The GLP-1-/GlP-/amylin-receptor tri-agonist is a compound that binds to each of the three GLP-1, GlP, and amylin receptors and is capable of activating each receptor GLP-1R, GlPR, and amylin receptor, thus eliciting a response at each receptor.

The compounds disclosed herein are agonists at each of the receptors GLP-1, GlP, and amylin. Hence the compound of the present invention is a GLP-1 receptor agonist and a GlP receptor agonist and an amylin receptor agonist. It is capable of activating or “agonising” all three of the GLP-1 receptor, the GlP receptor, and the amylin receptor: it is a “GLP-1-/GlP-/amylin-receptor tri-agonist”. The GLP-1-/GlP-/amylin-receptor tri-agonist may provide a similar level of activation of all three GLP-1, GlP, and amylin receptors; then it is referred to as a “balanced GLP-1-/GlP-/amylin-receptor tri-agonist” or in short a “balanced tri-agonist”.

A “receptor agonist” or “agonist” may be defined as a ligand, such as a compound, that binds to and activates a biological receptor to produce a biological response. A full agonist may be defined as one that elicits a response of the same magnitude as the natural ligand (see e.g., “Principles of Biochemistry”, A L Lehninger, D L Nelson, M M Cox, Second Edition, Worth Publishers, 1993, page 763). Receptors can be activated by either endogenous agonists, such as endogenous hormones, or exogenous agonists, such as pharmaceutical drugs.

In the context of the current invention, a “co-agonist” is a compound capable of binding to and activating two different biological receptors, e.g. a compound comprising two different ligands, each of which binds to a given biological receptor, to produce a biological response that is characteristic of the natural ligands. In an analogous way, a “triple agonist” or “tri-agonist” is a compound capable of binding to and activating three different biological receptors, e.g. a compound comprising three different ligands, each of which binds to a given biological receptor, to produce a biological response that is characteristic of the natural ligands.

A “GLP-1 receptor agonist” may be defined as a compound which is capable of binding to the GLP-1 receptor and capable of activating it. A “full” GLP-1 receptor agonist may be defined as a GLP-1 receptor agonist which is capable of eliciting a magnitude of GLP-1 receptor response that is similar to native glucagon like peptide 1 (GLP-1). Semaglutide, disclosed in WO 2006/097537, Example 4, is an example of an exogenous GLP-1 receptor agonist.

A “GlP receptor agonist” may be defined as a compound which is capable of binding to the GlP receptor and capable of activating it. A “full” GlP receptor agonist may be defined as a GlP receptor agonist which is capable of eliciting a magnitude of GlP receptor response that is similar to native glucose-dependent insulinotropic polypeptide (GlP).

A “GLP-1/GlP receptor co-agonist” may be defined as a compound which is capable of binding to both, the GLP-1 receptor, and the GlP receptor and capable of activating both receptors. An example of a GLP-1/GlP co-agonist is tirzepatide, which is described in WO 2016/111971.

An “amylin receptor agonist” may be defined as a compound which is capable of binding to the amylin receptors (AMYRs) and the calcitonin receptor (CTR) and capable of activating it. Amylin receptors consist of heterodimers of two components: the calcitonin receptor (CTR) and one of three receptor activity-modifying proteins (RAMP1-3), resulting in three possible complexes, AMYR1-3. Unless otherwise specified herein, “amylin receptor” at least refers to amylin receptor 3 (AMYR3). Nonetheless, some concomitant activity on the other receptors can be expected. A “full” amylin receptor agonist may be defined as an amylin receptor agonist which is capable of eliciting a magnitude of amylin receptor response that is similar to native amylin. An amylin receptor agonist will often also be a calcitonin receptor agonist. Examples of amylin receptor agonists are human amylin, pramlintide and cagrilintide (disclosed in WO 2012/168432). It is noted that all headings and sub-headings are used herein for convenience only and should not be construed as limiting the invention in any way.

The use of any and all examples, or exemplary language (e.g., “such as”) provided herein, is intended merely to better illuminate the invention, and does not pose a limitation on the scope of the invention unless otherwise claimed.

In order that the present invention may be more readily understood, certain terms are first defined.

In what follows, Greek letters may be represented by their symbol or the corresponding written name, for example: α=alpha; β=beta; γ=gamma; ε=epsilon; ω=omega; etc. Also, the Greek letter of μ may be represented by “u”, e.g. in μl=ul, or in μM=uM.

Unless otherwise indicated in the specification, terms presented in singular form generally also include the plural situation. The term “a” or “an” is intended to mean “one or more.”

The term “comprise” and variations thereof such as “comprises” and “comprising,” when preceding the recitation of a step or an element, are intended to mean that the addition of further steps or elements is optional and not excluded. As disclosed herein the open-ended terms like “comprises” and “comprising” might be replaced with closed terms such as “consists of’, “consisting of”, and the like.

The term “about” is used herein to mean approximately, roughly, or around. When the term “about” is used in conjunction with a numerical range, it modifies that range by extending the boundaries above and below the numerical values set forth. In general, the term “about” can modify a numerical value above and below the stated value by 10 percent, up or down (higher or lower).

Amino acids are molecules containing an amine group and a carboxylic acid group, and, optionally, one or more additional groups, often referred to as a side chain.

The term “amino acid” includes canonical amino acids (which are genetically encoded), and non-proteinogenic amino acids. Non-limiting examples of non-proteinogenic amino acids are Aib (α-aminoisobutyric acid or 2-aminoisobutyric acid), and the d-isomers of the canonical amino acids. All amino acid residues within the peptide for which the optical isomer is not stated is herein to be understood to mean the L-isomer, unless otherwise specified.

Herein, “amino acid substitution” or “substitution” refers to one or more amino acid(s) being replaced with the same number of amino acid(s) in the backbone of the peptide. Substitutions may be, but are not limited to, conservative substitutions. For example, an amino acid may be substituted to an amino acid with similar biochemical properties, for example, a basic amino acid may be substituted to another basic amino acid (e.g. lysine to arginine), an acidic amino acid may be substituted to another acidic amino acid (e.g. glutamate to aspartate), a neutral amino acid may be substituted to another neutral amino acid (e.g. threonine to serine), a charged amino acid may be substituted to another charged amino acid (e.g. glutamate to lysine), a hydrophilic amino acid may be substituted to another hydrophilic amino acid (e.g. asparagine to glutamine), a hydrophobic amino acid may be substituted to another hydrophobic amino acid (e.g. alanine to valine), a polar amino acid may be substituted to another polar amino acid (e.g. serine to threonine), an aromatic amino acid may be substituted to another aromatic amino acid (e.g. phenylalanine to tryptophan) and an aliphatic amino acid may be substituted to another aliphatic amino acid (e.g. leucine to isoleucine).

The term “excipient” as used herein broadly refers to any component other than the Active Pharmaceutical Ingredient (API).

The term “identity” or “sequence identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percentage of identical matches between the smaller of two or more sequences with gap alignments (if any) addressed by a particular mathematical model or computer program (i.e., “algorithms”). Identity of related polypeptides can be readily calculated by known methods, e.g. using Needleman (Needleman et al. J. Mol. Biol. 1970; 48:443-453) from EMBOSS-6.6.0 using the parameters 10 and 0.5 for gaps opening and extensions, respectively (gapopen=10, gapextend=0.5) or e.g. calculated by: (1) comparing two optimally aligned sequences over a window of comparison (e.g., the length of the longer sequence, the length of the shorter sequence, a specified window, etc.), (2) determining the number of positions containing identical monomers (e.g., same amino acids occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent “sequence identity”. For example, if peptides A and B are both 20 amino acids in length and have identical amino acids at all but 1 position, then peptide A and peptide B have 95% sequence identity.

The term “polypeptide” or “peptide” as used herein includes oligopeptides and refers to a single chain of amino acids connected by one or more amide (or peptide) bonds. The terms “polypeptide” and “peptide” shall be used interchangeably herein.

The term “half-life” or “plasma half-life” as used herein refers to the time required for half the quantity of a substance administered to a person to be metabolized or eliminated from the serum or plasma of the person by normal biological processes.

The term “treatment” and variations thereof, as used herein, refers to the medical therapy of any human subject in need thereof. The term includes administering a therapeutically effective amount of a peptide as disclosed herein sufficient to reduce or eliminate at least one symptom of the disorder in question. “Treatment”, however, need not be a cure. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

As used herein the terms “preventing”, “prevent” or “prevention” or variations thereof refers to protecting a subject from developing at least one symptom of a disease or reducing the severity of a symptom of a disorder.

The term “compound” is used herein to refer to a molecular entity, and “compounds” may thus have different structural elements besides the minimum element defined for each compound or group of compounds. It follows that a compound may be a peptide or a derivative thereof, as long as the compound comprises the defined structural and/or functional elements. The term “compound” is also meant to cover pharmaceutically relevant forms thereof, i.e. a compound as defined herein or a pharmaceutically acceptable salt, amide, or ester thereof.

The compound disclosed herein may be a potent GLP-1 receptor agonist.

The compound disclosed herein may be a potent GlP receptor agonist.

The compound disclosed herein may be a potent amylin receptor agonist.

The in vitro potency of the agonists may be measured as described in the assays of Example 4. The term “potency” is used to describe the effect of a given compound in assays where a sigmoidal relationship between log concentration and the effect of a compound has been established. Furthermore, the response should be variable from 0 to 100%. The potency of the compound may be described by means of its EC(effective concentration)₅₀ values. EC₅₀ represents the concentration of compound upon which 50% of its maximal effect is observed in the assay, e.g., as described in Example 4. The lower the EC₅₀ value, the more potent the compound.

The compound disclosed herein may provide a similar level of activation of all three GLP-1, GlP, and amylin receptors; that is, it may be “balanced” and is referred to as a “balanced GLP-1-/GlP-/amylin-receptor tri-agonist” or in short a “balanced tri-agonist”. Relatively “balanced” receptor activation is advantageous because the relative ratio of the compound's GLP-1, GlP, and amylin receptor agonist activities is locked to the molecule; it is not possible to titrate the three receptor agonists, relative to one another. Ultimately, where a molecule is “balanced”, it may be dosed such that all three hormone systems are activated without side-effects outweighing benefits.

A tri-agonist whose potency ratio (A/B) of the potency (A) of the receptor with lowest potency (i.e. the highest numerical EC₅₀ value) divided by the potency (B) of the receptor with highest potency (i.e. the lowest numerical EC₅₀ value) is less than 50, preferably less than 30, is defined as a “balanced tri-agonist” or “balanced GLP-1-/GlP-/amylin-receptor tri-agonist” (based on the assays in the absence of human serum albumin (HSA), as shown in Example 4 Table 11 and Table 12). For example, compound 183 has an EC₅₀ of 14.7 pM on the GLP-1 receptor, an EC₅₀ of 2.17 pM on the GlP receptor, and an EC₅₀ of 7.08 pM on the amylin receptor. Hence it has lowest potency on the hGLP-1 and (A) corresponds to 14.7 pM, and it has its highest potency on the GlP receptor with 2.17 pM, which corresponds to (B). Hence the potency ratio (NB) is 14.7 pM (A) divided by 2.17 pM (B) is equal to 7 (rounded accordingly), this means that compound 183 is a “balanced tri-agonist”.

A compound that is potent on one receptor and much less potent on the other(s) would be “unbalanced”. Such an “unbalanced tri-agonist” is defined as compound having a potency ratio (NB) of 50 or more, or alternatively, having a potency ratio (NB) of 30 or more. For example, reference compound 1 has a potency ratio (NB) of 256 (i.e., 1680 pM (=A) divided by 6.55 pM (=B), rounded accordingly). For example, this reference compound being potent (i.e. EC₅₀ value of <30 pM) on the amylin receptor, less potent (i.e. EC₅₀ value of 221 pM) on the GlP receptor, and even less potent (i.e. EC₅₀ value of 1680 pM) on the GLP-1 receptor, would be unable to achieve optimal efficacy from all three hormone systems.

### GLP-1-/GlP-Receptor Co-Agonist—Z1 Peptide

The present invention relates to a peptide according to Formula I comprising a peptide Z1, which is a GLP-1-/GlP-receptor co-agonist.

In one embodiment, the GLP-1-/GlP-receptor co-agonist, Z1, is a peptide having a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ is Aib.

In one embodiment, the GLP-1-/GlP-receptor co-agonist is a peptide comprising or consisting of an amino acid sequence according to Formula (111) (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E),
X₂₇ represents Leu (L) or I (Ile),
X₂₈ represents Ala (A) or Gln (Q), preferably Ala (A),
X₃₀ represents Gly (G) or Ala (A),
X₃₁ represents Gly (G), Gln (Q), Ala (A) or Pro (P), preferably Pro (P) or Ala (A),
X₃₃ represents Glu (E) or Ser (S), preferably Ser (S),
X₃₄ represents Gly (G) or Glu (E), preferably Gly (G).

In some embodiments, the peptide Z1 disclosed herein may have a maximum of 5 amino acid substitutions, relative to Formula II (SEQ ID NO: 1), wherein said substitution(s) can take place in any of the positions 1 to 34, preferably said substitution(s) take place in position 12, 17, 20, 24, 27, 28, 30, 31, 33 and/or 34, more preferably in positions 12, 17, 27, 30, 31, and/or 33. The present invention encompasses variants of the GLP-1-/GlP-/amylin-receptor tri-agonist, as disclosed herein, wherein peptide Z1 may comprise 1, 2, 3, 4 or 5 amino acid substitution(s) relative to Formula II (SEQ ID NO: 1).

Preferred substitutions include a conservative substitution, those which, instead of the amino acid residue, which appears in the sequence, comprises an amino acid with similar biochemical properties or a structural analogue of the amino acid residue.

In one embodiment, the Z1 peptide according to Formula III has a maximum of 5 substitutions relative to Formula II (SEQ ID NO: 1), relative to Formula II.

In one embodiment, the GLP-1-/GlP-receptor co-agonist, Z1, is a peptide having a maximum of 5 substitutions relative to Formula (II), (SEQ ID NO: 1):

(II)
YX₂EGTFTSDYSILLEEQAAREFIEWLLAGGPSKG,

wherein X₂ is Aib
and wherein Z1 comprises or consists of an amino acid sequence according to Formula (III) (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E),
X₂₇ represents Leu (L) or I (Ile),
X₂₈ represents Ala (A) or Gln (Q), preferably Ala (A),
X₃₀ represents Gly (G) or Ala (A),
X₃₁ represents Gly (G), Gln (Q), Ala (A) or Pro (P), preferably Ala (A), Gly (G) or Pro (P),
X₃₃ represents Glu (E) or Ser (S), preferably Ser (S),
X₃₄ represents Gly (G) or Glu (E), preferably Gly (G).

In one embodiment, the GLP-1-/GlP-receptor co-agonist, Z1, is a peptide having a maximum of 4 substitutions relative to Formula (II), (SEQ ID NO: 1):

(II)
YX₂EGTFTSDYSILLEEQAAREFIEWLLAGGPSKG,

wherein X₂ is Aib
and wherein Z1 comprises or consists of an amino acid sequence according to Formula (VII) (SEQ ID NO: 162):

(VII)
YX₂EGTFTSDYSX₁₂LLEEIAAX₂₀EFIX₂₄WLX₂₇X₂₈GGPSSX₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₄ represents Gly (G) or Glu (E).

In one embodiment, the Z1 peptide comprises or consists of an amino acid sequence according to Formula IV (SEQ ID NO: 169):

(IV)
YX₂EGTFTSDYSX₁₂LLEEIAAREFIEWLX₂₇AGX₃₀X₃₁SSG,

wherein
X₂ represents Aib,
X₁₂ represents Ile (I) or Lys (K),
X₂₇ represents Leu (L) or I (Ile),
X₃₀ represents Gly (G) or Ala (A),
X₃₁ represents Ala (A), Gly (G) or Pro (P).

In one embodiment, the Z1 peptide comprises or consists of a peptide having an amino acid sequence selected from the group consisting of sequences shown in Table 1:

TABLE 1
Specific Z1 peptides

| SEQ | |
|---|---|
| ID NO: | Z1 peptide |
| 20 | YX₂EGTFTSDYSILLEEIAAREFIEWLLAGGPSSG |
| 21 | YX₂EGTFTSDYSILLEEIAAREFIEWLIAGGPSSG |
| 22 | YX₂EGTFTSDYSKLLEEIAAREFIEWLLAGGPSSG |
| 23 | YX₂EGTFTSDYSKLLEEIAAREFIEWLIAGGPSSG |
| 24 | YX₂EGTFTSDYSILLEEIAAREFIEWLLAGGPSSE |
| 25 | YX₂EGTFTSDYSILLEEIAAREFIAWLIAGGPSSG |
| 26 | YX₂EGTFTSDYSILLEEIAAREFIQWLIAGGPSSG |
| 27 | YX₂EGTFTSDYSKLLEEIAAREFIEWLLAGGPSSE |
| 28 | YX₂EGTFTSDYSILLEEIAAQEFIEWLLQGGPSSG |
| 29 | YX₂EGTFTSDYSILLEEIAAREFIEWLLAGGASSG |
| 30 | YX₂EGTFTSDYSKLLEEIAAREFIEWLLAGGQSSG |
| 31 | YX₂EGTFTSDYSKLLEEIAAREFIEWLIAGAPSSG |
| 32 | YX₂EGTFTSDYSKLLEEIAAREFIEWLIAGGPSEG |
| 33 | YX₂EGTFTSDYSKLLEEIAAREFIEWLIAGGGSSG |
| 34 | YX₂EGTFTSDYSKLLEEIAAREFIEWLLAGGGSSG |

wherein X₂ is Aib.

In one embodiment, the Z1 peptide comprises or consists of SEQ ID NO: 22. In one embodiment, the Z1 peptide comprises or consists of SEQ ID NO: 29. In one embodiment, the Z1 peptide comprises or consists of SEQ ID NO: 31. In one embodiment, the Z1 peptide comprises or consists of SEQ ID NO: 34.

### L1 Peptide Linker

The GLP-1-/GlP-/amylin-receptor tri-agonist peptide backbone according to Formula I as disclosed herein comprise a peptide linker L1, which may comprise 1 to 14 amino acid residues, in particularly canonical amino acid residues. The peptide linker may comprise 1 to 10 amino acid residues, in particularly canonical amino acid residues, such as 2 to 10, 3 to 10, 4 to 10, 5 to 10, 6 to 10, 7 to 10, 8 to 10, 9 to 10, 1 to 9, 1 to 8, 1 to 7, 1 to 6, 1 to 5, 1 to 4, or 1 to 3 amino acid residues. Specifically, the peptide linker may comprise 1, 2, 3, 4, 5, 6, 7, 8, 9 or 10 amino acid residues, in particularly canonical amino acid residues.

The peptide linker L1 may be represented b Formula VIIIa:

(VIIIa)
X₁₄₁X₁₄₂X₁₄₃X₁₄₄X₁₄₅X₁₄₆X₁₄₇X₁₄₈X₁₄₉X₁₅₀X₁₅₁X₁₅₂X₁₅₃X₁₅₄,

wherein any of X₁₄₁₋₁₅₄ is independently selected from any naturally occurring, or canonical, amino acid residue(s), and wherein any of X₁₄₂₋₁₅₄ may be absent.

The GLP-1-/GlP-/amylin-receptor tri-agonist peptide backbone disclosed herein comprise a peptide linker L1, wherein the peptide linker L1 comprises or consists of the amino acid sequence according to Formula VIIIa. Thus, the GLP-1-/GlP-/amylin-receptor tri-agonist may comprise a peptide or consist of a peptide according to the amino acid sequence of SEQ ID NO: 5.

Any of X₁₄₁₋₁₅₄ may be selected from any nonaromatic amino acid residue. Any of X₁₄₁₋₁₅₄ may be a charged amino acid. Any of X₁₄₁₋₁₅₄ may be a polar amino acid. Any of X₁₄₁₋₁₅₄ may be a hydrophobic amino acid.

Any of X₁₄₁₋₁₅₄ may be independently selected from the group consisting of alanine (Ala, A), glutamic acid (Glu, E), glutamine (Gln, Q), glycine (Gly, G), leucine (Leu, L), phenylalanine (Phe, F), proline (Pro, P), serine (Ser, S), threonine (Thr, T), valine (Val, V) and asparagine (Asn, N). Preferably any of X₁₄₁₋₁₅₄ may be selected from the group consisting of alanine (Ala, A), glutamic acid (Glu, E), glutamine (Gln, Q), glycine (Gly, G), leucine (Leu, L) and proline (Pro, P).

In one embodiment, the peptide linker L1 may be represented by Formula VIIIa, or comprises or consists of the amino acid sequence according to Formula IV:

(VIIIa)
X₁₄₁X₁₄₂X₁₄₃X₁₄₄X₁₄₅X₁₄₆X₁₄₇X₁₄₈X₁₄₉X₁₅₀X₁₅₁X₁₅₂X₁₅₃X₁₅₄,

wherein
X₁₄₁ represents Ala (A), Glu (E), Gly (G),
X₁₄₂ represents Gln (Q), Glu (E), Gly (G), Leu (L), Pro (P) or is absent,
X₁₄₃ represents Ala (A), Gln (Q), Glu (E), Gly (G), Pro (P) or is absent,
X₁₄₄ represents Ala (A), Gln (Q), Glu (E), Gly (G), Pro (P) or is absent,
X₁₄₅ represents Glu (E), Gly (G), Pro (P), Ser (S), Thr (T) or is absent,
X₁₄₆ represents Glu (E), Gly (G), Leu (L), Gln (Q) or is absent,
X₁₄₇ represents Ala (A), Gln (Q), Glu (E), Gly (G), Phe (F) or is absent,
X₁₄₈ represents Ala (A), Gln (Q), Glu (E), Gly (G), Thr (T), Pro (P), Val (V) or is absent,
X₁₄₉ represents Glu (E), Asn (N), Pro (P), Thr (T) or is absent,
X₁₅₀ represents Ala (A), Gln (Q), Glu (E), Gly (G), Leu (L), Pro (P), Ser (S), Val (V) or is absent,
X₁₅₁ represents Ala (A) or is absent,
X₁₅₂ represents Gln (Q) or is absent,
X₁₅₃ represents Thr (T) or is absent,
X₁₅₄ represents Leu (L) or is absent.

In one embodiment, the peptide linker L1 comprises or consists of 1 to 10 amino acid residues and may be represented by Formula VIII:

(VIII)
X₄₁X₄₂X₄₃X₄₄X₄₅X₄₆X₄₇X₄₈X₄₉X₅₀,

wherein any of X₄₁₋₅₀ is independently selected from any naturally occurring, or canonical, amino acid residue(s), and wherein any of X₄₂₋₅₀ may be absent.

The GLP-1-/GlP-/amylin-receptor tri-agonist peptide backbone disclosed herein comprises a peptide linker L1, wherein the peptide linker L1 comprises or consists of the amino acid sequence according to Formula VIII. Thus, the GLP-1-/GlP-/amylin-receptor tri-agonist may comprise a peptide or consist of a peptide according to the amino acid sequence of SEQ ID NO: 10.

Any of X₄₁₋₅₀ may be selected from any nonaromatic amino acid residue. Any of X₄₁₋₅₀ may be a charged amino acid. Any of X₄₁₋₅₀ may be a polar amino acid. Any of X₄₁₋₅₀ may be a hydrophobic amino acid.

Any of X₄₁₋₅₀ may be independently selected from the group consisting of alanine (Ala, A), glutamic acid (Glu, E), glutamine (Gln, Q), glycine (Gly, G), leucine (Leu, L), proline (Pro, P), serine (Ser, S), and valine (Val, V). Preferably any of X₄₁₋₅₀ may be selected from the group consisting of alanine (Ala, A), glutamic acid (Glu, E), glycine (Gly, G), and proline (Pro, P).

In one embodiment the peptide linker L1 may be represented by Formula VIII, or comprises or consists of the amino acid sequence according to Formula VIII:

(VIII)
X₄₁X₄₂X₄₃X₄₄X₄₅X₄₆X₄₇X₄₈X₄₉X₅₀,

wherein
X₄₁ represents Ala (A), Glu (E) or Gly (G),
X₄₂ represents Glu (E), Gly (G) or is absent,
X₄₃ represents Glu (E), Gly (G), Gln (Q) or is absent,
X₄₄ represents Ala (A), Glu (E), Gly (G) or is absent,
X₄₅ represents Glu (E), Gly (G), Pro (P) or is absent,
X₄₆ represents Glu (E), Gly (G) or is absent,
X₄₇ represents Gln (Q), Glu (E) or is absent,
X₄₈ represents Ala (A), Glu (E) or is absent,
X₄₉ represents Glu (E), Pro (P) or is absent,
X₅₀ represents Ala (A), Glu (E), Gly (G), Leu (L), Pro (P), Ser (S), Val (V) or is absent.

In one embodiment, the peptide linker L1 may be represented by Formula VIII, or comprises or consists of the amino acid sequence according to Formula VIII:

(VIII)
X₄₁X₄₂X₄₃X₄₄X₄₅X₄₆X₄₇X₄₈X₄₉X₅₀,

wherein
X₄₁ represents Ala (A) or Glu (E),
X₄₂ represents Glu (E), Gly (G) or is absent,
X₄₃ represents Glu (E), Gly (G), Gln (Q) or is absent,
X₄₄ represents Aia (A), Glu (E), Gly (G) or is absent,
X₄₅ represents Glu (E), Gly (G), Pro (P) or is absent,
X₄₆ represents Glu (E), Gly (G) or is absent,
X₄₇ represents Gln (Q), Glu (E) or is absent,
X₄₈ represents Aia (A), Glu (E) or is absent,
X₄₉ represents Glu (E), Pro (P) or is absent,
X₅₀ represents Aia (A), Glu (E), Gly (G), Leu (L), Pro (P), Ser (S), Val (V) or is absent.

In one embodiment, the peptide linker L1 may be any one of the peptide linkers represented by SEQ ID NOs 125-159. The peptide linker L1 may be any one of the peptide linkers listed in Table 2.

TABLE 2
Specific peptide linkers L1

| SEQ | Peptide | SEQ | |
|---|---|---|---|
| ID NO | linker | ID NO | Peptide linker |
| — | A | 132 | ALAQTLFVNQ |
| — | E | 133 | ALAQTLGTNE |
| — | G | 134 | ALQAPGQAPG |
| — | AE | 135 | ALQAPGQAPL |
| — | GE | 136 | AGQAPGQAPG |
| 125 | APPE | 137 | AGQAPGQAPL |
| 126 | GGGE | 138 | GGGEGGGEGE |
| 127 | AGQAPG | 139 | GQAPGQAPGE |
| 128 | APPPSGGG | 140 | GQEPGQEPGE |
| 129 | APPPSGGGE | 141 | APPPSLAQTLAQTL |
| 130 | APPPSGGGG | 150 | EGQAPGQAPG |
| 131 | ALAQTLAQTL | 151 | AGQEPGQAPG |
| — | AG | 152 | AGQAEGQAPG |
| 142 | AGGGG | 153 | AGQAPEQAPG |
| 143 | AGEAPGQAPG | 154 | AGQAPGEAPG |
| 144 | AGEAPGEAPG | 155 | AGQAPGQEPG |
| 145 | AGQAPGQAPA | 156 | AGQAPGQAEG |
| 146 | AGQAPGQAPE | 157 | AGQEPGQEPG |
| 147 | AGQAPGQAPP | 158 | AGQAPGQAP |
| 148 | AGQAPGQAPS | 159 | AGQAPGEAPL |
| 149 | AGQAPGQAPV | | |

In one embodiment, the peptide linker L1 may be selected from the group consisting of A, E, G, AE, GE, AG, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159).

In one embodiment, the peptide linker L1 may be selected from the group consisting of E, AE, AG, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159).

In a preferred embodiment, the peptide linker L1 may be AG or AGEAPGEAPG (SEQ ID NO: 144).

In one embodiment, the linker L1 comprises or consists of 1 to 10 amino acids residues. In a preferred embodiment, the linker comprises or consists of 1, 2, 5, 9 or 10 amino acid residues, more preferably 2 or 10 amino acid residues.

### Amylin-Receptor Agonist—Z2 Peptide

The present invention relates to a peptide according to Formula I comprising a peptide Z2, which is an amylin-receptor agonist.

In one embodiment, the amylin-receptor agonist, Z2, is a C-terminal amide and having a maximum of 10 amino acid substitutions relative to Formula V (SEQ ID NO: 2):

(V)
ASELSTAALGRLSAELHELATLPRTETGSGSP.

In one embodiment, the amylin-receptor agonist is a peptide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).

In one embodiment, the amylin-receptor agonist, Z2, is a C-terminal amide and having a maximum of 10 amino acid substitutions relative to Formula V (SEQ ID NO: 2):

(V)
ASELSTAALGRLSAELHELATLPRTETGSGSP.

and wherein Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).

The GLP-1-/GlP-/amylin-receptor tri-agonist according to any one of the preceding embodiments, wherein Z2 is a C-terminal amide and comprises or consists of an amino acid sequence according to Formula IX (SEQ ID NO: 61):

(IX)
ASX₅₃LSTAX₅₈X₅₉X₆₀RLSAELHX₆₈LATLPRTETGSGSP,

wherein
X₅₃ represents Glu (E) or His (H),
X₅₈ represents Ala (A) or Gln (Q),
X₅₉ represents Leu (L) or Thr (T),
X₆₀ represents Gly (G) or Gln (Q),
X₆₈ represents Gln (Q), Glu (E), or Lys (K).

The peptide Z2 disclosed herein may have a maximum of 10 amino acid substitution, relative to Formula V (SEQ ID NO: 2), wherein said substitution(s) can take place in any of the positions 1 to 32, preferably said substitution(s) take place in position 2, 3, 7, 8, 9, 10, 15, 18, 20, 21, 22, 24 and/or 31, more preferably in positions 3, 8, 9, 10, and/or 18. The present invention encompasses variants of the GLP-1-/GlP-/amylin-receptor tri-agonist, as disclosed herein, wherein peptide Z2 may comprise 1, 2, 3, 4, 5, 6, 7, 8, 9 or 10 amino acid substitution(s) relative to Formula V (SEQ ID NO: 2). Preferred substitutions include a conservative substitution, as above explained.

In one embodiment, the Z2 peptide is a C-terminal amide and comprises or consists of a peptide having an amino acid sequence selected from the group consisting of:

TABLE 3
Specific Z2 peptides

| SEQ ID NO: | Z2 peptide |
|---|---|
| 40 | ASHLSTAQTQRLSAELHKLATLPRTETGSGSP |
| 41 | ASHLSTAQLGRLSAELHELATLPRTETGSGSP |
| 42 | ASHLSTAQLGRLSAELHQLATLPRTETGSGSP |
| 43 | ASHLSTAQTQRLSAELHELATLPRTETGSGSP |
| 44 | ASHLSTAALGRLSAELHELATLPRTETGSGSP |
| 45 | ASHLSTAQTARLSAELHKLATLPRTETGSGSP |
| 46 | ASQLSTAQTQRLSAELHKLATLPRTETGSGSP |
| 47 | ASELSTAQTQRLSAELHKLATLPRTETGSGSP |
| 48 | AGELSTAQTQRLSAELHKLATLPRTETGSGSP |
| 49 | ASELSTAQTARLSAELHKLATLPRTETGSGSP |
| 50 | ASHLSTAQTQRLSAELHKLATEPRTETGSGSP |
| 51 | ASELSTAQTQRLSAELHKLATEPRTETGSGSP |
| 2 | ASELSTAALGRLSAELHELATLPRTETGSGSP |
| 53 | ASELSTAALGRLSAELHQLATLPRTETGSGSP |
| 54 | ASELSTAALGRLSAELHQLATEPRTETGSGSP |
| 55 | ASELSTAQLGRLSAELHQLATEPRTETGSGSP |
| 56 | ASELSTAQTGRLSAELHQLATEPRTETGSGSP |
| 57 | ASELSTAQTQRLSAELHQLATEPRTETGSGSP |
| 58 | ASQLSTAQTQRLSAELHKLATEPRTETGSGSP |
| 59 | ASELSTAQLGRLSAELHQLATLPRTETGSGSP |
| 60 | ASHLSTAQLGRLSAELHQLATEPRTETGSGSP |

In one embodiment, the Z2 peptide comprises or consists of SEQ ID NO: 2. In one embodiment, the Z2 peptide comprises or consists of SEQ ID NO: 40. In one embodiment, the Z2 peptide comprises or consists of SEQ ID NO: 53. In one embodiment, the Z2 peptide comprises or consists of SEQ ID NO: 59.

GLP-GLP-1-/GlP-/Amylin-Receptor Tri-Agonist Peptide

In one aspect the invention relates to a GLP-1-/GlP-/amylin-receptor tri-agonist which is a peptide, also referred to as “GLP-1-/GlP-/amylin-receptor tri-agonist peptide. The GLP-1-/GlP-/amylin-receptor tri-agonist comprise or consist of a peptide, Z1-L1-Z2 comprising a peptide Z1, a peptide linker L1, and a peptide Z2.

The peptide Z1 is a GLP-1-/GlP receptor co-agonist which is capable of binding to both, the GLP-1 receptor, and the GlP receptor and capable of activating both receptors. The C-terminus of peptide Z1 is attached to the peptide linker L1 via a peptide bond.

L1 is a peptide linker. Its N-terminus is attached to the C-terminus of Z1 and its C-terminus is attached to the N-terminus of Z2 via a peptide bond.

The peptide Z2 is an amylin receptor agonist which is capable of binding to at least amylin receptor and capable of activating it. The N-terminus of Z2 is attached to the C-terminus of L1 via a peptide bond. The C-terminus of Z2 is modified with an amide group, which is considered essential for bioactivity. In a preferred embodiment, the amine group of the C-terminal amide is NH₂.

The molecular format may be a single chain peptide backbone comprising one lysine (Lys, K) residue. The one lysine (Lys, K) residue may be present in the peptide Z1 portion of the peptide backbone, or the one lysine (Lys, K) residue may be present in the peptide Z2 portion of the peptide backbone.

In one aspect, the present invention relates to a GLP-1-/GlP-/amylin-receptor tri-agonist comprising a peptide according to Formula I:

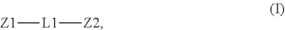

comprising one lysine (Lys, K) residue; wherein:
   Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃X₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).

In another aspect, the present invention relates to a GLP-1-/GlP-/amylin-receptor tri-agonist comprising a peptide according to Formula I:

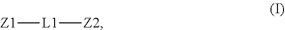

comprising one lysine (Lys, K) residue; wherein:
   Z1 is a peptide comprising or consisting of an amino acid sequence according to Formula XVII (SEQ ID NO: 62):

(XVII)
YX₂EGTFTSDYSX₁₂LLEEIAAREFIX₂₄WLX₂₇X₂₈GX₃₀X₃₁SX₃₃
X₃₄,

wherein
X₂ represents Aib,
X₁₂ represents Ile (I) or Lys (K),
X₂₄ represents Ala (A), Glu (E) or Gln (Q),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).

In another aspect, the present invention relates to a GLP-1-/GlP-/amylin receptor tri-agonist comprising a peptide according to Formula I:

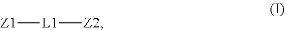

comprising one lysine (Lys, K) residue; wherein:
   Z1 is a peptide comprising a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ represents Aib;
   L1 is a peptide linker; and
   Z2 is a peptide comprising a C-terminal amide and a maximum of 10 amino acid substitutions relative to Formula V (SEQ ID NO: 2):

(V)
ASELSTAALGRLSAELHELATLPRTETGSGSP.

In another aspect, the invention relates to a GLP-1-/GlP-/amylin receptor tri-agonist comprising a peptide according to Formula I:

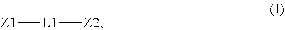

comprising one lysine (Lys, K) residue; wherein:
   Z1 is a peptide comprising a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E)
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide and a maximum of 10 amino acid substitutions relative to Formula V (SEQ ID NO: 2):

(V)
ASELSTAALGRLSAELHELATLPRTETGSGSP,

and wherein Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).

In another aspect the invention relates to a GLP-1-/GlP-/amylin-receptor tri-agonist comprising a peptide according to Formula I:

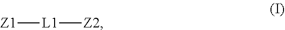

comprising one lysine (Lys, K) residue; wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein Z1 comprises or consists of an amino acid sequence according to Formula VII (SEQ ID NO: 162):

(VII)
YX₂EGTFTSDYSX₁₂LLEEIAAX₂₀EFIX₂₄WLX₂₇X₂₈GGPSSX₃₄,

wherein
   X₂ represents Aib,
   X₁₂ represents Ile (I) or Lys (K),
   X₂₀ represents Arg (R) or Gln (Q),
   X₂₄ represents Ala (A), Glu (E) or Gln (Q),
   X₂₇ represents Leu (L) or I (Ile),
   X₂₈ represents Ala (A) or Gln (Q),
   X₃₄ represents Gly (G) or Glu (E);
      L1 is a peptide linker; and
      Z2 is a peptide comprising a C-terminal amide, and having a maximum of 10 amino acid substitutions relative to Formula V (SEQ ID NO: 2):

(V)
ASELSTAALGRLSAELHELATLPRTETGSGSP,

and wherein Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
   X₅₂ represents Gly (G) or Ser (S),
   X₅₃ represents Gln (Q), Glu (E), or His (H),
   X₅₈ represents Ala (A) or Gln (Q),
   X₅₉ represents Leu (L) or Thr (T),
   X₆₀ represents Ala (A), Gly (G) or Gln (Q),
   X₆₈ represents Gln (Q), Glu (E), or Lys (K),
   X₇₂ represents Leu (L) or Glu (E).

In one embodiment the GLP-1-/GlP-/amylin-receptor tri-agonist of the present invention may comprise a peptide Z1 comprising or consisting of the amino acid sequence according to Formula VX (SEQ ID NO: 167):

(VX)
YX₂EGTFTSDYSX₁₂LLEEIAAREFIEWLX₂₇AGGPSSX₃₄,

wherein
X₂ represents Aib,
X₁₂ represents Ile (I) or Lys (K),
X₂₇ represents Leu (L) or I (Ile),
X₃₄ represents Gly (G) or Glu (E); and
a peptide Z2 comprising or consisting of the amino acid sequence according to SEQ ID NO: 40):

ASHLSTAQTQRLSAELHKLATLPRTETGSGSP.

In one embodiment, the GLP-1-/GlP-/amylin-receptor tri-agonist of the present invention may comprise a peptide Z1 comprising or consisting of an amino acid sequence according to Formula IV (SEQ ID NO: 169):

(IV)
YX₂EGTFTSDYSX₁₂LLEEIAAREFIEWLX₂₇AGX₃₀X₃₁SSG,

wherein
X₂ represents Aib,
X₁₂ represents Ile (I) or Lys (K),
X₂₇ represents Leu (L) or I (Ile),
X₃₀ represents Gly (G) or Ala (A),
X₃₁ represents Ala (A), Gly (G) or Pro (P); and
a peptide Z2 comprising or consisting of an amino acid sequence according to Formula IX (SEQ ID NO: 61):

(IX)
ASX₅₃LSTAX₅₈X₅₉X₆₀RLSAELHX₆₈LATLPRTETGSGSP,

wherein
X₅₃ represents Glu (E) or His (H),
X₅₈ represents Ala (A) or Gln (Q),
X₅₉ represents Leu (L) or Thr (T),
X₆₀ represents Gly (G) or Gln (Q),
X₆₈ represents Gln (Q), Glu (E), or Lys (K).

In one embodiment the backbone of the peptide Z1-L1-Z2 comprises or consists of 66 to 80 amino acid residues, such as 66 to 76 amino acid residues.

In one embodiment the backbone of the peptide Z1-L1-Z2 comprises or consists of 67, 68, 71, 75 or 76 amino acid residues.

In a preferred embodiment the backbone of the peptide Z1-L1-Z2 comprises or consists of 68 or 76 amino acid residues.

In one embodiment the backbone of the peptide Z1-L1-Z2 comprises or consists of an amino acid sequence according to SEQ ID NO: 10. In one embodiment, the backbone of the peptide Z1-L1-Z2 comprises or consists of an amino acid sequence according to SEQ ID NO: 5.

In one embodiment the GLP-1-/GlP-/amylin-receptor tri-agonist comprises a peptide according to Formula I: Z1-L1-Z2, wherein the peptide comprises or consists of an amino acid sequence selected from the group consisting of SEQ ID NOs 170 to 252 shown in Table 4:

TABLE 4
Amino acid sequences of the peptide backbone within compounds of the invention

| Backbone of | | |
|---|---|---|
| compound | SEQ ID | |
| no. | NO: | Amino acid sequence |
| 120, 160, 161, | 170 | YX₂EGTFTSDYSILLEEIAAREFIEWLLAGGPSSGAGQAPG |
| 192, 220 | | QAPGASHLSTAQTQRLSAELHKLATLPRTETGSGSP |
| 121 | 171 | YX₂EGTFTSDYSILLEEIAAREFIEWLIAGGPSSGAGQAPG |
| | | QAPGASHLSTAQTQRLSAELHKLATLPRTETGSGSP |
| 122 | 172 | YX₂EGTFTSDYSKLLEEIAAREFIEWLLAGGPSSGAGQAP |
| | | GQAPGASHLSTAQLGRLSAELHELATLPRTETGSGSP |
| 123 | 173 | YX₂EGTFTSDYSKLLEEIAAREFIEWLLAGGPSSGAGQAP |
| | | GQAPGASHLSTAQLGRLSAELHQLATLPRTETGSGSP |
| 124 | 174 | YX₂EGTFTSDYSKLLEEIAAREFIEWLLAGGPSSGAGQAP |
| | | GQAPGASHLSTAQTQRLSAELHELATLPRTETGSGSP |
| 125, 142, 221 | 175 | YX₂EGTFTSDYSILLEEIAAREFIEWLLAGGPSSGAGQAPG |
| | | QAPLASHLSTAQTQRLSAELHKLATLPRTETGSGSP |
| 126 | 176 | YX₂EGTFTSDYSKLLEEIAAREFIEWLLAGGPSSGAGQAP |
| | | GQAPGASHLSTAALGRLSAELHELATLPRTETGSGSP |
| 127 | 177 | YX₂EGTFTSDYSKLLEEIAAREFIEWLIAGGPSSGAGQAPG |
| | | QAPGASHLSTAALGRLSAELHELATLPRTETGSGSP |
| 128 | 178 | YX₂EGTFTSDYSILLEEIAAREFIEWLLAGGPSSGAGQAPG |
| | | QAPLASHLSTAQTARLSAELHKLATLPRTETGSGSP |
| 129 | 179 | YX₂EGTFTSDYSKLLEEIAAREFIEWLLAGGPSSGAGQAP |
| | | GQAPLASHLSTAQLGRLSAELHQLATLPRTETGSGSP |
| 130 | 180 | YX₂EGTFTSDYSKLLEEIAAREFIEWLLAGGPSSGAGASHL |
| | | STAQLGRLSAELHQLATLPRTETGSGSP |
| 131, 193 | 181 | YX₂EGTFTSDYSILLEEIAAREFIEWLLAGGPSSGAGASHL |
| | | STAQTQRLSAELHKLATLPRTETGSGSP |
| 132 | 182 | YX₂EGTFTSDYSILLEEIAAREFIEWLLAGGPSSGAGQAPG |
| | | QAPLASQLSTAQTQRLSAELHKLATLPRTETGSGSP |
| 133 | 183 | YX₂EGTFTSDYSILLEEIAAREFIEWLLAGGPSSGAGQAPG |
| | | QAPLASELSTAQTQRLSAELHKLATLPRTETGSGSP |
| 134 | 184 | YX₂EGTFTSDYSILLEEIAAREFIEWLLAGGPSSGEASHLST |
| | | AQTQRLSAELHKLATLPRTETGSGSP |
| 135 | 185 | YX₂EGTFTSDYSILLEEIAAREFIEWLLAGGPSSGAGQAPG |
| | | QAPAASHLSTAQTQRLSAELHKLATLPRTETGSGSP |
| 136 | 186 | YX₂EGTFTSDYSILLEEIAAREFIEWLLAGGPSSGAGQAPG |
| | | QAPEASHLSTAQTQRLSAELHKLATLPRTETGSGSP |
| 137 | 187 | YX₂EGTFTSDYSILLEEIAAREFIEWLLAGGPSSGAGQAPG |
| | | QAPPASHLSTAQTQRLSAELHKLATLPRTETGSGSP |
| 138 | 188 | YX₂EGTFTSDYSILLEEIAAREFIEWLLAGGPSSGAGQAPG |
| | | QAPSASHLSTAQTQRLSAELHKLATLPRTETGSGSP |
| 139 | 189 | YX₂EGTFTSDYSILLEEIAAREFIEWLLAGGPSSGAGQAPG |
| | | QAPVASHLSTAQTQRLSAELHKLATLPRTETGSGSP |
| 140 | 190 | YX₂EGTFTSDYSILLEEIAAREFIEWLLAGGPSSGAGGGGA |
| | | SHLSTAQTQRLSAELHKLATLPRTETGSGSP |
| 141 | 191 | YX₂EGTFTSDYSILLEEIAAREFIEWLLAGGPSSGAGGGGA |
| | | GELSTAQTQRLSAELHKLATLPRTETGSGSP |
| 143 | 192 | YX₂EGTFTSDYSILLEEIAAREFIEWLIAGGPSSGAGQAPG |
| | | QAPLASHLSTAQTQRLSAELHKLATLPRTETGSGSP |
| 144 | 193 | YX₂EGTFTSDYSILLEEIAAREFIEWLLAGGPSSGAGQAPG |
| | | QAPGASELSTAQTQRLSAELHKLATLPRTETGSGSP |
| 145 | 194 | YX₂EGTFTSDYSILLEEIAAREFIEWLLAGGPSSGAGQAPG |
| | | QAPGASELSTAQTARLSAELHKLATLPRTETGSGSP |
| 146,211 | 195 | YX₂EGTFTSDYSILLEEIAAREFIEWLLAGGPSSEAGQAPG |
| | | QAPGASHLSTAQTQRLSAELHKLATLPRTETGSGSP |
| 147, 212 | 196 | YX₂EGTFTSDYSILLEEIAAREFIEWLLAGGPSSGEGQAPG |
| | | QAPGASHLSTAQTQRLSAELHKLATLPRTETGSGSP |
| 148, 213 | 197 | YX₂EGTFTSDYSILLEEIAAREFIEWLLAGGPSSGAGEAPG |
| | | QAPGASHLSTAQTQRLSAELHKLATLPRTETGSGSP |
| 149, 214 | 198 | YX₂EGTFTSDYSILLEEIAAREFIEWLLAGGPSSGAGQEPG |
| | | QAPGASHLSTAQTQRLSAELHKLATLPRTETGSGSP |
| 150, 215 | 199 | YX₂EGTFTSDYSILLEEIAAREFIEWLLAGGPSSGAGQAEG |
| | | QAPGASHLSTAQTQRLSAELHKLATLPRTETGSGSP |
| 151,216 | 200 | YX₂EGTFTSDYSILLEEIAAREFIEWLLAGGPSSGAGQAPE |
| | | QAPGASHLSTAQTQRLSAELHKLATLPRTETGSGSP |
| 152, 217 | 201 | YX₂EGTFTSDYSILLEEIAAREFIEWLLAGGPSSGAGQAPG |
| | | EAPGASHLSTAQTQRLSAELHKLATLPRTETGSGSP |
| 153, 218 | 202 | YX₂EGTFTSDYSILLEEIAAREFIEWLLAGGPSSGAGQAPG |
| | | QEPGASHLSTAQTQRLSAELHKLATLPRTETGSGSP |
| 154, 219 | 203 | YX₂EGTFTSDYSILLEEIAAREFIEWLLAGGPSSGAGQAPG |
| | | QAEGASHLSTAQTQRLSAELHKLATLPRTETGSGSP |
| 155 | 204 | YX₂EGTFTSDYSILLEEIAAREFIEWLIAGGPSSGAGQEPG |
| | | QEPGASHLSTAQTQRLSAELHKLATLPRTETGSGSP |
| 156 | 205 | YX₂EGTFTSDYSILLEEIAAREFIAWLIAGGPSSGAGQEPG |
| | | QEPGASHLSTAQTQRLSAELHKLATLPRTETGSGSP |
| 157 | 206 | YX₂EGTFTSDYSILLEEIAAREFIQWLIAGGPSSGAGQEPG |
| | | QEPGASHLSTAQTQRLSAELHKLATLPRTETGSGSP |
| 158 | 207 | YX₂EGTFTSDYSILLEEIAAREFIEWLIAGGPSSGAGASHLS |
| | | TAQTQRLSAELHKLATLPRTETGSGSP |
| 159 | 208 | YX₂EGTFTSDYSILLEEIAAREFIEWLLAGGPSSGAGQAPG |
| | | QAPASHLSTAQTQRLSAELHKLATLPRTETGSGSP |
| 162 | 209 | YX₂EGTFTSDYSILLEEIAAREFIEWLLAGGPSSGAGQAPG |
| | | QAPGASHLSTAQTQRLSAELHKLATEPRTETGSGSP |
| 163 | 210 | YX₂EGTFTSDYSILLEEIAAREFIEWLLAGGPSSGAGQAPG |
| | | QAPGASELSTAQTQRLSAELHKLATEPRTETGSGSP |
| 164 | 211 | YX₂EGTFTSDYSILLEEIAAREFIEWLLAGGPSSGAGQAPG |
| | | EAPGASHLSTAQTQRLSAELHKLATEPRTETGSGSP |
| 165 | 212 | YX₂EGTFTSDYSILLEEIAAREFIEWLLAGGPSSEAGQAPG |
| | | QAPGASHLSTAQTQRLSAELHKLATEPRTETGSGSP |
| 166 | 213 | YX₂EGTFTSDYSILLEEIAAREFIEWLLAGGPSSGAGQAPG |
| | | EAPGASELSTAQTQRLSAELHKLATLPRTETGSGSP |
| 167 | 214 | YX₂EGTFTSDYSILLEEIAAREFIEWLLAGGPSSEAGQAPG |
| | | QAPGASELSTAQTQRLSAELHKLATLPRTETGSGSP |
| 168 | 215 | YX₂EGTFTSDYSILLEEIAAREFIEWLLAGGPSSEAGQAPG |
| | | EAPGASHLSTAQTQRLSAELHKLATLPRTETGSGSP |
| 169 | 216 | YX₂EGTFTSDYSKLLEEIAAREFIEWLLAGGPSSGAGQAP |
| | | GQAPGASELSTAALGRLSAELHELATLPRTETGSGSP |
| 170 | 217 | YX₂EGTFTSDYSKLLEEIAAREFIEWLLAGGPSSGAGQAP |
| | | GQAPGASELSTAALGRLSAELHQLATLPRTETGSGSP |
| 171 | 218 | YX₂EGTFTSDYSKLLEEIAAREFIEWLLAGGPSSGAGQAP |
| | | GEAPGASELSTAALGRLSAELHQLATLPRTETGSGSP |
| 172 | 219 | YX₂EGTFTSDYSKLLEEIAAREFIEWLLAGGPSSGAGQAP |
| | | GEAPGASELSTAALGRLSAELHQLATEPRTETGSGSP |
| 173 | 220 | YX₂EGTFTSDYSKLLEEIAAREFIEWLLAGGPSSGAGQAP |
| | | GEAPGASELSTAQLGRLSAELHQLATEPRTETGSGSP |
| 174 | 221 | YX₂EGTFTSDYSKLLEEIAAREFIEWLLAGGPSSGAGQAP |
| | | GEAPGASELSTAQTGRLSAELHQLATEPRTETGSGSP |
| 175 | 222 | YX₂EGTFTSDYSKLLEEIAAREFIEWLLAGGPSSGAGQAP |
| | | GEAPGASELSTAQTQRLSAELHQLATEPRTETGSGSP |
| 176 | 223 | YX₂EGTFTSDYSILLEEIAAREFIEWLLAGGPSSGAGASEL |
| | | STAQTQRLSAELHKLATLPRTETGSGSP |
| 177 | 224 | YX₂EGTFTSDYSILLEEIAAREFIEWLLAGGPSSEAGASHLS |
| | | TAQTQRLSAELHKLATLPRTETGSGSP |
| 178 | 225 | YX₂EGTFTSDYSILLEEIAAREFIEWLLAGGPSSEAGASELS |
| | | TAQTQRLSAELHKLATLPRTETGSGSP |
| 179 | 226 | YX₂EGTFTSDYSILLEEIAAREFIEWLLAGGPSSGAGASHL |
| | | STAQTQRLSAELHKLATEPRTETGSGSP |
| 180 | 227 | YX₂EGTFTSDYSILLEEIAAREFIEWLLAGGPSSGAGASQL |
| | | STAQTQRLSAELHKLATEPRTETGSGSP |
| 181 | 228 | YX₂EGTFTSDYSILLEEIAAREFIEWLLAGGPSSEAGASQL |
| | | STAQTQRLSAELHKLATLPRTETGSGSP |
| 182 | 229 | YX₂EGTFTSDYSILLEEIAAREFIEWLLAGGPSSEAGASQL |
| | | STAQTQRLSAELHKLATEPRTETGSGSP |
| 183 | 230 | YX₂EGTFTSDYSKLLEEIAAREFIEWLLAGGPSSGAGASEL |
| | | STAQLGRLSAELHQLATLPRTETGSGSP |
| 184 | 231 | YX₂EGTFTSDYSKLLEEIAAREFIEWLLAGGPSSGAGASHL |
| | | STAQLGRLSAELHQLATEPRTETGSGSP |
| 185 | 232 | YX₂EGTFTSDYSKLLEEIAAREFIEWLLAGGPSSEAGASHL |
| | | STAQLGRLSAELHQLATEPRTETGSGSP |
| 186 | 233 | YX₂EGTFTSDYSKLLEEIAAREFIEWLLAGGPSSGAGASEL |
| | | STAQLGRLSAELHQLATEPRTETGSGSP |
| 187 | 234 | YX₂EGTFTSDYSKLLEEIAAREFIEWLLAGGPSSEAGASEL |
| | | STAQLGRLSAELHQLATLPRTETGSGSP |
| 188 | 235 | YX₂EGTFTSDYSKLLEEIAAREFIEWLLAGGPSSEAGASEL |
| | | STAQLGRLSAELHQLATEPRTETGSGSP |
| 189 | 236 | YX₂EGTFTSDYSILLEEIAAREFIEWLLAGGPSSGAGEAPG |
| | | EAPGASHLSTAQTQRLSAELHKLATLPRTETGSGSP |
| 190 | 237 | YX₂EGTFTSDYSILLEEIAAREFIEWLIAGGPSSGAGEAPG |
| | | QAPGASQLSTAQTQRLSAELHKLATLPRTETGSGSP |
| 191 | 238 | YX₂EGTFTSDYSILLEEIAAREFIEWLIAGGPSSGAGEAPG |
| | | QAPGASELSTAQTQRLSAELHKLATLPRTETGSGSP |
| 194 | 239 | YX₂EGTFTSDYSILLEEIAAQEFIEWLLQGGPSSGAGASEL |
| | | STAQTQRLSAELHKLATLPRTETGSGSP |
| 195 | 240 | YX₂EGTFTSDYSILLEEIAAQEFIEWLLQGGPSSGAGEAPG |
| | | QAPGASELSTAQTQRLSAELHKLATLPRTETGSGSP |
| 196 | 241 | YX₂EGTFTSDYSILLEEIAAREFIEWLLAGGPSSGAGQAPG |
| | | EAPLASHLSTAQTQRLSAELHKLATLPRTETGSGSP |
| 197 | 242 | YX₂EGTFTSDYSILLEEIAAREFIEWLLAGGPSSGAGQAPG |
| | | QAPLASHLSTAQTQRLSAELHKLATEPRTETGSGSP |
| 104, 114 | 243 | YX₂EGTFTSDYSILLEEIAAREFIEWLLAGGASSGAGEAPG |
| | | EAPGASHLSTAQTQRLSAELHKLATLPRTETGSGSP |
| 105, 115 | 244 | YX₂EGTFTSDYSKLLEEIAAREFIEWLLAGGPSSGAGASEL |
| | | STAALGRLSAELHQLATLPRTETGSGSP |
| 106 | 245 | YX₂EGTFTSDYSKLLEEIAAREFIEWLLAGGQSSGAGASEL |
| | | STAQLGRLSAELHQLATLPRTETGSGSP |
| 107, 116 | 246 | YX₂EGTFTSDYSKLLEEIAAREFIEWLIAGAPSSGAGASEL |
| | | STAALGRLSAELHQLATLPRTETGSGSP |
| 108 | 247 | YX₂EGTFTSDYSKLLEEIAAREFIEWLLAGGPSSGAEASEL |
| | | STAQLGRLSAELHQLATLPRTETGSGSP |
| 109 | 248 | YX₂EGTFTSDYSKLLEEIAAREFIEWLIAGGPSEGAGASEL |
| | | STAQLGRLSAELHQLATLPRTETGSGSP |
| 110 | 249 | YX₂EGTFTSDYSKLLEEIAAREFIEWLIAGGGSSGAGASEL |
| | | STAALGRLSAELHELATLPRTETGSGSP |
| 111, 118 | 250 | YX₂EGTFTSDYSKLLEEIAAREFIEWLIAGAPSSGAGASEL |
| | | STAALGRLSAELHELATLPRTETGSGSP |
| 112, 119 | 251 | YX₂EGTFTSDYSKLLEEIAAREFIEWLLAGGGSSGAGASEL |
| | | STAALGRLSAELHELATLPRTETGSGSP |
| 113, 117 | 252 | YX₂EGTFTSDYSKLLEEIAAREFIEWLLAGGPSSGAGASEL |
| | | STAALGRLSAELHELATLPRTETGSGSP |

wherein X₂ represents Aib.

In a particular embodiment, the invention relates to a GLP-1-/GlP-/amylin-receptor tri-agonist peptide, wherein the peptide comprises or consists of an amino acid sequence selected from the list consisting of: SEQ ID NO: 230, SEQ ID NO: 243, SEQ ID NO: 244, SEQ ID NO: 246, SEQ ID NO: 250, SEQ ID NO: 251 and SEQ ID NO: 252.

In some embodiments of the present invention, the peptide Z1 comprises an amino acid sequence which has at least 87, 88, 89, 90, 91, 92, 93, 94, 95, 96, 97, 98 or 99% identity (i.e., a sequence identity) to Formula II (SEQ ID NO: 1), and the peptide Z2 comprises an amino acid sequence which has at least 70, 71, 72, 73, 74, 75, 76, 77, 78, 79, 80, 81, 82, 83, 84, 85, 86, 87, 88, 89 90, 91, 92, 93, 94, 95, 96, 97, 98 or 99% identity (i.e., a sequence identity) relative to Formula V (SEQ ID NO: 2).

In some embodiments of the invention the GLP-1-/GlP-/amylin-receptor tri-agonist disclosed herein does not comprise a cysteine (Cys, C) residue and/or does not comprise a disulfide bridge. The term “disulfide bridge” in reference to human amylin and analogues thereof, refers to a functional group with the structure R—S—S—R′ and may also be referred to as an “SS-bond”.

The GLP-1-/GlP-/amylin-receptor tri-agonist may exhibit a variety of properties rendering it useful as a medicament, as described herein.

### Protraction Moiety

In one aspect the invention relates to a GLP-1-/GlP-/amylin-receptor tri-agonist which may further comprise a protraction moiety. In such cases the tri-agonist is referred to as a “peptide derivative” or a “GLP-1-/GlP-/amylin-receptor tri-agonist derivative”. The addition of the term “derivative” thus means that a protraction moiety is present and a compound or compounds comprising a protraction moiety are referred to as “derivative” or “derivatives”.

The molecular format may be a single chain peptide backbone comprising one lysine (Lys, K) residue. The one lysine (Lys, K) residue may be present in the peptide Z1 portion of the peptide backbone, or the one lysine (Lys, K) residue may be present in the peptide Z2 portion of the peptide backbone. The one lysine residue may be covalently bound to a protraction moiety, which may be referred to herein as “L_{P}-P”, wherein “L_{P}” is an optional linker and “P” is a protractor. The peptide backbone of the GLP-1-/GlP-/amylin-receptor tri-agonist of the present invention typically comprises about 66 to about 76 amino acid residues linked together by peptide bonds.

The term “protraction moiety” as used herein refers to a moiety having half-life extending properties and comprising a “protractor P” and an optional “linker L_{P}”, and may be represented by the general formula “L_{P}-P”, in which L_{P} is said optional linker and P is said protractor.

The term “protractor” as used herein refers to a molecule which is capable of increasing the plasma half-life of the peptide to which it is attached. The term “protraction” thus refers to half-life extension and the terms “protractor”, “protraction moiety” or “half-life extending moiety” thus serve the purpose of extending the plasma half-life of the peptides as disclosed herein.

Furthermore, the GLP-1-/GlP-/amylin-receptor tri-agonist of the present invention have a long plasma half-life relative to dosing interval, thus reducing the variability in steady state exposure, and thus making a once weekly administration possible. The compound disclosed herein may be orally bioavailable, hence suitable for oral administration of subjects in need thereof. Both the peptide backbone and the protraction moiety have been engineered and refined in order to achieve a compound having all of the above properties.

Each protraction moiety L_{P}-P covalently attaches to the epsilon amino group of a lysine residue in the peptide backbone of the inventive GLP-1-/GlP-/amylin-receptor tri-agonist. The attachment point is generally referred to as R1.

In one embodiment, the protraction moiety L_{P}-P may attach to the epsilon position (i.e., amino group) of the one lysine (Lys, K) residue. In one embodiment, the protraction moiety L_{P}-P may attach to the epsilon position (i.e., amino group) of a lysine (Lys, K) residue in the peptide Z1 portion of the peptide backbone (the “Z1” in Z1-L1-Z2), such as a lysine (Lys, K) residue at position 12 of the Z1 peptide (X₁₂) of e.g. Formula III (SEQ ID NO: 168), Formula IV (SEQ ID NO: 169), Formula VII (SEQ ID NO: 162), Formula XV (SEQ ID NO: 167), or Formula XVII (SEQ ID NO: 62). In one embodiment, the protraction moiety L_{P}-P may attach to the epsilon position (i.e., amino group) of a lysine (Lys, K) residue in SEQ ID NO: 22, SEQ ID NO: 23, SEQ ID NO: 27, SEQ ID NOs: 30-34, preferably in SEQ ID NO: 22 or SEQ ID NO: 31.

In one embodiment, the protraction moiety L_{P}-P may attach to the epsilon position (i.e. amino group) of a lysine (Lys, K) residue in the peptide Z2 portion of the peptide backbone (the “Z2” in Z1-L1-Z2), such as a lysine (Lys, K) residue at position 18 (Xe) of Formula VI (SEQ ID NO: 165), Formula IX (SEQ ID NO: 61), Formula X (SEQ ID NO: 166), or Formula XIV (SEQ ID NO: 4). In one embodiment, the protraction moiety L_{P}-P may attach to the epsilon position (i.e., amino group) of a lysine (Lys, K) residue in SEQ ID NO: 40, SEQ ID NOs: 45-51, or SEQ ID NO: 58, preferably SEQ ID NO: 40.

In one embodiment, the protraction moiety L_{P}-P may attach to the epsilon position (i.e., amino group) of a lysine (Lys, K) residue in the GLP-1-/GlP-/amylin-receptor tri-agonist peptide of SEQ ID NO: 230, SEQ ID NO: 243, SEQ ID NO: 244, SEQ ID NO: 246, SEQ ID NO: 250, SEQ ID NO: 251 or SEQ ID NO: 252.

When the optional linker L_{P} is present, the protraction moiety L_{P}-P covalently attaches to the peptide backbone via the linker L_{P}. When the linker L_{P} is absent, P covalently attaches to the peptide backbone.

The GLP-1-/GlP-/amylin-receptor tri-agonist of the present invention, as disclosed herein, comprises or consists of a peptide comprising one lysine (Lys, K) residue, to which a single protraction moiety is covalently attached/conjugated (at the epsilon amino group). A protraction moiety may consist of one protractor P. A protraction moiety may comprise one linker L_{P} and one protractor P. A protraction moiety may comprise one linker L_{P} and two or more protractors (in this case referred to as P1, P2 or P3 and so forth). The two protractors (P1 and P2) may be identical, or the two protractors (P1 and P2) may be non-identical. Where the peptide derivative comprises two or three protractors (P1, P2, P3), the protractors are preferably similar, more preferably substantially identical, or, most preferably, identical.

In the context of chemical moieties such as the protraction moieties disclosed herein, similarity and/or identity may be determined using any suitable computer program and/or algorithm known in the art.

The protraction moiety may be capable of non-covalently binding to albumin, thereby promoting the circulation of the peptide derivative of the present invention in the blood stream and prolonging its plasma half-life. Thus, the skilled person may also refer to the protraction moiety as being an “albumin binding moiety”.

### Protractor P

The protractor P may comprise an acyl group. The acyl group may be branched or unbranched. The acyl group may be saturated or unsaturated. The protractor P may comprise a fatty acid acyl group. The fatty acid acyl group may be branched or unbranched.

The fatty acid acyl group may be saturated or unsaturated.

The protractor P may comprise a distal carboxylic acid group.

The protractor P may comprise a fatty acid group.

The protractor P may comprise a fatty acid group and an amide group.

The protractor P may comprise a distal carboxylic acid group and an amide group.

The protractor P may comprise an alkyl group.

The protractor P may comprise an aryl group.

The protractor P may comprise a tetrazole group.

The protractor P may comprise a sulfonic acid group.

The protractor P may comprise a phenoxy group.

The protractor P may comprise a benzoic acid group.

The protractor P may comprise a phosphonic acid group.

The protractor may comprise a group defined by:

Chem. 1a: HOOC—(CH₂)ₙ—CO—* wherein n is an integer in the range of 6-30, which may also be referred to as a C₍ₙ₊₂₎ diacid (e.g. C₁₈ diacid) or as

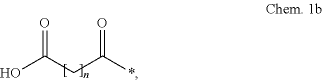

wherein n is an integer in the range of 6-30. The asterisk (*) shows the point of attachment of the radical.

The protractor P may comprise 8-32 carbon atoms. The protractor may comprise 8, 9, 10, 11, 12, 13, 14, 15, 16, 17, 18, 19, 20, 21, 22, 23, 24, 25, 26, 27, 28, 29, 30, 31 or 32 carbon atoms.

The protractor P may comprise 6-30 consecutive —CH₂— groups. The protractor P may comprise a carbon chain comprising at least 6, 7, 8, 9, 10, 11, 12, 13, 14, 15, 16, 17, 18, 19, 20, 21, 22, 23, 24, 25, 26, 27, 28, 29 or 30 consecutive —CH₂— groups.

The protractor P may comprise 12-26 carbon atoms. The protractor P may comprise 12, 13, 14, 15, 16, 17, 18, 19, 20, 21, 22, 23, 24, 25 or 26 carbon atoms.

The protractor P may comprise 10-26 consecutive —CH₂— groups. The protractor P may comprise a carbon chain comprising 10, 11, 12, 13, 14, 15, 16, 17, 18, 19, 20, 21, 22, 23, 24, 25 or 26 consecutive —CH₂— groups.

The protractor P may comprise 16-22 carbon atoms. The peptide derivative of the present invention may comprise a single protraction moiety with a protractor P comprising a carbon chain a side chain comprising 16, 17, 18, 19, 20, 21 or 22 carbon atoms.

The protractor P may comprise 14-20 consecutive —CH₂— groups. The protractor P may comprise a carbon chain comprising 14, 15, 16, 17, 18, 19 or 20 consecutive —CH₂— groups.

The protractor may P comprise 16-22 consecutive carbon atoms and 14-20 consecutive —CH₂— groups.

The protractor P may comprise 16 consecutive carbon atoms and 14 consecutive —CH₂— groups. The protractor P may be a C₁₆ diacid, which may be defined by the formula HOOC—(CH₂)₁₄—CO—*.

The protractor P may comprise 18 consecutive carbon atoms and 16 consecutive —CH₂— groups. The protractor P may be a C₁₆ diacid, which may be defined by the formula HOOC—(CH₂)₁₄—CO—*.

The protractor P may comprise 20 consecutive carbon atoms and 18 consecutive —CH₂— groups. The protractor P may be a C₂₀ diacid, which may be defined by the formula HOOC—(CH₂)₁₈—CO—*.

The protractor P may comprise 22 consecutive carbon atoms and 20 consecutive —CH₂— groups. The protractor P may be a C₂₂ diacid, which may be defined by the formula HOOC—(CH₂)₂₀—CO—*.

The term “fatty acid” refers to aliphatic mono- or dicarboxylic acids having from 4 to 28 carbon atoms, it may be branched or un-branched, it is preferably un-branched, and it may be saturated or unsaturated, it is preferably saturated.

As described above, the peptide derivative disclosed herein comprise one lysine (Lys, K) residue and hence one protraction moiety (L_{P}-P), wherein the protraction moiety is attached to the peptide backbone described herein via the epsilon position (i.e., amino group) of the lysine (Lys, K) residue (via an amide bond formed between a carboxylic acid group in the protraction moiety and the epsilon amino group of the lysine residue). The protraction moiety may be attached to the epsilon position of the one lysine (Lys, K) residue in the peptide backbone.

In one embodiment the protraction moiety may attach to the epsilon position of the lysine (Lys, K) residue in the peptide Z1 of the peptide backbone (the “Z1” in Z1-L1-Z2). In particular, the protraction moiety may attach to the epsilon position of the lysine (Lys, K) at position X₁₂ of Formula III (SEQ ID NO: 168) or Formula VII (SEQ ID NO: 162) of peptide Z1. In particular, the protraction moiety may attach to the epsilon position of the lysine (Lys, K) at position 12 of peptide Z1.

In one embodiment the protraction moiety may attach to the epsilon position of the lysine (Lys, K) residue in the linker L1 portion of the peptide backbone (the “L1” in Z1-L1-Z2).

In one embodiment the protraction moiety may attach to the epsilon position of the lysine (Lys, K) residue in the peptide Z2 of the peptide backbone (the “Z2” in Z1-L1-Z2). In particular, the protraction moiety may attach to the epsilon position of the lysine (Lys, K) at position X₆₈ of Formula VI (SEQ ID NO: 165) of peptide Z2. In particular, the protraction moiety may attach to the epsilon position of the lysine (Lys, K) residue at position 18 of peptide Z2. In a preferred embodiment the protraction moiety may attach to the epsilon position of the lysine (Lys, K) residue at position 18 of peptide Z2.

In some embodiments the GLP-1-/GlP-/amylin-receptor tri-agonist comprising a peptide derivative as disclosed herein may comprise a protractor P, which is selected from any one of those depicted in

Table 5. R1 represents the site of attachment to (a) the backbone of the peptide derivative—more specifically the epsilon amino group of the lysine or (b) the present optional linker L_{P}. Based on the disclosure herein, the skilled person will be able to determine also other chemical moieties for use as a protractor in a specific peptide derivative as disclosed herein, optionally after some limited routine experiments.

TABLE 5
Examples of protractors (P)

| Protractor ID | Structure |
|---|---|
| Chem. 2 | 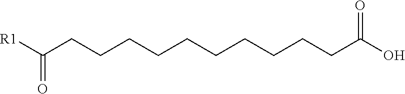 |
| | C₁₂ diacid |
| Chem. 3 | 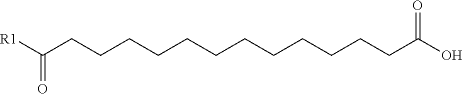 |
| | C₁₄ diacid |
| Chem. 4 | 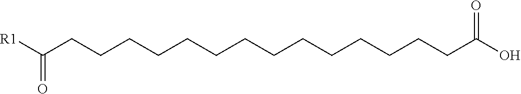 |
| | C₁₆ diacid |
| Chem. 5 | 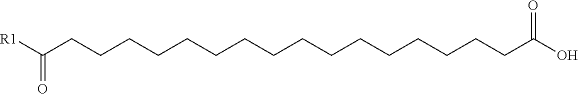 |
| | C₁₈ diacid |
| Chem. 6 | 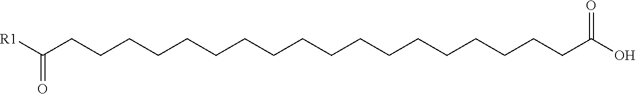 |
| | C₂₀ diacid |
| Chem. 7 | 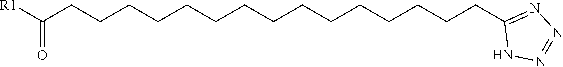 |
| | C₁₇ tetrazole |
| Chem. 8 | 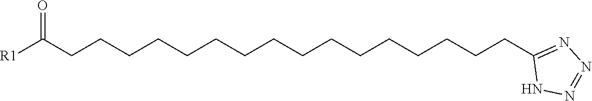 |
| | C₁₈ tetrazole |
| Chem. 32 | 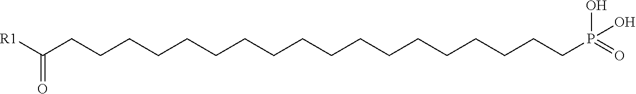 |
| | C₁₉ phosphonic acid |

In one embodiment the GLP-1-/GlP-/amylin-receptor tri-agonist comprises a peptide derivative comprising a protractor P, being a C₁₂-C₂₀ diacid.

In one embodiment the GLP-1-/GlP-/amylin-receptor tri-agonist comprises a peptide derivative comprising a protractor P selected from the group consisting of C₁₆ diacid, C₁₈ diacid, and C₂₀ diacid.

In a preferred embodiment, the GLP-1-/GlP-/amylin-receptor tri-agonist comprises a peptide derivative comprising a protractor P being a C₁₈ diacid (Chem. 5), a C₂₀ diacid (Chem. 6), or a C₁₉ phosphonic acid (Chem. 32).

### Linker L_{P}

In one embodiment the protractor is attached/conjugated directly onto the backbone of the peptide derivative, i.e., without use of a linker L_{P} (i.e., by way of a covalent bond, e.g. an amide bond).

In other embodiments the protractor is covalently conjugated to the peptide derivative using a linker L_{P}, hence, as described above, the protraction moiety (L_{P}-P) comprises an optional linker L_{P}. The linker L_{P} may comprise several “linker elements”. The linker elements may be selected so that they improve the overall properties of the molecule, e.g., so that they improve the oral bioavailability, the conversion of half-life or the protracting effect, thus improving the overall exposure profile upon oral administration of the compound.

The linker L_{P} may comprise Ado, Aeep or Aeeep, Ahx, Ala, ε-Lys, Glu, γGlu, Gly, Ser, sulfonamide, Thr and/or Trx.

The linker L_{P} may comprise at least a moiety which may be represented by the following chemical formula (wherein the asterisks (*) show the points of attachment of the radicals):

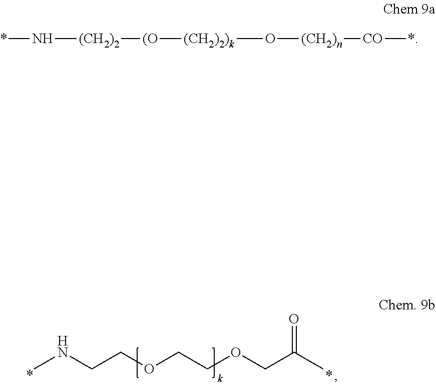

wherein k is an integer in the range of 1-5, and n is an integer in the range of 1-5.

When k=1 and n=1, the linker element may be designated Ado, or 8-amino-3,6-dioxaoctanoyl, which may be represented by the following chemical formula:

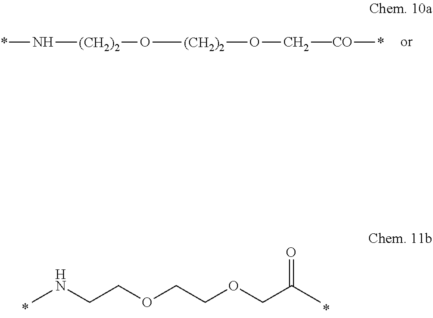

When k=1 and n=2, the linker element may be designated Aeep, which may be represented by the following chemical formula:

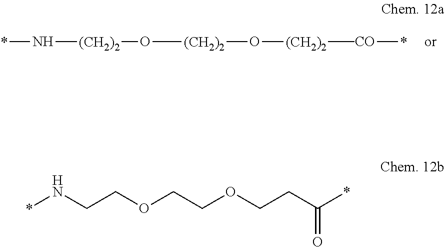

When k=2 and n=2, the linker element may be designated Aeeep, which may be represented by the following chemical formula:

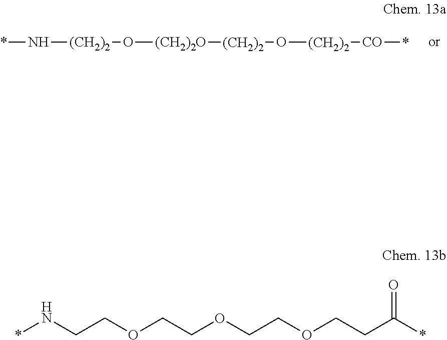

The linker L_{P} may comprise a sulfonamide-C4 moiety. A sulfonamide-C4 group is a sulfonamide group attached to a 4-butanoyl group, having the following chemical formula:

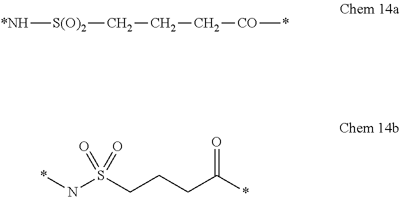

The linker L_{P} may comprise Trx. Trx is also referred to as Tranexamic acid, trans-4-(aminomethyl)cyclohexanecarboxylic acid, having the following chemical formula:

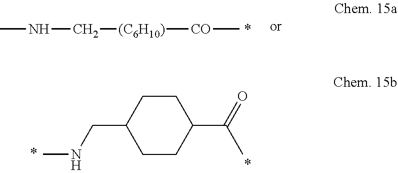

The linker L_{P} may comprise Ahx. Ahx is also referred to as Aminocaproic acid, 6-aminohexanoic acid having the following chemical formula:

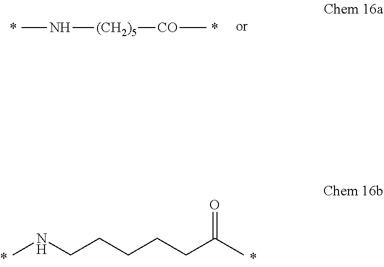

The linker L_{P} may comprise epsilon-lysine (ε-Lys).

The linker L_{P} may comprise lysine (Lys).

The linker L_{P} may comprise alanine (Ala).

The linker L_{P} may comprise glycine (Gly).

The linker L_{P} may comprise serine (Ser).

The linker L_{P} may comprise glutamic acid (Glu).

The linker L_{P} may comprise a Glu di-radical, such as

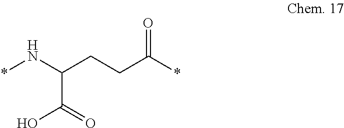 [SMILES: *NC(CCC(*)=O)C(=O)O]

wherein the Glu di-radical may be included p times, where p is an integer in the range of 1-3. Anyone of above disclosed amino acids, which are used as linker L_{P} or as part of linker L_{P}, may be used as L-isomer or as D-isomer.

Chem. 17 may also be referred to as gamma-Glu, or briefly γGlu, due to the fact that it is the gamma carboxy group of the amino acid glutamic acid which is here used for connection to the epsilon amino group of lysine. As described above, the other linker element may, for example, be another Glu residue, or an Ado molecule. The amino group of Glu in turn forms an amide bond with the carboxy group of the protracting moiety, or with the carboxy group of, e.g., an Ado molecule, if present, or with the gamma-carboxy group of, e.g., another Glu, if present.

The peptide derivative disclosed herein may comprise a linker L_{P} which is selected from any one of those depicted in Table 6 below. R1 represents the residue in the peptide backbone to which the protraction moiety is attached to, and P represents the protractor.

In some embodiments the peptide derivative comprises a protraction moiety, wherein the protractor Chem. 4 or Chem. 5 or Chem. 6 or Chem. 32 is attached to the peptide backbone using the linker designated L_{P}1, L_{P}2, L_{P}3, L_{P}4, L_{P}5 or L_{P}6 in Table 6 below.

In some embodiments the peptide derivative comprises a protraction moiety, wherein the protractor Chem. 5 is attached to the peptide backbone using the linker designated L_{P}1, L_{P}2, L_{P}3 L_{P}4, L_{P}5 or L_{P}6 in Table 6 below, thus wherein said protraction moiety comprises Chem. 18, Chem. 19, Chem. 20, Chem. 21, Chem. 33 or Chem. 34 as linker L_{P} and Chem. 5 as protractor P.

In some embodiments the peptide derivative comprises a protraction moiety, wherein the protractor Chem. 6 is attached to the peptide backbone using the linker designated L_{P}1, L_{P}2, L_{P}3 L_{P}4, L_{P}5 or L_{P}6 in Table 6 below, thus wherein said protraction moiety comprises Chem. 18, Chem. 19, Chem. 20, Chem. 21, Chem. 33 or Chem. 34 as linker L_{P} and Chem. 6 as protractor P.

In some embodiments the peptide derivative comprises a protraction moiety, wherein the protractor Chem. 32 is attached to the peptide backbone using the linker designated L_{P}1, L_{P}2, L_{P}3 L_{P}4, L_{P}5 or L_{P}6 in Table 6 below, thus wherein said protraction moiety comprises Chem. 18, Chem. 19, Chem. 20, Chem. 21, Chem. 33 or Chem. 34 as linker L_{P} and Chem. 32 as protractor P.

In a preferred embodiment the peptide derivative comprises a protraction moiety, wherein said protraction moiety comprises Chem. 20 or Chem. 21 as linker L_{P} and Chem. 5 or Chem. 6 as protractor P.

In a preferred embodiment the peptide derivative comprises a protraction moiety, wherein the protractor Chem. 5 is attached to the peptide backbone using the linker designated L_{P}3 in Table 6 below, thus wherein said protraction moiety comprises Chem. 20 as linker L_{P} and Chem. 5 as protractor P.

In a preferred embodiment the peptide derivative comprises a protraction moiety, wherein the protractor Chem. 6 is attached to the peptide backbone using the linker designated L_{P}3 in Table 6 below, thus wherein said protraction moiety comprises Chem. 20 as linker L_{P} and Chem. 6 as protractor P.

Based on the disclosure herein, the skilled person will be able to determine the optimal L_{P} linker for use in a specific peptide derivative as disclosed herein, optionally after some limited routine experiments.

TABLE 6
Examples of optional linkers (Lp) of the protraction moiety

| Linker L_{P} | |
|---|---|
| ID | Structure |
| L_{P}1 (Chem. 18) | 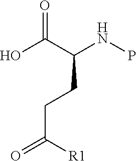 |
| L_{P}2 (Chem. 19) | 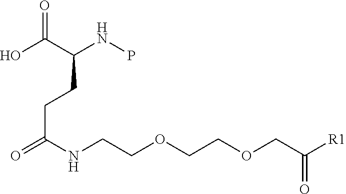 |
| L_{P}3 (Chem. 20) | 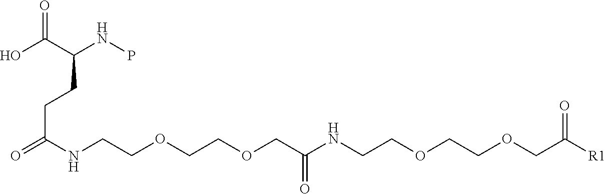 |
| L_{P}4 (Chem. 21) | 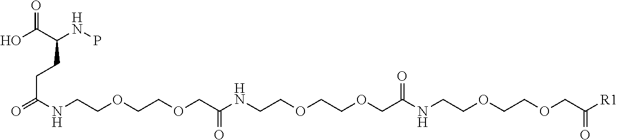 |
| L_{P}5 (Chem. 33) | 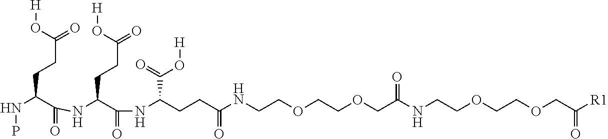 |
| L_{P}6 (Chem. 34) | 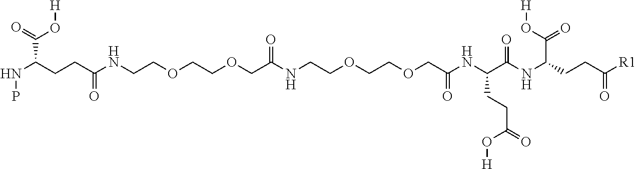 |

In some embodiments the peptide derivative comprises a protraction moiety which is selected from the group presented in Table 7. R1 represents the residue in the peptide backbone to which the protraction moiety is attached to.

TABLE 7
Examples of protraction moieties

| Protraction | |
|---|---|
| moiety | Structure |
| C₂₀ diacid gamma-Glu (Chem. 22) | 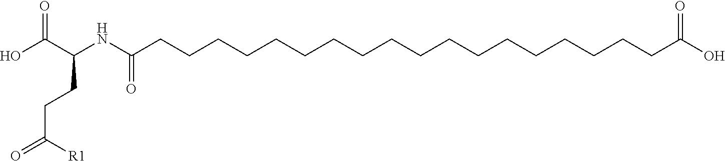 |
| C₂₀ diacid gamma-Glu gamma-Glu (Chem. 23) | 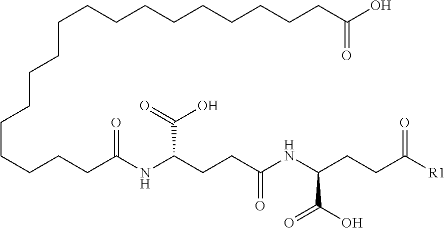 |
| C₂₀ diacid gamma-Glu gamma-Glu gamma-Glu (Chem. 24) | 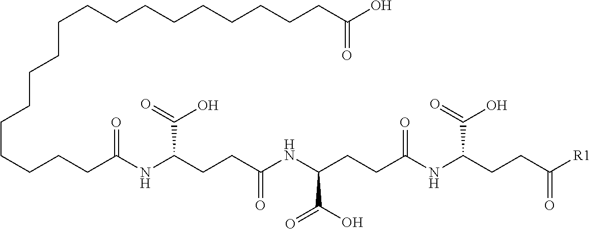 |
| C₂₀ diacid Ado (Chem. 25) | 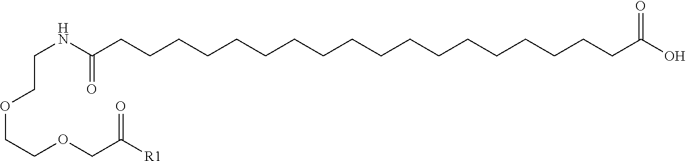 |
| C₂₀ diacid gamma-Glu Ado (Chem. 26) | 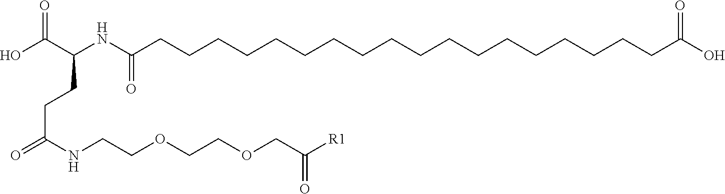 |
| C₂₀ diacid gamma-Glu 2xAdo (Chem. 27) | 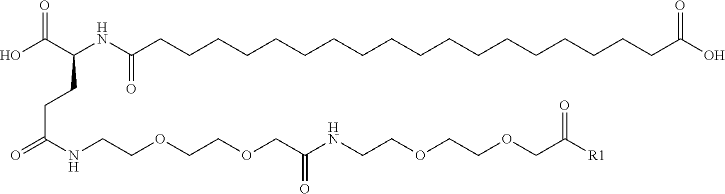 |
| C₁₈ diacid gamma-Glu 2xAdo (Chem. 28) | 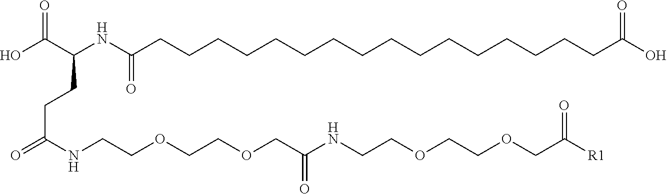 |
| C₁₆ diacid gamma-Glu 2xAdo (Chem. 29) | 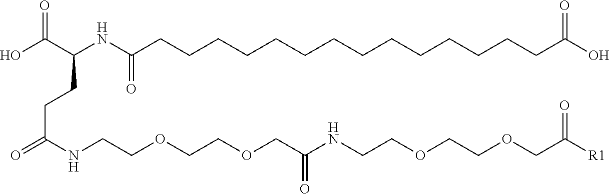 |
| C₁₈ diacid gamma-Glu (Chem. 30) | 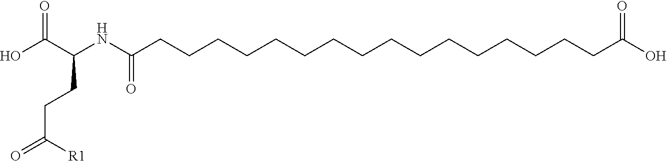 |
| C₁₈ diacid gamma-Glu 3xAdo (Chem. 31) | 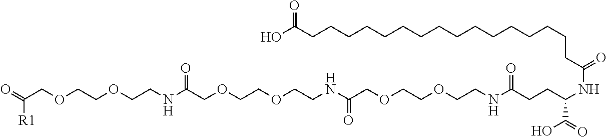 |
| C₁₉ phosphonic acid gamma-Glu 2xAdo (Chem. 35) | 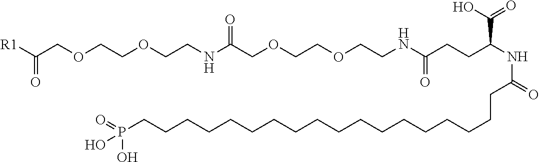 |
| (Chem. 36) C₂₀ diacid 2×Glu gamma-Glu 2xAdo | 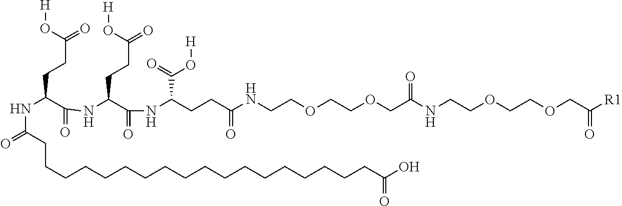 |
| (Chem. 37) C₂₀ diacid gamma-Glu 2xAdo Glu gamma-Glu | 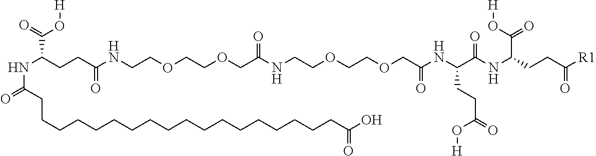 |

In one embodiment, the protraction moiety is selected from the list consisting of Chem. 27, Chem. 35, Chem. 36, and Chem. 36. In a preferred embodiment, the protraction moiety is Chem 27.

In one embodiment the free lysine may serve as a conjugation site for attaching one C₁₆ diacid gamma-Glu 2×Ado fatty acid moiety (IUPAC name [2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₆ diacid (S) gamma-Glu 2×Ado fatty acid moiety.

In one embodiment a free lysine may serve as a conjugation site for attaching one C₁₈ diacid gamma-Glu 2×Ado fatty acid moiety (IUPAC name [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₈ diacid (S) gamma-Glu 2×Ado fatty acid moiety.

In one embodiment a free lysine may serve as a conjugation site for attaching one C₂₀ diacid gamma-Glu 2×Ado fatty acid moiety (IUPAC name [2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₂₀ diacid (S) gamma-Glu 2×Ado fatty acid moiety.

In one embodiment a free lysine may serve as a conjugation site for attaching one C₁₈ diacid gamma-Glu fatty acid moiety (IUPAC name [(4S)-4-carboxy-4-(17-carboxy-heptadecanoylamino)butanoyl]), and thus the peptide derivative of the present invention comprises a protraction moiety, wherein said protraction moiety is a C₁₈ diacid (S) gamma-Glu fatty acid moiety.

In one embodiment a free lysine may serve as a conjugation site for attaching one C₂₀ diacid gamma-Glu fatty acid moiety (IUPAC name [(4S)-4-carboxy-4-(19-carboxy-nonadecanoylamino)butanoyl]), and thus the peptide derivative of the present invention comprises a protraction moiety, wherein said protraction moiety is a C₂₀ diacid (S) gamma-Glu fatty acid moiety.

In a preferred embodiment a free lysine may serve as a conjugation site for attaching one C₁₈ diacid gamma-Glu 2×Ado fatty acid moiety (IUPAC name [2-[2-[2-[[2-[2-[2-[[(4S)-4-carboxy-4-(17-carboxypentadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₈ diacid (S) gamma-Glu 2×Ado fatty acid moiety.

In a preferred embodiment a free lysine may serve as a conjugation site for attaching one C₂₀ diacid gamma-Glu 2×Ado fatty acid moiety (IUPAC name [2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₂₀ diacid (S) gamma-Glu 2×Ado fatty acid moiety.

In a most preferred embodiment a free lysine may serve as a conjugation site for attaching one C₁₈ diacid gamma-Glu 2×Ado fatty acid moiety (IUPAC name [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₈ diacid (S) gamma-Glu 2×Ado fatty acid moiety.

### GLP-1-/GlP-/Amylin-Receptor Tri-Agonist Derivative

As described above, the GLP-1-/GlP-/amylin-receptor tri-agonist of the present invention may comprise a peptide linker and further comprise a protraction moiety. Therefore, the present invention relates in another aspect to a GLP-1-/GlP-/amylin-receptor tri-agonist comprising a peptide according to Formula I:

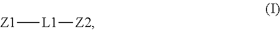

comprising one lysine (Lys, K) residue; wherein:
   Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E),
   wherein the peptide is a peptide derivative comprising a protraction moiety selected from Table 7.

In another aspect, the present invention relates to a GLP-1-/GlP-/amylin-receptor tri-agonist comprising a peptide according to Formula I:

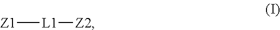

comprising one lysine (Lys, K) residue; wherein:
   Z1 is a peptide comprising or consisting of an amino acid sequence according to Formula XVII (SEQ ID NO: 62):

(XVII)
YX₂EGTFTSDYSX₁₂LLEEIAAREFIX₂₄WLX₂₇X₂₈GX₃₀X₃₁SX₃₃
X₃₄,

wherein
X₂ represents Aib,
X₁₂ represents Ile (I) or Lys (K),
X₂₄ represents Ala (A), Glu (E) or Gln (Q),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide comprising or consisting of an amino acid sequence according to Formula VI (SEQ ID NO: 165):

(VI)
AX52X53LSTAX58X59X60RLSAELHX68LATX72PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E),
   wherein the peptide is a peptide derivative comprising a protraction moiety selected from Table 7.

In another aspect, the invention relates to a GLP-1-/GlP-/amylin receptor tri-agonist comprising a peptide according to Formula I:

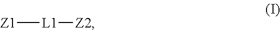

comprising one lysine (Lys, K) residue; wherein:
   Z1 is a peptide comprising a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E)
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide and comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E),
wherein the peptide is a peptide derivative comprising a protraction moiety selected from Table 7.

In another aspect, the GLP-1-/GlP-/amylin-receptor tri-agonist of the invention relates to a GLP-1-/GlP-/amylin-receptor tri-agonist comprising a peptide according to Formula I:

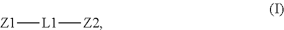

comprising one lysine (Lys, K) residue; wherein:
   Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃
X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E),
X₂₇ represents Leu (L) or I (Ile),
X₂₈ represents Ala (A) or Gln (Q), preferably Ala (A),
X₃₀ represents Gly (G) or Ala (A),
X₃₁ represents Gly (G), Gln (Q), Ala (A) or Pro (P), preferably Pro (P) or Ala (A),
X₃₃ represents Glu (E) or Ser (S), preferably Ser (S),
X₃₄ represents Gly (G) or Glu (E); preferably Gly (G), and
L1 is a peptide linker selected from the group consisting of: E, AE, AG, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159), preferably AG or AGEAPGEAPG (SEQ ID NO: 144); and
Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E),
   wherein the peptide is a peptide derivative comprising a protraction moiety selected from Table 7, preferably Chem. 27.

In another aspect, the present invention relates to a GLP-1-/GlP-/amylin-receptor tri-agonist comprising a peptide according to Formula I:

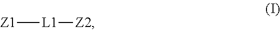

comprising one lysine (Lys, K) residue; wherein:
   Z1 is a peptide comprising or consisting of an amino acid sequence according to Formula XVII (SEQ ID NO: 62):

(XVII)
YX₂EGTFTSDYSX₁₂LLEEIAAREFIX₂₄WLX₂₇X₂₈GX₃₀X₃₁SX₃₃
X₃₄,

wherein
X₂ represents Aib,
X₁₂ represents Ile (I) or Lys (K),
X₂₄ represents Ala (A), Glu (E) or Gln (Q), preferably Glu (E),
X₂₇ represents Leu (L) or I (Ile),
X₂₈ represents Ala (A) or Gln (Q), preferably Ala (A),
X₃₀ represents Gly (G) or Ala (A),
X₃₁ represents Gly (G), Gln (Q), Ala (A) or Pro (P)
X₃₃ represents Glu (E) or Ser (S), preferably Ser (S),
X₃₄ represents Gly (G) or Glu (E); preferably Gly (G); and
L1 is a peptide linker selected from the group consisting of: E, AE, AG, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159), preferably AG or AGEAPGEAPG (SEQ ID NO: 144); and
Z2 is a peptide comprising a C-terminal amide comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S), preferably Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q), preferably Gly (G) or Gln (Q),
X₆₈ represents Gln (Q), Glu (E), or Lys (K),
X₇₂ represents Leu (L) or Glu (E), preferably Leu (L),
   wherein the peptide is a peptide derivative comprising a protraction moiety selected from Table 7, preferably Chem. 27.

In another aspect, the invention relates to a GLP-1-/GlP-/amylin receptor tri-agonist comprising a peptide according to Formula I:

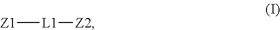

comprising one lysine (Lys, K) residue; wherein:
   Z1 is a peptide comprising a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E),
X₂₇ represents Leu (L) or I (Ile),
X₂₈ represents Ala (A) or Gln (Q), preferably Ala (A),
X₃₀ represents Gly (G) or Ala (A),
X₃₁ represents Gly (G), Gln (Q), Ala (A) or Pro (P), preferably Pro (P) or Ala (A),
X₃₃ represents Glu (E) or Ser (S), preferably Ser (S),
X₃₄ represents Gly (G) or Glu (E), preferably Gly (G); and
L1 is a peptide linker selected from the group consisting of: E, AE, AG, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159), preferably AG or AGEAPGEAPG (SEQ ID NO: 144); and
Z2 is a peptide comprising a C-terminal amide and comprising or consisting of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S), preferably Ser (S),
X₅₃ represents Gln (Q), Glu (E), or His (H), preferably Glu (E) or His (H),
X₅₈ represents Ala (A) or Gln (Q),
X₅₉ represents Leu (L) or Thr (T),
X₆₀ represents Ala (A), Gly (G) or Gln (Q), preferably Gly (G) or Gln (Q),
X₆₈ represents Gln (Q), Glu (E), or Lys (K),
X₇₂ represents Leu (L) or Glu (E), preferably Leu (L); and
wherein the peptide is a peptide derivative comprising a protraction moiety selected from Table 7, preferably Chem. 27.

In yet another aspect, the invention relates to a GLP-1-/GlP-/amylin-receptor tri-agonist comprising a peptide according to Formula I:

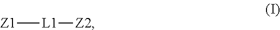

comprising one lysine (Lys, K) residue; wherein:
   Z1 is a peptide having a maximum of 4 amino acid substitutions relative to Formula II (SEQ ID NO: 1):

(II)
YX2EGTFTSDYSILLEEQAAREFIEWLLAGGPSKG,

wherein the amino acid at position X₂ represents Aib,
and
   Z1 comprises or consists of an amino acid sequence according to Formula VII (SEQ ID NO: 162):

(VII)
YX₂EGTFTSDYSX₁₂LLEEIAAX₂₀EFIX₂₄WLX₂₇X₂₈GGPSSX₃₄,

wherein
   X₂ represents Aib,
   X₁₂ represents Ile (I) or Lys (K),
   X₂₀ represents Arg (R) or Gln (Q),
   X₂₄ represents Ala (A), Glu (E) or Gln (Q),
   X₂₇ represents Leu (L) or I (Ile),
   X₂a represents Ala (A) or Gln (Q),
   X₃₄ represents Gly (G) or Glu (E);
   L1 is a peptide linker selected from the group consisting of E, GE, APPPSGGGE (SEQ ID NO: 129), AGQAPGQAPG (SEQ ID NO: 136), AGQAPGQAPL (SEQ ID NO: 137) and AGQAPGEAPG (SEQ ID NO: 154);
   Z2 is a peptide comprising a C-terminal amide, and having a maximum of 10 amino acid substitutions relative to Formula V (SEQ ID NO: 2):

(V)
ASELSTAALGRLSAELHELATLPRTETGSGSP,

and
   Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
   X₅₂ represents Gly (G) or Ser (S),
   X₅₃ represents Gln (Q), Glu (E), or His (H),
   X₅₈ represents Ala (A) or Gln (Q),
   X₅₉ represents Leu (L) or Thr (T),
   X₆₀ represents Ala (A), Gly (G) or Gln (Q),
   X₆₈ represents Gln (Q), Glu (E), or Lys (K),
   X₇₂ represents Leu (L) or Glu (E); and
wherein the peptide is a peptide derivative comprising a protraction moiety.

The GLP-1-/GlP-/amylin-receptor tri-agonists of the present invention may be a peptide derivative which comprise any one of the above disclosed peptides Z1, any one of the above disclosed peptides Z2, any one of the above disclosed peptide linkers L1 and any one of the above disclosed protraction moieties, and based on the disclosure herein, the skilled person will be able to determine the optimal combination to come to a specific peptide derivative which is a potent GLP-1-/GlP-/amylin-receptor tri-agonist having specific properties as described below.

The GLP-1-/GlP-/amylin-receptor tri-agonists of the invention may be selected from compound no. 104-197 as described in Example 2 herein. Preferred GLP-1-/GlP-/amylin-receptor tri-agonists of the present invention are

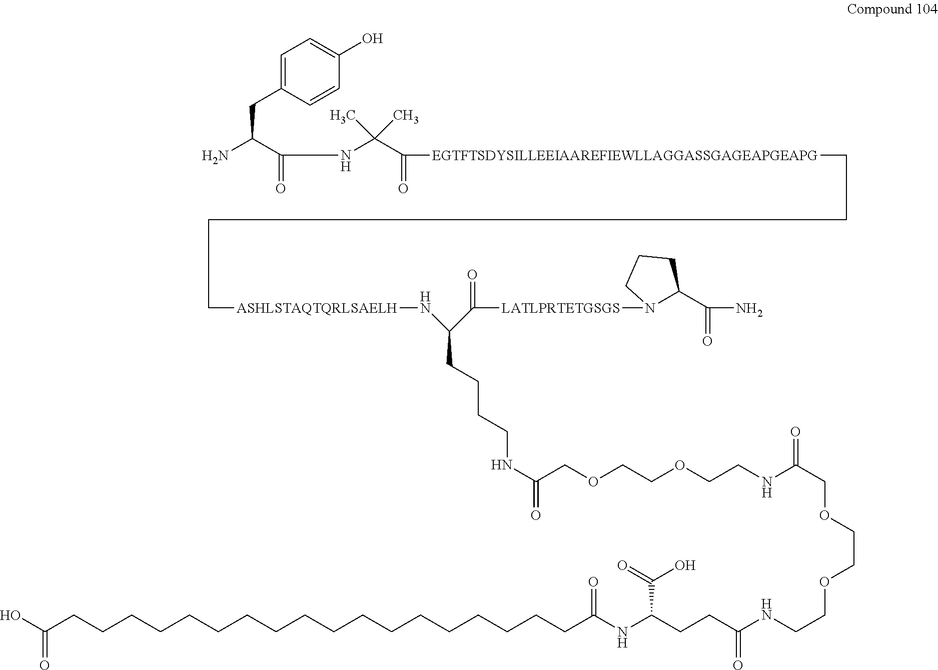

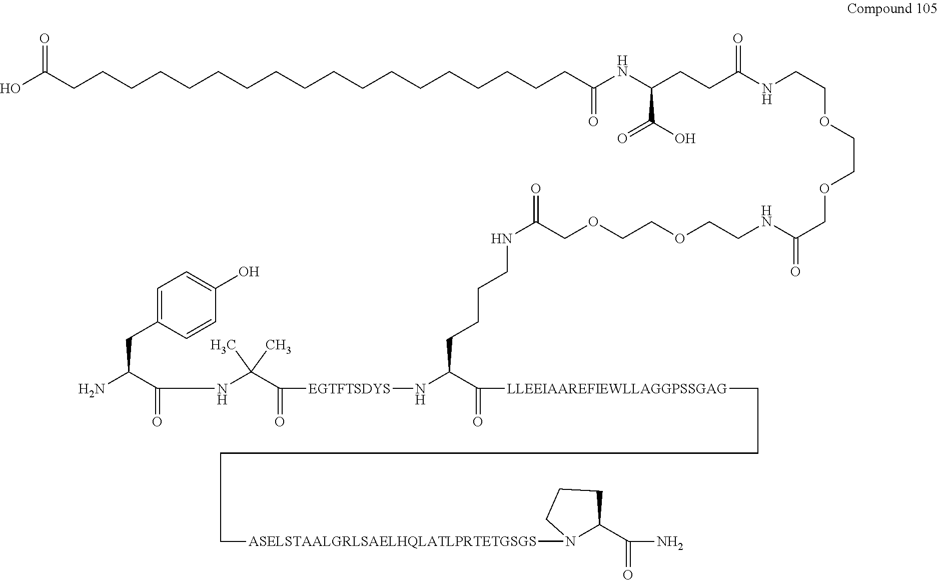

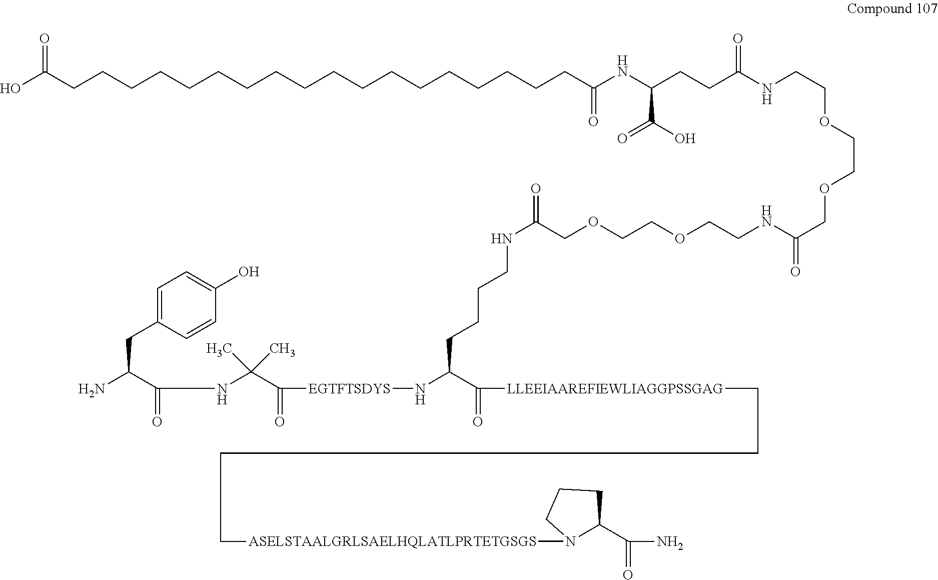

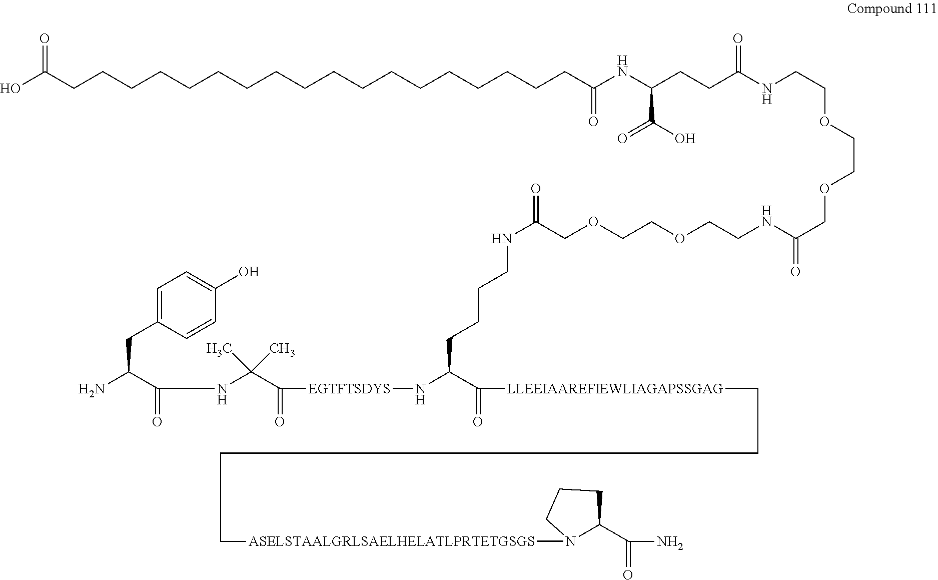

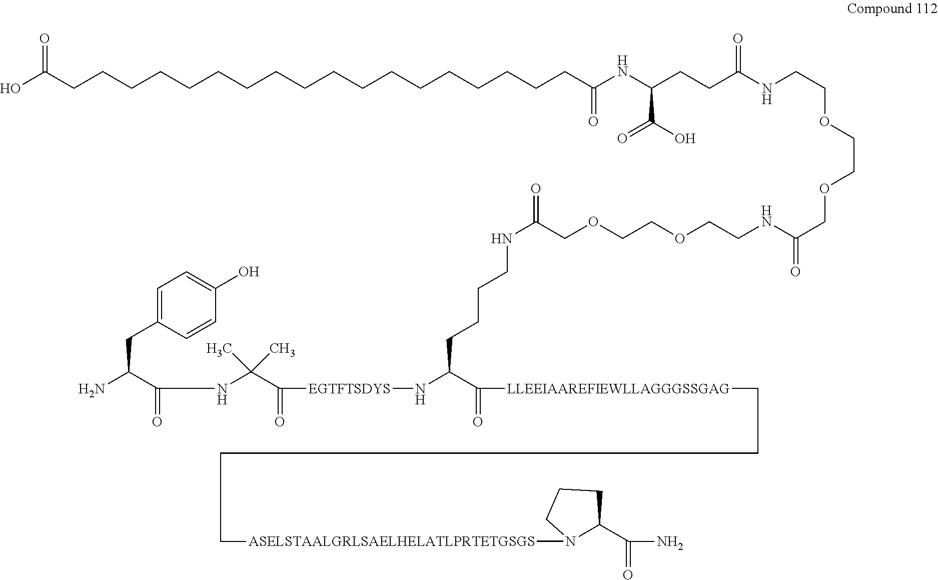

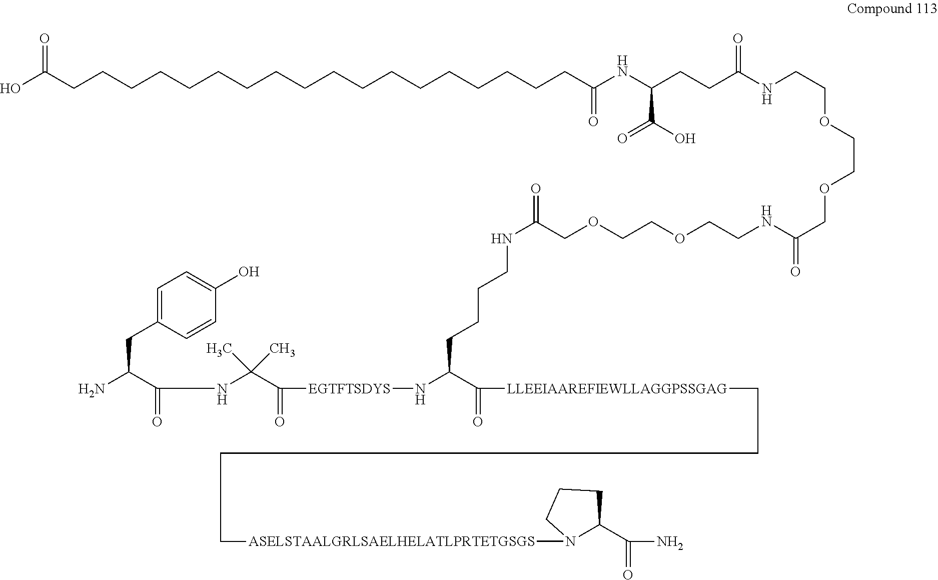

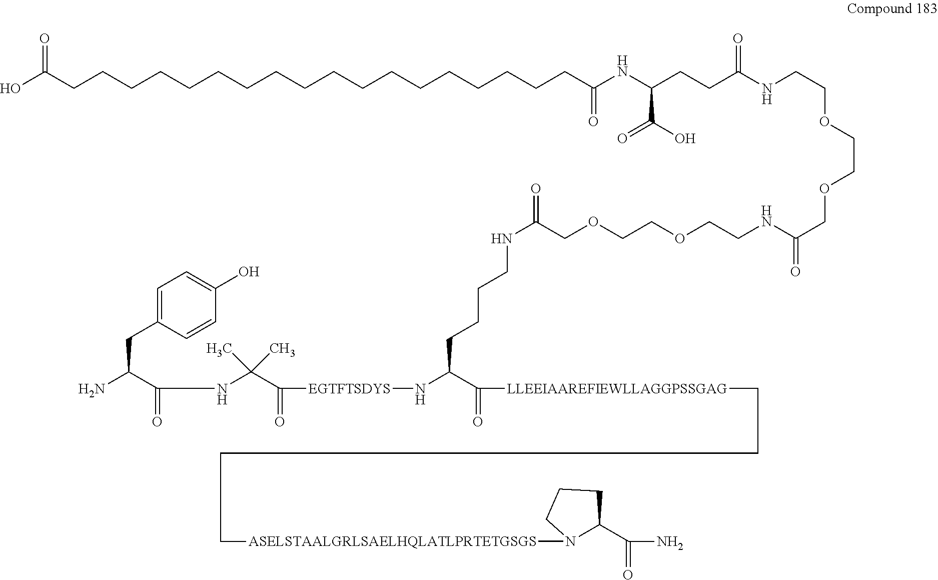

### Pharmaceutically Acceptable Salt

The compounds of the invention may be in the form of a pharmaceutically acceptable salt, or amide.

Salts are formed by a chemical reaction between a base and an acid, e.g.: 2 NH₃+H₂SO₄→(NH₄)₂SO₄.

The salt may be a basic salt, an acid salt, or it may be neither nor (i.e., a neutral salt). Basic salts produce hydroxide ions and acid salts produce hydronium ions in water.

The salts of the compounds of the invention may be formed with added cations or anions between anionic or cationic groups, respectively. These groups may be situated in the peptide moiety, and/or in the protraction moiety of the compounds of the invention.

Non-limiting examples of anionic groups of the compounds of the invention include free carboxylic groups in the protraction moiety, if any, as well as in the peptide backbone.

The peptide backbone may include free carboxylic groups at internal amino acid residues such as Asp (D) and Glu (E).

Non-limiting examples of cationic groups in the peptide backbone include the free amino group at the N-terminus, if present, as well as any free amino group of internal basic amino acid residues such as His (H), Arg (R), and Lys (K).

The amide of the compound of the invention may, e.g., be formed during peptide synthesis (based on the used resin) or by the reaction of a free carboxylic acid group with an amine or a substituted amine, or by reaction of a free or substituted amino group with a carboxylic acid. The amide formation may be at any free carboxylic group in the protraction moiety, the free amino group at the N-terminus of the peptide, and/or any free or substituted amino group in the peptide backbone.

In one aspect, the derivative of the invention is in the form of a pharmaceutically acceptable salt, preferably in the form of a trifluoroacetate salt.

### Functional Properties

In the first functional aspect, the GLP-1-/GlP-/amylin-receptor tri-agonist of the invention of the invention have good potency at each of the GLP-1-/GlP-/amylin-receptors. Preferably, they are potent GLP-1-/GlP-/amylin-receptor agonists as is reflected by their ability to activate each of the GLP-1/GlP-/amylin-receptors, Also, or alternatively, in a second functional aspect, the GLP-1-/GlP-/amylin-receptor tri-agonist of the invention are balanced tri-agonists. Also, or alternatively, in a third functional aspect, the GLP-1-/GlP-/amylin-receptor tri-agonist of the invention reduces food intake in vivo. Also, or alternatively, in a fourth functional aspect, the GLP-1-/GlP-/amylin-receptor tri-agonist of the invention have improved pharmacokinetic properties. Also, or alternatively, in a fifth functional aspect, the GLP-1-/GlP-/amylin-receptor tri-agonist of the invention are chemically stable.

### Biological Activity—In Vitro Potency

According to a first functional aspect, the GLP-1-/GlP-/amylin-receptor tri-agonist of the invention are biologically active, or potent at each of the GLP-1-/GlP-/amylin-receptors, i.e. a GLP-1-/GlP-/amylin-receptor tri-agonist peptide which is capable of activating the human GlP, GLP-1, and amylin receptors in vitro.

When tested as described in “GLP-1 receptor assay” (in the absence of HSA), the GLP-1-/GlP-/amylin-receptor tri-agonist disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

When tested as described in “GlP receptor assay” (in the absence of HSA), the GLP-1-/GlP-/amylin-receptor tri-agonist peptide disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

When tested as described in “Amylin receptor assay” (in the absence of HSA), the GLP-1-/GlP-/amylin-receptor tri-agonist peptide disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM. The GLP-1-/GlP-/amylin-receptor tri-agonist disclosed herein agonise, or activate, the amylin receptor.

The GLP-1-/GlP-/amylin-receptor tri-agonist disclosed herein may be tested for GLP-1, GlP and/or amylin activity as described in Example 4.

The more potent the compound, the lower its EC₅₀ value. The GLP-1-/GlP-/amylin-receptor tri-agonist may have an EC₅₀ in a human GLP-1 receptor functional assay (see Example 4) of about 100 pM or less. The GLP-1-/GlP-/amylin-receptor tri-agonist may have an EC₅₀ in a human GLP-1 receptor functional assay of about 90 pM or less. The GLP-1-/GlP-/amylin-receptor tri-agonist may have an EC₅₀ in a human GLP-1 receptor functional assay of about 80 pM or less. The GLP-1-/GlP-/amylin-receptor tri-agonist may have an EC₅₀ in a human GLP-1 receptor functional assay of about 75 pM or less. The GLP-1-/GlP-/amylin-receptor tri-agonist may have an EC₅₀ in a human GLP-1 receptor functional assay of about 70 pM or less. The GLP-1-/GlP-/amylin-receptor tri-agonist may have an EC₅₀ in a human GLP-1 receptor functional assay of about 60 pM or less. The GLP-1-/GlP-/amylin-receptor tri-agonist may have an EC₅₀ in a human GLP-1 receptor functional assay of about 50 pM or less. The GLP-1-/GlP-/amylin-receptor tri-agonist may have an EC₅₀ in a human GLP-1 receptor functional assay of about 40 pM or less. The GLP-1-/GlP-/amylin-receptor tri-agonist may have an EC₅₀ in a human GLP-1 receptor functional assay of about 30 pM or less. The GLP-1-/GlP-/amylin-receptor tri-agonist may have an EC₅₀ in a human GLP-1 receptor functional assay of about 25 pM or less. The GLP-1-/GlP-/amylin-receptor tri-agonist may have an EC₅₀ in a human GLP-1 receptor functional assay of about 20 pM or less. The GLP-1-/GlP-/amylin-receptor tri-agonist may have an EC₅₀ in a human GLP-1 receptor functional assay of about 15 pM or less. The GLP-1-/GlP-/amylin-receptor tri-agonist may have an EC₅₀ in a human GLP-1 receptor functional assay of about 10 pM or less. The GLP-1-/GlP-/amylin-receptor tri-agonist may have an EC₅₀ in a human GLP-1 receptor functional assay of about 5 pM or less. The GLP-1-/GlP-/amylin-receptor tri-agonist disclosed herein may have a similar potency as that of semaglutide or tirzepatide.

The GLP-1-/GlP-/amylin-receptor tri-agonist may have an EC₅₀ in a human GlP receptor functional assay (see Example 4) of about 125 pM or less. The GLP-1-/GlP-/amylin-receptor tri-agonist may have an EC₅₀ in a human GlP receptor functional assay of about 100 pM or less. The GLP-1-/GlP-/amylin-receptor tri-agonist may have an EC₅₀ in a human GlP receptor functional assay of about 90 pM or less. The GLP-1-/GlP-/amylin-receptor tri-agonist may have an EC₅₀ in a human GlP receptor functional assay of about 80 pM or less. The GLP-1-/GlP-/amylin-receptor tri-agonist may have an EC₅₀ in a human GlP receptor functional assay of about 75 pM or less. The GLP-1-/GlP-/amylin-receptor tri-agonist may have an EC₅₀ in a human GlP receptor functional assay of about 70 pM or less. The GLP-1-/GlP-/amylin-receptor tri-agonist may have an EC₅₀ in a human GlP receptor functional assay of about 60 pM or less. The GLP-1-/GlP-/amylin-receptor tri-agonist may have an EC₅₀ in a human GlP receptor functional assay of about 50 pM or less. The GLP-1-/GlP-/amylin-receptor tri-agonist may have an EC₅₀ in a human GlP receptor functional assay of about 40 pM or less. The GLP-1-/GlP-/amylin-receptor tri-agonist may have an EC₅₀ in a human GlP receptor functional assay of about 30 pM or less. The GLP-1-/GlP-/amylin-receptor tri-agonist may have an EC₅₀ in a human GlP receptor functional assay of about 25 pM or less. The GLP-1-/GlP-/amylin-receptor tri-agonist may have an EC₅₀ in a human GlP receptor functional assay of about 20 pM or less. The GLP-1-/GlP-/amylin-receptor tri-agonist may have an EC₅₀ in a human GlP receptor functional assay of about 15 pM or less. The GLP-1-/GlP-/amylin-receptor tri-agonist may have an EC₅₀ in a human GlP receptor functional assay of about 10 pM or less. The GLP-1-/GlP-/amylin-receptor tri-agonist may have an EC₅₀ in a human GlP receptor functional assay of about 9 pM or less. The GLP-1-/GlP-/amylin-receptor tri-agonist may have an EC₅₀ in a human GlP receptor functional assay of about 8 pM or less. The GLP-1-/GlP-/amylin-receptor tri-agonist may have an EC₅₀ in a human GlP receptor functional assay of about 7 pM or less. The GLP-1-/GlP-/amylin-receptor tri-agonist may have an EC₅₀ in a human GlP receptor functional assay of about 6 pM or less. The GLP-1-/GlP-/amylin-receptor tri-agonist may have an EC₅₀ in a human GlP receptor functional assay of about 5 pM or less. The GLP-1-/GlP-/amylin-receptor tri-agonist disclosed herein may have a similar potency as that of tirzepatide.

The GLP-1-/GlP-/amylin-receptor tri-agonist may have an EC₅₀ in a human amylin receptor functional assay (see Example 4) of about 125 pM or less. The GLP-1-/GlP-/amylin-receptor tri-agonist may have an EC₅₀ in a human amylin receptor functional assay of about 100 pM or less. The GLP-1-/GlP-/amylin-receptor tri-agonist may have an EC₅₀ in a human amylin receptor functional assay of about 90 pM or less. The GLP-1-/GlP-/amylin-receptor tri-agonist may have an EC₅₀ in a human amylin receptor functional assay of about 80 pM or less. The GLP-1-/GlP-/amylin-receptor tri-agonist may have an EC₅₀ in a human amylin receptor functional assay of about 75 pM or less. The GLP-1-/GlP-/amylin-receptor tri-agonist may have an EC₅₀ in a human amylin receptor functional assay of about 70 pM or less. The GLP-1-/GlP-/amylin-receptor tri-agonist may have an EC₅₀ in a human amylin receptor functional assay of about 60 pM or less. The GLP-1-/GlP-/amylin-receptor tri-agonist may have an EC₅₀ in a human amylin receptor functional assay of about 50 pM or less. The GLP-1-/GlP-/amylin-receptor tri-agonist may have an EC₅₀ in a human amylin receptor functional assay of about 40 pM or less. The GLP-1-/GlP-/amylin-receptor tri-agonist may have an EC₅₀ in a human amylin receptor functional assay of about 30 pM or less. The GLP-1-/GlP-/amylin-receptor tri-agonist may have an EC₅₀ in a human amylin receptor functional assay of about 25 pM or less. The GLP-1-/GlP-/amylin-receptor tri-agonist may have an EC₅₀ in a human amylin receptor functional assay of about 20 pM or less. The GLP-1-/GlP-/amylin-receptor tri-agonist may have an EC₅₀ in a human amylin receptor functional assay of about 15 pM or less. The GLP-1-/GlP-/amylin-receptor tri-agonist may have an EC₅₀ in a human amylin receptor functional assay of about 10 pM or less. The GLP-1-/GlP-/amylin-receptor tri-agonist may have an EC₅₀ in a human amylin receptor functional assay of about 9 pM or less. The GLP-1-/GlP-/amylin-receptor tri-agonist may have an EC₅₀ in a human amylin receptor functional assay of about 8 pM or less. The GLP-1-/GlP-/amylin-receptor tri-agonist may have an EC₅₀ in a human amylin receptor functional assay of about 7 pM or less. The GLP-1-/GlP-/amylin-receptor tri-agonist may have an EC₅₀ in a human amylin receptor functional assay of about 6 pM or less. The GLP-1-/GlP-/amylin-receptor tri-agonist may have an EC₅₀ in a human amylin receptor functional assay of about 5 pM or less. The GLP-1-/GlP-/amylin-receptor tri-agonist disclosed herein may have a similar potency as that of cagrilintide.

### Biological Activity—Balanced GLP-1-/GlP-/Amylin-Receptor Tri-Agonists

In a second functional aspect, the GLP-1-/GlP-/amylin-receptor tri-agonist of the invention activates the human GlP, GLP-1 and amylin receptors in vitro, when measured without HSA in assays as described in Example 4, and which has a potency ratio (NB) of less than 50, preferably less than 30.

In one embodiment, the GLP-1-/GlP-/amylin-receptor tri-agonist activates the human GlP, GLP-1, and amylin receptors in vitro, when measured without HSA in assays as described in Example 4, and has a potency ratio NB of less than 50. In one embodiment, the GLP-1-/GlP-/amylin-receptor tri-agonist activates the human GlP, GLP-1, and amylin receptors in vitro, when measured without HSA in assays as described in Example 4, and has a potency ratio NB of less than 30.

The balanced GLP-1-/GlP-/amylin-receptor tri-agonist of the present invention activates the human GlP, GLP-1, and amylin receptors in vitro, when measured without HSA in assays as described in Example 4, and has a potency ratio (A/B), i.e., potency (A) of the receptor with lowest potency divided by potency (B) of the receptor with highest potency, of less than 50, preferably less than 30. The tri-agonists disclosed herein may have a potency ratio (NB) of less than 50, preferably less than 30 or 20, such as less than 19, less than 18, less than 17, less than 16, even more preferred less than 15, such as less than 14, less than 13, less than 12, and most preferred less than 11, such as less than 10, less than 9, less than 8 and less than 7.

### Biological Activity—In Vivo Pharmacology

In as third functional aspect, the GLP-1-/GlP-/amylin-receptor tri-agonist disclosed herein may reduce food intake in a subject, e.g., normal weight rats or SD rats. Administration of the GLP-1-/GlP-/amylin-receptor tri-agonist disclosed herein may result in an acute reduction in the intake of food. The in vivo effect of the GLP-1-/GlP-/amylin-receptor tri-agonist on food intake in rats may be assessed as described in Example 5. A reduction of food intake of 100% (hypothetical value), relative or compared to vehicle, means that the rat does not eat.

In one embodiment, the GLP-1-/GlP-/amylin-receptor tri-agonist disclosed herein, after a single subcutaneous administration of 10 nmol/kg, may reduce food intake at day 1 (0-24 hours) by at least 10% compared to vehicle, preferably by at least 30 or 50% compared to vehicle, such as at least 70% compared to vehicle. The GLP-1-/GlP-/amylin-receptor tri-agonist disclosed herein, after a single subcutaneous administration of 10 nmol/kg, may reduce food intake at day 1 (0-24 hours) by 1% to 100%, when compared to vehicle, such as 15% to 95%, preferably by 40% to 85% when compared to vehicle, even more preferably by 50% to 80%, when compared to vehicle.

In one embodiment, the GLP-1-/GlP-/amylin-receptor tri-agonist disclosed herein, after a single subcutaneous administration of 10 nmol/kg, may reduce food intake at day 2 (24-48 hours) by at least 15% compared to vehicle, preferably by at least 30 or 50% compared to vehicle, such as at least 70% compared to vehicle. The GLP-1-/GlP-/amylin-receptor tri-agonist disclosed herein, after a single subcutaneous administration of 10 nmol/kg, may reduce food intake at day 2 (24-48 hours) by 1% to 100%, when compared to vehicle, such as 15% to 95%, preferably by 40% to 95% when compared to vehicle, even more preferably by 70% to 95%, when compared to vehicle.

In one embodiment, the GLP-1-/GlP-/amylin-receptor tri-agonist disclosed herein, after a single subcutaneous administration of 30 nmol/kg, may reduce food intake at day 1 (0-24 hours) by at least 15% compared to vehicle, preferably by at least 35% compared to vehicle. The GLP-1-/GlP-/amylin-receptor tri-agonist disclosed herein, after a single subcutaneous administration of 30 nmol/kg, may reduce food intake at day 2 (24-48 hours) by at least 15% compared to vehicle, preferably by at least 35% compared to vehicle.

### Pharmacokinetic Profile

In a fourth functional aspect, the GLP-1-/GlP-/amylin-receptor tri-agonist of the invention have improved pharmacokinetic properties such as increased terminal half-life.

Half-life is an important parameter as a long half-life indicates that less frequent administration of a compound may be possible. Based on the disclosure herein, the skilled person will be able to determine the protraction moiety for use in a specific peptide derivative as disclosed herein, optionally after some limited routine experiments. The GLP-1-/GlP-/amylin-receptor tri-agonists or the peptide derivatives of the present invention have a long half-life relative to dosing interval, thus reducing the variability in steady state exposure.

The in vivo pharmacology, including half-life, of the GLP-1-/GlP-/amylin-receptor tri-agonist described herein may be assessed as described in Example 6. In some embodiments, the half-life is half-life (t½) in vivo in minipigs or in rats after i.v. administration, e.g., as described in Example 6. The half-life of the GLP-1-/GlP-/amylin-receptor tri-agonist in minipigs may be as long as about 90 hours, or longer. The half-life of the GLP-1-/GlP-/amylin-receptor tri-agonist in minipigs may be at least 80 hours, preferably at least 90 hours. The half-life of the GLP-1-/GlP-/amylin-receptor tri-agonist in minipigs may be more than 80, 85, 90, 95, 100, 105, 110, 115, 120, 125, 130, 135, 140, 150, 160, or 170 hours. The half-life of the GLP-1-/GlP-/amylin-receptor tri-agonist in minipigs may be 80-200 hours, such as 90-190 hours, such as 95-185 hours. The half-life of the GLP-1-/GlP-/amylin-receptor tri-agonist in rats may be as long as about 5 hours, or longer. The half-life of the GLP-1-/GlP-/amylin-receptor tri-agonist in rats may be at least 8 hours, preferably at least 10 hours. The half-life of the GLP-1-/GlP-/amylin-receptor tri-agonist in rats may be more than 5, 6, 7, 8, 9, 10, 12, 14, 16, 20, or 25 hours. The half-life of the GLP-1-/GlP-/amylin-receptor tri-agonist in rats may be 5-50 hours, such as 10-35 hours, such as 12-30 hours.

In one embodiment, the present invention relates to a GLP-1-/GlP-/amylin-receptor tri-agonist which is suitable for once weekly administration. The GLP-1-/GlP-/amylin-receptor tri-agonists or the peptide derivatives of the present invention have a long half-life relative to dosing interval, thus reducing the variability in steady state exposure, and thus making a once weekly administration possible.

In a one embodiment, the present invention relates to a GLP-1-/GlP-/amylin-receptor tri-agonist which is suitable for oral administration. The GLP-1-/GlP-/amylin-receptor tri-agonist described herein may be orally bioavailable; that is, present in the bloodstream following per oral administration. Therefore, the compound is suitable for oral administration of subjects in need thereof.

### Chemical Properties

In the fifth aspect, the present invention relates to a GLP-1-/GlP-/amylin-receptor tri-agonist having improved chemical stability. The term “chemical stability” refers to chemical (in particular covalent) changes in the polypeptide structure leading to formation of chemical degradation products, such as high molecular weight proteins (HMWPs), deamidation, isomerization and hydrolysis products potentially having a reduced biological potency, and/or increased immunogenic effect as compared to the intact polypeptide. The chemical stability may be determined by measuring the purity loss, e.g., by measuring the amount of chemical degradation products at various time-points after exposure to different environmental conditions, e.g., by SEC-HPLC, and/or LCMS, e.g., as described in Example 7 herein. The GLP-1-/GlP-/amylin-receptor tri-agonist of the invention has a purity loss per week of less than 10.0 percent, preferably less than 5.0 percent, such as 4.0 or 3.0 percent, more preferably less than 2.0 percent, and most preferred less than 1.5 percent, upon incubation at 37° C. and as determined in Example 7 described herein.

Both the peptide backbone and the protraction moiety have been engineered and refined to achieve a peptide derivative having all of the above properties.

### Method of Production

The tri-agonists disclosed herein may be produced by classical peptide synthesis, e.g. solid phase peptide synthesis using t-Boc or Fmoc chemistry, or other well established techniques, see e.g. Greene and Wuts, “Protective Groups in Organic Synthesis”, John Wiley & Sons, 1999; Florencio Zaragoza Dorwald, “Organic Synthesis on Solid Phase”, Wiley-VCH Verlag GmbH, 2000; and “Fmoc Solid Phase Peptide Synthesis”, Edited by W. C. Chan and P. D. White, Oxford University Press, 2000. In some embodiments, methods for preparing the tri-agonists are described herein. In some embodiments, the methods for preparing the tri-agonists as described herein comprises a step of solid phase peptide synthesis.

Also, or alternatively, the compounds, the peptide sequence or parts of the peptide sequences may be produced by recombinant methods, e.g., by culturing a host cell containing a DNA sequence encoding the tri-agonist peptide sequence and capable of expressing the peptide, in a suitable nutrient medium under conditions permitting the expression of the peptide. Non-limiting examples of host cells suitable for expression of these peptides are: Escherichia coli, Saccharomyces cerevisiae as well as mammalian BHK or CHO cell lines.

The tri-agonists that include non-natural amino acids and/or covalently attached substituents (protraction moieties) may be produced as described under ‘General method for peptide synthesis’ in the experimental part. Or see e.g., Hodgson et al: “The synthesis of peptides and proteins containing non-natural amino acids”, Chemical Society Reviews, vol. 33, no. 7 (2004), p. 422-430.

The tri-agonists as described herein which include a protraction moiety may, e.g., be produced as described under ‘General method for peptide synthesis’ in the experimental part. In some embodiments, the protraction moiety is built as part of the solid phase peptide synthesis or produced separately and attached via the one lysine residue after the solid phase peptide synthesis.

Specific examples of methods of preparing a number of the tri-agonists as described herein are provided below.

A further aspect of the invention relates to a method for preparing the receptor tri-agonists described herein.

In one embodiment, the method for preparing a compound as described herein comprises a step of solid phase peptide synthesis. The protraction moiety may be built sequentially as part of the solid phase peptide synthesis or produced separately and attached via the lysine residue after peptide synthesis.

### Pharmaceutical Compositions

In a further aspect the present invention relates to a pharmaceutical composition comprising said GLP-1-/GlP-/amylin-receptor tri-agonist. Disclosed herein is a pharmaceutical composition comprising the GLP-1-/GlP-/amylin-receptor tri-agonist as disclosed herein, and one or more pharmaceutically acceptable excipients. Pharmaceutical composition comprising the GLP-1-/GlP-/amylin-receptor tri-agonist as disclosed herein, and one or more pharmaceutically acceptable excipients, may be prepared using methods known to the person skilled in the art.

The term “pharmaceutically acceptable excipient” refers to any ingredient in the pharmaceutical composition which is not the active pharmaceutical ingredient, or the GLP-1-/GlP-/amylin-receptor tri-agonist as disclosed herein. The term “pharmaceutically acceptable excipient” means an excipient that is useful in preparing a pharmaceutical composition that is generally safe, non-toxic and includes excipients that are acceptable for human pharmaceutical use. Such excipients can for example be solid, liquid or semisolid.

The excipient may be functional or inert and may serve various purposes, e.g. as a buffer, an isotonicity agent, a carrier, a vehicle, a filler, a binder, a lubricant, a glidant, a disintegrant, a flow control agent, a crystallization inhibitor, a solubilizer, a stabilizer, a colouring agent, a flavouring agent, a surfactant, emulsifier, or combinations thereof and/or to improve administration, and/or absorption of the active pharmaceutical ingredient(s). The amount of each excipient used may vary within ranges conventional in the art.

Techniques and excipients which may be used are described in e.g., Handbook of Pharmaceutical Excipients (e.g., 8^{th} edition, Sheskey et al., Eds., American Pharmaceuticals Association and Pharmaceutical Press, publications department of the Royal Pharmaceutical Society of Great Britain (2017) and later editions) and Remington: The Science and Practice of Pharmacy (e.g., 23^{rd} edition, Remington and Allen, Eds., Pharmaceutical Press (2021) and later editions).

The pharmaceutical composition comprising the GLP-1-/GlP-/amylin-receptor tri-agonist as disclosed herein may be for oral administration.

The pharmaceutical composition comprising the GLP-1-/GlP-/amylin-receptor tri-agonist as disclosed herein may be a solid pharmaceutical composition (e.g., a tablet or capsule) containing the active pharmaceutical ingredient, for example as a freeze-dried or spray-dried composition, and may be used as is, dissolved prior to use, or combined with excipients in the formulation.

The pharmaceutical composition may be a solid pharmaceutical composition comprising the compound disclosed herein, a salt of N-[8-(2-hydroxybenzoyl)amino]caprylate, preferably sodium N-(8-(2-hydroxybenzoyl)amino)caprylate, and one or more further excipients, as is described in the art. For example, the solid pharmaceutical composition may be as described in WO 2012/080471, WO 2013/139694, WO 2013/189988, WO 2019/149880, WO 2019/215063, WO 2021/219710 or WO 2023/012263 A1.

Alternatively, the pharmaceutical composition comprising the GLP-1-/GlP-/amylin-receptor tri-agonist as disclosed herein may be a liquid composition, such as an aqueous composition. Such liquid compositions may be suitable for oral administration or for parenteral administration, for example intravenous, intramuscular, or subcutaneous administration.

Liquid compositions that are suitable for injection can be prepared using conventional techniques of the pharmaceutical industry which involve dissolving and mixing the ingredients as appropriate to give the desired end product. Thus, according to one procedure, the compound described herein is dissolved in a suitable buffer at a suitable pH. The composition may be sterilized, for example, by sterile filtration. Techniques and excipients which may be used to prepare liquid formulations are described in in e.g., Handbook of Pharmaceutical Excipients (e.g., 8^{th} edition, Sheskey et al., Eds., American Pharmaceuticals Association and Pharmaceutical Press, publications department of the Royal Pharmaceutical Society of Great Britain (2017) and later editions) and Remington: The Science and Practice of Pharmacy (e.g., 23^{rd} edition, Remington and Allen, Eds., Pharmaceutical Press (2021) and later editions). Preferably, in an embodiment wherein pharmaceutical composition is in a liquid formulation, the liquid formulation provides an improved stability.

The pharmaceutical compositions are typically administered to a subject already suffering from a disease, such as the indications described below, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease as well as the weight and general state of the subject.

In some embodiments the dose of the compounds to be delivered by subcutaneous administration may be from about 0.1 mg to 500 mg of the compound per day, preferably from about 0.5 mg to 150 mg per day, per every second day, per every third day, per every fourth day, per every fifth day, per every sixth day or once weekly depending on the severity of the condition.

A suitable dose may also be adjusted for a particular compound based on the properties of that compound, including its in vivo half-life or mean residence time and its biological activity. For example, compounds to be delivered could in one embodiment be administered once daily, or in another embodiment once weekly. Accordingly, the pharmaceutical compositions may be used for dosing approximately once daily, such as once every 12-36 hours, such as once every 18-30 hours, such as approximately once every 24 hours, or may be used for dosing approximately once weekly, such as once every 6-8 days.

In one embodiment the present invention relates to an injection device comprising said pharmaceutical composition.

### Pharmaceutical Indications

In a further aspect the present invention relates to the GLP-1-/GlP-/amylin-receptor triple agonist as disclosed herein for use as a medicament.

The GLP-1-/GlP-/amylin-receptor triple agonist as disclosed herein may be used for the following medical treatments or indications:
(i) prevention and/or treatment of all forms of diabetes, such as hyperglycaemia, type 2 diabetes, impaired glucose tolerance, type 1 diabetes, non-insulin dependent diabetes, MODY (maturity onset diabetes of the young), gestational diabetes, and/or for reduction of HbA1c;
(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;
(iii) prevention and/or treatment of eating disorders, such as obesity, e.g., by decreasing food intake, reducing body weight, suppressing appetite, inducing satiety; treating or preventing binge eating disorder, food cravings, bulimia nervosa and/or obesity induced by administration of an antipsychotic or a steroid; reduction of gastric motility; and/or delaying gastric emptying;
(iv) weight maintenance after successful weight loss (either drug induced or by diet and exercise)—i.e., prevention of weight gain after successful weight loss;
(v) prevention and/or treatment of cardiovascular diseases, such as delaying or reducing development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure.
(vi) prevention and/or treatment of non-alcoholic fatty liver disease (NAFLD, otherwise known as metabolic-dysfunction associated fatty liver disease, MAFLD) and/or non-alcoholic steatohepatitis (NASH, otherwise known as metabolic dysfunction-associated steatohepatitis, MASH);
(vii) prevention and/or treatment of cognitive impairment, such as that caused by Alzheimer's disease;
(viii) the prevention and/or treatment of chronic kidney disease;
(ix) the prevention and/or treatment of obstructive sleep apnoea.

In some embodiments the indication is (i). In some embodiments the indication is (ii). In a still further particular aspect the indication is (iii). In some embodiments the indication is (iv). In some embodiments the indication is (v). In some embodiments the indication is (vi). In some embodiments the indication is (vii). In some embodiments the indication is (viii). In some embodiments the indication is (ix). In some embodiments the indication is type 2 diabetes. In some embodiments the indication is overweight or obesity.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic (preventive), palliative, symptomatic and/or curative.

In some embodiments the indication is (i) and (iii). In some embodiments the indication is (ii) and (iii).

The World Health Organisation (WHO) defines overweight and obesity as being the abnormal or excessive accumulation of body fat that present a risk to an individual's overall health. Generally, all subjects suffering from obesity are also considered to be suffering from overweight. The subject suffering from obesity may be a human being, such as an adult human or a paediatric human, wherein “paediatric human” includes the infant, the child and the adolescents. Alternative to overweight, also the term pre-obesity is used in this field. The WHO considers body mass index (BMI) to be the most convenient population-level measure of overweight and obesity. Body mass index (BMI) is a measure of body fat based on height and weight. The formula for calculation is BMI=weight in kilograms (kg)/height in meters squared (m²).

For adults, the WHO defines overweight, and obesity as follows: overweight means having a BMI greater than or equal to 25; obesity means having a BMI greater than or equal to 30.

For children, the WHO considers age when defining overweight and obesity. For children under the age of five, overweight means having a weight-for-height greater than two standard deviations above the WHO Child Growth Standards median; and obesity means having a weight-for-height greater than three standard deviations above the WHO Child Growth Standards median. Overweight and obesity are defined as follows for children aged five to nineteen: overweight means having a BMI-for-age that is greater than one standard deviation above the WHO Growth Reference median; and obesity means having a BMI-for-age that is greater than two standard deviations above the WHO Growth Reference median.

Nonetheless, the diagnostic criteria for underweight, the normal range, pre-obesity/overweight and obesity can differ between countries/populations, as illustrated in Table 8 below for adults.

TABLE 8
Definitions of underweight, the normal range,
pre-obesity/overweight and obesity in adults

| | BMI (kg/m²) | | | | |
|---|---|---|---|---|---|
| | International/ | | | | |
| | European/US | Chinese | Japanese | Taiwanese | Korean |
| Nutritional | classi- | popu- | classi- | classi- | classi- |
| status | fication | lation | fication | fication | fication |
| Underweight | <18.5 | <18.5 | <18.5 | <18.5 | |
| Normal | >/=18.5 | >/=18.5 | >/=18.5 | >/=18.5 | |
| weight | and <25 | and <23 | and <25 | and <24 | |
| Overweight | >/=25 | >/=23 | | >/=24 | >/=23 |
| (pre-obesity) | and <30 | and <28 | | and <27 | and <25 |
| Obesity | >/=30 | >/=28 | >/=25 | >/=27 | >/=25 |
| Obesity | >/=30 | | >/=25 | >/=27 | >/=25 |
| class I | and <35 | | and <30 | and <30 | and <30 |
| Obesity | >/=35.0 | | >/=30 | >/=30 | >/=30 |
| class II | and <40 | | and <35 | and <35 | and <35 |
| Obesity | >/=40 | | >/=35 | >/=35 | >/=35 |
| class III | | | and <40 | | |
| Obesity | | | >/=40 | | |
| class IV | | | | | |

Guidelines for the Asian population were published by Misra A et al. J Assoc Physicians India. 2009; 57:163-70. Guidelines for the Chinese population were issued in the 2006 edition of the Guidelines for Prevention and Control of Overweight and Obesity in Chinese Adults, compiled by the Chinese Working Group on Obesity. Guidelines for the Japanese population were issued, in 2016, by the Japanese Society for the Study of Obesity (JASSO) in Guidelines for the management of obesity disease. Guidelines for the Taiwanese population were issued by the Taiwanese governments Health Promotion Administration (HPA), Ministry of Health and Welfare in 2023, in the 2^{nd} edition of its “Evidence-Based Guideline on Adult Obesity Prevention and Management”.

In some embodiments the subject suffering from obesity is human, such as an adult human or a paediatric human (including infants, children, and adolescents). A human subject suffering from obesity thus may have a BMI of 25 or more, or 27 or more, or 28 or more, or 30 or more; this subject may also be referred to as being obese. The obesity may be class I, class II, class Ill or class IV obesity (as defined in Table 8). In some embodiments the human subject suffering from obesity may have a BMI of ≥35 or a BMI in the range of ≥30 to <40. In some embodiments the obesity is severe obesity or morbid obesity, wherein the human subject may have a BMI of ≥40.

In some embodiments the invention relates to a method for treatment or prevention of overweight, optionally in the presence of at least one weight-related co-morbidity. In one embodiment the GLP-1-/GlP-/amylin-receptor triple agonist as disclosed herein is for use in the treatment of a subject with an initial body mass index (BMI) of 25 or more, 27 or more, or 28 or more, or 30 or more; optionally in the presence of at least one weight-related co-morbidity.

In some embodiments the invention relates to use of the formulation for treatment or prevention of overweight, optionally in the presence of at least one weight-related co-morbidity. In some embodiments the subject suffering from overweight is human, such as an adult human or a paediatric human (including infants, children, and adolescents). In some embodiments an adult human subject suffering from overweight may have a BMI of 23 or more, or 24 or more, or 25 or more, or 27 or more. In some embodiments a human subject suffering from overweight has a BMI in the range of 24 to <27, in the range of 24 to <28, in the range of 25 to <30 or in the range of 27 to <30. In some embodiments the weight-related co-morbidity is selected from the group consisting of hypertension, dysglycemia (prediabetes or type 2 diabetes), dyslipidaemia, high cholesterol, cardiovascular disease and obstructive sleep apnoea.

In some embodiments, the tri-agonist as disclosed herein relates to a method for weight management. In some embodiments, the tri-agonist as disclosed herein relates to a method for reduction of appetite. In some embodiments, the tri-agonist as disclosed herein relates to a method for reduction of food intake. In some embodiments, the tri-agonist as disclosed herein relates to a method of preventing or treating overweight in a subject.

The term “reduction of body weight” may include treatment or prevention of obesity and/or overweight.

Administration of the compound disclosed herein may be as an adjunct to a reduced-calorie diet and increased physical activity for chronic weight management in an adult subject suffering from obesity, i.e. with an initial body mass index (BMI) of 25 or more, or 27 or more, or 28 or more, or 30 or more; or in an adult subject suffering from overweight, i.e. with an initial body mass index (BMI) of 23 or more, or 24 or more, or 25 or more, or 27 or more; optionally in the presence of at least one weight-related co-morbidity (e.g., hypertension, dysglycemia (prediabetes or type 2 diabetes), dyslipidaemia, high cholesterol, cardiovascular disease or obstructive sleep apnoea).

### PARTICULAR EMBODIMENTS

1. A GLP-1-/GlP-/amylin-receptor tri-agonist comprising a peptide according to Formula I:

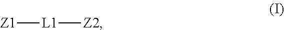

comprising one lysine (Lys, K) residue; wherein:
   Z1 is a peptide comprising a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ represents Aib;
   L1 is a peptide linker; and
   Z2 is a peptide comprising a C-terminal amide and a maximum of 10 amino acid substitutions relative to Formula V (SEQ ID NO: 2):

(V)
ASELSTAALGRLSAELHELATLPRTETGSGSP.

2. A GLP-1-/GlP-/amylin-receptor tri-agonist comprising a peptide according to Formula I:

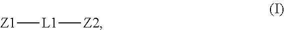

comprising one lysine (Lys, K) residue; wherein:
   Z1 is a peptide comprising or consisting of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃X₃₄,

wherein
X₂ represents Aib,
X₁₂ represents Ile (I) or Lys (K),
X₂₀ represents Arg (R) or Gln (Q), preferably Arg (R),
X₂₄ represents Ala (A), Glu (E) or Gln (Q), preferably Glu (E),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide comprising or consisting of an amino acid sequence according to Formula VI SEQ ID NO: 165:

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).
3. The GLP-1-/GlP-/amylin-receptor tri-agonist according to any one of the preceding embodiments comprising a peptide according to Formula I:

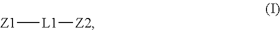

comprising one lysine (Lys, K) residue; wherein:
   Z1 is a peptide having a maximum of 5 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula III (SEQ ID NO: 168):

(III)
YX₂EGTFTSDYSX₁₂LLEEIAAX₂₀EFIX₂₄WLX₂₇X₂₈GX₃₀X₃₁SX₃₃X₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₀ represents Gly (G) or Ala (A),
X₃₁ represents Gly (G), Gln (Q), Ala (A) or Pro (P)
X₃₃ represents Glu (E) or Ser (S),
X₃₄ represents Gly (G) or Glu (E);
L1 is a peptide linker; and
Z2 is a peptide comprising a C-terminal amide, and Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).
4. The GLP-1-/GlP-/amylin-receptor tri-agonist according to any one of the preceding embodiments wherein Z1 is a peptide having a maximum of 4 amino acid substitutions relative to Formula II (SEQ ID NO: 1).
5. A GLP-1-/GlP-/amylin-receptor tri-agonist comprising a peptide according to Formula I:

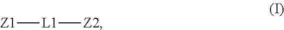

comprising one lysine (Lys, K) residue; wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and
Z1 comprises or consists of an amino acid sequence according to Formula VII (SEQ ID NO: 162):

(VII)
YX₂EGTFTSDYSX₁₂LLEEIAAX₂₀EFIX₂₄WLX₂₇X₂₈GGPSSX₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₄ represents Gly (G) or Glu (E);
L1 is a peptide linker; and
Z2 is a peptide comprising a C-terminal amide, and having a maximum of 10 amino acid substitutions relative to Formula V (SEQ ID NO: 2):

(V)
ASELSTAALGRLSAELHELATLPRTETGSGSP,

and
Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).
6. The GLP-1-/GlP-/amylin-receptor tri-agonist according to any one of the preceding embodiments, wherein the one lysine (Lys, K) residue is present in the peptide Z1 or in the peptide Z2.
7. The GLP-1/GlP-/amylin-receptor tri-agonist according to any one of the preceding embodiments, wherein the one lysine (Lys, K) residue is present in the peptide Z1.
8. The GLP-1/GlP-/amylin-receptor tri-agonist according to any one of embodiments 1-6, wherein the one lysine (Lys, K) residue is present in the peptide Z2.
9. The GLP-1-/GlP-/amylin-receptor tri-agonist according to any one of the preceding embodiments, wherein Formula I comprises no more than one lysine (Lys, K) residue.
10. The GLP-1-/GlP-/amylin-receptor tri-agonist according to any one of the preceding embodiments, wherein the GLP-1-/GlP-/amylin-receptor triple agonist does not comprise a cysteine (Cys, C) residue.
11. The GLP-1-/GlP-/amylin-receptor tri-agonist according to any one of the preceding embodiments, wherein the GLP-1-/GlP-/amylin-receptor triple agonist does not comprise a disulfide bridge.
12. The GLP-1-/GlP-/amylin-receptor tri-agonist according to any one of the preceding embodiments, wherein X₁₂ is Lys (K).
13. The GLP-1-/GlP-/amylin-receptor tri-agonist according to any one of embodiments 1-11, wherein X₁₂ is Ile (I).
14. The GLP-1-/GlP-/amylin-receptor tri-agonist according to any one of the preceding embodiments, wherein X₂₀ is Arg (R).
15. The GLP-1-/GlP-/amylin-receptor tri-agonist according to any one of embodiments 1-13, wherein X₂₀ is Gln (Q).
16. The GLP-1-/GlP-/amylin-receptor tri-agonist according to any one of the preceding embodiments, wherein X₂₄ is Glu (E).
17. The GLP-1-/GlP-/amylin-receptor tri-agonist according to any one of embodiments 1-15, wherein X₂₄ is Ala (A).
18. The GLP-1-/GlP-/amylin-receptor tri-agonist according to any one of embodiments 1-15, wherein X₂₄ is Gln (Q).
19. The GLP-1-/GlP-/amylin-receptor tri-agonist according to any one of the preceding embodiments, wherein X₂₇ is Leu (L).
20. The GLP-1-/GlP-/amylin-receptor tri-agonist according to any one of embodiments 1-18, wherein X₂₇ is Ile (I).
21. The GLP-1-/GlP-/amylin-receptor tri-agonist according to any one of the preceding embodiments, wherein X₂n is Ala (A).
22. The GLP-1-/GlP-/amylin-receptor tri-agonist according to any one of embodiments 1-20, wherein X₂n is Gln (Q).
23. The GLP-1-/GlP-/amylin-receptor tri-agonist according to any one of the preceding embodiments, wherein X₃₀ is Gly (G).
24. The GLP-1-/GlP-/amylin-receptor tri-agonist according to any one of embodiments 1-22, wherein X₃₀ is Ala (A).
25. The GLP-1-/GlP-/amylin-receptor tri-agonist according to any one of the preceding embodiments, wherein X₃₁ is Pro (P).
26. The GLP-1-/GlP-/amylin-receptor tri-agonist according to any one of embodiments 1-24, wherein X₃₁ is Ala (A).
27. The GLP-1-/GlP-/amylin-receptor tri-agonist according to any one of embodiments 1-24, wherein X₃₁ is Gly (G).
28. The GLP-1-/GlP-/amylin-receptor tri-agonist according to any one of embodiments 1-24, wherein X₃₁ is Gln (Q).
29. The GLP-1-/GlP-/amylin-receptor tri-agonist according to any one of the preceding embodiments, wherein X₃₃ is Ser (S).
30. The GLP-1-/GlP-/amylin-receptor tri-agonist according to any one of embodiments 1-28, wherein X₃₃ is Glu (E).
31. The GLP-1-/GlP-/amylin-receptor tri-agonist according to any one of the preceding embodiments, wherein X₃₄ is Gly (G).
32. The GLP-1-/GlP-/amylin-receptor tri-agonist according to any one of embodiments 1-30, wherein X₃₄ is Glu (E).
33. The GLP-1-/GlP-/amylin-receptor tri-agonist according to any one of the preceding embodiments, wherein X₃₀X₃₁ is Gly-Pro (GP).
34. The GLP-1-/GlP-/amylin-receptor tri-agonist according to any one of embodiments 1-32, wherein X₃₀X₃₁ is Gly-Ala (GA).
35. The GLP-1-/GlP-/amylin-receptor tri-agonist according to any one of embodiments 1-32, wherein X₃₀X₃₁ is Ala-Pro (AP).
36. The GLP-1-/GlP-/amylin-receptor tri-agonist according to any one of embodiments 1-32, wherein X₃₀X₃₁ is Gly-Gly (GG).
37. The GLP-1/GlP-/amylin-receptor tri-agonist according to any one of embodiments 1-32, wherein X₃₁ represents Gly (G), Gln (Q), or Ala (A).
38. The GLP-1-/GlP-/amylin-receptor tri-agonist according to any one of the preceding embodiments, wherein Z1 comprises or consists of an amino acid sequence according to Formula IV (SEQ ID NO: 169):

(IV)
YX₂EGTFTSDYSX₁₂LLEEIAAREFIEWLX₂₇AGX₃₀X₃₁SSG,

wherein
X₂ represents Aib,
X₁₂ represents Ile (I) or Lys (K),
X₂₇ represents Leu (L) or I (Ile),
X₃₀ represents Gly (G) or Ala (A),
X₃₁ represents Ala (A), Gly (G) or Pro (P).
39. The GLP-1-/GlP-/amylin-receptor tri-agonist according to any one of the preceding embodiments, wherein Z1 comprises or consists of an amino acid sequence according to Formula XI (SEQ ID NO: 163):

(XI)
YX₂EGTFTSDYSX₁₂LLEEIAAREFIEWLLAGGPSSG,

wherein
X₂ represents Aib,
X₁₂ represents Ile (I) or Lys (K).
40. The GLP-1-/GlP-/amylin-receptor tri-agonist according to any one of the preceding embodiments, wherein Z1 comprises or consists of an amino acid sequence selected from the list consisting of:

| SEQ ID NO: | Z1 peptide |
|---|---|
| 20 | YX₂EGTFTSDYSILLEEIAAREFIEWLLAGGPSSG |
| 21 | YX₂EGTFTSDYSILLEEIAAREFIEWLIAGGPSSG |
| 22 | YX₂EGTFTSDYSKLLEEIAAREFIEWLLAGGPSSG |
| 23 | YX₂EGTFTSDYSKLLEEIAAREFIEWLIAGGPSSG |
| 24 | YX₂EGTFTSDYSILLEEIAAREFIEWLLAGGPSSE |
| 25 | YX₂EGTFTSDYSILLEEIAAREFIAWLIAGGPSSG |
| 26 | YX₂EGTFTSDYSILLEEIAAREFIQWLIAGGPSSG |
| 27 | YX₂EGTFTSDYSKLLEEIAAREFIEWLLAGGPSSE |
| 28 | YX₂EGTFTSDYSILLEEIAAQEFIEWLLQGGPSSG |
| 29 | YX₂EGTFTSDYSILLEEIAAREFIEWLLAGGASSG |
| 30 | YX₂EGTFTSDYSKLLEEIAAREFIEWLLAGGQSSG |
| 31 | YX₂EGTFTSDYSKLLEEIAAREFIEWLIAGAPSSG |
| 32 | YX₂EGTFTSDYSKLLEEIAAREFIEWLIAGGPSEG |
| 33 | YX₂EGTFTSDYSKLLEEIAAREFIEWLIAGGGSSG |
| 34 | YX₂EGTFTSDYSKLLEEIAAREFIEWLLAGGGSSG |

wherein X₂ is Alb.
41. The GLP-1-/GlP-/amylin-receptor tri-agonist according to any one of the preceding embodiments, wherein Z1 comprises or consists of an amino acid sequence selected from the list consisting of:

| SEQ ID NO: | Z1 peptide |
|---|---|
| 22 | YX₂EGTFTSDYSKLLEEIAAREFIEWLLAGGPSSG |
| 29 | YX₂EGTFTSDYSILLEEIAAREFIEWLLAGGASSG |
| 31 | YX₂EGTFTSDYSKLLEEIAAREFIEWLIAGAPSSG |
| 34 | YX₂EGTFTSDYSKLLEEIAAREFIEWLLAGGGSSG |

wherein X₂ is Aib.
42. The GLP-1-/GlP-amylin-receptor tri-agonist according to any one of the preceding embodiments, wherein Z1 comprises or consists of an amino acid sequence is SEQ ID NO: 22.
43. The GLP-1-/GlP-amylin-receptor tri-agonist according to any one of the embodiments 1-41, wherein Z1 comprises or consists of an amino acid sequence is SEQ ID NO: 29.
44. The GLP-1-/GlP-amylin-receptor tri-agonist according to any one of the embodiments 1-41, wherein Z1 comprises or consists of an amino acid sequence is SEQ ID NO: 31.
45. The GLP-1-/GlP-amylin-receptor tri-agonist according to any one of the embodiments 1-41, wherein Z1 comprises or consists of an amino acid sequence is SEQ ID NO: 34.
46. The GLP-1-/GlP-/amylin-receptor tri-agonist according to any one of the preceding embodiments, wherein the peptide linker L1 comprises 1 to 14, 1 to 10, or 2 to 9, or 5 amino acid residues.
47. The GLP-1-/GlP-/amylin-receptor tri-agonist according to any one of the preceding embodiments, wherein the peptide linker L1 comprises 1 to 14 amino acid residues selected from the group consisting of Ala (A), Glu (E), Gln (Q), Gly (G), Leu (L), Phe (F), Pro (P), Ser (S), Thr (T), Val (V), Asn (N).
48. The GLP-1-/GlP-/amylin-receptor tri-agonist according to any one of the preceding embodiments, wherein the peptide linker L1 comprises or consists of the amino acid sequence according to Formula VIIIa:

(VIIIa)
X₁₄₁X₁₄₂X₁₄₃X₁₄₄X₁₄₅X₁₄₆X₁₄₇X₁₄₈X₁₄₉
X₁₅₀X₁₅₁X₁₅₂X₁₅₃X₁₅₄,

wherein
X₁₄₁ represents Ala (A), Glu (E), Gly (G),
X₁₄₂ represents Gln (Q), Glu (E), Gly (G), Leu (L), Pro (P) or is absent,
X₁₄₃ represents Ala (A), Gln (Q), Glu (E), Gly (G), Pro (P) or absent,
X₁₄₄ represents Ala (A), Gln (Q), Glu (E), Gly (G), Pro (P) or absent,
X₁₄₅ represents Glu (E), Gly (G), Pro (P), Ser (S), Thr (T) or is absent,
X₁₄₆ represents Glu (E), Gly (G), Leu (L), Gln (Q) or is absent,
X₁₄₇ represents Ala (A), Gln (Q), Glu (E), Gly (G), Phe (F) or is absent,
X₁₄₈ represents Ala (A), Gln (Q), Glu (E), Gly (G), Thr (T), Pro (P), Val (V) or is absent,
X₁₄₉ represents Glu (E), Asn (N), Pro (P), Thr (T) or is absent,
X₁₅₀ represents Ala (A), Gln (Q), Glu (E), Gly (G), Leu (L), Pro (P), Ser (S), Val (V) or is absent,
X₁₅₁ represents Ala (A) or is absent,
X₁₅₂ represents Gln (Q) or is absent,
X₁₅₃ represents Thr (T) or is absent,
X₁₅₄ represents Leu (L) or is absent.
49. The GLP-1-/GlP-/amylin-receptor tri-agonist according to any one of the preceding embodiments, wherein the peptide linker L1 comprises 1 to 10 amino acid residues selected from the group consisting of Ala (A), Glu (E), Gln (Q), Gly (G), Leu (L), Pro (P), Ser (S), Val (V).
50. The GLP-1-/GlP-/amylin-receptor tri-agonist according to any one of the preceding embodiments, wherein the peptide linker L1 comprises or consists of the amino acid sequence according to Formula VIII:

(VIII)
X₄₁X₄₂X₄₃X₄₄X₄₅X₄₆X₄₇X₄₈X₄₉X₅₀,

wherein
   X₄₁ represents Ala (A), Gly (G) or Glu (E),
   X₄₂ represents Glu (E), Gly (G) or is absent,
   X₄₃ represents Glu (E), Gly (G), Gln (Q) or absent,
   X₄₄ represents Ala (A), Glu (E), Gly (G) or absent,
   X₄₅ represents Glu (E), Gly (G), Pro (P) or is absent,
   X₄₆ represents Glu (E), Gly (G) or is absent,
   X₄₇ represents Gln (Q), Glu (E) or is absent,
   X₄₈ represents Ala (A), Glu (E) or is absent,
   X₄₉ represents Glu (E), Pro (P) or is absent,
   X₅₀ represents Ala (A), Glu (E), Gly (G), Leu (L), Pro (P), Ser (S), Val (V) or is absent.
51. The GLP-1-/GlP-/amylin-receptor tri-agonist according to any one of the preceding embodiments, wherein the peptide linker L1 comprises or consists of the amino acid sequence according to Formula VIII:

(VIII)
X₄₁X₄₂X₄₃XMAX₄₅X₄₆X₄₇X₄₈X₄₉X₅₀,

wherein
   X₄₁ represents Ala (A) or Glu (E),
   X₄₂ represents Glu (E), Gly (G) or is absent,
   X₄₃ represents Glu (E), Gly (G), Gln (Q) or absent,
   X₄₄ represents Ala (A), Glu (E), Gly (G) or absent,
   X₄₅ represents Glu (E), Gly (G), Pro (P) or is absent,
   X₄₆ represents Glu (E), Gly (G) or is absent,
   X₄₇ represents Gln (Q), Glu (E) or is absent,
   X₄₈ represents Ala (A), Glu (E) or is absent,
   X₄₉ represents Glu (E), Pro (P) or is absent,
   X₅₀ represents Ala (A), Glu (E), Gly (G), Leu (L), Pro (P), Ser (S), Val (V) or is absent.
52. The GLP-1-/GlP-/amylin-receptor tri-agonist according to any one of the preceding embodiments, wherein the peptide linker L1 is selected from the group consisting of A, E, G, AE, AG, GE, APPE (SEQ ID NO: 125), GGGE (SEQ ID NO: 126), AGQAPG (SEQ ID NO: 127), APPPSGGG (SEQ ID NO: 128), APPPSGGGE (SEQ ID NO: 129), APPPSGGGG (SEQ ID NO: 130), ALAQTLAQTL (SEQ ID NO: 131), ALAQTLFVNQ (SEQ ID NO: 132), ALAQTLGTNE (SEQ ID NO: 133), ALQAPGQAPG (SEQ ID NO: 134), ALQAPGQAPL (SEQ ID NO: 135), AGQAPGQAPG (SEQ ID NO: 136), AGQAPGQAPL (SEQ ID NO: 137), GGGEGGGEGE (SEQ ID NO: 138), GQAPGQAPGE (SEQ ID NO: 139), GQEPGQEPGE (SEQ ID NO: 140), APPPSLAQTLAQTL (SEQ ID NO: 141), AGGGG (SEQ ID NO: 142), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159).
53. The GLP-1-/GlP-/amylin-receptor tri-agonist according to any one of the preceding embodiments,
   wherein the peptide linker L1 is selected from the group consisting of A, E, G, AE, AG, GE, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159).
54. The GLP-1-/GlP-/amylin-receptor tri-agonist according to any one of the preceding embodiments,
   wherein the peptide linker L1 is selected from the group consisting of E, AE, AG, AGGGG (SEQ ID NO: 142), AGQAPGQAPG (SEQ ID NO: 136), AGQAPGQAPL (SEQ ID NO: 137), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159).
55. The GLP-1-/GlP-/amylin-receptor tri-agonist according to any one of the preceding embodiments, wherein the peptide linker L1 is selected from the group consisting of AG and AGEAPGEAPG (SEQ ID NO: 144).
56. The GLP-1-/GlP-/amylin-receptor tri-agonist according to any one of the preceding embodiments, wherein X₅₂ is Ser (S).
57. The GLP-1-/GlP-/amylin-receptor tri-agonist according to any one of embodiments 1-55, wherein X₅₂ is Gly (G).
58. The GLP-1-/GlP-/amylin-receptor tri-agonist according to any one of the preceding embodiments, wherein X₅₃ is Glu (E).
59. The GLP-1-/GlP-/amylin-receptor tri-agonist according to any one of embodiments 1-57, wherein X₅₃ is His (H).
60. The GLP-1-/GlP-/amylin-receptor tri-agonist according to any one of embodiments 1-57, wherein X₅₃ is Gln (Q).
61. The GLP-1-/GlP-/amylin-receptor tri-agonist according to any one of the preceding embodiments, wherein X₅₈ is Ala (A).
62. The GLP-1-/GlP-/amylin-receptor tri-agonist according to any one of embodiments 1-60, wherein X₅₈ is Gln (Q).
63. The GLP-1-/GlP-/amylin-receptor tri-agonist according to any one of the preceding embodiments, wherein X₅₉ is Leu (L).
64. The GLP-1-/GlP-/amylin-receptor tri-agonist according to any one of embodiments 1-62, wherein X₅₉ is Thr (T).
65. The GLP-1-/GlP-/amylin-receptor tri-agonist according to any one of the preceding embodiments, wherein X₆₀ is Gly (G).
66. The GLP-1-/GlP-/amylin-receptor tri-agonist according to any one of embodiments 1-64, wherein X₆₀ is Gln (Q).
67. The GLP-1-/GlP-/amylin-receptor tri-agonist according to any one of embodiments 1-64, wherein X₆₀ is Ala (A).
68. The GLP-1-/GlP-/amylin-receptor tri-agonist according to any one of the preceding embodiments, wherein X₆₈ is Gln (Q).
69. The GLP-1-/GlP-/amylin-receptor tri-agonist according to any one of embodiments 1-67, wherein X₆₈ is Glu (E).
70. The GLP-1-/GlP-/amylin-receptor tri-agonist according to any one of embodiments 1-67, wherein X₆₈ is Lys (K).
71. The GLP-1-/GlP-/amylin-receptor tri-agonist according to any one of the preceding embodiments, wherein X₇₂ is Leu (L).
72. The GLP-1-/GlP-/amylin-receptor tri-agonist according to any one of embodiments 1-70, wherein X₇₂ is Glu (E).
73. The GLP-1-/GlP-/amylin-receptor tri-agonist according to any one of the preceding embodiments, wherein Z2 comprises or consists of an amino acid sequence according to Formula IX (SEQ ID NO: 61):

(IX)
ASX₅₃LSTAX₅₈X₅₉X₆₀RLSAELHX₆₈LATLPRTETGSGSP,

wherein
X₅₃ represents Glu (E) or His (H),
X₅₈ represents Ala (A) or Gln (Q),
X₅₉ represents Leu (L) or Thr (T),
X₆₀ represents Gly (G) or Gln (Q),
X₆₈ represents Gln (Q), Glu (E), or Lys (K).
74. The GLP-1-/GlP-/amylin-receptor tri-agonist according to any one of the preceding embodiments, wherein Z2 comprises or consists of an amino acid sequence according to Formula X (SEQ ID NO: 166):

(X)
ASX₅₃LSTAQTQRLSAELHKLATLPRTETGSGSP,

wherein
X₅₃ represents Glu (E) or His (H).
75. The GLP-1-/GlP-/amylin-receptor tri-agonist according to any one of the preceding embodiments, wherein Z2 comprises or consists of an amino acid sequence selected from the group consisting of:

| SEQ | |
|---|---|
| ID | |
| NO: | Z2 peptide |
| 40 | ASHLSTAQTQRLSAELHKLATLPRTETGSGSP |
| 41 | ASHLSTAQLGRLSAELHELATLPRTETGSGSP |
| 42 | ASHLSTAQLGRLSAELHQLATLPRTETGSGSP |
| 43 | ASHLSTAQTQRLSAELHELATLPRTETGSGSP |
| 44 | ASHLSTAALGRLSAELHELATLPRTETGSGSP |
| 45 | ASHLSTAQTARLSAELHKLATLPRTETGSGSP |
| 46 | ASQLSTAQTQRLSAELHKLATLPRTETGSGSP |
| 47 | ASELSTAQTQRLSAELHKLATLPRTETGSGSP |
| 48 | AGELSTAQTQRLSAELHKLATLPRTETGSGSP |
| 49 | ASELSTAQTARLSAELHKLATLPRTETGSGSP |
| 50 | ASHLSTAQTQRLSAELHKLATEPRTETGSGSP |
| 51 | ASELSTAQTQRLSAELHKLATEPRTETGSGSP |
| 2 | ASELSTAALGRLSAELHELATLPRTETGSGSP |
| 53 | ASELSTAALGRLSAELHQLATLPRTETGSGSP |
| 54 | ASELSTAALGRLSAELHQLATEPRTETGSGSP |
| 55 | ASELSTAQLGRLSAELHQLATEPRTETGSGSP |
| 56 | ASELSTAQTGRLSAELHQLATEPRTETGSGSP |
| 57 | ASELSTAQTQRLSAELHQLATEPRTETGSGSP |
| 58 | ASQLSTAQTQRLSAELHKLATEPRTETGSGSP |
| 59 | ASELSTAQLGRLSAELHQLATLPRTETGSGSP |
| 60 | ASHLSTAQLGRLSAELHQLATEPRTETGSGSP |

76. The GLP-1-/GlP-/amylin-receptor tri-agonist according to any one of the preceding embodiments, wherein Z2 comprises or consists of an amino acid sequence selected from the group consisting of:

| SEQ ID NO: | Z2 peptide |
|---|---|
| 59 | ASELSTAQLGRLSAELHQLATLPRTETGSGSP |
| 40 | ASHLSTAQTQRLSAELHKLATLPRTETGSGSP |
| 53 | ASELSTAALGRLSAELHQLATLPRTETGSGSP |
| 2 | ASELSTAALGRLSAELHELATLPRTETGSGSP |

77. The GLP-1-/GlP-amylin-receptor tri-agonist according to any one of the preceding embodiments, wherein Z2 comprises or consists of an amino acid sequence is SEQ ID NO: 59.
78. The GLP-1-/GlP-amylin-receptor tri-agonist according to any one of embodiments 1-76, wherein Z2 comprises or consists of an amino acid sequence is SEQ ID NO: 40.
79. The GLP-1-/GlP-amylin-receptor tri-agonist according to any one of embodiments 1-76, wherein Z2 comprises or consists of an amino acid sequence is SEQ ID NO: 53.
80. The GLP-1-/GlP-amylin-receptor tri-agonist according to any one of embodiments 1-76, wherein Z2 comprises or consists of an amino acid sequence is SEQ ID NO: 2.
81. The GLP-1-/GlP-/amylin-receptor tr-agonist according to any one of the preceding embodiments, wherein the peptide Z1-L1-Z2 comprises or consists of an amino acid sequence wherein:
   Z1 is selected from the group consisting of:

| SEQ ID NO: | Z1 peptide |
|---|---|
| 20 | YX₂EGTFTSDYSILLEEIAAREFIEWLLAGGPSSG |
| 21 | YX₂EGTFTSDYSILLEEIAAREFIEWLIAGGPSSG |
| 22 | YX₂EGTFTSDYSKLLEEIAAREFIEWLLAGGPSSG |
| 23 | YX₂EGTFTSDYSKLLEEIAAREFIEWLIAGGPSSG |
| 24 | YX₂EGTFTSDYSILLEEIAAREFIEWLLAGGPSSE |
| 25 | YX₂EGTFTSDYSILLEEIAAREFIAWLIAGGPSSG |
| 26 | YX₂EGTFTSDYSILLEEIAAREFIQWLIAGGPSSG |
| 27 | YX₂EGTFTSDYSKLLEEIAAREFIEWLLAGGPSSE |
| 28 | YX₂EGTFTSDYSILLEEIAAQEFIEWLLQGGPSSG |
| 29 | YX₂EGTFTSDYSILLEEIAAREFIEWLLAGGASSG |
| 30 | YX₂EGTFTSDYSKLLEEIAAREFIEWLLAGGQSSG |
| 31 | YX₂EGTFTSDYSKLLEEIAAREFIEWLIAGAPSSG |
| 32 | YX₂EGTFTSDYSKLLEEIAAREFIEWLIAGGPSEG |
| 33 | YX₂EGTFTSDYSKLLEEIAAREFIEWLIAGGGSSG |
| 34 | YX₂EGTFTSDYSKLLEEIAAREFIEWLLAGGGSSG |

L1 is selected from the group consisting of:

(SEQ ID NO: 142)
E, AE, AG, AGGGG,
(SEQ ID NO: 136)
AGQAPGQAPG,
(SEQ ID NO: 137)
AGQAPGQAPL,
(SEQ ID NO: 143)
AGEAPGQAPG,
(SEQ ID NO: 144)
AGEAPGEAPG,
(SEQ ID NO: 145)
AGQAPGQAPA,
(SEQ ID NO: 146)
AGQAPGQAPE,
(SEQ ID NO: 147)
AGQAPGQAPP,
(SEQ ID NO: 148)
AGQAPGQAPS,
(SEQ ID NO: 149)
AGQAPGQAPV,
(SEQ ID NO: 150)
EGQAPGQAPG,
(SEQ ID NO: 151)
AGQEPGQAPG,
(SEQ ID NO: 152)
AGQAEGQAPG,
(SEQ ID NO: 153)
AGQAPEQAPG,
(SEQ ID NO: 154)
AGQAPGEAPG,
(SEQ ID NO: 155)
AGQAPGQEPG,
(SEQ ID NO: 156)
AGQAPGQAEG,
(SEQ ID NO: 157)
AGQEPGQEPG,
(SEQ ID NO: 158)
AGQAPGQAP
and
(SEQ ID NO: 159)
AGQAPGEAPL,

and
Z2 is selected from the group consisting of:

| SEQ ID NO: | Z2 peptide |
|---|---|
| 40 | ASHLSTAQTQRLSAELHKLATLPRTETGSGSP |
| 41 | ASHLSTAQLGRLSAELHELATLPRTETGSGSP |
| 42 | ASHLSTAQLGRLSAELHQLATLPRTETGSGSP |
| 43 | ASHLSTAQTQRLSAELHELATLPRTETGSGSP |
| 44 | ASHLSTAALGRLSAELHELATLPRTETGSGSP |
| 45 | ASHLSTAQTARLSAELHKLATLPRTETGSGSP |
| 46 | ASQLSTAQTQRLSAELHKLATLPRTETGSGSP |
| 47 | ASELSTAQTQRLSAELHKLATLPRTETGSGSP |
| 48 | AGELSTAQTQRLSAELHKLATLPRTETGSGSP |
| 49 | ASELSTAQTARLSAELHKLATLPRTETGSGSP |
| 50 | ASHLSTAQTQRLSAELHKLATEPRTETGSGSP |
| 51 | ASELSTAQTQRLSAELHKLATEPRTETGSGSP |
| 2 | ASELSTAALGRLSAELHELATLPRTETGSGSP |
| 53 | ASELSTAALGRLSAELHQLATLPRTETGSGSP |
| 54 | ASELSTAALGRLSAELHQLATEPRTETGSGSP |
| 55 | ASELSTAQLGRLSAELHQLATEPRTETGSGSP |
| 56 | ASELSTAQTGRLSAELHQLATEPRTETGSGSP |
| 57 | ASELSTAQTQRLSAELHQLATEPRTETGSGSP |
| 58 | ASQLSTAQTQRLSAELHKLATEPRTETGSGSP |
| 59 | ASELSTAQLGRLSAELHQLATLPRTETGSGSP |
| 60 | ASHLSTAQLGRLSAELHQLATEPRTETGSGSP |

82. The GLP-1-/GlP-/amylin-receptor tr-agonist according to any one of the preceding embodiments, wherein the peptide Z1-L1-Z2 comprises or consists of an amino acid sequence wherein:
   Z1 is selected from the group consisting of:

| SEQ ID NO: | Z1 peptide |
|---|---|
| 22 | YX₂EGTFTSDYSKLLEEIAAREFIEWLLAGGPSSG |
| 29 | YX₂EGTFTSDYSILLEEIAAREFIEWLLAGGASSG |
| 31 | YX₂EGTFTSDYSKLLEEIAAREFIEWLIAGAPSSG |
| 34 | YX₂EGTFTSDYSKLLEEIAAREFIEWLLAGGGSSG |

L1 is selected from the group consisting of AG and AGEAPGEAPG (SEQ ID NO: 144),
Z2 is selected from the group consisting of

| SEQ ID NO: | Z2 peptide |
|---|---|
| 59 | ASELSTAQLGRLSAELHQLATLPRTETGSGSP |
| 40 | ASHLSTAQTQRLSAELHKLATLPRTETGSGSP |
| 53 | ASELSTAALGRLSAELHQLATLPRTETGSGSP |
| 2 | ASELSTAALGRLSAELHELATLPRTETGSGSP |

83. The GLP-1-/GlP-/amylin-receptor tri-agonist according to any one of the preceding embodiments, wherein the peptide Z1-L1-Z2 comprises or consists of an amino acid sequence selected from the group consisting of SEQ ID NO: 170 to 252.
84. The GLP-1-/GlP-/amylin-receptor tri-agonist according to any one of the preceding embodiments, wherein peptide Z1-L1-Z2 comprises or consists of an amino acid sequence selected from the group consisting of:

(SEQ ID NO: 230)
YX₂EGTFTSDYSKLLEEIAAREFIEWLLAGGPSSGAGASELSTAQLGRL
SAELHQLATLPRTETGSGSP,
or
(SEQ ID NO: 243)
YX₂EGTFTSDYSILLEEIAAREFIEWLLAGGASSGAGEAPGEAPGASHL
STAQTQRLSAELHKLATLPRTETGSGSP,
or
(SEQ ID NO: 244)
YX₂EGTFTSDYSKLLEEIAAREFIEWLLAGGPSSGAGASELSTAALGRL
SAELHQLATLPRTETGSGSP,
or
(SEQ ID NO: 246)
YX₂EGTFTSDYSKLLEEIAAREFIEWLIAGAPSSGAGASELSTAALGRL
SAELHQLATLPRTETGSGSP,
or
(SEQ ID NO: 250)
YX₂EGTFTSDYSKLLEEIAAREFIEWLIAGAPSSGAGASELSTAALGRL
SAELHELATLPRTETGSGSP,
or
(SEQ ID NO: 251)
YX₂EGTFTSDYSKLLEEIAAREFIEWLLAGGGSSGAGASELSTAALGRL
SAELHELATLPRTETGSGSP,
or
(SEQ ID NO: 252)
YX₂EGTFTSDYSKLLEEIAAREFIEWLLAGGPSSGAGASELSTAALGRL
SAELHELATLPRTETGSGSP, wherein in each amino acid
X₂ is Aib.

85. The GLP-1-/GlP-/amylin-receptor tri-agonist according to any one of the preceding embodiments, wherein the backbone of the peptide Z1-L1-Z2 comprises or consists of 66 to 80 amino acid residues, such as 66 to 76 amino acid residues or 67 to 75 amino acid residues.
86. The GLP-1-/GlP-/amylin-receptor tri-agonist according to any one of the preceding embodiments, wherein the backbone of the peptide Z1-L1-Z2 comprises or consists of 67, 68, 71, 75, or 76 amino acid residues.
87. The GLP-1-/GlP-/amylin-receptor tri-agonist according to any one of the preceding embodiments, wherein the backbone of the peptide Z1-L1-Z2 comprises or consists of 68 amino acid residues.
88. The GLP-1-/GlP-/amylin-receptor tri-agonist according to any one of the preceding embodiments, wherein the backbone of the peptide Z1-L1-Z2 comprises or consists of 76 amino acid residues.
89. The GLP-1-/GlP-/amylin-receptor tri-agonist according to any one of the preceding embodiments, wherein the peptide Z1-L1-Z2 is a peptide derivative comprising a protraction moiety.
90. The GLP-1-/GlP-/amylin-receptor tri-agonist according to any one of the preceding embodiments, wherein the peptide Z1-L1-Z2 is a peptide derivative comprising a protraction moiety, wherein said protraction moiety comprises a protractor P being a C₁₂-C₂₀ diacid.
91. The GLP-1-/GlP-/amylin-receptor tri-agonist according to any one of the preceding embodiments, wherein the peptide Z1-L1-Z2 is a peptide derivative comprising a protraction moiety, wherein said protraction moiety comprises a protractor P selected from the group consisting of:

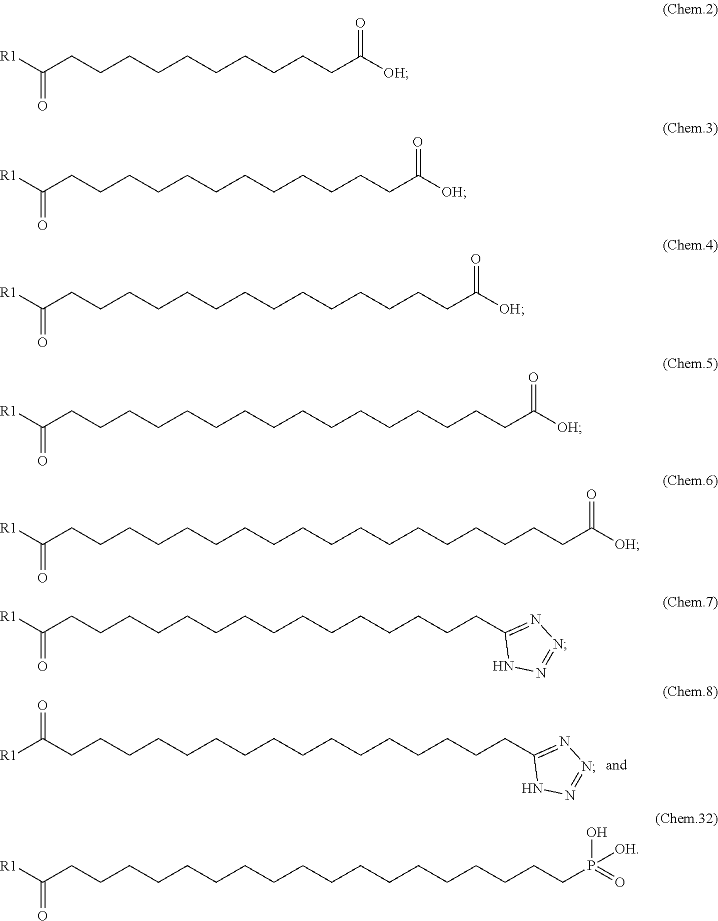

92. The GLP-1-/GlP-/amylin-receptor tri-agonist according to any one of the preceding embodiments, wherein the peptide Z1-L1-Z2 is a peptide derivative comprising a protraction moiety, wherein the protraction moiety comprises a protractor P selected from the group consisting of C₁₈ diacid, C₂₀ diacid, and C₁₉ phosphonic acid; preferably the protractor P is a C₂₀ diacid (Chem. 6).
93. The GLP-1-/GlP-/amylin-receptor tri-agonist according to any one of the preceding embodiments, wherein the peptide Z1-L1-Z2 is a peptide derivative comprising a protraction moiety, wherein the protraction moiety is attached to the epsilon position of the one lysine (Lys, K) residue.
94. The GLP-1-/GlP-/amylin-receptor tri-agonist according to any one of the preceding embodiments, wherein the peptide Z1-L1-Z2 is a peptide derivative comprising a protraction moiety, wherein the protraction moiety is attached to the epsilon amino group of the one lysine (Lys, K) residue in the peptide Z1 or to the epsilon amino group of the lysine (Lys, K) residue in the peptide Z2.
95. The GLP-1-/GlP-/amylin-receptor tri-agonist according to any one of the preceding embodiments, wherein the peptide Z1-L1-Z2 is a peptide derivative comprising a protraction moiety, wherein the protraction moiety is attached to the epsilon amino group of the one lysine (Lys, K) at position 12 of peptide Z1 (X₁₂).
96. The GLP-1-/GlP-/amylin-receptor tri-agonist according to any one of the preceding embodiments, wherein the peptide Z1-L1-Z2 is a peptide derivative comprising a protraction moiety, wherein the protraction moiety is attached to the epsilon amino group of the one lysine (Lys, K) at position 18 of peptide Z2 (X₆₈).
97. The GLP-1-/GlP-/amylin-receptor tri-agonist according to any one of the preceding embodiments, wherein the peptide Z1-L1-Z2 is a peptide derivative comprising a protraction moiety, wherein said protraction moiety further comprises a linker L_{P} selected from the group consisting of:

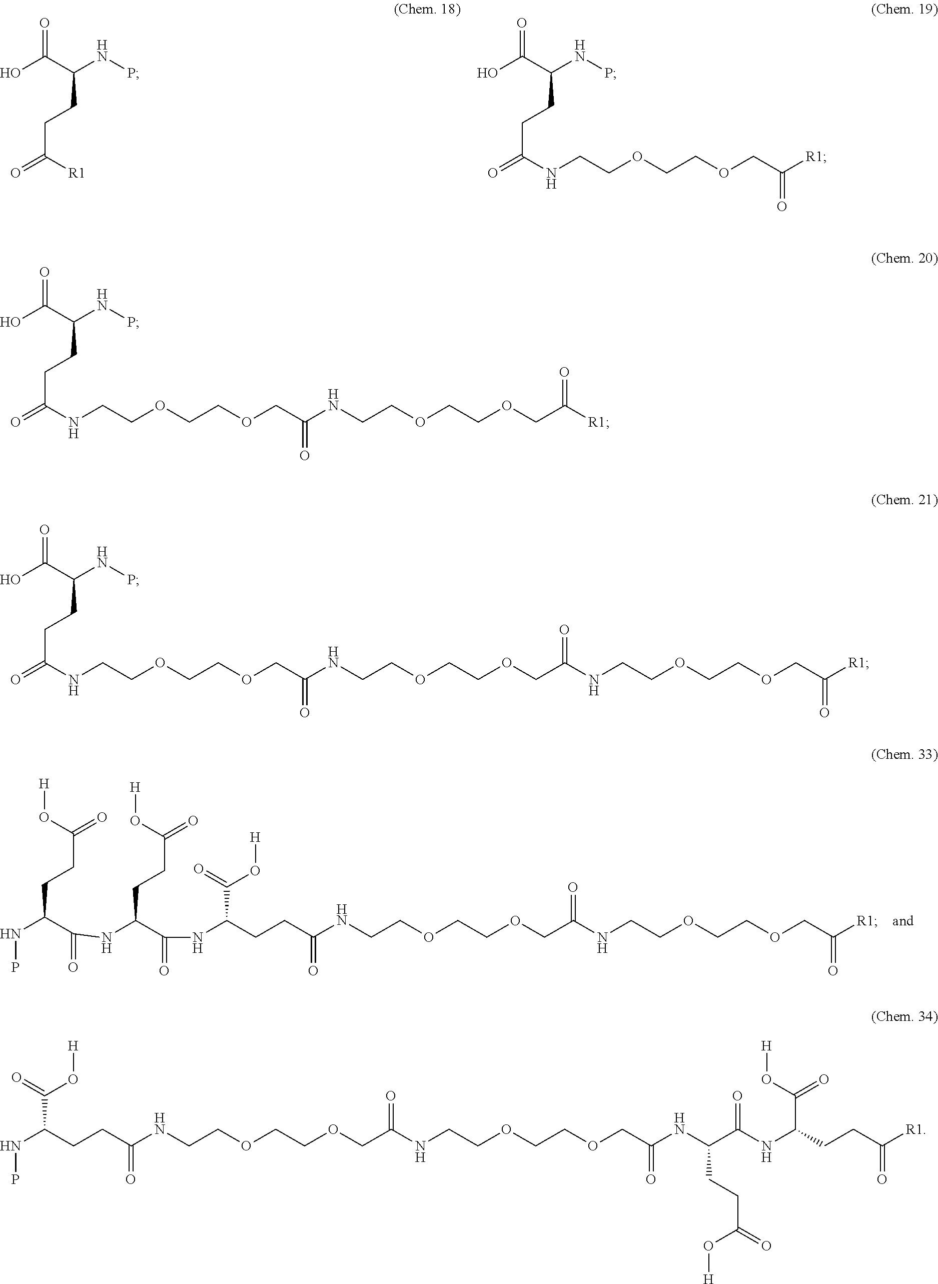

98. The GLP-1-/GlP-/amylin-receptor tri-agonist according to any one of the preceding embodiments, wherein the peptide Z1-L1-Z2 is a peptide derivative comprising a protraction moiety, wherein said protraction moiety further comprises a linker L_{P} selected from the group consisting of: Chem. 20, Chem. 33, and Chem. 34, preferably the linker L_{P} is Chem. 20.
99. The GLP-1-/GlP-/amylin-receptor tri-agonist according to any one of the preceding embodiments, wherein the peptide Z1-L1-Z2 is a peptide derivative comprising a protraction moiety, wherein said protraction moiety is selected from the group consisting of:

Structure

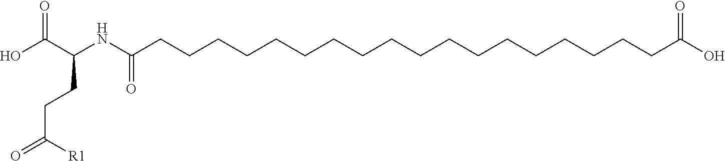

(Chem. 22)

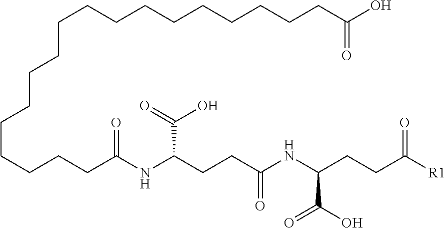

(Chem. 23)

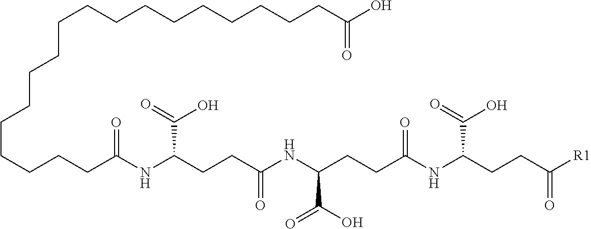

(Chem. 24)

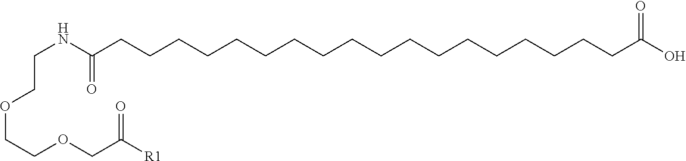

(Chem. 25)

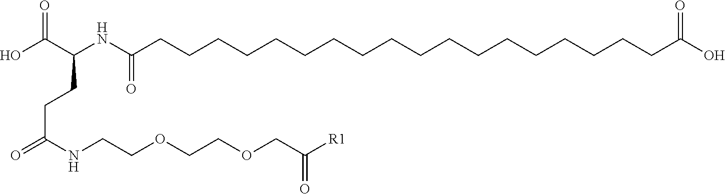

(Chem. 26)

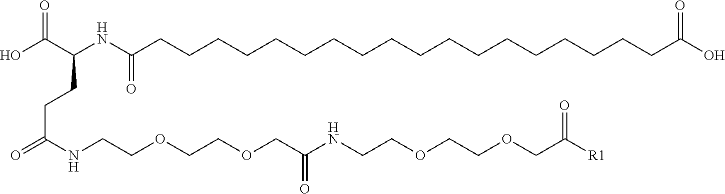

(Chem. 27)

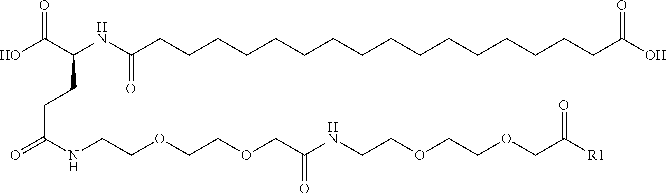

(Chem. 28)

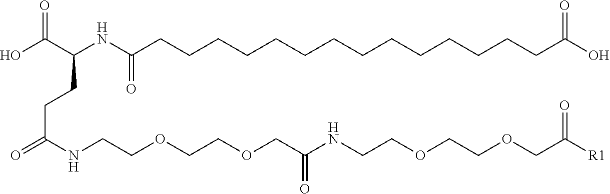

(Chem. 29)

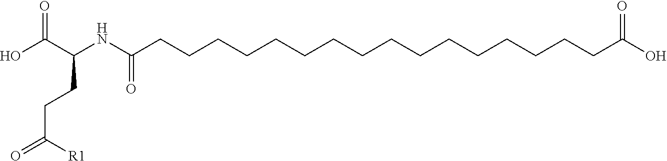

(Chem. 30)

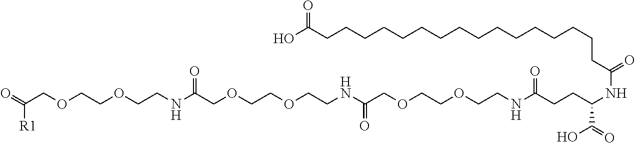

(Chem. 31)

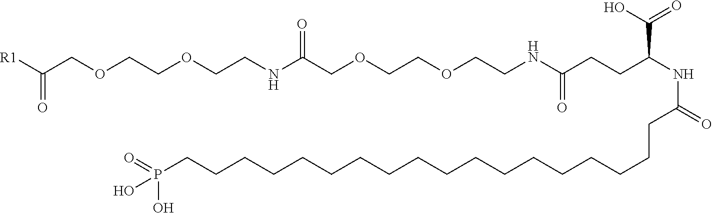

(Chem. 35)

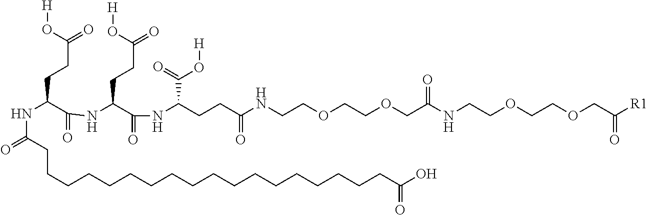

(Chem. 36)

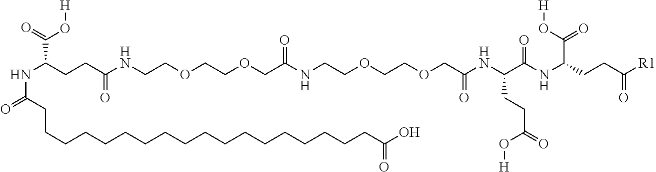

(Chem. 37)

wherein R1 is the point of attachment an epsilon amino group of a lysine (Lys, K).
100. The GLP-1-/GlP-/amylin-receptor tri-agonist according to any one of the preceding embodiments, wherein the peptide Z1-L1-Z2 is a peptide derivative comprising a protraction moiety, wherein said protraction moiety is selected from the group consisting of: Chem. 27, Chem. 35, Chem. 36, and Chem. 37.
101. The GLP-1-/GlP-/amylin-receptor tri-agonist according to any one of the preceding embodiments, wherein the peptide Z1-L1-Z2 is a peptide derivative comprising a protraction moiety, wherein said protraction moiety is Chem. 27.
102. The GLP-1-/GlP-/amylin-receptor tri-agonist according to any one of the preceding embodiments, wherein the peptide Z1-L1-Z2 is a peptide derivative selected from the group consisting of compound no.'s 104-197 of Example 2 herein.
103. The GLP-1-/GlP-/amylin-receptor tri-agonist according to any one of the preceding embodiments, wherein the peptide Z1-L1-Z2 is a peptide derivative selected from the group consisting of compound no.'s 104-193 and 196-197 of Example 2 herein.
104. The GLP-1-/GlP-/amylin-receptor tri-agonist according to any one of the preceding embodiments which is Compound 104 of Example 2 herein

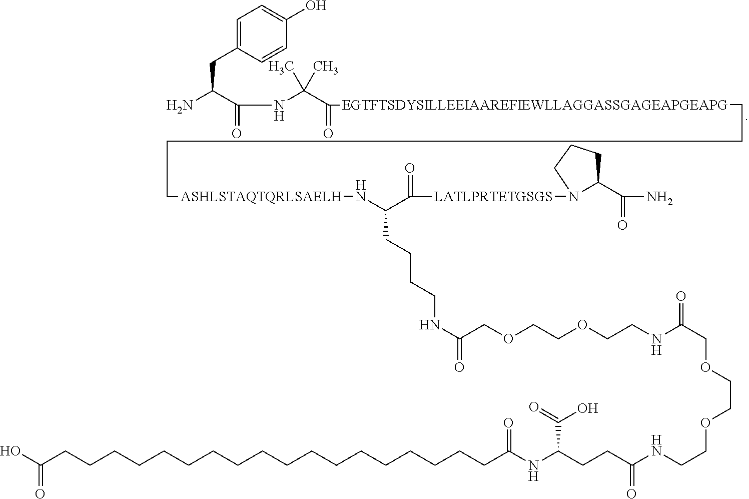

105. The GLP-1-/GlP-/amylin-receptor tri-agonist according to any one of embodiments 1-103 which is Compound 105 of Example 2 herein:

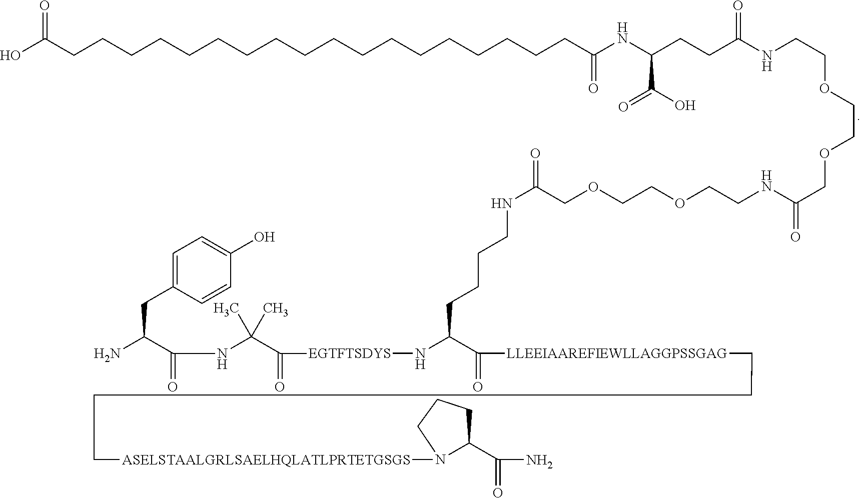

106. The GLP-1-/GlP-/amylin-receptor tri-agonist according to any one of embodiments 1-103 which is Compound 107 of Example 2 herein:

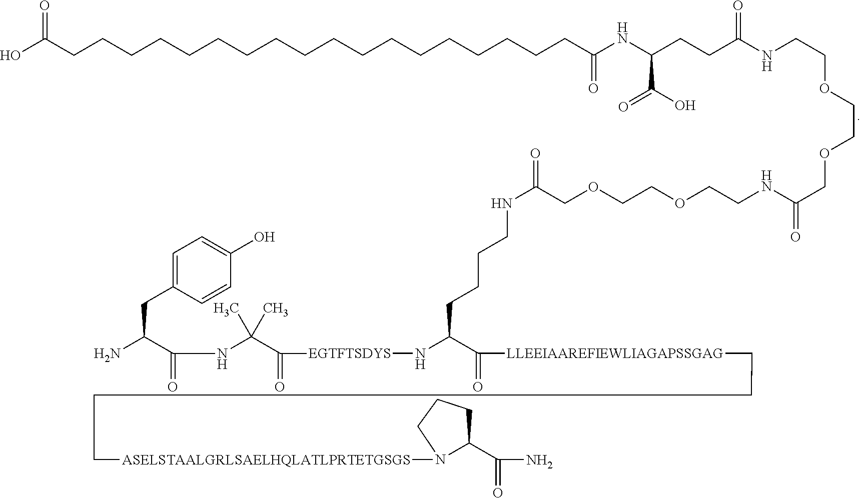

107. The GLP-1-/GlP-/amylin-receptor tri-agonist according to any one of embodiments 1-103 which is Compound 111 of Example 2 herein:

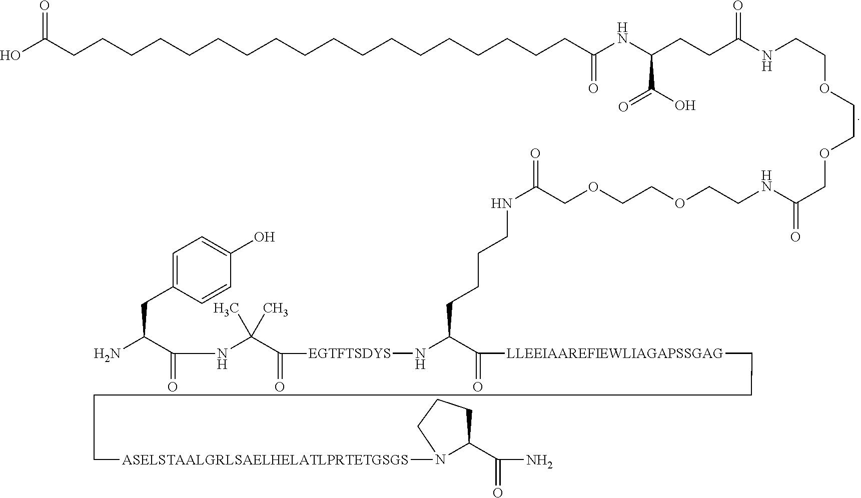

108. The GLP-1-/GlP-/amylin-receptor tri-agonist according to any one of embodiments 1-103 which is Compound 112 of Example 2 herein:

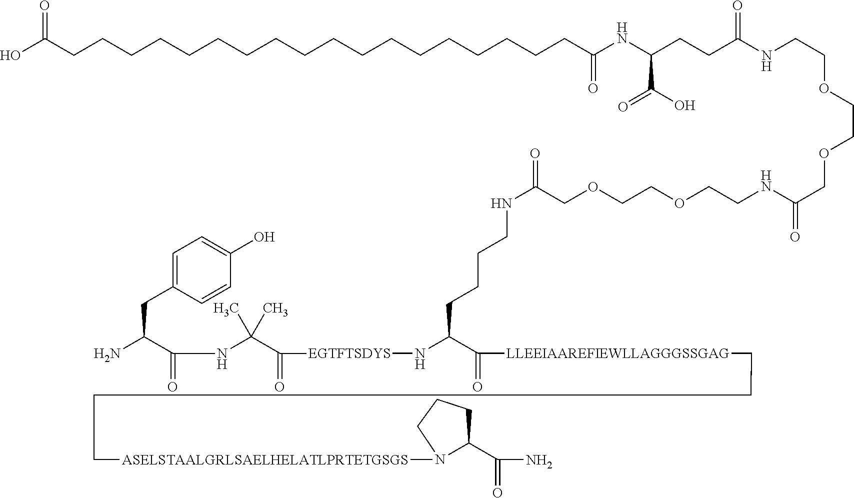

109. The GLP-1-/GlP-/amylin-receptor tri-agonist according to any one of embodiments 1-103 which is Compound 113 of Example 2 herein:

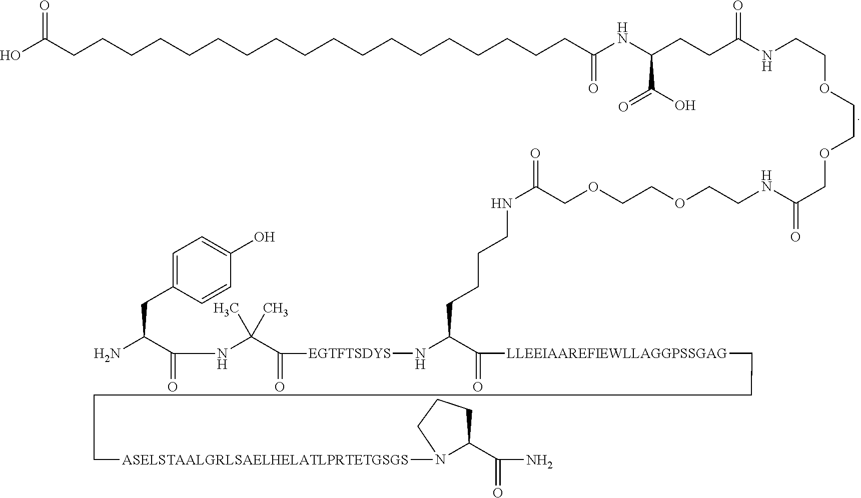

110. The GLP-1-/GlP-/amylin-receptor tri-agonist according to any one of embodiments 1-103 which is Compound 183 of Example 2 herein:

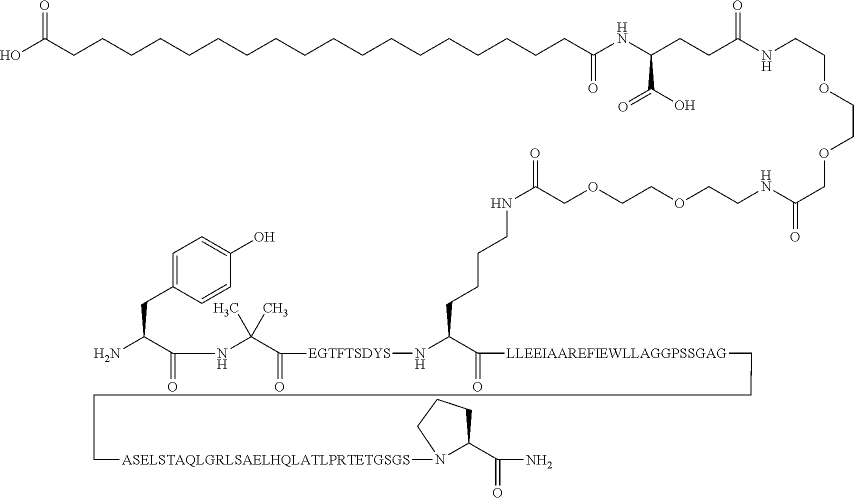

111. The GLP-1-/GlP-/amylin-receptor tri-agonist according to any one of the preceding embodiments wherein the peptide has the amide modification of the C-terminus.
112. The GLP-1-/GlP-/amylin-receptor tri-agonist according to any one of the preceding embodiments which is capable of activating the human GlP receptor.
113. The GLP-1-/GlP-/amylin-receptor tri-agonist according to any one of the preceding embodiments which is capable of activating the human GlP receptor in an assay with whole cells expressing the human GlP receptor.
114. The GLP-1-/GlP-/amylin-receptor tri-agonist according to any one of the preceding embodiments which activates the human GlP receptor in vitro, preferably with an EC₅₀ of less than 125 pM, even more preferably with an EC₅₀ of less than 100 pM, and most preferably with an EC₅₀ of less than 50 pM, when measured without HSA in the assay as described in Example 4.
115. The GLP-1-/GlP-/amylin-receptor tri-agonist according to any one of the preceding embodiments which is capable of activating the human GLP-1 receptor.
116. The GLP-1-/GlP-/amylin-receptor tri-agonist according to any one of the preceding embodiments which is capable of activating the human GLP-1 receptor in an assay with whole cells expressing the human GLP-1 receptor.
117. The GLP-1-/GlP-/amylin-receptor tri-agonist according to any one of the preceding embodiments which activates the human GLP-1 receptor in vitro, preferably with an EC₅₀ of less than 125 pM, even more preferably with an EC₅₀ of less than 100 pM, and most preferably with an EC₅₀ of less than 50 pM, when measured without HSA in the assay as described in Example 4.
118. The GLP-1-/GlP-/amylin-receptor tri-agonist according to any one of the preceding embodiments which is capable of activating the human amylin receptor.
119. The GLP-1-/GlP-/amylin-receptor tri-agonist according to any one of the preceding embodiments which is capable of activating the human amylin receptor in an assay with whole cells expressing the human amylin receptor.
120. The GLP-1-/GlP-/amylin-receptor tri-agonist according to any one of the preceding embodiments which activates the human amylin receptor in vitro, preferably with an EC₅₀ of less than 125 pM, even more preferably with an EC₅₀ of less than 100 pM, and most preferably with an EC₅₀ of less than 50 pM, when measured without HSA in the assay as described in Example 4.
121. The GLP-1-/GlP-/amylin-receptor tri-agonist according to any one of the preceding embodiments which is capable of activating the human GlP, GLP-1, and amylin receptors.
122. The GLP-1-/GlP-/amylin-receptor tri-agonist according to any one of the preceding embodiments which is capable of activating the human GlP, GLP-1, and amylin receptors in assays with whole cells expressing the human GlP receptor, GLP-1, and amylin receptors.
123. The GLP-1-/GlP-/amylin-receptor tri-agonist according to any one of the preceding embodiments which activates the human GlP, GLP-1 and amylin receptors in vitro, preferably with an EC₅₀ of less than 125 pM, even more preferably with an EC₅₀ of less than 100 pM, and most preferably with an EC₅₀ of less than 50 pM, when measured without HSA as described in Example 4.
124. The GLP-1-/GlP-/amylin-receptor tri-agonist according to any one of the preceding embodiments, which activates the human GlP, GLP-1 and amylin receptors in vitro, when measured without HSA as described in Example 4, and which has a potency ratio of less than 50, such as less than 30.
125. The GLP-1-/GlP-/amylin-receptor tri-agonist according to any one of the preceding embodiments, wherein the potency ratio is less than 20.
126. The GLP-1-/GlP-/amylin-receptor tri-agonist according to any one of the preceding embodiments, wherein the potency ratio is less than 15, and most preferred less than 10.
127. The GLP-1-/GlP-/amylin-receptor tri-agonist according to any one of the preceding embodiments which has improved pharmacokinetic properties.
128. The GLP-1-/GlP-/amylin-receptor tri-agonist according to any one of the preceding embodiments which has an increased half-life.
129. The GLP-1-/GlP-/amylin-receptor tri-agonist according to any one of the preceding embodiments, which has an increased half-life of 80-200 hours, when determined in minipigs as described in Example 6, preferably of 90-190 hours, even more preferably 95-185 hours.
130. The GLP-1-/GlP-/amylin-receptor tri-agonist according to any one of the preceding embodiments, which has an increased half-life of 40 hours to 145 hours, when determined in minipigs as described in Example 6, preferably of 90 hours to 140 hours, even more preferably 85 hours to 125 hours.
131. The GLP-1-/GlP-/amylin-receptor tri-agonist according to any one of the preceding embodiments, which has an increased half-life of 5 hours to 50 hours, when determined in rats as described in Example 6, preferably 10 hours to 35 hours, even more preferably 12 hours to 30 hours.
132. The GLP-1-/GlP-/amylin-receptor tri-agonist according to any one of the preceding embodiments, which has improved chemical stability.
133. The GLP-1-/GlP-/amylin-receptor tri-agonist according to any one of the preceding embodiments, which has improved chemical stability and which has a purity loss of no more than 5.0 percent per week, such as determined in Example 7 described herein, preferably a purity loss of less than 3.0 percent per week, even more preferably a purity loss of less than 2.0 percent per week.
134. The GLP-1-/GlP-/amylin-receptor tri-agonist according to any one of the preceding embodiments which has the effect in vivo of reducing food intake in normal weight rats, as determined in the experimental protocol for efficacy testing on appetite, such as Example 5 described herein.
135. The GLP-1-/GlP-/amylin-receptor tri-agonist according to any one of the preceding embodiments which is a pharmaceutically acceptable salt of the GLP-1-/GlP-/amylin-receptor triple agonist.
136. A pharmaceutical composition comprising a GLP-1-/GlP-/amylin-receptor triple agonist according to any one of the preceding embodiments, and one or more pharmaceutically acceptable excipients.
137. The pharmaceutical composition according to embodiment 136, which is for oral or for subcutaneous administration.
138. The pharmaceutical composition according to any one of the embodiments 136 or 137, which is a solid pharmaceutical composition.
139. The solid pharmaceutical composition according to embodiment 138, which is a tablet.
140. The solid pharmaceutical composition according to any one of embodiments 138 or 139, comprising a salt of N-[8-(2-hydroxybenzoyl)amino]caprylate, preferably sodium N-(8-(2-hydroxybenzoyl)amino)caprylate, and magnesium stearate.
141. The solid pharmaceutical composition according to any one of the embodiments 138 to 140, comprising 75-600 mg sodium N-(8-(2-hydroxybenzoyl)amino)caprylate and 7-8.5 mg magnesium stearate.
142. The pharmaceutical composition according to any one of embodiments 136 to 141, which is for dosing approximately once daily, such as once every 12-36 hours, such as once every 18-30 hours, such as approximately once every 24 hours; or which is for dosing approximately once weekly, such as once every 6-8 days.
143. An injection device comprising the GLP-1-/GlP-/amylin-receptor tri-agonist according to any one of embodiments 1 to 135, or the pharmaceutical composition according to any one of embodiments 136 to 142.
144. The GLP-1-/GlP-/amylin-receptor tri-agonist peptide according to any of embodiments 1 to 88 for use as an intermediate in the manufacture of a GLP-1-/GlP-/amylin-receptor tri-agonist peptide derivative according to any of embodiments 89 to 135.
145. The GLP-1-/GlP-/amylin-receptor triple agonist according to any one of embodiments 1 to 135 or the pharmaceutical composition according to any one of embodiments 136 to 142 for use as a medicament.
146. The GLP-1-/GlP-/amylin-receptor triple agonist according to any one of embodiments 1 to 135 or the pharmaceutical composition according to any one of embodiments 136 to 142 for use in the treatment of type 2 diabetes, obesity, metabolic dysfunction-associated steatohepatitis (MASH), and/or cardiovascular disease.
147. The GLP-1-/GlP-/amylin-receptor triple agonist according to any one of embodiments 1 to 135 or the pharmaceutical composition according to any one of embodiments 136 to 142, for use in the treatment of a subject with an initial body mass index (BMI) of 25 or more, 27 or more, or 28 or more, or 30 or more.
148. The GLP-1-/GlP-/amylin-receptor triple agonist according to any one of embodiments 1 to 135 or the pharmaceutical composition according to any one of embodiments 136 to 142, for use in the treatment of a subject with an initial body mass index (BMI) of 25 or more, 27 or more, or 28 or more, 30 or more; and in the presence of at least one weight-related co-morbidity.
149. The GLP-1-/GlP-/amylin-receptor triple agonist according to any one of embodiments 1 to 135 or the pharmaceutical composition according to any one of embodiments 136 to 142, for use as an adjunct to a reduced-calorie diet and increased physical activity for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more.
150. The GLP-1-/GlP-/amylin-receptor triple agonist according to any one of embodiments 1 to 135 or the pharmaceutical composition according to any one of embodiments 136 to 124, for use as an adjunct to a reduced-calorie diet and increased physical activity for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more; and in the presence of at least one weight-related co-morbidity.
151. The use according to embodiment 148 or embodiment 150, wherein the at least one weight-related co-morbidity is selected from the group consisting of hypertension, dysglycemia (prediabetes or type 2 diabetes), dyslipidaemia, high cholesterol, cardiovascular disease and obstructive sleep apnoea.
152. Use of a GLP-1-/GlP-/amylin-receptor triple agonist according to any one of embodiments 1 to 135 or the pharmaceutical composition according to any one of embodiments 136 to 142, in the manufacture of a medicament for the treatment of type 2 diabetes, obesity, metabolic dysfunction-associated steatohepatitis (MASH), and/or cardiovascular disease.
153. Use of a GLP-1-/GlP-/amylin-receptor triple agonist according to any one of embodiments 1 to 135 or the pharmaceutical composition according to any one of embodiments 136 to 142, in the manufacture of a medicament for the treatment of a subject with an initial body mass index (BMI) of 25 or more, 27 or more, or 28 or more, 30 or more; and optionally in the presence of at least one weight-related co-morbidity.
154. Use of a GLP-1-/GlP-/amylin-receptor triple agonist according to any one of embodiments 1 to 135 or the pharmaceutical composition according to any one of embodiments 136 to 142, in the manufacture of a medicament for the treatment of an adult subject suffering from obesity, i.e. with an initial body mass index (BMI) of 25 or more, or 27 or more, or 28 or more, 30 or more; or an adult subject suffering from overweight, i.e. with an initial body mass index (BMI) of 23 or more, or 24 or more, or 25 or more, or 27 or more; and optionally in the presence of at least one weight-related co-morbidity.
155. Use of a GLP-1-/GlP-/amylin-receptor triple agonist according to any one of embodiments 1 to 135 or the pharmaceutical composition according to any one of embodiments 136 to 142, in the manufacture of a medicament for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more; and optionally in the presence of at least one weight-related co-morbidity.
156. The use according to embodiment 154 or embodiment 155, wherein the medicament is an adjunct to a reduced-calorie diet and increased physical activity for chronic weight management in an adult subject suffering from obesity or overweight.
157. A method for treating type 2 diabetes, obesity, metabolic dysfunction-associated steatohepatitis (MASH), and/or cardiovascular disease comprising administering a pharmaceutically relevant amount of a GLP-1-/GlP-/amylin-receptor triple agonist according to any one of embodiments 1 to 135 to a subject in need thereof.
158. A method of treating a human subject with an initial body mass index (BMI) of 25 or more, 27 or more, or 28 or more, or 30 or more, comprising administering to said human subject a pharmaceutically relevant amount of a GLP-1-/GlP-/amylin-receptor triple agonist according to any one of embodiments 1 to 135.
159. A method for reducing excess body weight in a human subject, in combination with a reduced-calorie diet and increased physical activity, comprising administering to said human subject a pharmaceutically relevant amount of a GLP-1-/GlP-/amylin-receptor triple agonist according to any one of embodiments 1 to 135.
160. The method according to embodiment 158 or embodiment 159, wherein said human subject is an adult subject, suffering from overweight and has an initial body mass index (BMI) of 23 or more, or 24 or more, or 25 or more, or 27 or more.
161. The method according to embodiment 158 or embodiment 159, wherein said human subject is an adult subject, suffering from obesity and has an initial body mass index (BMI) of 25 or more, or 27 or more, or 28 or more, 30 or more.
162. The method according to any one of embodiments 158 to 161, wherein said human subject has at least one weight-related comorbidity selected from the group consisting of hypertension, dysglycemia (prediabetes or type 2 diabetes), dyslipidaemia, high cholesterol, cardiovascular disease and obstructive sleep apnoea.
163. A method for preparing the GLP-1-/GlP-/amylin-receptor triple agonist according to any one of embodiments 1 to 135.
164. The method according to embodiment 163, comprises a step of solid phase peptide synthesis.

### PARTICULAR ALTERNATIVE EMBODIMENTS

1. A GLP-1-/GlP-/amylin-receptor tri-agonist comprising a peptide according to Formula I:

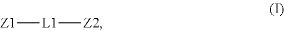

comprising one lysine (Lys, K) residue; wherein:
   Z1 is a peptide comprising a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib;
   L1 is a peptide linker; and
   Z2 is a peptide comprising a C-terminal amide and a maximum of 10 amino acid substitutions relative to Formula V (SEQ ID NO: 2):

(V)
ASELSTAALGRLSAELHELATLPRTETGSGSP.

2. The GLP-1-/GlP-/amylin-receptor tri-agonist according to alternative embodiment 1, wherein peptide Z1 comprises an amino acid sequence which has at least 87, 88, 89, 90, 91, 92, 93, 94, 95, 96, 97, 98 or 99% identity to Formula II (SEQ ID NO: 1), and peptide Z2 comprises an amino acid sequence which has at least 70, 71, 72, 73, 74, 75, 76, 77, 78, 79, 80, 81, 82, 83, 84, 85, 86, 87, 88, 89 90, 91, 92, 93, 94, 95, 96, 97, 98 or 99% identity relative to Formula V (SEQ ID NO: 2).
3. The GLP-1-/GlP-/amylin-receptor tri-agonist according to alternative embodiments 1 to 2, wherein the one lysine (Lys, K) residue is present in the peptide Z1 or in the peptide Z2.
4. The GLP-1-/GlP-/amylin-receptor tri-agonist according to alternative embodiments 1 to 3, wherein the GLP-1-/GlP-/amylin-receptor triple agonist does not comprise a cysteine (Cys, C) residue.
5. The GLP-1-/GlP-/amylin-receptor tri-agonist according to alternative embodiments 1 to 4, wherein the GLP-1-/GlP-/amylin-receptor triple agonist does not comprise a disulfide bridge.
6. The GLP-1-/GlP-/amylin-receptor tri-agonist according to alternative embodiments 1 to 5, wherein:
   Z1 is a peptide having a maximum of 4 amino acid substitutions relative to Formula II (SEO ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula VII (SEQ ID NO: 162):

(VII)
YX₂EGTFTSDYSX₁₂LLEEIAAX₂₀EFIX₂₄WLX₂₇X₂₈GGPSSX₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₄ represents Gly (G) or Glu (E);
L1 is a peptide linker; and
Z2 is a peptide comprising a C-terminal amide, and having a maximum of 10 amino acid substitutions relative to Formula V (SEQ ID NO: 2):

(V)
ASELSTAALGRLSAELHELATLPRTETGSGSP,

and
Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E).
7. The GLP-1-/GlP-/amylin-receptor tri-agonist according to any one of alternative embodiments 1 to 6, wherein the peptide linker L1 comprises 1 to 14, 1 to 10, 4 to 10 or 9 to 10 amino acid residues.
8. The GLP-1-/GlP-/amylin-receptor tri-agonist according to any one of alternative embodiments 1 to 7, wherein the peptide linker L1 comprises 1 to 14 amino acid residues selected from the group consisting of Ala (A), Glu (E), Gln (Q), Gly (G), Leu (L), Phe (F), Pro (P), Ser (S), Thr (T), Val (V), Asn (N).
9. The GLP-1-/GlP-/amylin-receptor tri-agonist according to any one of alternative embodiments 1 to 8, wherein the peptide linker L1 comprises or consists of the amino acid sequence according to Formula VIIIa:

(VIIIa)
X₁₄₁X₁₄₂X₁₄₃X₁₄₄X₁4₅X₁₄₆X₁₄₇X₁₄₈X₁₄₉X₁₅₀X₁₅₁X₁₅₂X₁₅₃X₁₅₄,

wherein
X₁₄₁ represents Ala (A), Glu (E), Gly (G),
X₁₄₂ represents Gln (Q), Glu (E), Gly (G), Leu (L), Pro (P) or is absent,
X₁₄₃ represents Ala (A), Gln (Q), Glu (E), Gly (G), Pro (P) or absent,
X₁₄₄ represents Ala (A), Gln (Q), Glu (E), Gly (G), Pro (P) or absent,
X₁₄₅ represents Glu (E), Gly (G), Pro (P), Ser (S), Thr (T) or is absent,
X₁₄₆ represents Glu (E), Gly (G), Leu (L), Gln (Q) or is absent,
X₁₄₇ represents Ala (A), Gln (Q), Glu (E), Gly (G), Phe (F) or is absent,
X₁₄₈ represents Ala (A), Gln (Q), Glu (E), Gly (G), Thr (T), Pro (P), Val (V) or is absent,
X₁₄₉ represents Glu (E), Asn (N), Pro (P), Thr (T) or is absent,
X₁₅₀ represents Ala (A), Gln (Q), Glu (E), Gly (G), Leu (L), Pro (P), Ser (S), Val (V) or is absent,
X₁₅₁ represents Ala (A) or is absent,
X₁₅₂ represents Gln (Q) or is absent,
X₁₅₃ represents Thr (T) or is absent,
X₁₅₄ represents Leu (L) or is absent.
10. The GLP-1-/GlP-/amylin-receptor tri-agonist according to any one of alternative embodiments 1 to 9,
   wherein the peptide linker L1 comprises or consists of the amino acid sequence according selected from the group consisting of A, E, G, AE, AG, GE, APPE (SEQ ID NO: 125), GGGE (SEQ ID NO: 126), AGQAPG (SEQ ID NO: 127), APPPSGGG (SEQ ID NO: 128), APPPSGGGE (SEQ ID NO: 129), APPPSGGGG (SEQ ID NO: 130), ALAQTLAQTL (SEQ ID NO: 131), ALAQTLFVNQ (SEQ ID NO: 132), ALAQTLGTNE (SEQ ID NO: 133), ALQAPGQAPG (SEQ ID NO: 134), ALQAPGQAPL (SEQ ID NO: 135), AGQAPGQAPG (SEQ ID NO: 136), AGQAPGQAPL (SEQ ID NO: 137), GGGEGGGEGE (SEQ ID NO: 138), GQAPGQAPGE (SEQ ID NO: 139), GQEPGQEPGE (SEQ ID NO: 140), APPPSLAQTLAQTL (SEQ ID NO: 141), AGGGG (SEQ ID NO: 142), AGEAPGQAPG (SEQ ID NO: 143), AGEAPGEAPG (SEQ ID NO: 144), AGQAPGQAPA (SEQ ID NO: 145), AGQAPGQAPE (SEQ ID NO: 146), AGQAPGQAPP (SEQ ID NO: 147), AGQAPGQAPS (SEQ ID NO: 148), AGQAPGQAPV (SEQ ID NO: 149), EGQAPGQAPG (SEQ ID NO: 150), AGQEPGQAPG (SEQ ID NO: 151), AGQAEGQAPG (SEQ ID NO: 152), AGQAPEQAPG (SEQ ID NO: 153), AGQAPGEAPG (SEQ ID NO: 154), AGQAPGQEPG (SEQ ID NO: 155), AGQAPGQAEG (SEQ ID NO: 156), AGQEPGQEPG (SEQ ID NO: 157), AGQAPGQAP (SEQ ID NO: 158) and AGQAPGEAPL (SEQ ID NO: 159).
11. The GLP-1-/GlP-/amylin-receptor tri-agonist according to alternative embodiment 10, wherein the peptide linker L1 is selected from the group consisting of E, GE, APPPSGGGE (SEQ ID NO: 129), AGQAPGQAPG (SEQ ID NO: 136) AGQAPGQAPL (SEQ ID NO: 137), and AGQAPGEAPG (SEQ ID NO: 154).
12. The GLP-1-/GlP-/amylin-receptor tri-agonist according to any one of alternative embodiments 1 to 5 and 7 to 11,
   wherein the peptide Z2 comprises or consists of the amino acid sequence according to Formula XIV (SEQ ID NO: 4):

(XIV)
ASX₅₃LSTAX₅₈X₅₉X₆₀RLSAX₆₅LHX₆₈LX₇₀X₇₁LPX₇₄TETGSGX₈₁P,

wherein
X₅₃ represents Lys (K),
X₅₈ represents Ala (A) or Gln (Q),
X₅₉ represents Gln (Q), Leu (L) or Thr (T),
X₆₀ represents Ala (A), Gly (G) or Gln (Q),
X₆₅ represents Glu (E),
X₆₈ represents Arg (R), Gln (Q), Glu (E), Gly (G), His (H), Thr (T) or Tyr (Y),
X₇₀ represents Ala (A),
X₇₁ represents Asp (D) or Thr (T),
X₇₄ represents Arg (R),
X₈₁ represents Ala (A) or Ser (S);
or
X₅₃ represents Gln (Q), Glu (E), or His (H),
X₅₈ represents Ala (A) or Gln (Q),
X₅₉ represents Gln (Q), Leu (L) or Thr (T),
X₆₀ represents Ala (A), Gly (G) or Gln (Q),
X₆₅ represents Lys (K),
X₆₈ represents Arg (R), Gln (Q), Glu (E), Gly (G), His (H), Thr (T) or Tyr (Y),
X₇₀ represents Ala (A),
X₇₁ represents Asp (D) or Thr (T),
X₇₄ represents Arg (R),
X₈₁ represents Ala (A) or Ser (S);
or
X₅₃ represents Gln (Q), Glu (E) or His (H),
X₅₈ represents Ala (A) or Gln (Q),
X₅₉ represents Gln (Q), Leu (L) or Thr (T),
X₆₀ represents Ala (A), Gly (G) or Gln (Q),
X₆₅ represents Glu (E),
X₆₈ represents Lys (K),
X₇₀ represents Ala (A),
X₇₁ represents Asp (D) or Thr (T),
X₇₄ represents Arg (R),
X₈₁ represents Ala (A) or Ser (S);
or
X₅₃ represents Gln (Q), Glu (E) or His (H),
X₅₈ represents Ala (A) or Gln (Q),
X₅₉ represents Gln (Q), Leu (L) or Thr (T),
X₆₀ represents Ala (A), Gly (G) or Gln (Q),
X₆₅ represents Glu (E),
X₆₈ represents Arg (R), Gln (Q), Glu (E), Gly (G), His (H), Thr (T) or Tyr (Y),
X₇₀ represents Lys (K),
X₇₁ represents Asp (D) or Thr (T),
X₇₄ represents Arg (R),
X₈₁ represents Ala (A) or Ser (S);
or
X₅₃ represents Gln (Q), Glu (E) or His (H),
X₅₈ represents Ala (A) or Gln (Q),
X₅₉ represents Gln (Q), Leu (L) or Thr (T),
X₆₀ represents Ala (A), Gly (G) or Gln (Q),
X₆₅ represents Glu (E),
X₆₈ represents Arg (R), Gln (Q), Glu (E), Gly (G), His (H), Thr (T) or Tyr (Y),
X₇₀ represents Ala (A),
X₇₁ represents Asp (D) or Thr (T),
X₇₄ represents Lys (K),
X₈₁ represents Ala (A) or Ser (S).
13. The GLP-1-/GlP-/amylin-receptor tri-agonist according to alternative embodiment 6, wherein the peptide Z1 comprises or consists of the amino acid sequence according to Formula VX (SEQ ID NO: 167):

(VX)
YX₂EGTFTSDYSX₁₂LLEEIAAREFIEWLX₂₇AGGPSSX₃₄,

wherein
X₂ represents Aib,
X₁₂ represents Ile (I) or Lys (K),
X₂₇ represents Leu (L) or I (Ile),
X₃₄ represents Gly (G) or Glu (E).
14. The GLP-1-/GlP-/amylin-receptor tri-agonist according to alternative embodiment 6 or alternative embodiment 13, wherein the peptide Z2 comprises or consists of the amino acid sequence

(SEQ ID NO: 40)
ASHLSTAQTQRLSAELHKLATLPRTETGSGSP.,

15. The GLP-1-/GlP-/amylin-receptor tri-agonist according to any one of alternative embodiments 6, 13 or 14,
   wherein the peptide Z1 comprises or consists of the amino acid sequence according to Formula XV (SEQ ID NO: 167):

(XV)
YX₂EGTFTSDYSX₁₂LLEEIAAREFIEWLX₂₇AGGPSSX₃₄,

wherein
X₂ represents Aib,
X₁₂ represents Ile (I) or Lys (K),
X₂₇ represents Leu (L) or I (Ile),
X₃₄ represents Gly (G) or Glu (E); and
wherein the peptide Z2 comprises or consists of the amino acid sequence

(SEQ ID NO: 40)
ASHLSTAQTQRLSAELHKLATLPRTETGSGSP

16. The GLP-1-/GlP-/amylin-receptor tri-agonist according to any one of alternative embodiments 6 or 13-15,
   wherein the peptide Z1 comprises or consists of the amino acid sequence according to Formula XI (SEQ ID NO: 163):

(XI)
YX₂EGTFTSDYSX₁₂LLEEIAAREFIEWLLAGGPSSG,

wherein
   X₂ represents Aib,
   X₁₂ represents Ile (I) or Lys (K)
17. The GLP-1-/GlP-/amylin-receptor tri-agonist according to any one of alternative embodiments 6 or 13-16,
   wherein the peptide Z2 comprises or consists of the amino acid sequence according to Formula X (SEQ ID NO: 166):

(X)
ASX₅₉LSTAQTQRLSAELHKLATLPRTETGSGSP,

wherein
X₅₉ represents Glu (E) or His (H).
18. The GLP-1-/GlP-/amylin-receptor tri-agonist according to any one of alternative embodiments 1 to 5 and 7-11,
   wherein the peptide Z1-L1-Z2 comprises or consists of an amino acid sequence selected from the group consisting of SEQ ID NOs 170 to 242, wherein X₂ represents Aib.
19. The GLP-1-/GlP-/amylin-receptor tri-agonist according to any one of the preceding alternative embodiments wherein the backbone of the peptide Z1-L1-Z2 comprises 66 to 80 amino acid residues.
20. The GLP-1-/GlP-/amylin-receptor tri-agonist according to any one of the preceding alternative embodiments wherein the backbone of the peptide Z1-L1-Z2 comprises 67, 68, 75 or 76 amino acid residues, preferably 76 amino acid residues.
21. The GLP-1-/GlP-/amylin-receptor tri-agonist according to any one of the preceding alternative embodiments wherein the peptide is a peptide derivative comprising a protraction moiety.
22. The GLP-1-/GlP-/amylin-receptor tri-agonist according to any one of the preceding alternative embodiments wherein the peptide is a peptide derivative comprising a protraction moiety, wherein said protraction moiety comprises a protractor P being a C₁₂-C₂₀ diacid.
23. The GLP-1-/GlP-/amylin-receptor tri-agonist according to any one of the preceding alternative embodiments, wherein the peptide is a peptide derivative comprising a protraction moiety, wherein said protraction moiety comprise a protractor P selected from the group consisting of:

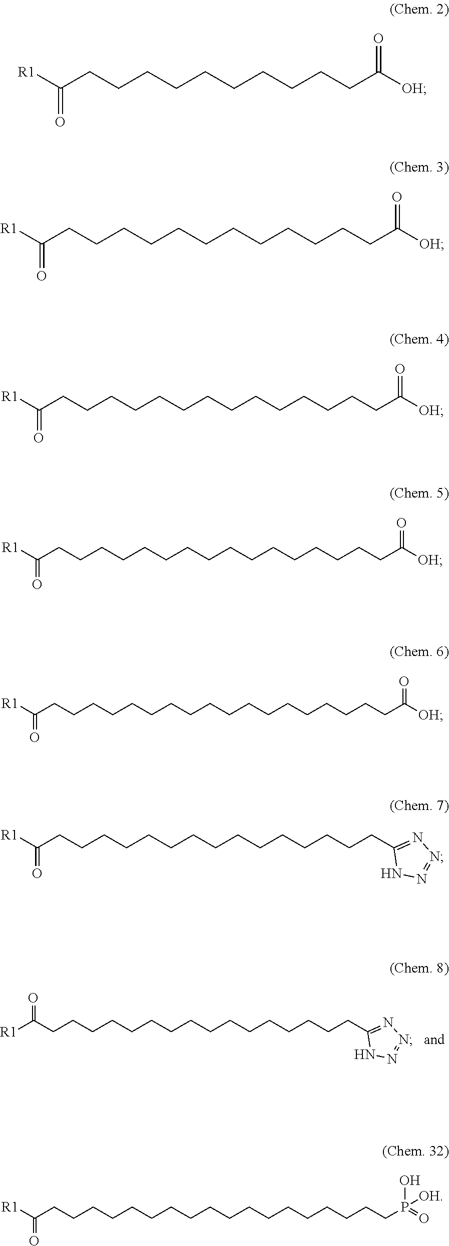

24. The GLP-1-/GlP-/amylin-receptor tri-agonist according to any one of the preceding alternative embodiments, wherein the peptide is a peptide derivative comprising a protraction moiety, wherein said protraction moiety comprises a protractor P selected from the group consisting of C₁₆ diacid, C₁₈ diacid, C₂₀ diacid, and C₁₉ phosphonic acid; preferably the protractor P is a C₁₈ diacid or a C₂₀ diacid.
25. The GLP-1-/GlP-/amylin-receptor tri-agonist according to any one of the preceding alternative embodiments, wherein the peptide is a peptide derivative comprising a protraction moiety, wherein the protraction moiety is attached to the epsilon position of the one lysine (Lys, K) residue.
26. The GLP-1-/GlP-/amylin-receptor tri-agonist according to any one of the preceding alternative embodiments, wherein the peptide is a peptide derivative comprising a protraction moiety, wherein the protraction moiety is attached to the epsilon position of the lysine (Lys, K) residue in the peptide Z1 or to the epsilon position of the lysine (Lys, K) residue in the peptide Z2.
27. The GLP-1-/GlP-/amylin-receptor tri-agonist according to any one of the preceding alternative embodiments, wherein the peptide is a peptide derivative comprising a protraction moiety, wherein the protraction moiety is attached to the epsilon position of the lysine (Lys, K) residue at position 12 or position 33 or position 34 of peptide Z1, preferably at position 12 or position 33 of peptide Z1.
28. The GLP-1-/GlP-/amylin-receptor tri-agonist according to any one of the preceding alternative embodiments, wherein the peptide is a peptide derivative comprising a protraction moiety, wherein the protraction moiety is attached to the epsilon position of the lysine (Lys, K) residue at positions 3, 15, 18, 20, or 24 of peptide Z2.
29. The GLP-1-/GlP-/amylin-receptor tri-agonist according to any one of the preceding alternative embodiments, wherein the peptide is a peptide derivative comprising a protraction moiety, wherein the protraction moiety is attached to the epsilon position of the lysine (Lys, K) residue at position 15 of peptide Z2 or at position 18 of peptide Z2.
30. The GLP-1-/GlP-/amylin-receptor tri-agonist according to any one of the preceding alternative embodiments, wherein the peptide is a peptide derivative comprising a protraction moiety, wherein said protraction moiety further comprises a linker L_{P} selected from the group consisting of:

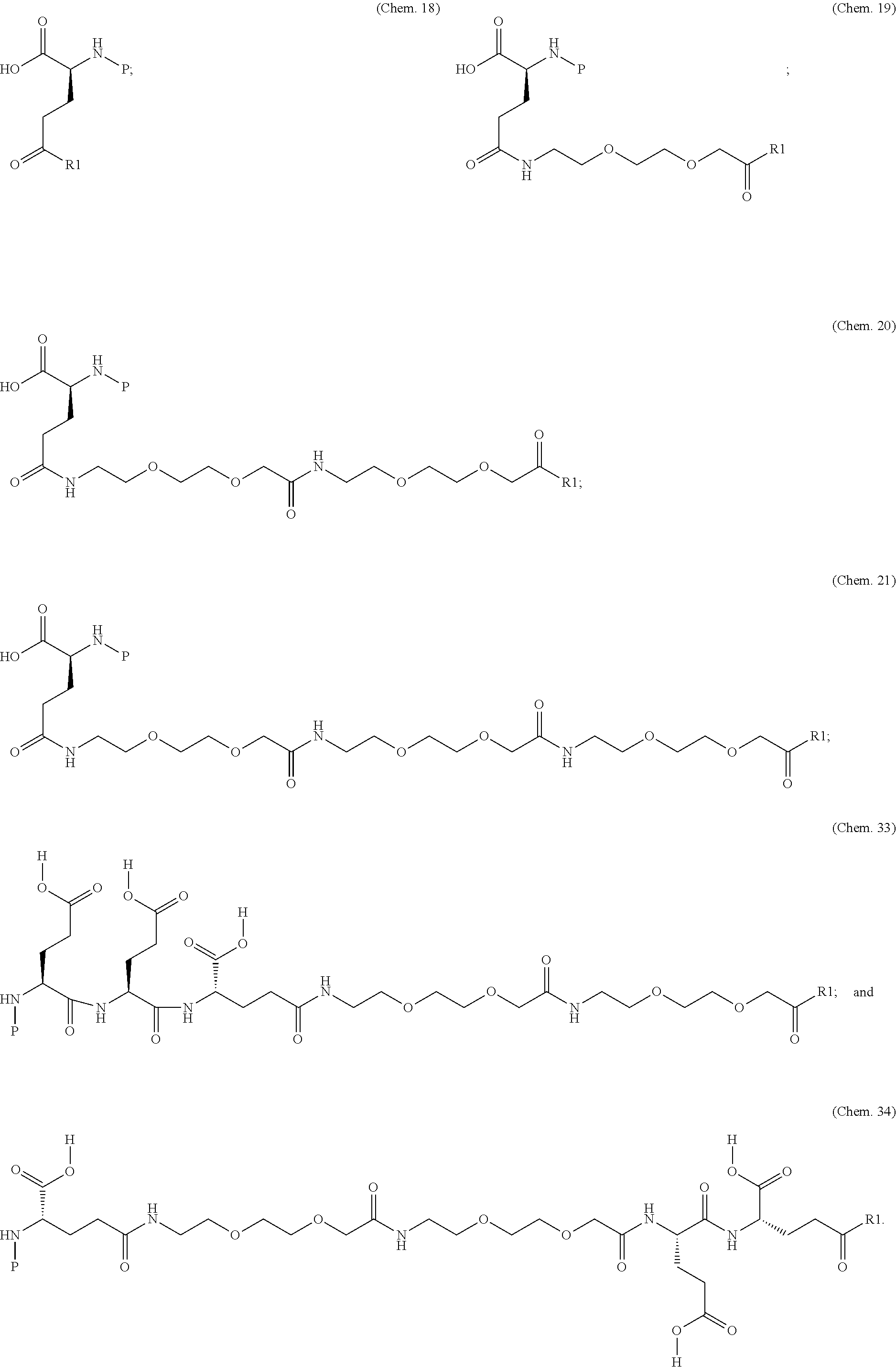

31. The GLP-1-/GlP-/amylin-receptor tr-agonist according to any one of the preceding alternative embodiments, wherein the peptide is a peptide derivative comprising a protraction moiety, wherein said protraction moiety comprises Chem. 20 or Chem. 21 as linker L_{P} and Chem. 5 or Chem. 6 as protractor P.
32. The GLP-1-/GlP-/amylin-receptor tri-agonist according to any one of the preceding alternative embodiments, wherein the peptide is a peptide derivative comprising a protraction moiety,
   wherein said protraction moiety consists of (i) a linker L_{P} selected from the group presented in Table 6 and (ii) a protractor P selected from the group presented in Table 5, preferably said protraction moiety is selected from the group presented in Table 7.
33. The GLP-1-/GlP-/amylin-receptor tri-agonist according to any one of the preceding alternative embodiments, wherein the peptide is a peptide derivative comprising a protraction moiety, wherein said protraction moiety is a C₁₈ diacid (S) gamma-Glu 2×Ado fatty acid moiety (Chem. 28) or a C₂₀ diacid (S) gamma-Glu 2×Ado fatty acid moiety (Chem. 27).
34. The GLP-1-/GlP-/amylin-receptor tri-agonist according to any one of the alternative embodiments 1 to 33,
   wherein the peptide Z1 comprises or consists of the amino acid sequence according to Formula VII (SEQ ID NO: 162):

(VII)
YX₂EGTFTSDYSX₁₂LLEEIAAX₂₀EFIX₂₄WLX₂₇X₂₈GGPSSX₃₄,

wherein
X₂ represents Aib,
X₁₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₄ represents Gly (G) or Glu (E);
wherein the peptide linker L1 comprises or consists of the amino acid sequence selected from the group consisting of E, AG, AGQAPGQAPG (SEQ ID NO: 136), AGQAPGQAPL (SEQ ID NO: 137), AGGGG (SEQ ID NO: 142), AGEAPGQAPG (SEQ ID NO: 143), and AGQAPGEAPG (SEQ ID NO: 154);
wherein the peptide Z2 comprises or consists of the amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E); and
wherein the peptide is a peptide derivative comprising a protraction moiety.
35. The GLP-1-/GlP-/amylin-receptor tri-agonist according to alternative embodiment 34, wherein the peptide is a peptide derivative comprising a protraction moiety, wherein said protraction moiety comprises a protractor P being a C₁₂-C₂₀ diacid.
36. The GLP-1-/GlP-/amylin-receptor tri-agonist according to alternative embodiment 35, wherein the peptide is a peptide derivative comprising a protraction moiety, wherein said protraction moiety further comprises a linker L_{P} selected from the group presented in Table 6.
37. The GLP-1-/GlP-/amylin-receptor tri-agonist according to any one of the alternative embodiments 34 to 36, wherein the peptide Z1 comprises or consists of the amino acid sequence according to Formula XI (SEQ ID NO: 163):

(XI)
YX₂EGTFTSDYSX₁₂LLEEIAAREFIEWLLAGGPSSG,

wherein
X₂ represents Aib,
X₁₂ represents Ile (I) or Lys (K).
38. The GLP-1-/GlP-/amylin-receptor tri-agonist according to any one of the alternative embodiments 34 to 37, wherein the peptide Z2 comprises or consists of the amino acid sequence according to Formula X (SEQ ID NO: 166):

(X)
ASX₅₉LSTAQTQRLSAELHKLATLPRTETGSGSP,

wherein
X₅₉ represents Glu (E) or His (H).
39. A GLP-1-/GlP-/amylin-receptor tri-agonist comprising a peptide according to Formula I:

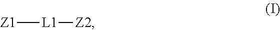

comprising one lysine (Lys, K) residue; wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises or consists of an amino acid sequence according to Formula VII (SEQ ID NO: 162):

(VII)
YX₂EGTFTSDYSX₁₂LLEEIAAX₂₀EFIX₂₄WLX₂₇X₂₈GGPSSX₃₄,

wherein
X₂ represents Aib,
X₂₂ represents Ile (I) or Lys (K),
X₂₀ represents Arg (R) or Gln (Q),
X₂₄ represents Ala (A), Glu (E) or Gln (Q),
X₂₇ represents Leu (L) or I (Ile),
X₂₈ represents Ala (A) or Gln (Q),
X₃₄ represents Gly (G) or Glu (E);
   L1 is a peptide linker comprising or consisting of 1 to 14, 1 to 10, 4 to 10 or 9 to 10 amino acid residues; and
   Z2 is a peptide comprising a C-terminal amide, and having a maximum of 10 amino acid substitutions relative to Formula V (SEQ ID NO: 2):

(V)
ASELSTAALGRLSAELHELATLPRTETGSGSP,

and
Z2 comprises or consists of an amino acid sequence according to Formula VI (SEQ ID NO: 165):

(VI)
AX₅₂X₅₃LSTAX₅₈X₅₉X₆₀RLSAELHX₆₈LATX₇₂PRTETGSGSP,

wherein
X₅₂ represents Gly (G) or Ser (S),
X₅₃ represents Gln (Q), Glu (E), or His (H),
X₅₈ represents Ala (A) or Gln (Q),
X₅₉ represents Leu (L) or Thr (T),
X₆₀ represents Ala (A), Gly (G) or Gln (Q),
X₆₈ represents Gln (Q), Glu (E), or Lys (K),
X₇₂ represents Leu (L) or Glu (E);
wherein the peptide is a peptide derivative comprising a protraction moiety, wherein said protraction moiety comprises a protractor P being a C₁₆-C₂₀ diacid and further comprises a linker L_{P} selected from the group presented in Table 6.
40. The GLP-1-/GlP-/amylin-receptor tri-agonist according to any one of the preceding alternative embodiments wherein the compound is selected from compound no. 120-197 and 211-221 of Example 2 herein.
41. The GLP-1-/GlP-/amylin-receptor tri-agonist according to any one of the preceding alternative embodiments wherein the peptide has the amide modification of the C-terminus.
42. The GLP-1-/GlP-/amylin-receptor tri-agonist according to any one of the preceding alternative embodiments which is capable of activating the human GlP receptor.
43. The GLP-1-/GlP-/amylin-receptor tri-agonist according to any one of the preceding alternative embodiments which is capable of activating the human GlP receptor in an assay with whole cells expressing the human GlP receptor.
44. The GLP-1-/GlP-/amylin-receptor tri-agonist according to any one of the preceding alternative embodiments which activates the human GlP receptor in vitro, preferably with an EC₅₀ of less than 125 pM, even more preferably with an EC₅₀ of less than 100 pM, and most preferably with an EC₅₀ of less than 50 pM, when measured without HSA in an assay as described in Example 4.
45. The GLP-1-/GlP-/amylin-receptor tri-agonist according to any one of the preceding alternative embodiments which is capable of activating the human GLP-1 receptor.
46. The GLP-1-/GlP-/amylin-receptor tri-agonist according to any one of the preceding alternative embodiments which is capable of activating the human GLP-1 receptor in an assay with whole cells expressing the human GLP-1 receptor.
47. The GLP-1-/GlP-/amylin-receptor tri-agonist according to any one of the preceding alternative embodiments which activates the human GLP-1 receptor in vitro, preferably with an EC₅₀ of less than 125 pM, even more preferably with an EC₅₀ of less than 100 pM, and most preferably with an EC₅₀ of less than 50 pM, when measured without HSA in an assay as described in Example 4.
48. The GLP-1-/GlP-/amylin-receptor tri-agonist according to any one of the preceding alternative embodiments which is capable of activating the human amylin receptor.
49. The GLP-1-/GlP-/amylin-receptor tri-agonist according to any one of the preceding alternative embodiments which is capable of activating the human amylin receptor in an assay with whole cells expressing the human amylin receptor.
50. The GLP-1-/GlP-/amylin-receptor tri-agonist according to any one of the preceding alternative embodiments which activates the human amylin receptor in vitro, preferably with an EC₅₀ of less than 125 pM, even more preferably with an EC₅₀ of less than 100 pM, and most preferably with an EC₅₀ of less than 50 pM, when measured without HSA in an assay as described in Example 4.
51. The GLP-1-/GlP-/amylin-receptor tri-agonist according to any one of the preceding alternative embodiments which is capable of activating the human GlP, GLP-1, and amylin receptors.
52. The GLP-1-/GlP-/amylin-receptor tri-agonist according to any one of the preceding alternative embodiments which is capable of activating the human GlP, GLP-1, and amylin receptors in assays with whole cells expressing the human GlP receptor, GLP-1, and amylin receptors.
53. The GLP-1-/GlP-/amylin-receptor tri-agonist according to any one of the preceding alternative embodiments which activates the human GlP, GLP-1 and amylin receptors in vitro, preferably with an EC₅₀ of less than 125 pM, even more preferably with an EC₅₀ of less than 100 pM, and most preferably with an EC₅₀ of less than 50 pM, when measured without HSA in assays as described in Example 4.
54. The GLP-1-/GlP-/amylin-receptor tri-agonist according to any one of the preceding alternative embodiments, which activates the human GlP, GLP-1 and amylin receptors in vitro, when measured without HSA in assays as described in Example 4, and which has a potency ratio of less than 50.
55. The GLP-1-/GlP-/amylin-receptor tri-agonist according to alternative embodiment 54, wherein the potency ratio is less than 20.
56. The GLP-1-/GlP-/amylin-receptor tri-agonist according to alternative embodiment 54 or 55, wherein the potency ratio is less than 15, and most preferred less than 11.
57. The GLP-1-/GlP-/amylin-receptor tri-agonist according to any one of the alternative embodiments 21 to 41 which has improved pharmacokinetic properties.
58. The GLP-1-/GlP-/amylin-receptor tri-agonist according to any one of the alternative embodiments 21 to 41 which has an increased half-life.
59. The GLP-1-/GlP-/amylin-receptor tri-agonist according to alternative embodiment 58, which has an increased half-life of 40 hours to 145 hours, when determined in minipigs, preferably of 90 hours to 140 hours, even more preferably 85 hours to 125 hours.
60. The GLP-1-/GlP-/amylin-receptor tri-agonist according to any one of the alternative embodiments 21 to 59, which has improved chemical stability.
61. The GLP-1-/GlP-/amylin-receptor tri-agonist according to alternative embodiment 60, which has improved chemical stability and which has a purity loss of no more than 6.0 percent per week, such as determined in Example 7 described herein, preferably a purity loss of less than 3.0 percent per week, such as determined in Example 7 described herein.
62. The GLP-1-/GlP-/amylin-receptor tri-agonist according to any one of the alternative embodiments 21 to 61 which has the effect in vivo of reducing food intake in normal weight rats, as determined in the experimental protocol for efficacy testing on appetite, such as Example 5 described herein.
63. A pharmaceutically acceptable salt of the GLP-1-/GlP-/amylin-receptor triple agonist according to any one of the preceding alternative embodiments.
64. A pharmaceutical composition comprising a GLP-1-/GlP-/amylin-receptor triple agonist according to any one of the preceding alternative embodiments, and one or more pharmaceutically acceptable excipients.
65. The pharmaceutical composition according to alternative embodiment 64, which is for oral or for subcutaneous administration.
66. The pharmaceutical composition according to the alternative embodiments 64 or 65, which is a solid pharmaceutical composition.
67. The solid pharmaceutical composition according to alternative embodiment 66, which is a tablet.
68. The solid pharmaceutical composition according to alternative embodiment 66 or 67, comprising a salt of N-[8-(2-hydroxybenzoyl)amino]caprylate, preferably sodium N-(8-(2-hydroxybenzoyl)amino)caprylate and magnesium stearate.
69. The solid pharmaceutical composition according to any one of the alternative embodiments 66 to 68, comprising 75-600 mg sodium N-(8-(2-hydroxybenzoyl)amino)caprylate and 7-8.5 mg magnesium stearate.
70. The pharmaceutical composition according to any one of alternative embodiments 64 to 69, which is for dosing approximately once daily, such as once every 12-36 hours, such as once every 18-30 hours, such as approximately once every 24 hours; or which is for dosing approximately once weekly, such as once every 6-8 days.
71. An injection device comprising the GLP-1-/GlP-/amylin-receptor tri-agonist according to any one of alternative embodiments 1 to 63, or the pharmaceutical composition according to any one of embodiments 64 to 70.
72. The GLP-1-/GlP-/amylin-receptor tri-agonist peptide according to any of alternative embodiments 1 to 19 for use as an intermediate in the manufacture of a GLP-1-/GlP-/amylin-receptor tri-agonist peptide derivative according to any of embodiments 22 to 64.
73. The GLP-1-/GlP-/amylin-receptor triple agonist according to any one of alternative embodiments 1 to 63 or the pharmaceutical composition according to any one of embodiments 64 to 70 for use as a medicament.
74. The GLP-1-/GlP-/amylin-receptor triple agonist according to any one of alternative embodiments 1 to 63 or the pharmaceutical composition according to any one of embodiments 64 to 70 for use in the treatment of type 2 diabetes, obesity, metabolic dysfunction-associated steatohepatitis (MASH), and/or cardiovascular disease.
75. The GLP-1-/GlP-/amylin-receptor triple agonist according to any one of alternative embodiments 1 to 63 or the pharmaceutical composition according to any one of embodiments 64 to 70, for use in the treatment of a subject with an initial body mass index (BMI) of 25 or more, 27 or more, or 28 or more, or 30 or more.
76. The GLP-1-/GlP-/amylin-receptor triple agonist according to any one of alternative embodiments 1 to 63 or the pharmaceutical composition according to any one of embodiments 64 to 70, for use in the treatment of a subject with an initial body mass index (BMI) of 25 or more, 27 or more, or 28 or more, 30 or more; and in the presence of at least one weight-related co-morbidity.
77. The GLP-1-/GlP-/amylin-receptor triple agonist according to any one of alternative embodiments 1 to 63 or the pharmaceutical composition according to any one of embodiments 64 to 70, for use as an adjunct to a reduced-calorie diet and increased physical activity for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more.
78. The GLP-1-/GlP-/amylin-receptor triple agonist according to any one of alternative embodiments 1 to 63 or the pharmaceutical composition according to any one of embodiments 64 to 70, for use as an adjunct to a reduced-calorie diet and increased physical activity for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more; and in the presence of at least one weight-related co-morbidity.
79. The use according to alternative embodiment 76 or alternative embodiment 78, wherein the at least one weight-related co-morbidity is selected from the group consisting of hypertension, dysglycemia (prediabetes or type 2 diabetes), dyslipidaemia, high cholesterol, cardiovascular disease and obstructive sleep apnoea.
80. Use of a GLP-1-/GlP-/amylin-receptor triple agonist according to any one of alternative embodiments 1 to 63 in the manufacture of a medicament for the treatment of type 2 diabetes, obesity, metabolic dysfunction-associated steatohepatitis (MASH), and/or cardiovascular disease.
81. Use of a GLP-1-/GlP-/amylin-receptor triple agonist according to any one of alternative embodiments 1 to 63 in the manufacture of a medicament for the treatment of a subject with an initial body mass index (BMI) of 25 or more, 27 or more, or 28 or more, 30 or more; and optionally in the presence of at least one weight-related co-morbidity.
82. Use of a GLP-1-/GlP-/amylin-receptor triple agonist according to any one of alternative embodiments 1 to 63 in the manufacture of a medicament for the treatment of an adult subject suffering from obesity, i.e. with an initial body mass index (BMI) of 25 or more, or 27 or more, or 28 or more, 30 or more; or an adult subject suffering from overweight, i.e. with an initial body mass index (BMI) of 23 or more, or 24 or more, or 25 or more, or 27 or more; and optionally in the presence of at least one weight-related co-morbidity.
83. Use of a GLP-1-/GlP-/amylin-receptor triple agonist according to any one of alternative embodiments 1 to 63 in the manufacture of a medicament for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more; and optionally in the presence of at least one weight-related co-morbidity.
84. The use according to alternative embodiment 82, wherein the medicament is an adjunct to a reduced-calorie diet and increased physical activity for chronic weight management in an adult subject suffering from obesity or overweight.
85. A method for treating type 2 diabetes, obesity, metabolic dysfunction-associated steatohepatitis (MASH), and/or cardiovascular disease comprising administering a pharmaceutically relevant amount of a GLP-1-/GlP-/amylin-receptor triple agonist according to any one of alternative embodiments 1 to 63 to a subject in need thereof.
86. A method of treating a human subject with an initial body mass index (BMI) of 25 or more, 27 or more, or 28 or more, or 30 or more, comprising administering to said human subject a pharmaceutically relevant amount of a GLP-1-/GlP-/amylin-receptor triple agonist according to any one of alternative embodiments 1 to 63.
87. A method for reducing excess body weight in a human subject, in combination with a reduced-calorie diet and increased physical activity, comprising administering to said human subject a pharmaceutically relevant amount of a GLP-1-/GlP-/amylin-receptor triple agonist according to any one of alternative embodiments 1 to 63.
88. The method according to alternative embodiment 86 or alternative embodiment 87, wherein said human subject is an adult subject, suffering from overweight and has an initial body mass index (BMI) of 23 or more, or 24 or more, or 25 or more, or 27 or more.
89. The method according to alternative embodiment 86 or alternative embodiment 87, wherein said human subject is an adult subject, suffering from obesity and has an initial body mass index (BMI) of 25 or more, or 27 or more, or 28 or more, 30 or more.
90. The method according to any one of alternative embodiments 86 to 89, wherein said human subject has at least one weight-related comorbidity selected from the group consisting of hypertension, dysglycemia (prediabetes or type 2 diabetes), dyslipidaemia, high cholesterol, cardiovascular disease and obstructive sleep apnoea.
91. A method for preparing the GLP-1-/GlP-/amylin-receptor triple agonist according to any one of alternative embodiments 1 to 63.
92. The method according to alternative embodiment 91, comprises a step of solid phase peptide synthesis.

### EXAMPLES

### Materials and Methods

### List of Abbreviations

The following abbreviations are used in the following, in alphabetical order:
Ado: 8-amino-3,6-dioxaoctanoic acid
Aib: 2-aminoisobutyric acid
amu: atomic mass unit
BHK Baby Hamster Kidney
Boc: t-butyloxycarbonyl
CAD: Charged Aerosol Detector
cAMP: cyclic adenosine monophosphate
CRE: cAMP response element
DCM: dichloromethane
DIC: N,N-diisopropylcarbodiimide
DIO: Diet Induced Obese
DMB: 2,4-dimethoxybenzyl
DMEM: Dulbecco's Modified Eagle's Medium
DMF: N,N-dimethyl formamide
DTT: 1,4-dithiothreitol
EC₅₀: half maximal effective concentration
EDTA: ethylenediaminetetraacetic acid
ES: Electrospray
FBS: Fetal Bovine Serum
Fmoc: 9-fluorenylmethyloxycarbonyl
FWHM: Full Width at Half Maximum
GlP: Glucose-dependent Insulinotropic Polypeptide
GLP-1: Glucagon-Like Peptide-1
hAMYR3: human Amylin receptor 3
hGlPR: human Glucose-dependent Insulinotropic Polypeptide Receptor
hGLP-1R: human Glucagon-Like Peptide-1 Receptor
HEPES: N-(2-Hydroxyethyl)piperazine-N-(2-ethanesulfonic acid)
HFIP: 1,1,1,3,3,3-hexafluoro-2-propanol or hexafluoroisopropanol
HPLC: High Performance Liquid Chromatography
HSA: Human Serum Albumin
i.v.: intravenously
LCMS or LC-MS: Liquid Chromatography Mass Spectrometry
LLoQ: Lower limit of Quantitation
Luc: luciferase
MeCN: acetonitrile
MRI: Magnetic resonance imaging
MS: Mass Spectrometry
Mtt: 4-methyltrityl
NCA: non-compartmental pharmacokinetic method
nd: not determined
OtBu: tert-butoxy
Oxyma Pure®: cyano-hydroxyimino-acetic acid ethyl ester
Pbf: 2,2,4,6,7-pentamethyldihydrobenzofuran-5-sulfonyl
PBS: phosphate-buffered saline
PK: pharmacokinetic
QD: quaque die (once a day)
QTof: Quadrupole Time of Flight
RAMP3: receptor modifying protein 3
RT: Room Temperature
s.c.: subcutaneously
SD: Sprague Dawley
SEM: Standard Error of Mean
SPPS: Solid Phase Peptide Synthesis
tBu: tert-butyl
TFA: trifluoroacetic acid
TIPS: triisopropylsilane
TQ: Triple Quadrupole
Trt: triphenylmethyl or trityl
UPLC: Ultra Performance Liquid Chromatography
UV: Ultraviolet

### Fatty Diacid and Special Amino Acid Building Blocks

For synthesis of octadecanedioic acid mono-tert-butyl ester (C₁₈ diacid mono-tert-butyl ester): see patent application WO 2010/102886 (pages 27-28). The corresponding mono-tert-butyl esters of C₁₂-C₂₀ diacid, in particular C₁₆ diacid and C₂₀ diacid, can be prepared accordingly.

Fmoc-Leu-Ser(ψ^{Me},^{Me}pro)-OH, Fmoc-Tyr(tBu)-Ser(ψ^{Me},^{Me}pro)-OH, and Fmoc-Gly-(DMB)Gly-OH were commercially available from TechnoComm Ltd.

### General Method for Peptide Synthesis

The preparation of the peptides (for reference compounds and compounds of the invention) was carried out with SPPS using Fmoc based chemistry on a Symphony X from Protein Technologies, a PurePep Chorus from Protein Technologies, a MultiPep 2 from CEM, a Vapourtec RS-500 from Vapourtec or a CS136XT from CSBio. The Fmoc-protected amino acids used in the methods were the standard recommended: Fmoc-Ala-OH, Fmoc-Arg(Pbf)-OH, Fmoc-Asp(OtBu)-OH, Fmoc-Asn(Trt)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Lys(Mtt)-OH, Boc-His(Trt)-OH, Fmoc-Aib-OH, Fmoc-Glu-OtBu, and Fmoc-Ado-OH supplied from e.g. Gyros Protein Technologies, Bachem, Iris Biotech, or NovabioChem. The building blocks Fmoc-Leu-Ser(ψ^{Me,Me}pro)-OH, Fmoc-Tyr(tBu)-Ser(ψ^{Me,Me}-pro)-OH, and Fmoc-Gly-(DMB)Gly-OH, e.g. commercially available from TechnoComm Ltd, were introduced where applicable.

Fmoc-PAL AM resin or Rink-Amide AM resin were used, which were commercially available from NovabioChem. The subsequent amino acids were introduced in a stepwise procedure by the Symphony X peptide synthesizer following the SPPS principles.

Fmoc-deprotection was achieved with 20% piperidine in DMF with 0.1 M Oxyma Pure for 2×10 min. Introduction of the substituent (i.e. protraction moiety comprising a “protractor” P and an optional “linker L_{P}”) at the alpha-position of the N-terminal amino acid was accomplished using a standard Fmoc-protected amino acid. The peptide couplings were performed with DIC and collidine. Amino acid/Oxyma Pure solutions (0.3 M/0.3 M in DMF at a molar excess of 5-10-fold) was added to the resin first. Then, the same molar equivalent of DIC was added (1.5 M in DMF), followed by collidine (1.5 M in DMF). Most commonly, it was mixed for 1 hour. In some cases, the coupling time was increased, additional DIC was added, or the coupling step was repeated. Afterwards, a capping step was performed with 1 M acetic anhydride in DMF and collidine. Introduction of the protraction moiety at the epsilon-nitrogen of a lysine (Lys, K) within the sequence was achieved using Fmoc-Lys(Mtt)-OH. Following the synthesis of the peptide backbone sequence the Mtt group was removed by treatment with HFIP/DCM/TIPS (75:23:2) (5 min), followed by a wash with DCM. The resin was then resuspended in HFIP/DCM/TIPS (75:23:2) (2×25 min), and subsequently washed with DCM and DMF. The protraction moieties were introduced in a stepwise procedure as described above, using suitably protected building blocks for the linker L_{P}, such as the standard Fmoc-protected amino acids such as Fmoc-8-amino-3,6-dioxaoctanoic acid or Fmoc-Glu-OtBu. Introduction of the protractor, fatty acid group, was achieved using the suitable building block, such as but not limited to, octadecanedioic acid mono-tert-butyl-ester or eicosanedioic acid mono-tert-butyl-ester.

### General Cleavage Method

The peptides were cleaved from the resin with TFA/TIPS/H₂O/DTT (90:4:3:3) for 2-3 hours. Hereafter, the peptide was drained into cold diethyl ether, and centrifuged. The ether was decanted off, and the peptide precipitate was washed with ether two additional times.

### General Method for Purification and Quantification of the Derivative

The crude peptide was dissolved in acetic acid/MeCN/Milli-Q water (45:10:45 or 40:20:40) and orthogonally purified by reversed-phase preparative HPLC (Waters Delta Prep 4000) on a column comprising C18-silica gel. The first elution was performed with an increasing gradient from 20-50% of MeCN in Milli-Q water comprising 1% ammonium bicarbonate. Relevant fractions were analysed with UPLC. Fractions containing the target peptide were pooled and diluted with Milli-Q water (1:1) prior to a second reversed-phase preparative HPLC. The second elution was performed with an increasing gradient from 20-50% of MeCN in Milli-Q water comprising 0.1% TFA. Relevant fractions were analysed with UPLC. Fractions containing the pure target peptide were pooled. The resulting solution was analysed (UPLC, LCMS) and the peptide derivative was quantified using a CAD specific HPLC detector (Thermo-Fischer Vanquish HPLC-CAD). The product was dispensed into glass vials. The vials were capped with Millipore glass fibre prefilters. Freeze-drying afforded the trifluoroacetate salt of the derivative as a white solid.

Compounds synthesized below have been prepared using the methods described above.

### Example 1: Reference Compounds

### Reference Compound 1

### (GLP-1-/GlP-/Amylin-Receptor Tri-Agonist Disclosed in WO 2023/288313, Example 1, Peptide/Compound No. 16; SEQ ID NO: 11)

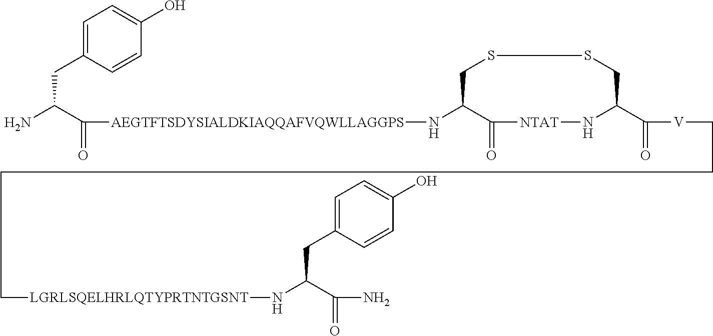

### Reference Compound 2

### (Tri-Agonist Based on Conjugation of Tirzepatide and Cagrilintide, SEQ ID NO: 12)

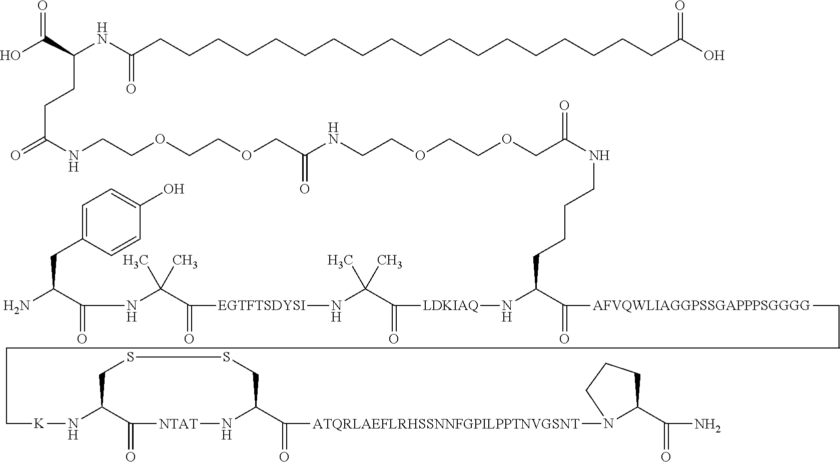

### Reference Compound 3

### (Tri-Agonist Based on Conjugation of Tirzepatide and Amylin Receptor Agonist Disclosed in Example 21 in WO 2016/034604, SEQ ID NO: 13)

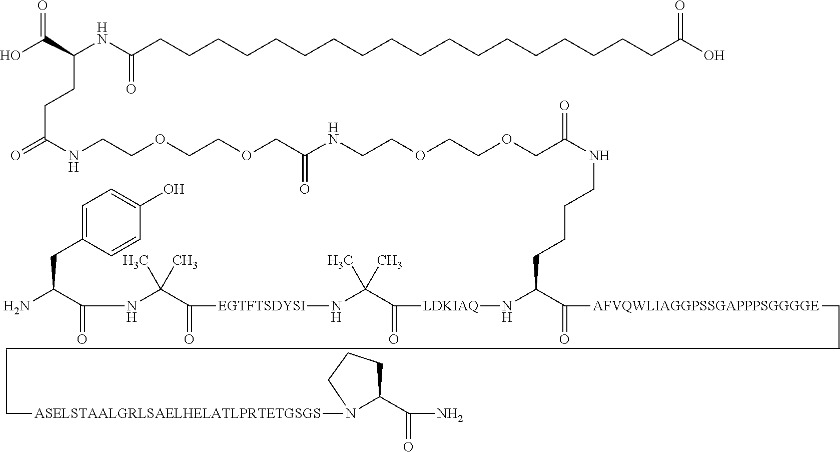

### Reference Compound 4

### (GlP Receptor Agonist Disclosed in WO 2019/211451, Ex. 1, Compound 31, SEQ ID NO: 14)

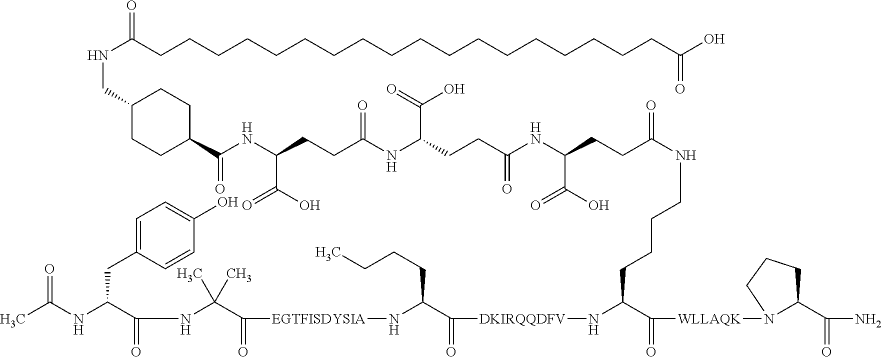

### Reference Compound 5

### (GLP-1/GlP Co-Agonist Tirzepatide, SEQ ID NO: 15)

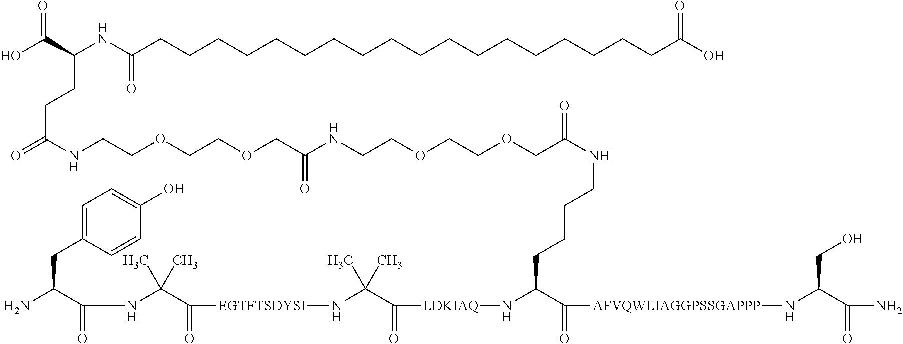

### Reference Compound 6

### (Amylin Receptor Agonist Cagrilintide, WO 2012/168432, Example 53, SEQ ID NO: 16)

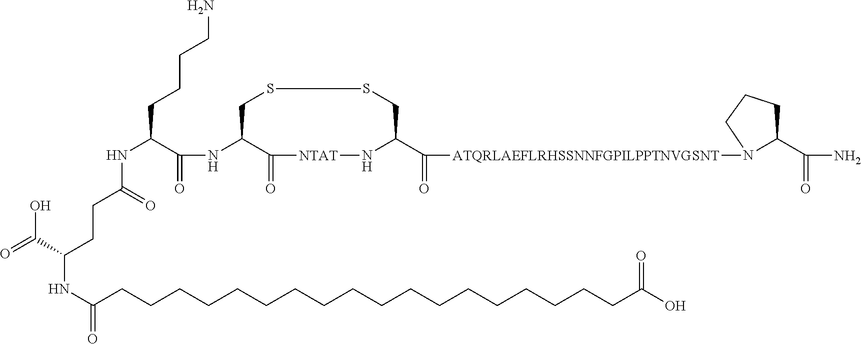

### Reference Compound 7

### (GLP-1 Receptor Agonist Semaglutide, WO 2006/097537, Example 4, SEQ ID NO: 17)

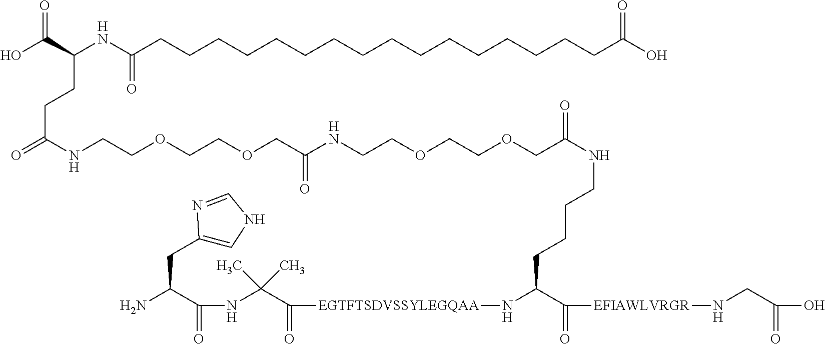

### Example 2: GLP-1-/GlP-/Amylin-Receptor Tri-Agonists According to the Invention

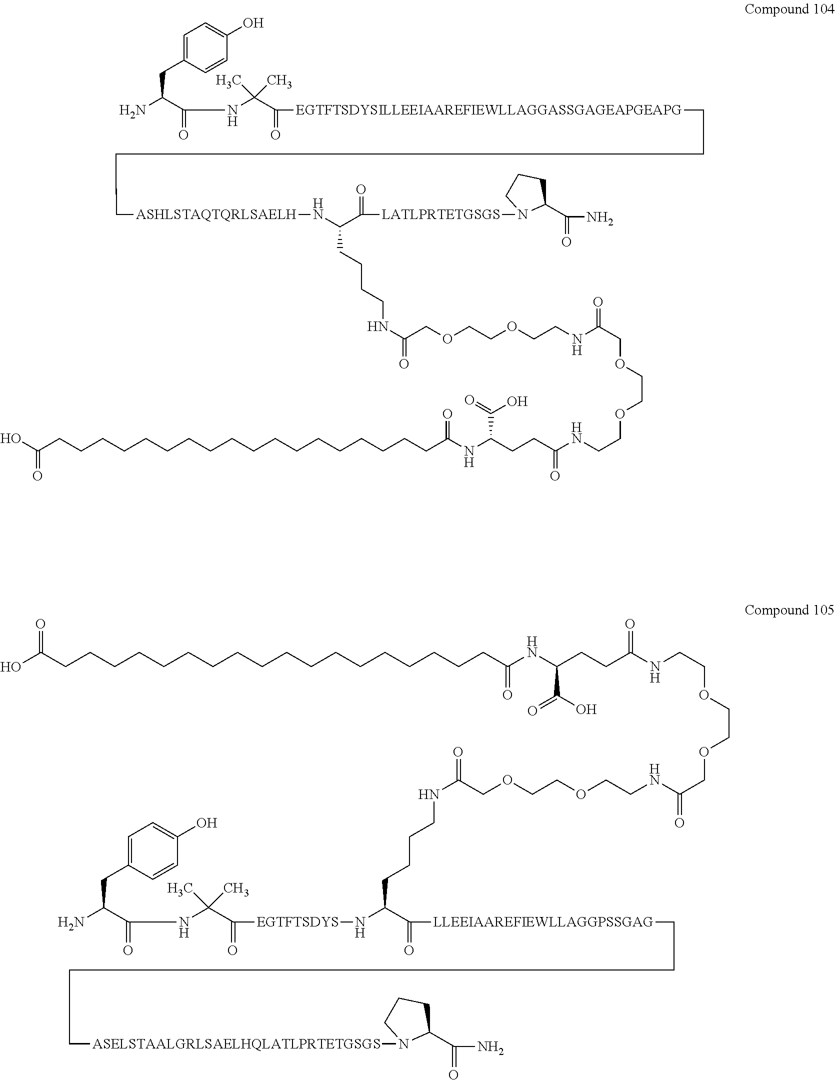

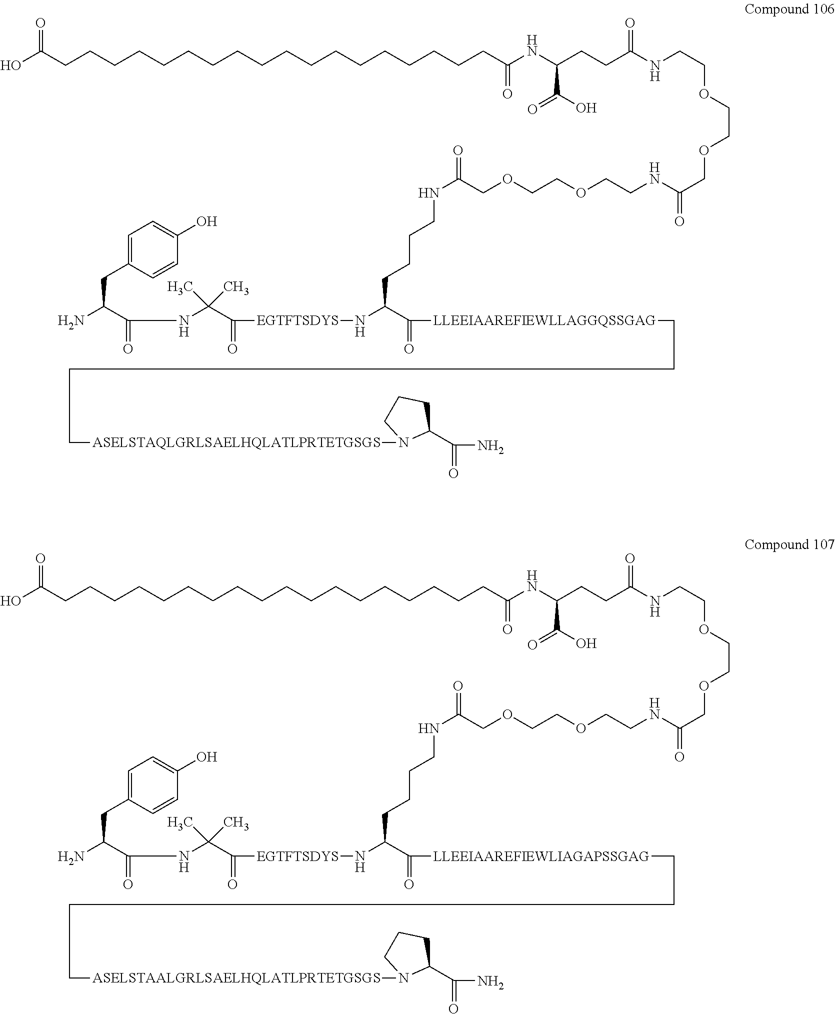

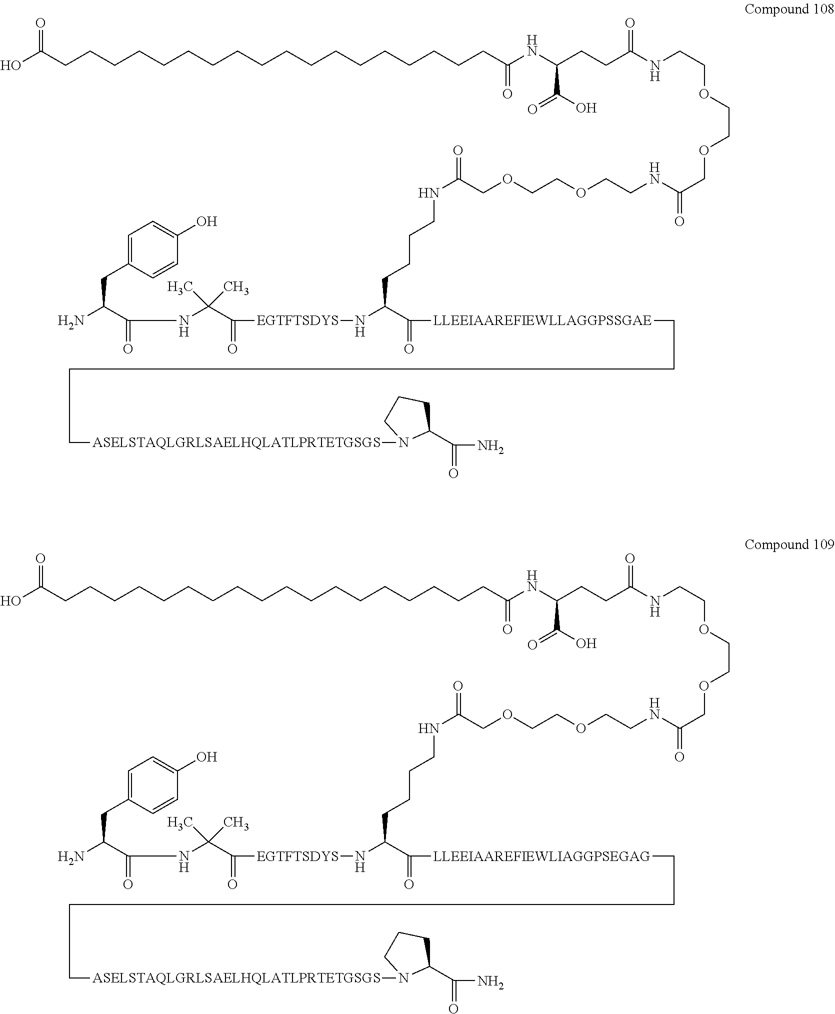

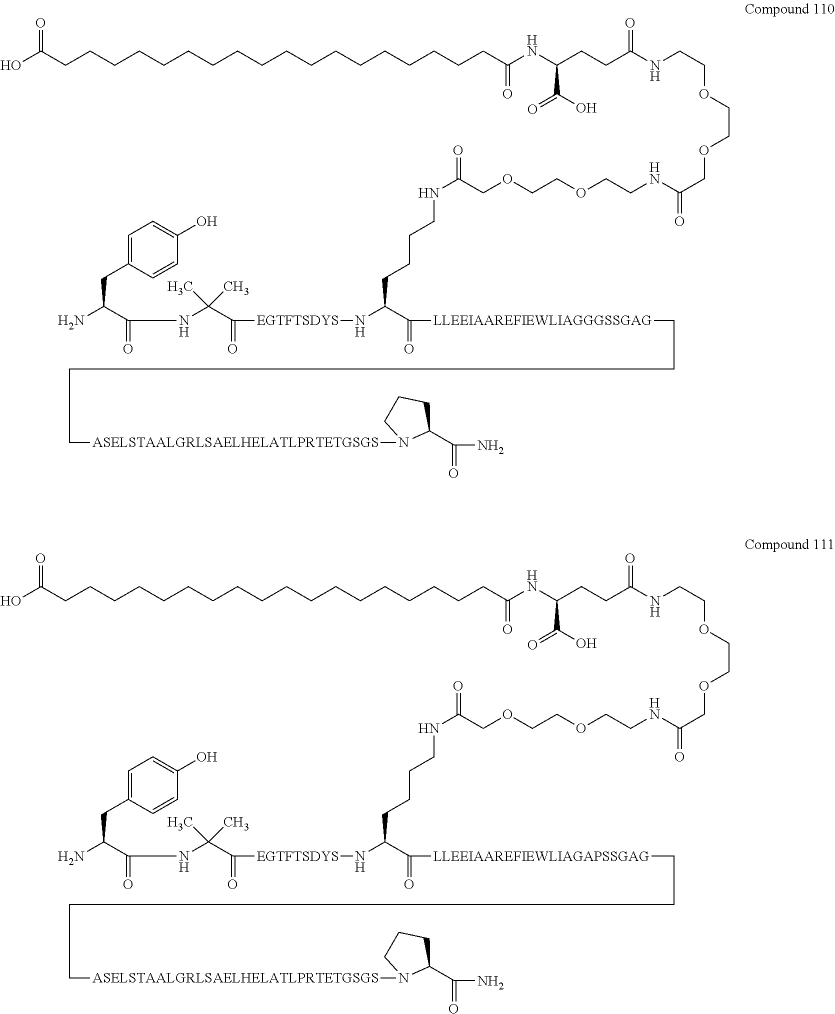

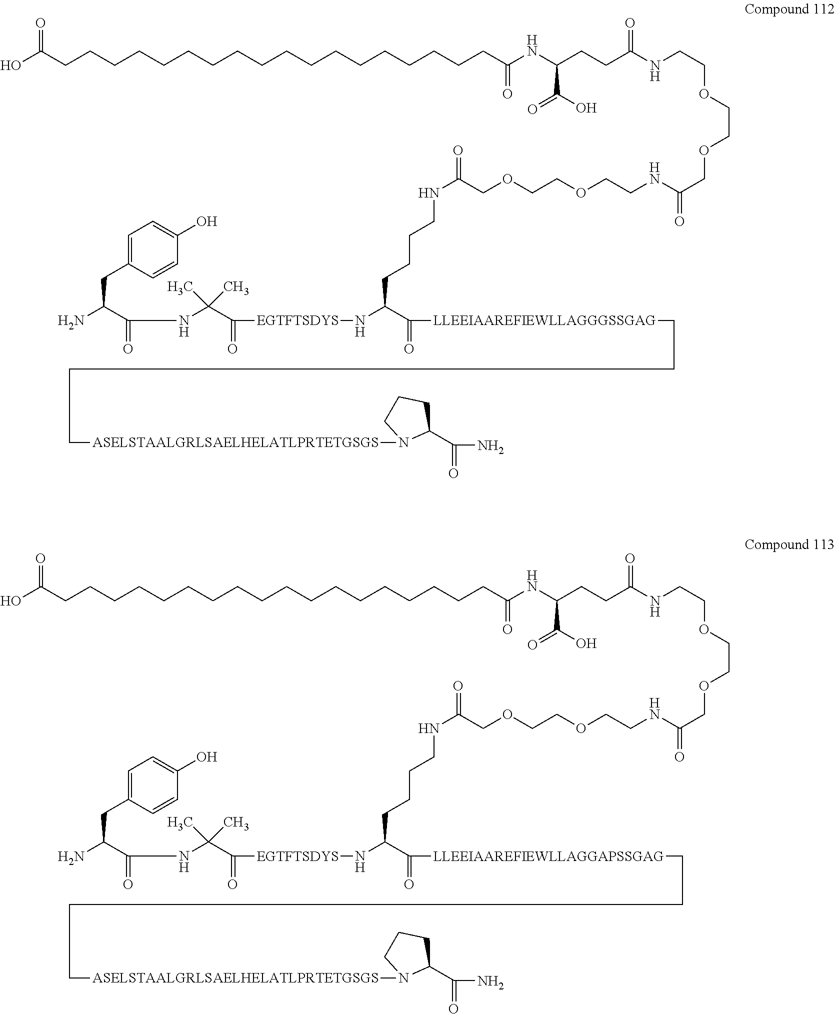

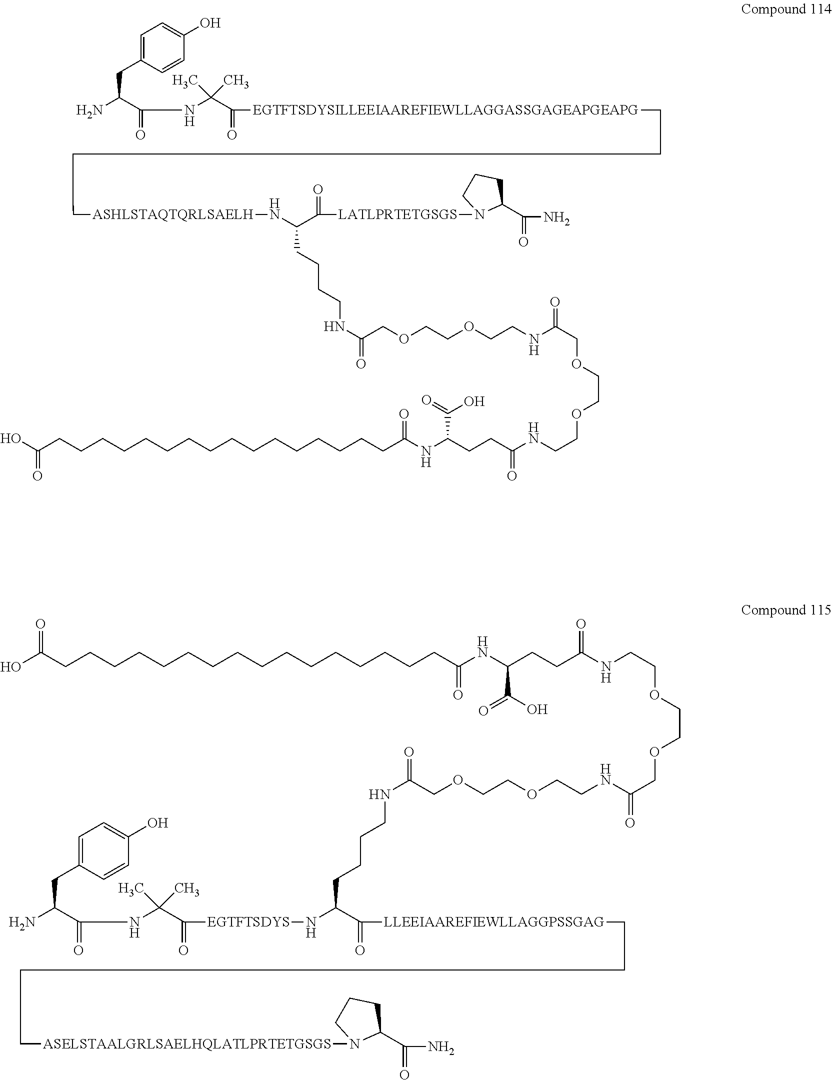

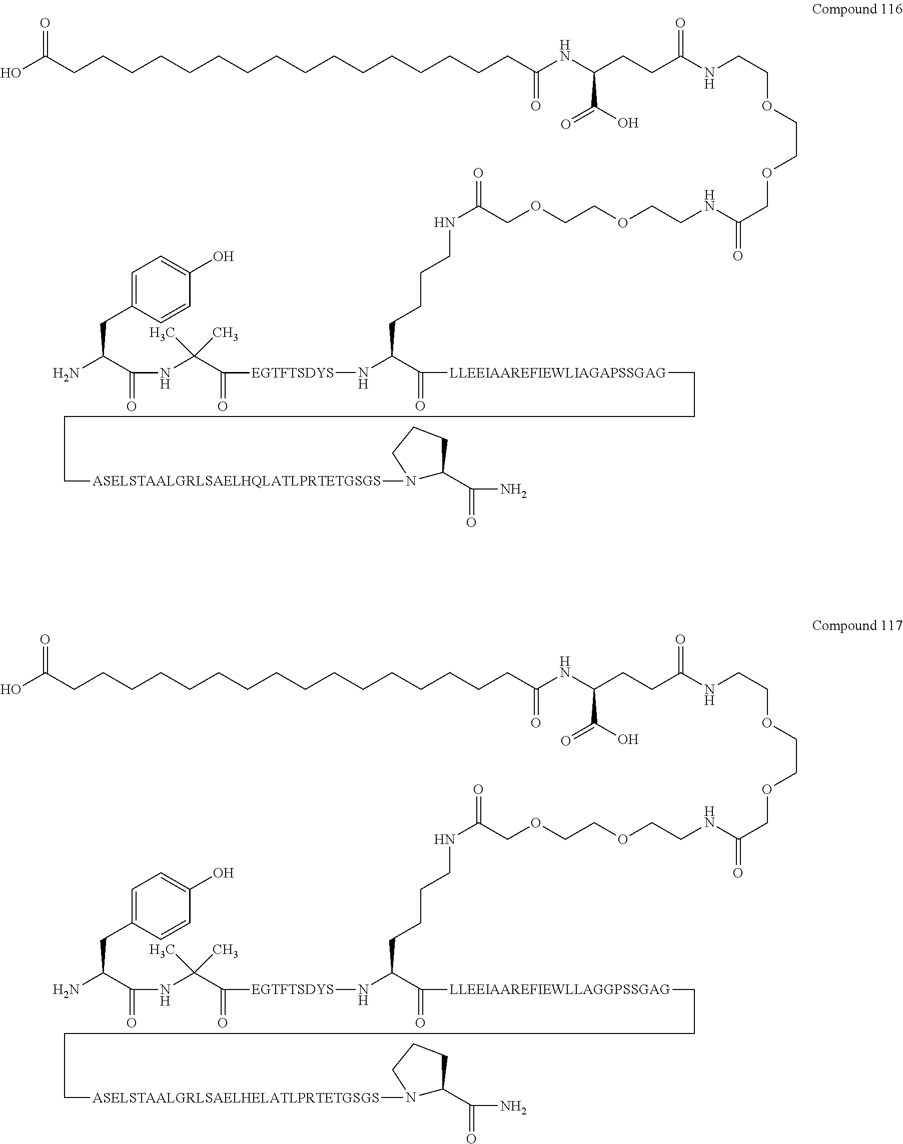

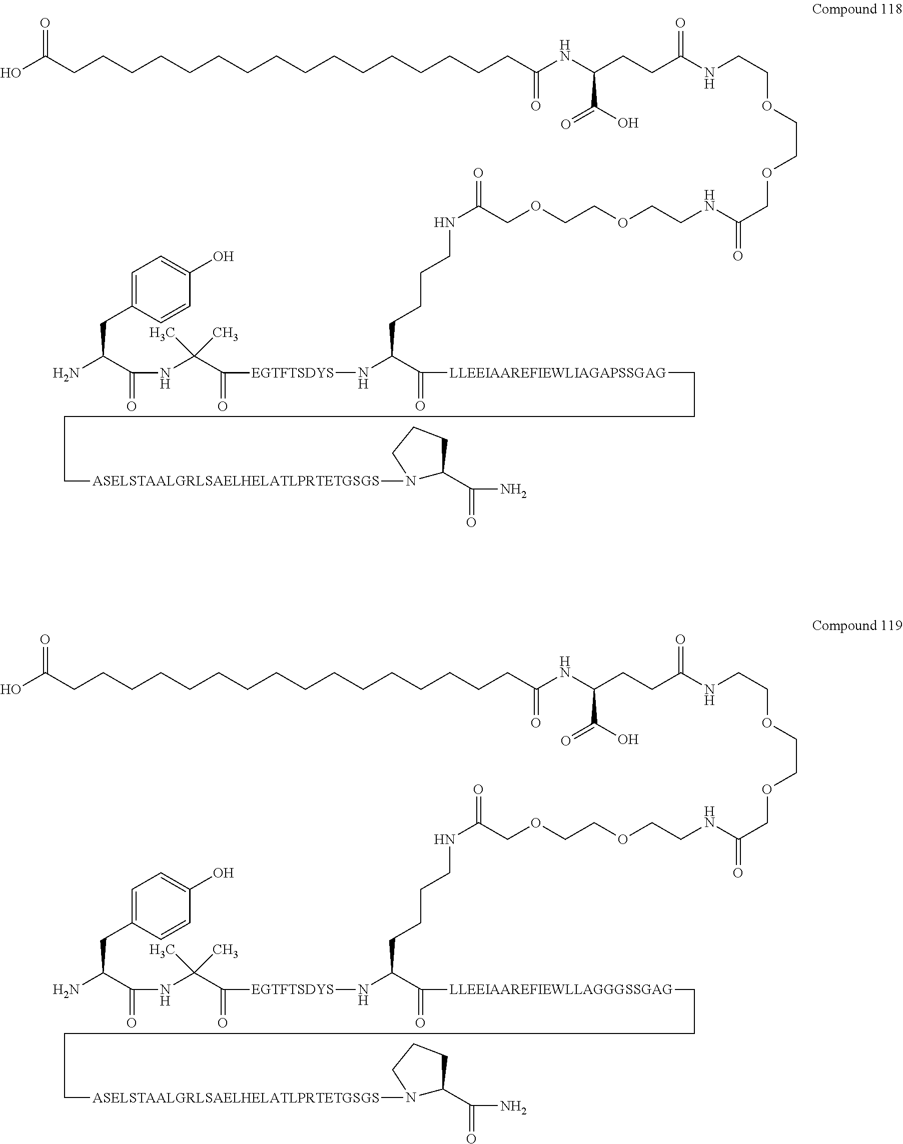

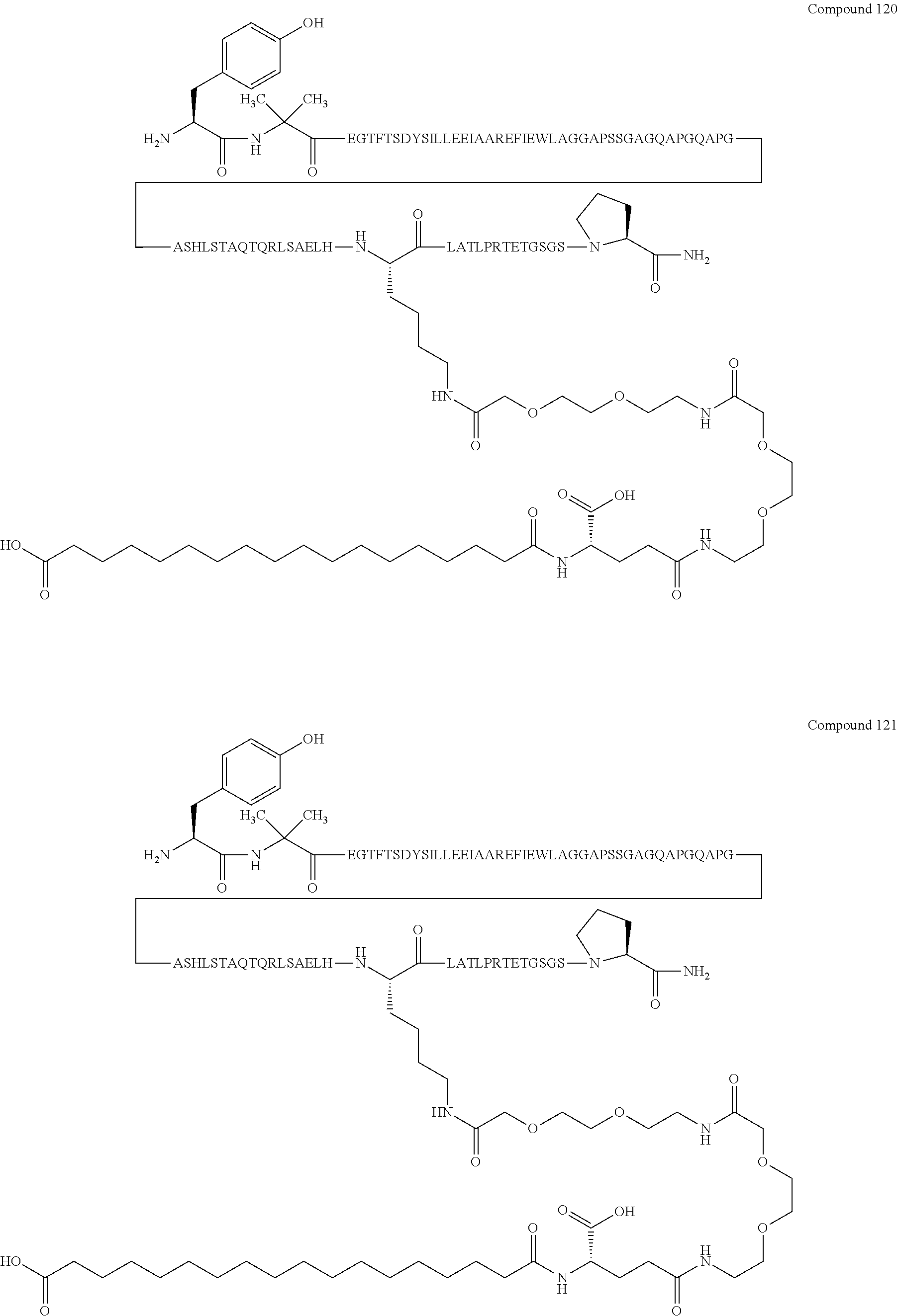

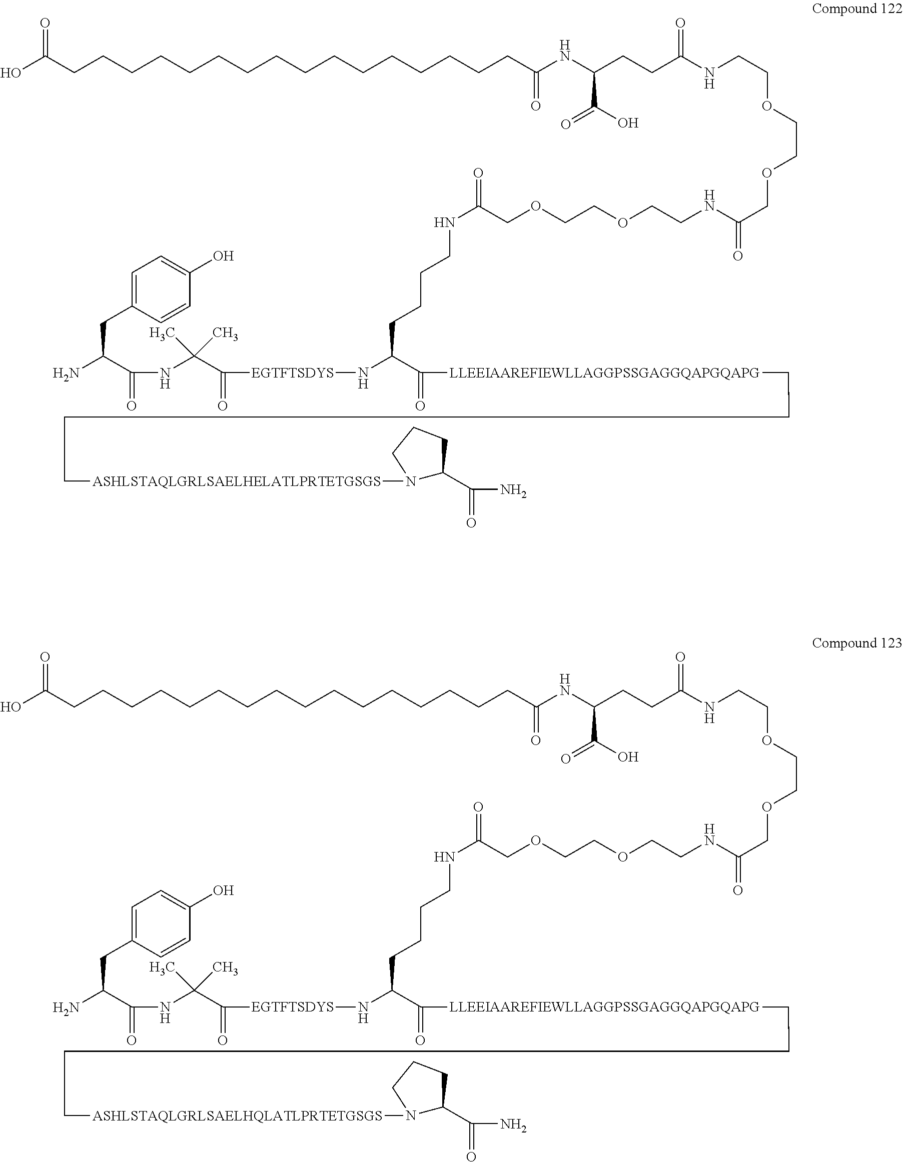

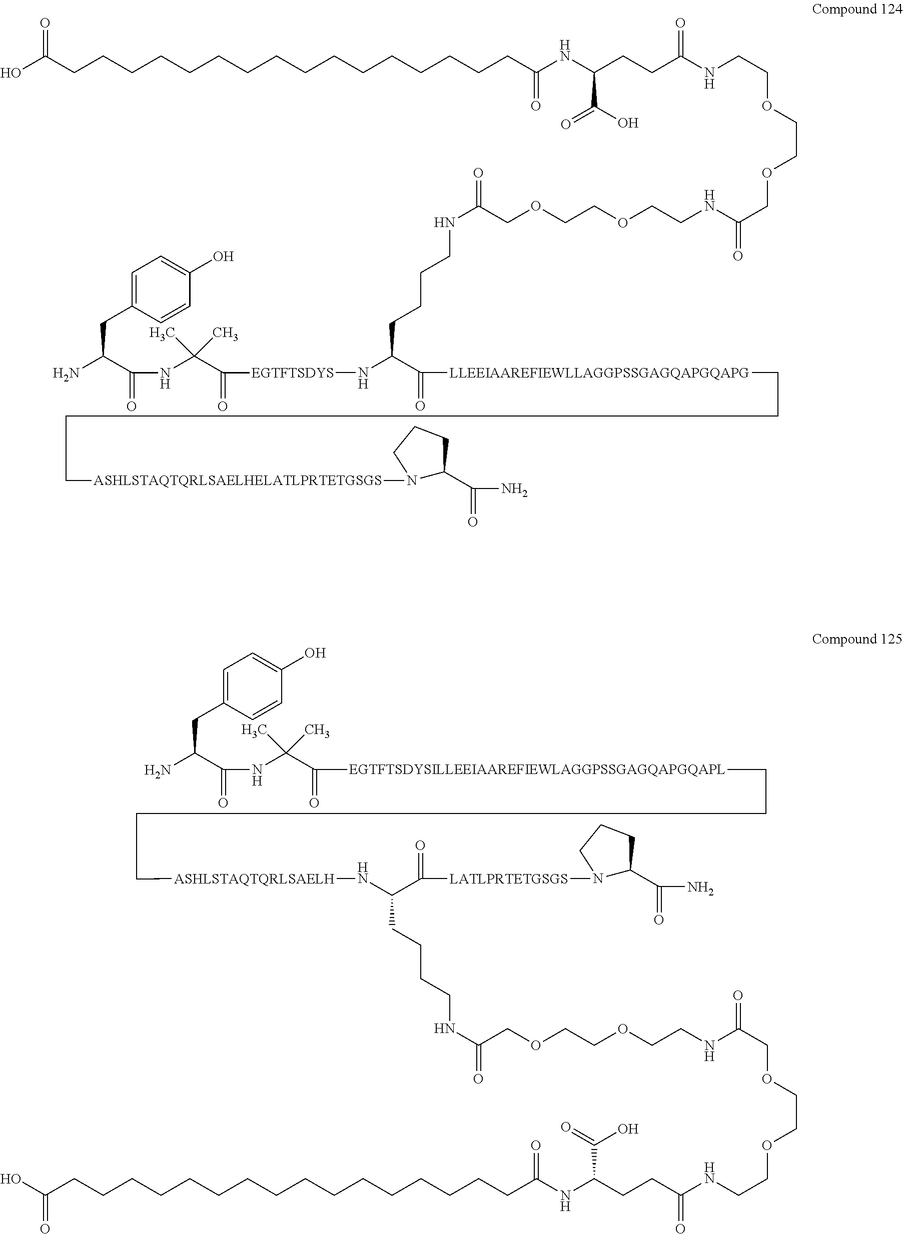

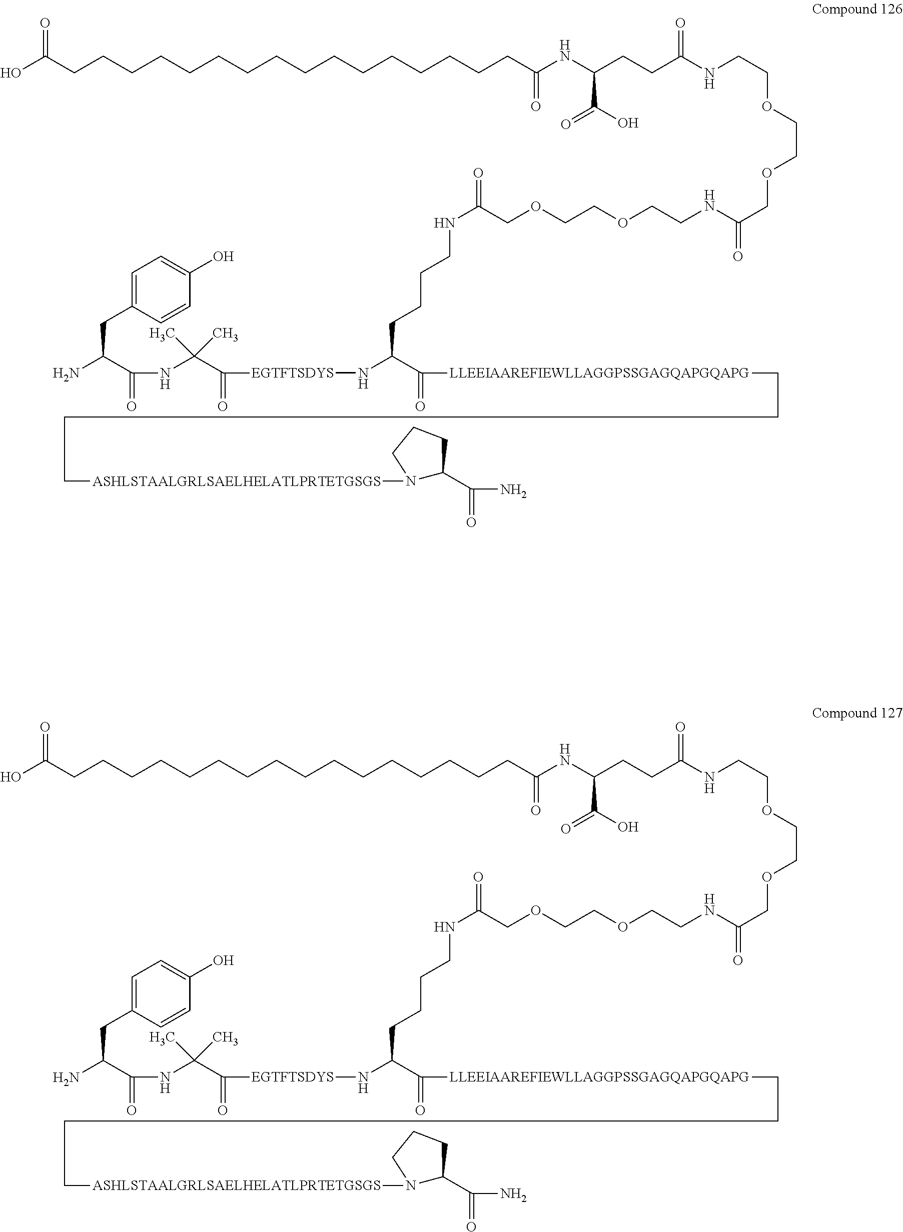

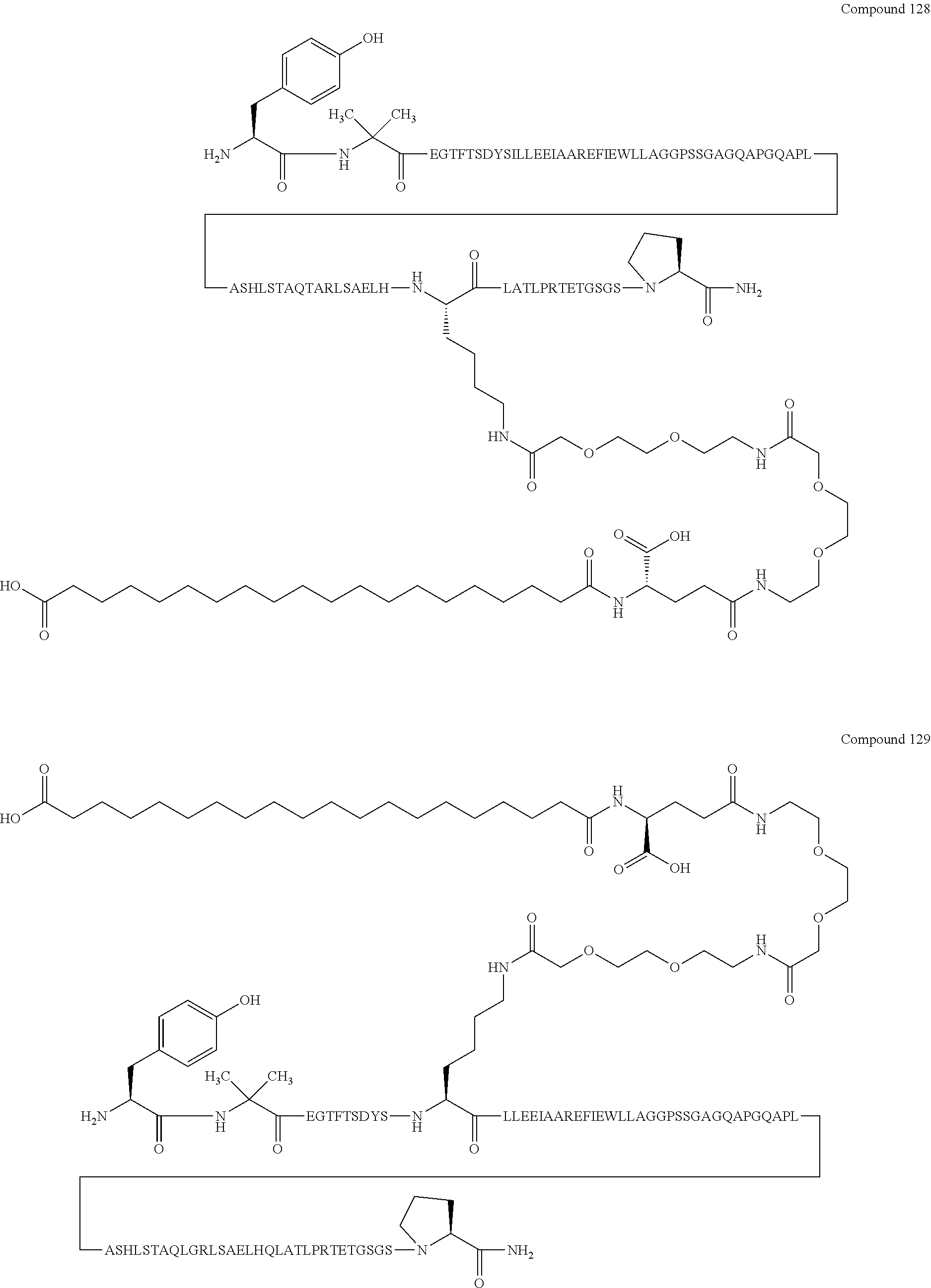

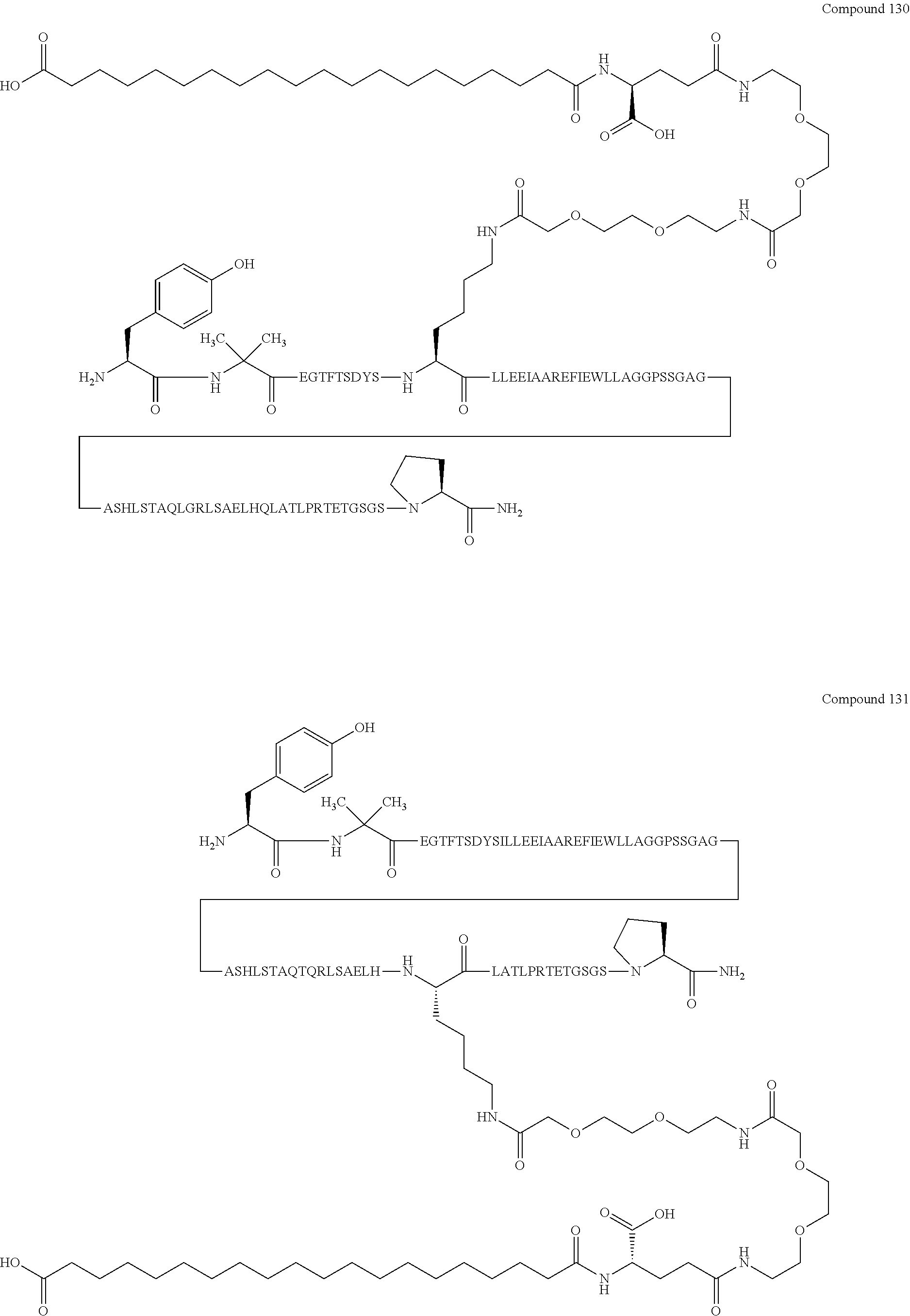

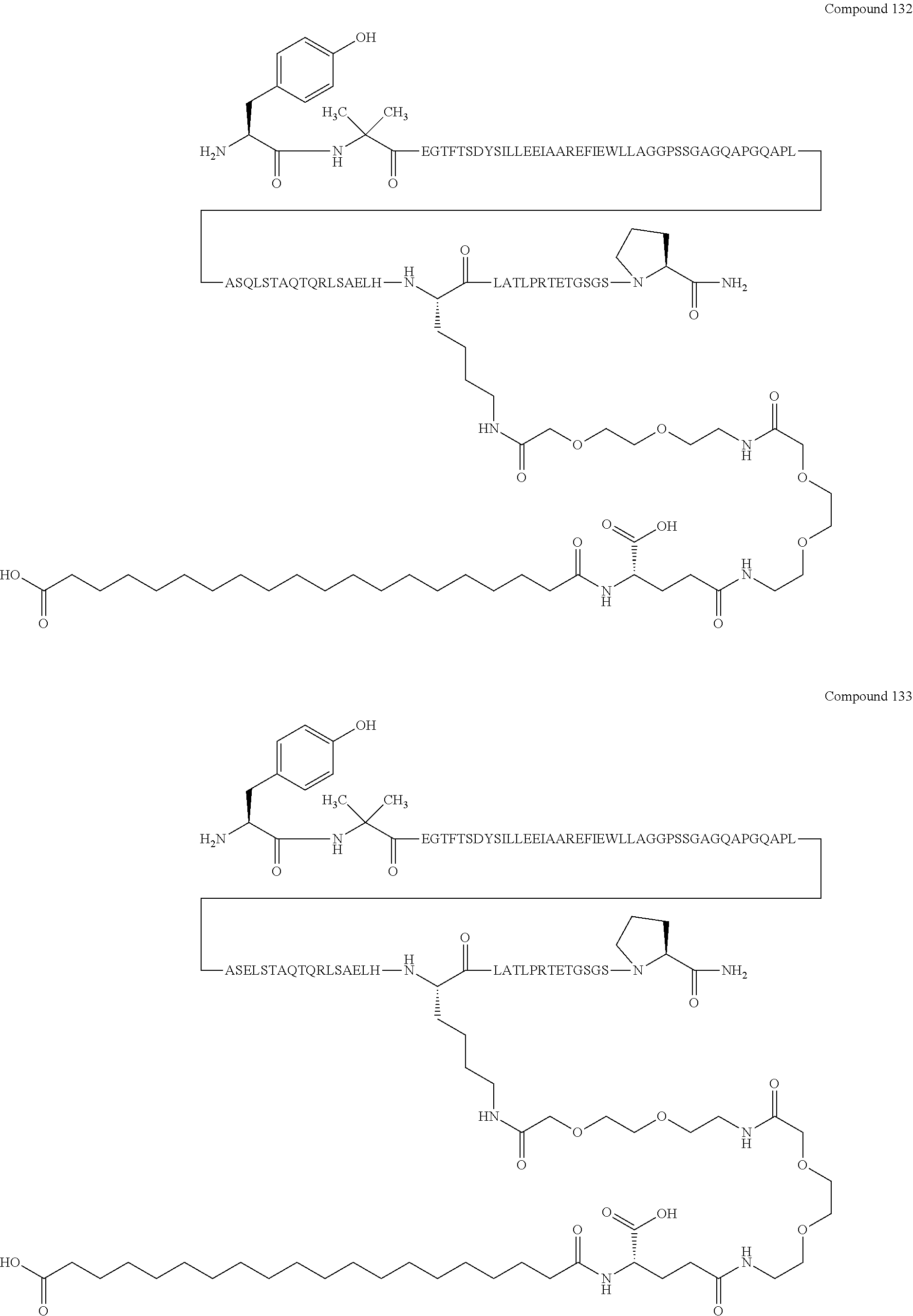

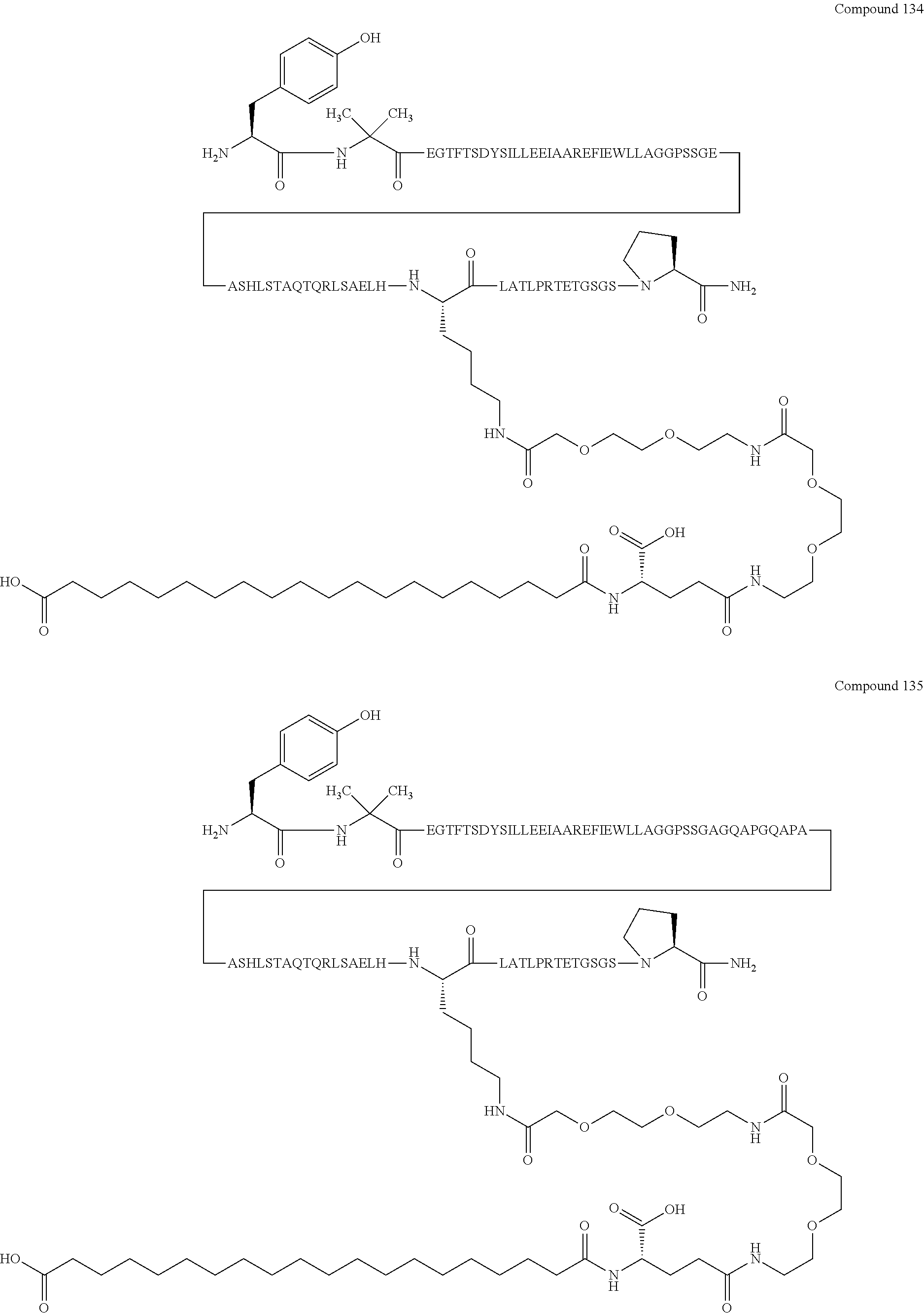

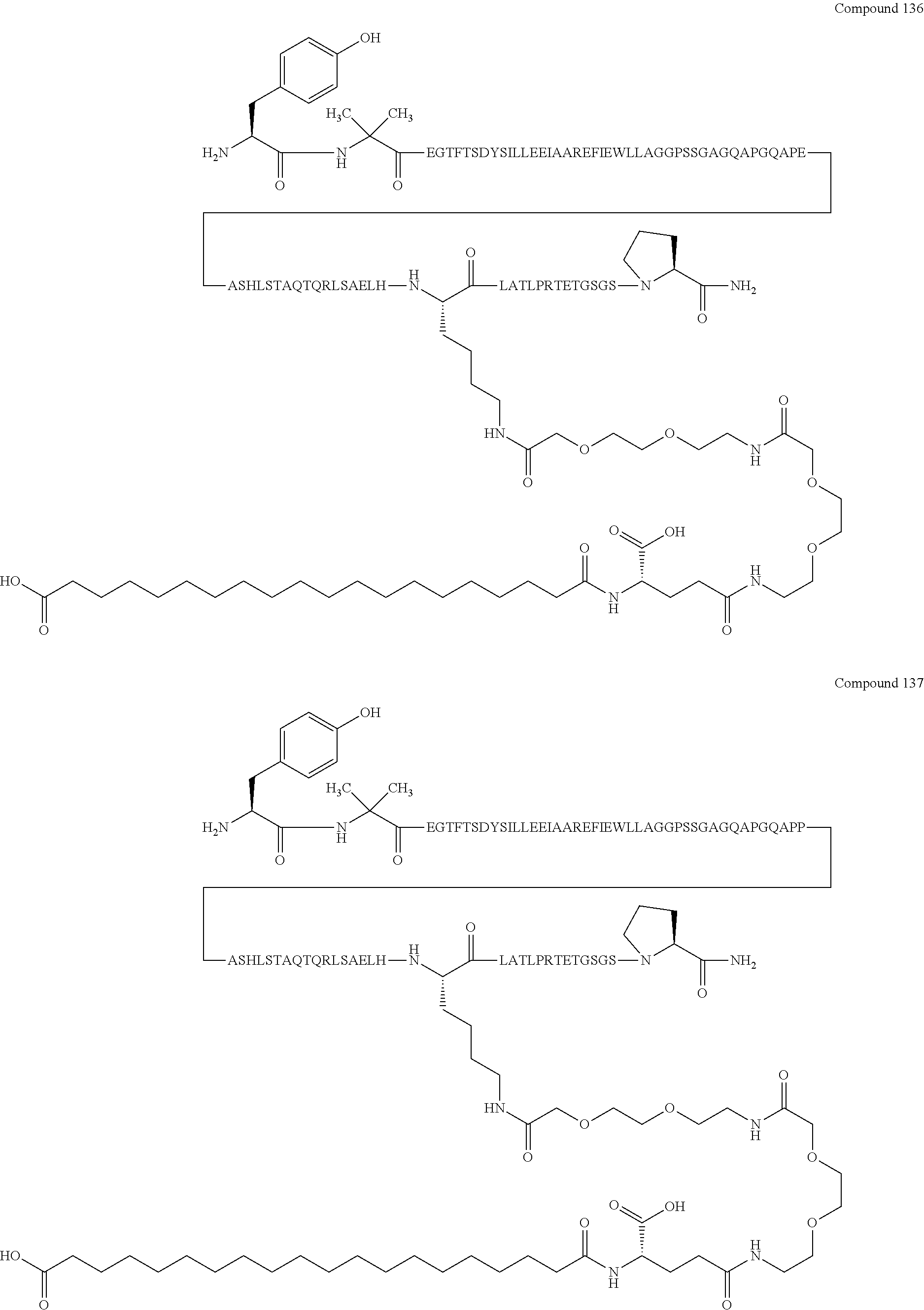

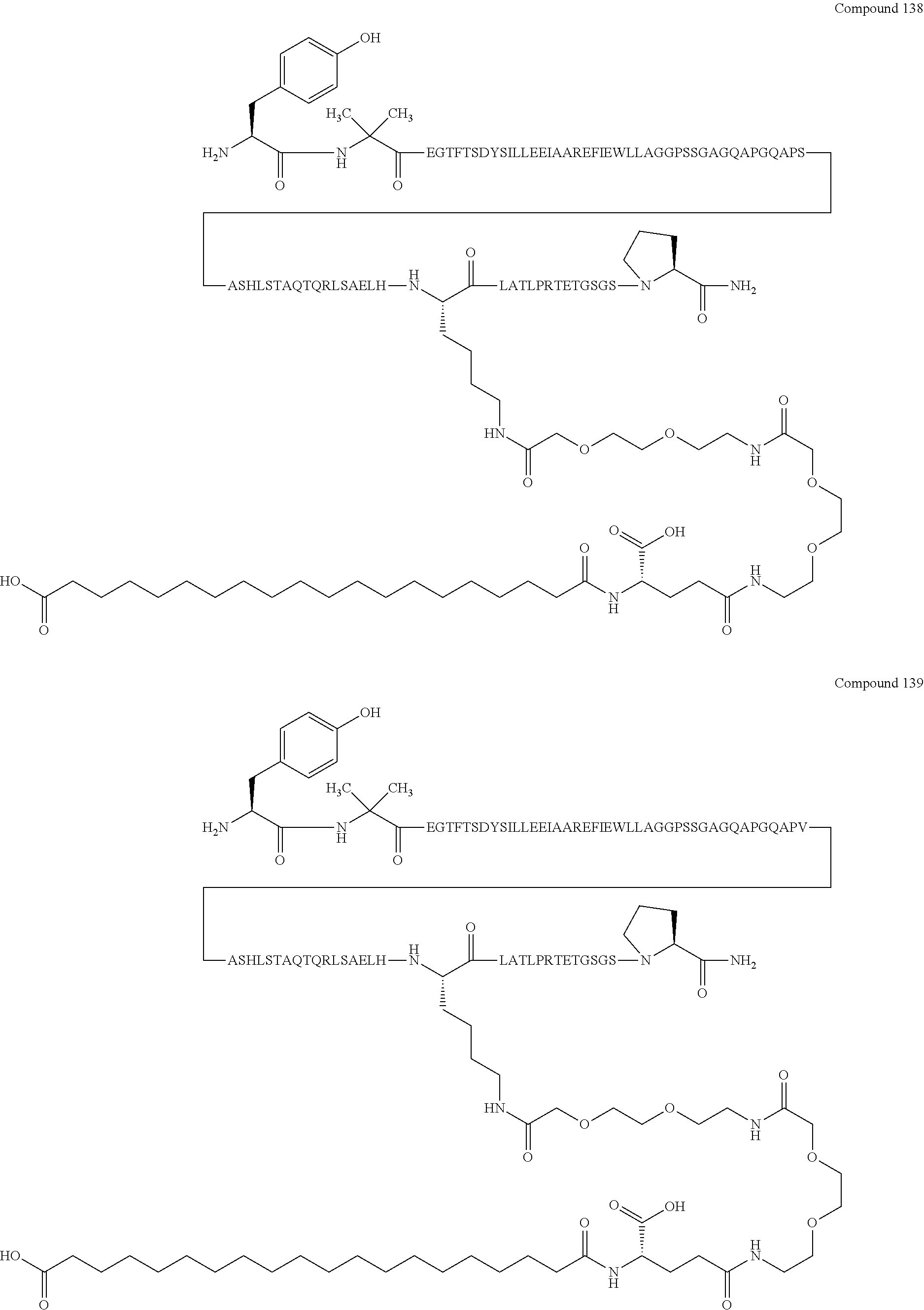

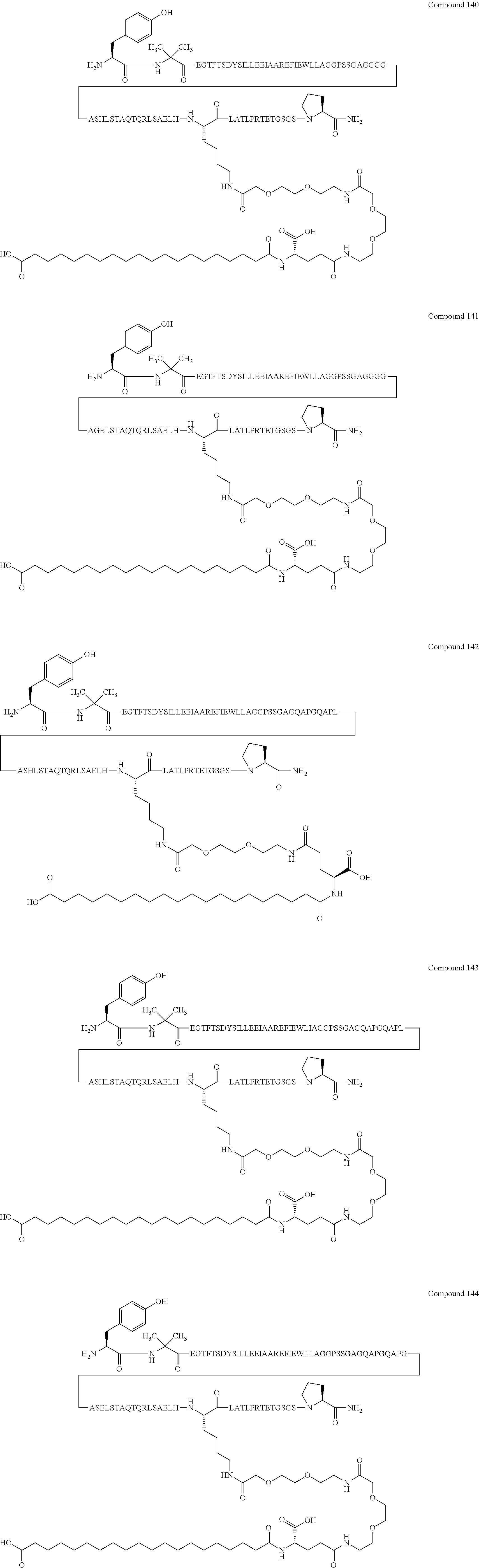

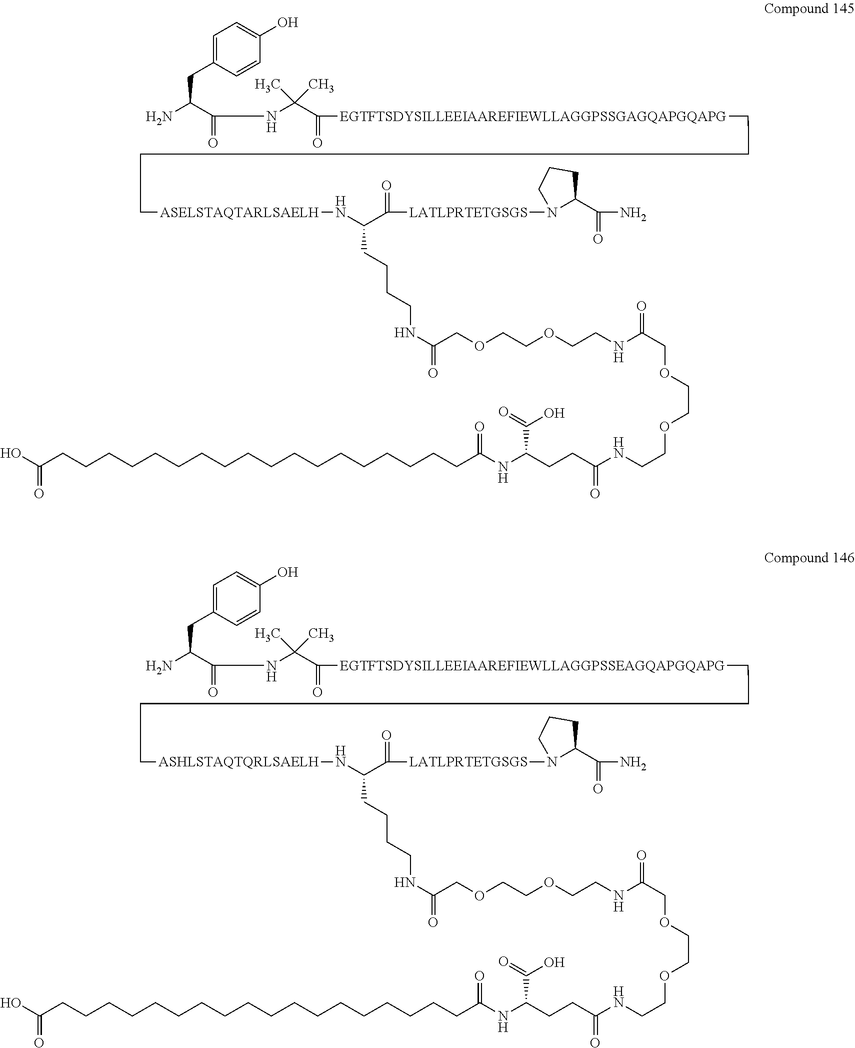

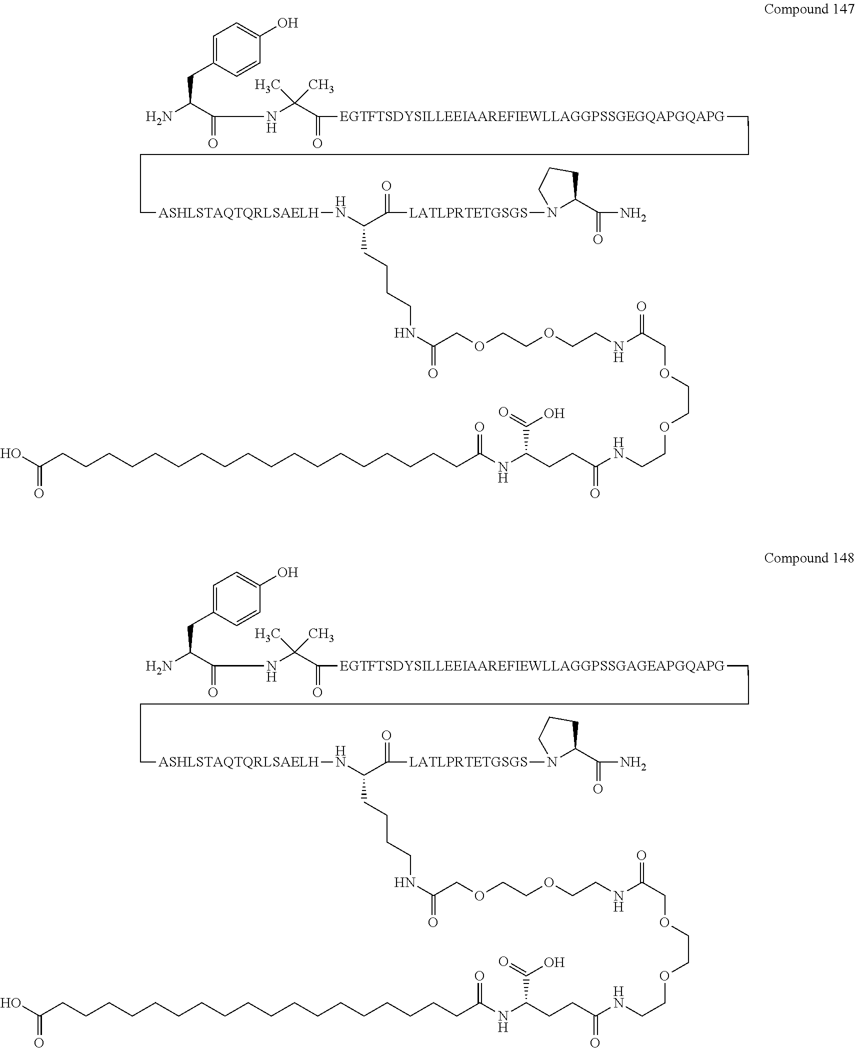

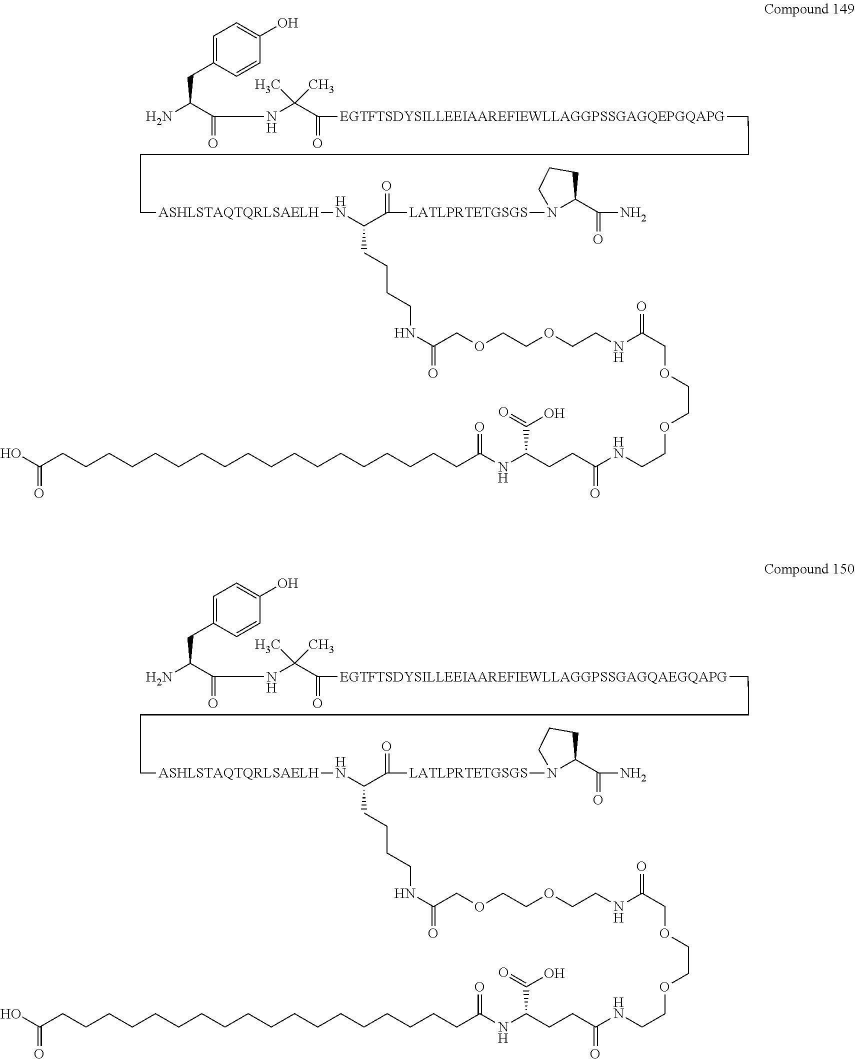

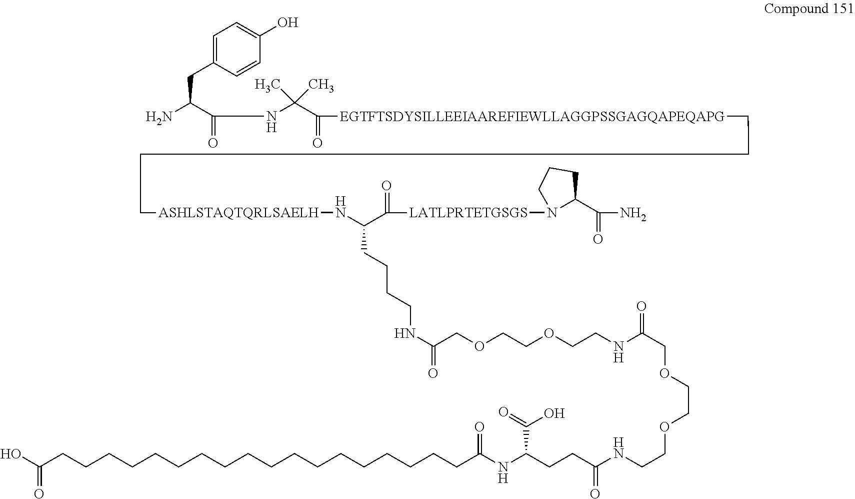

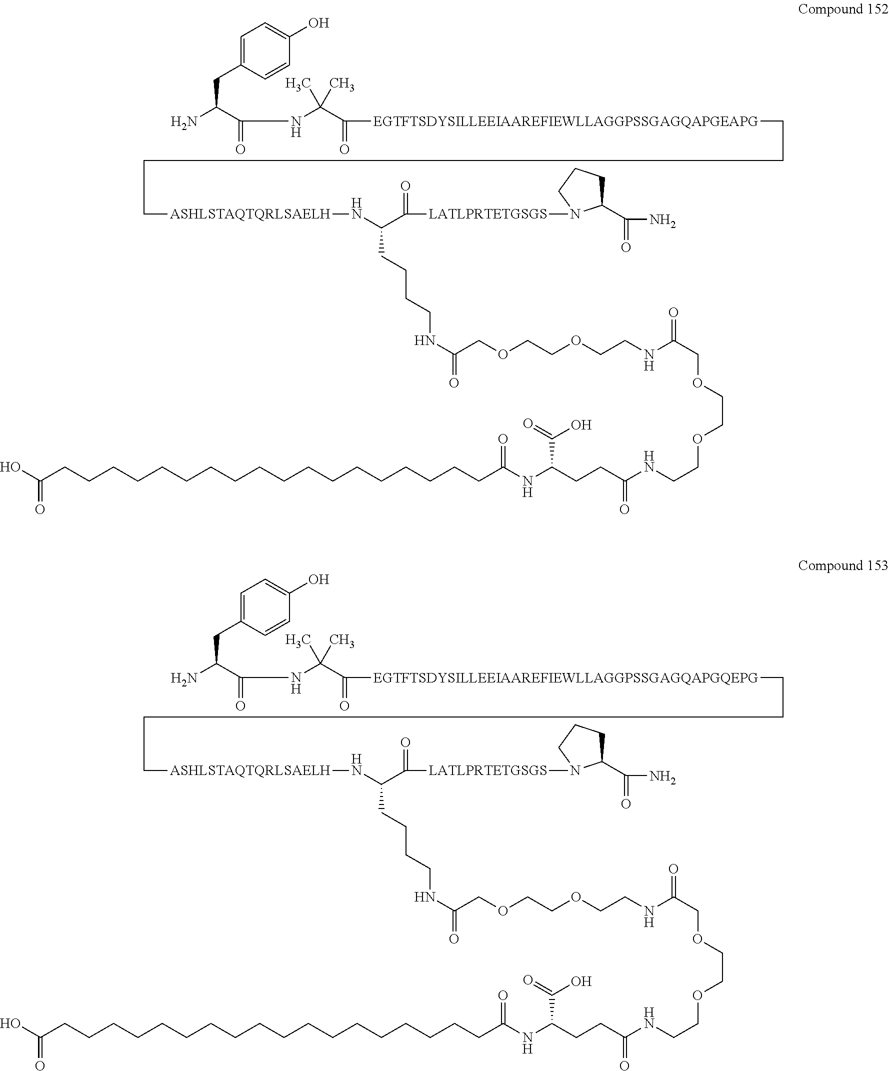

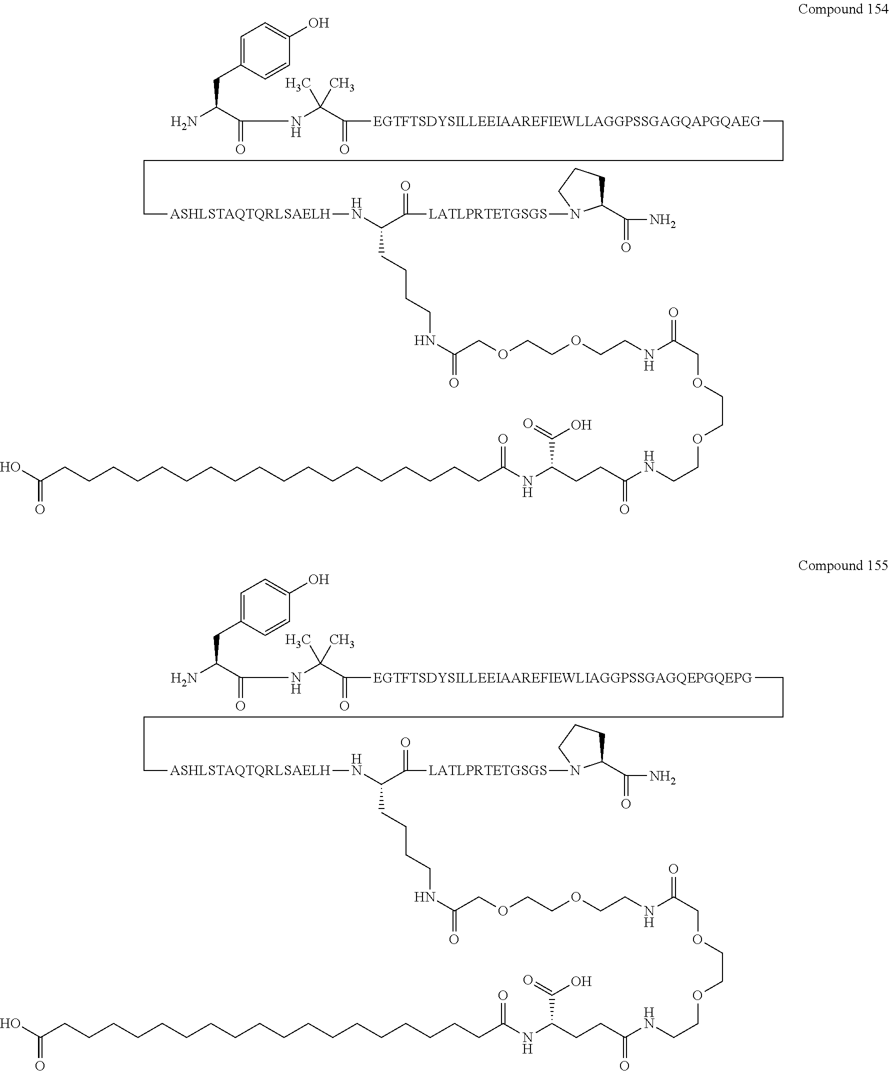

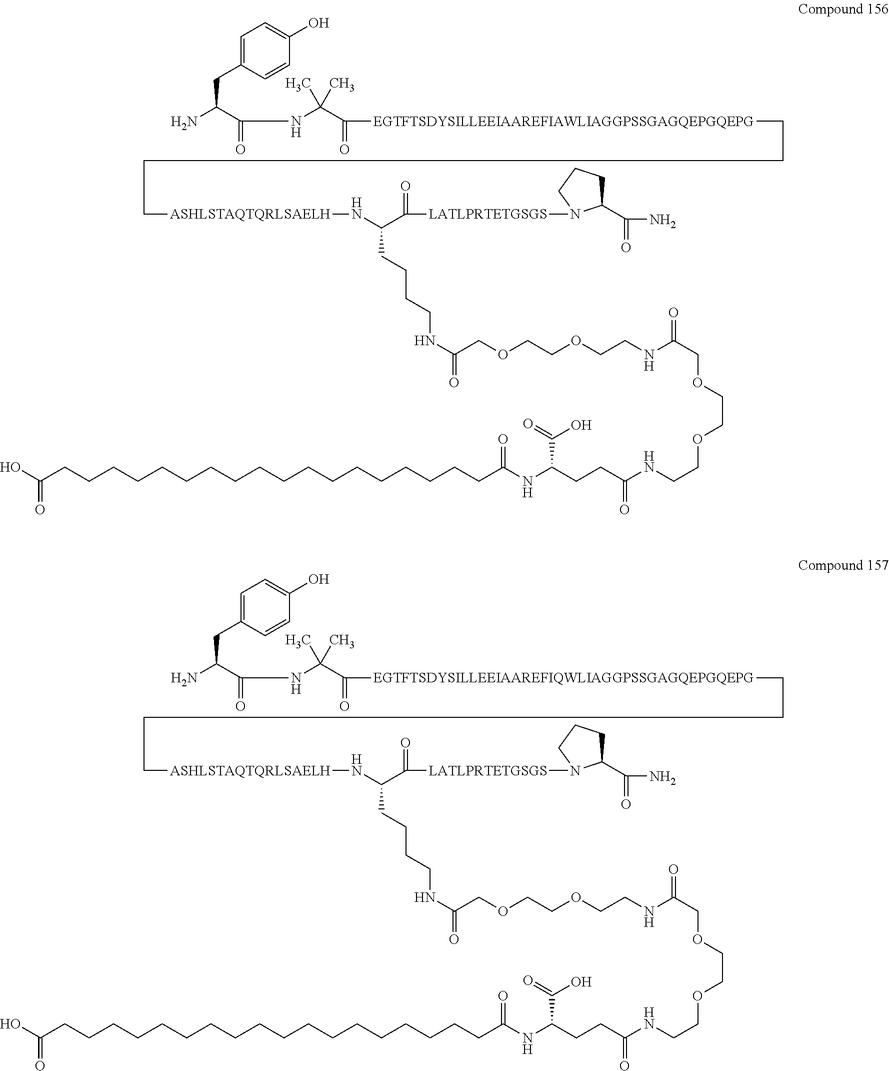

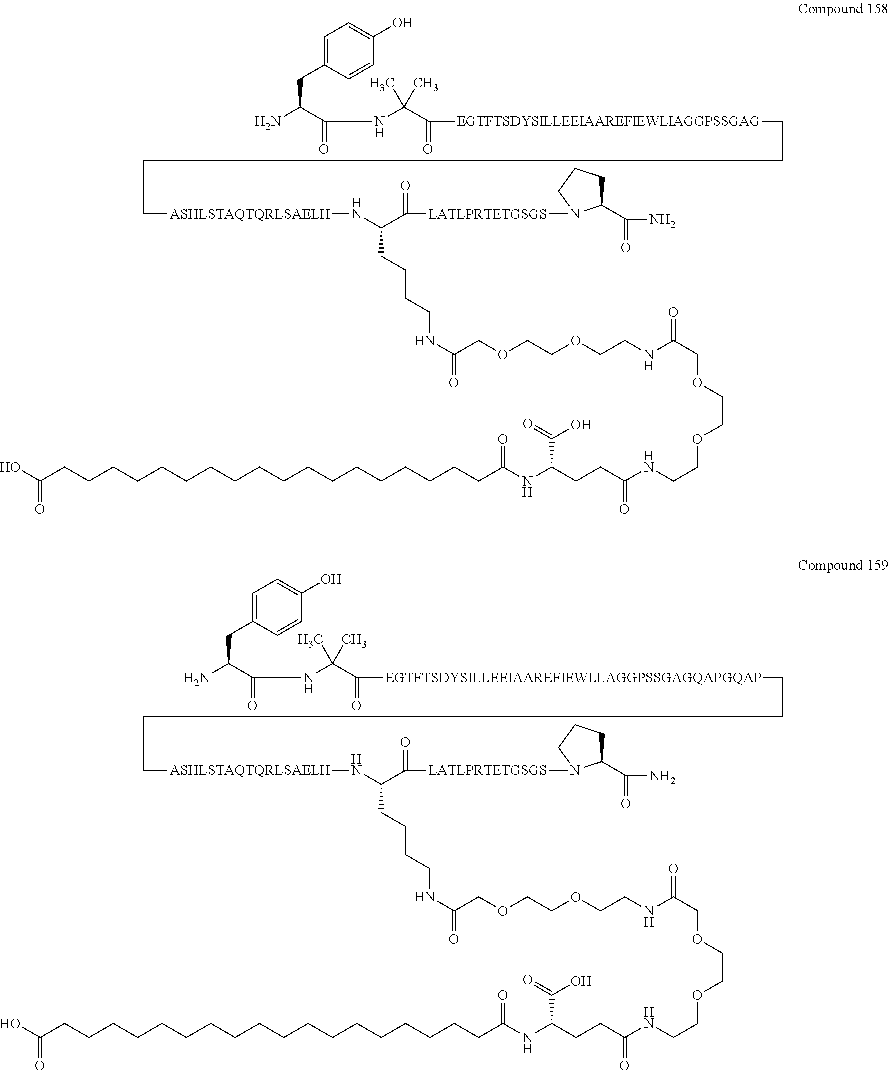

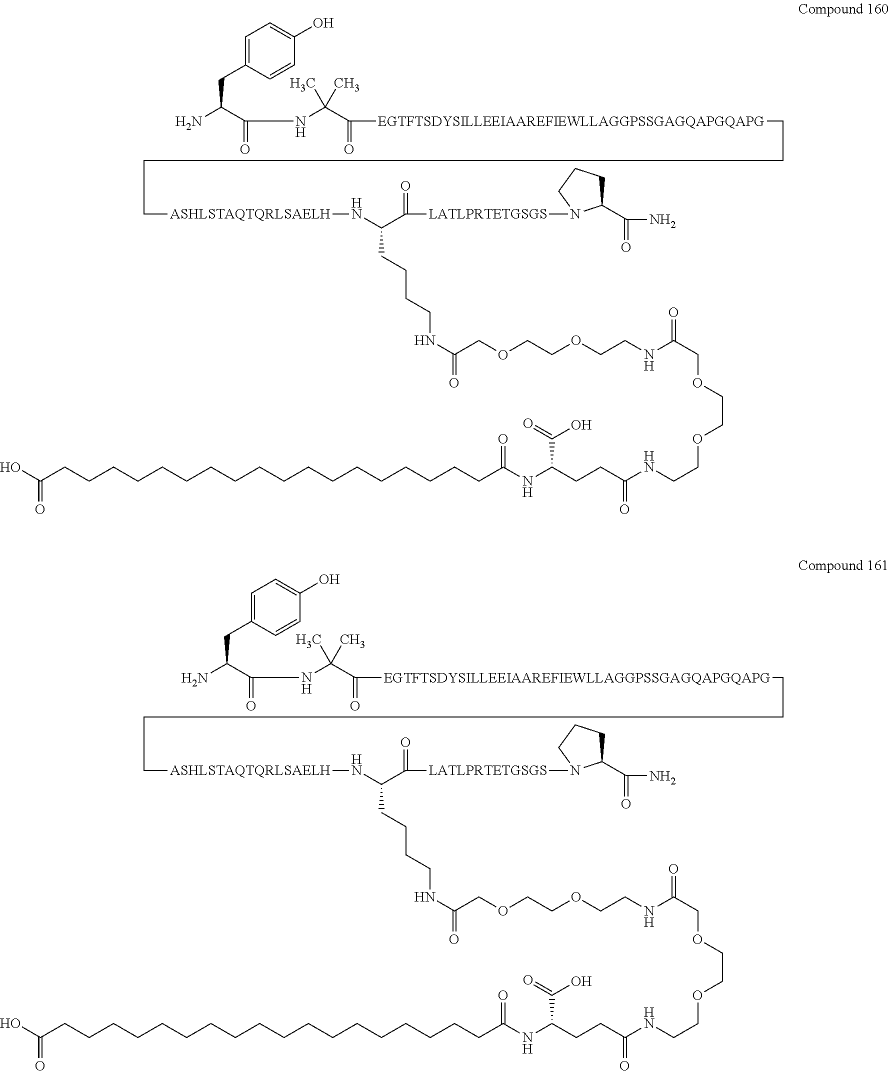

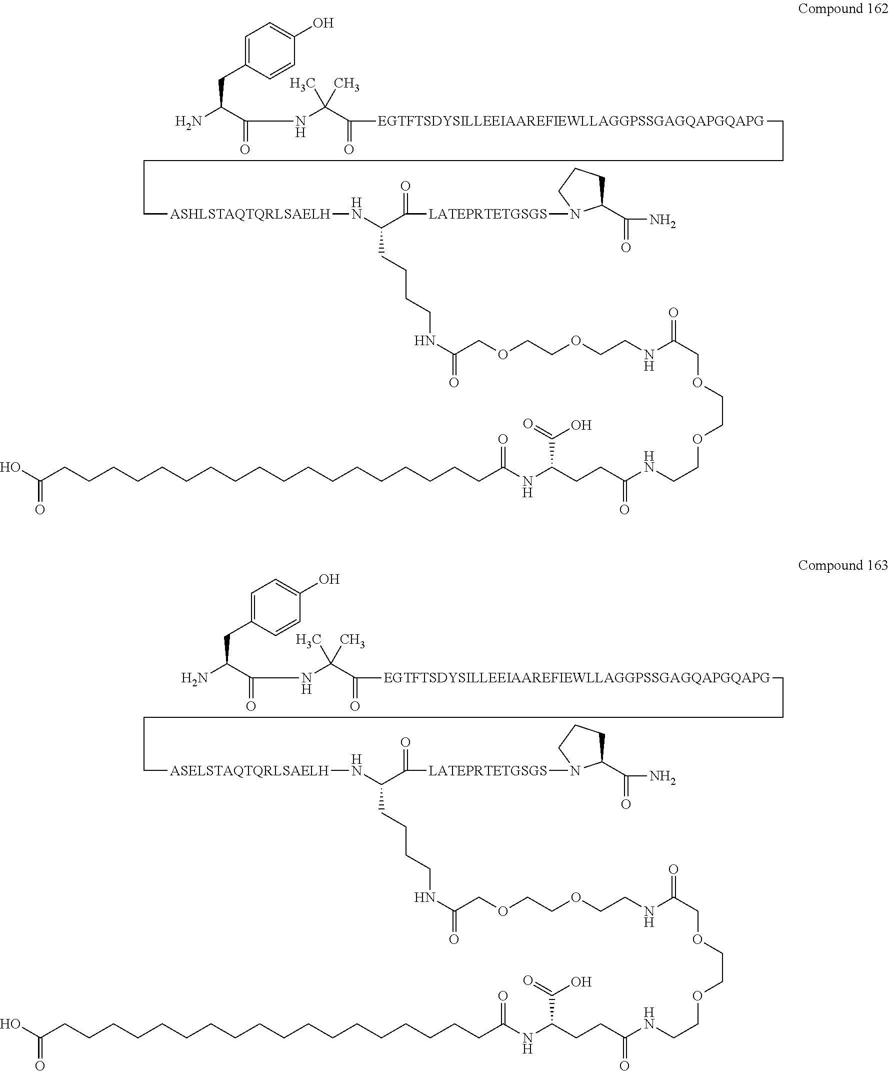

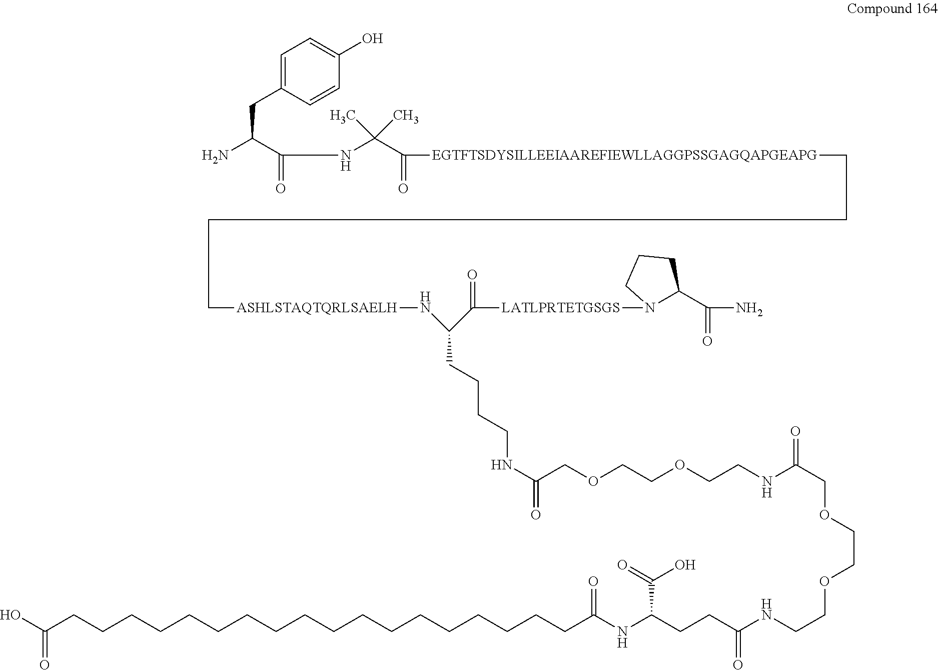

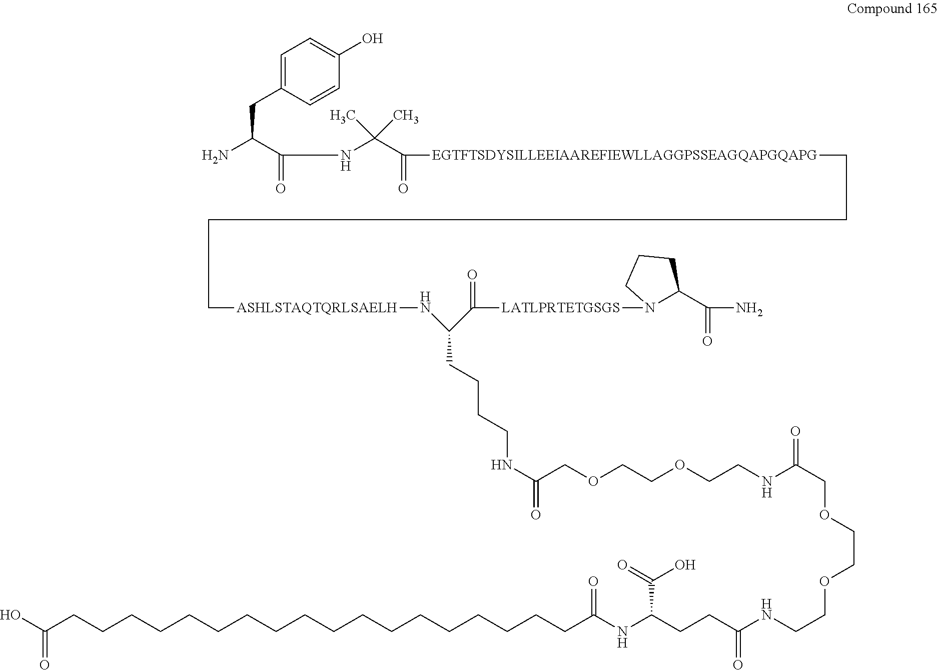

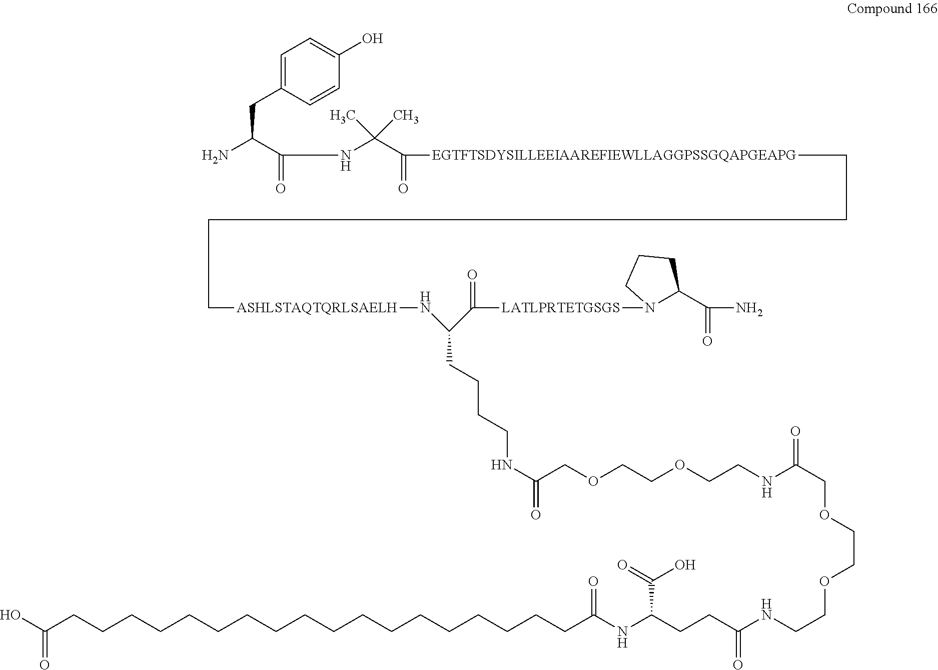

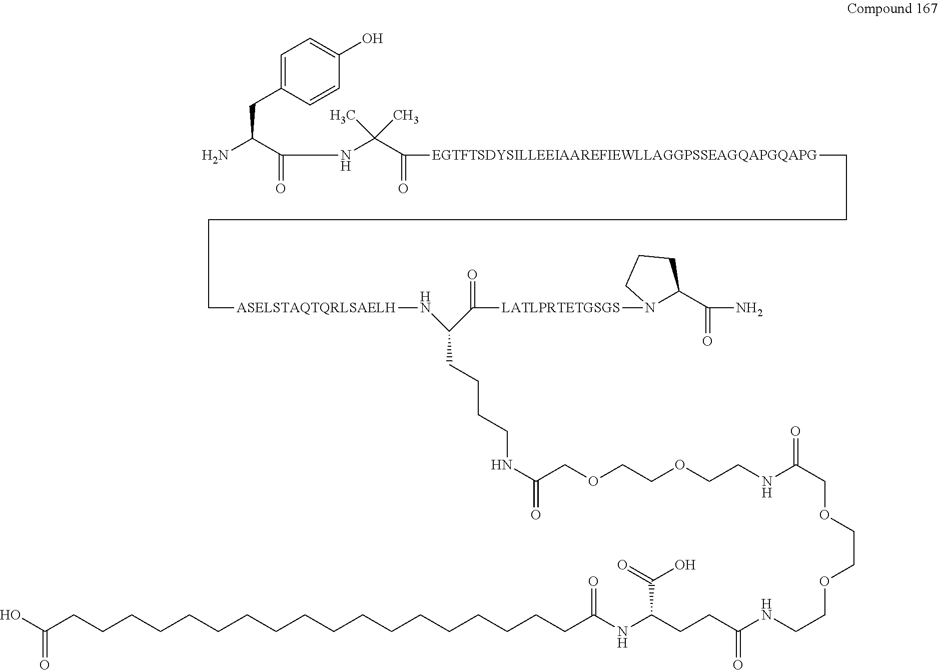

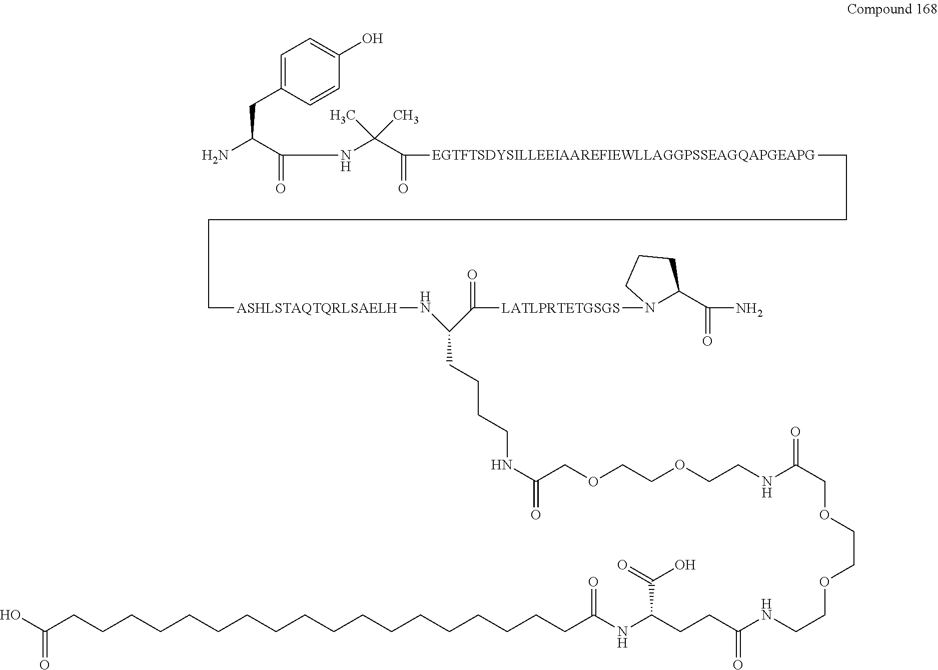

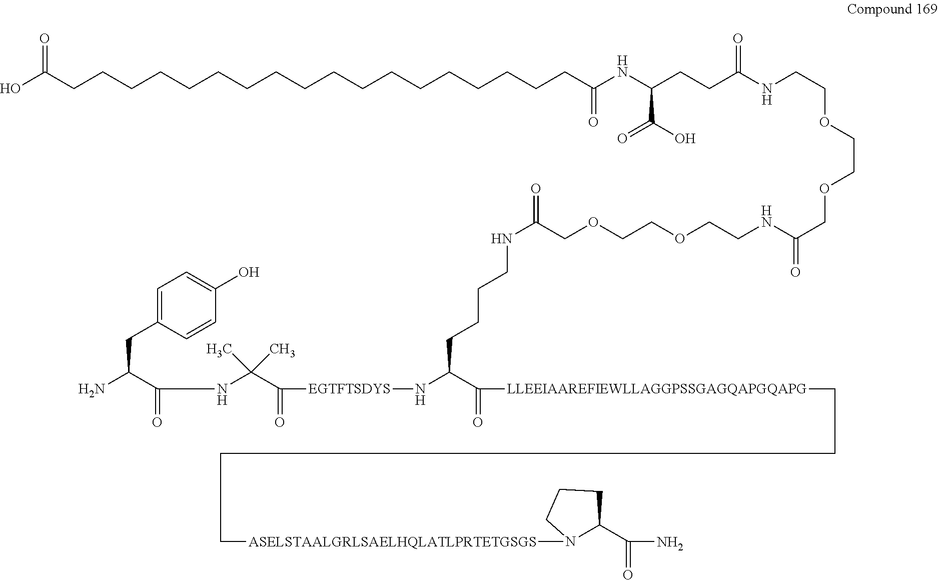

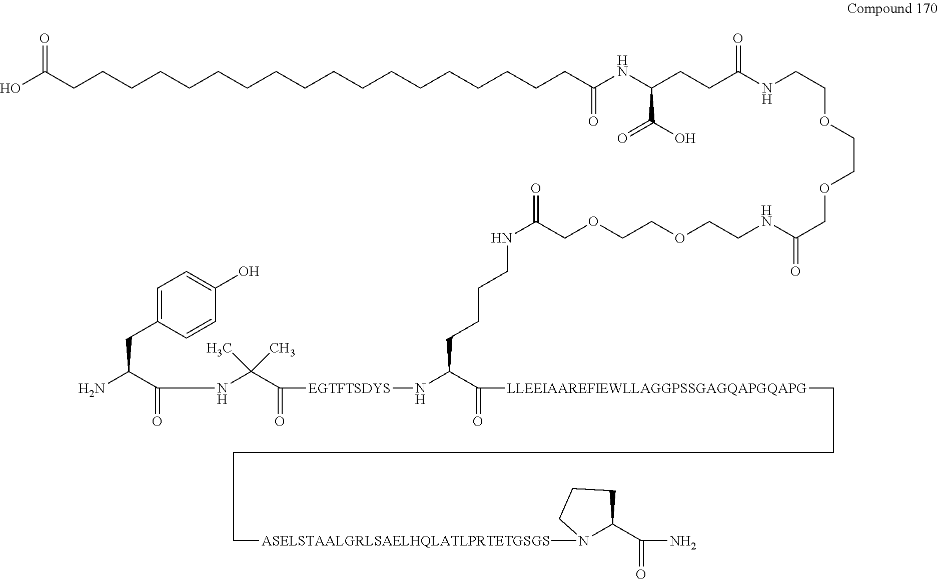

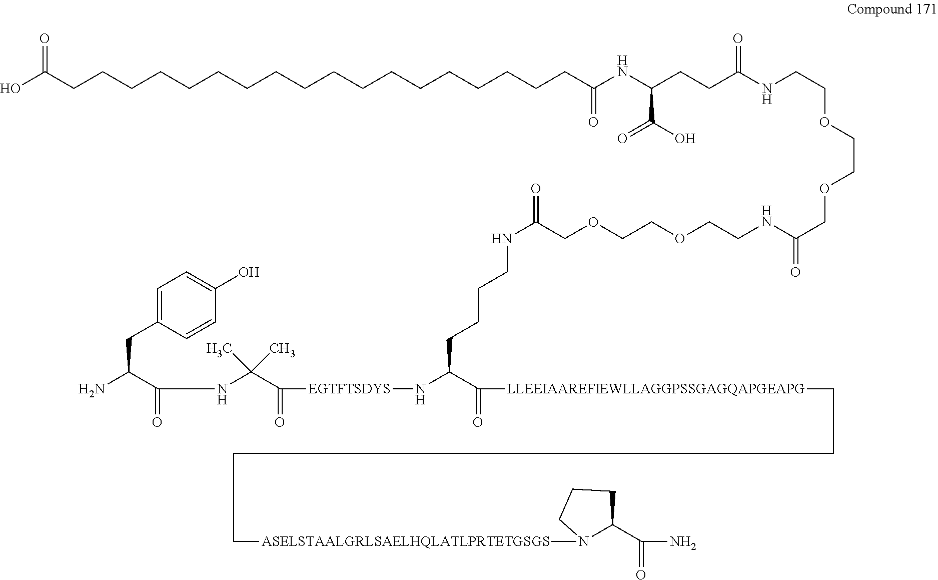

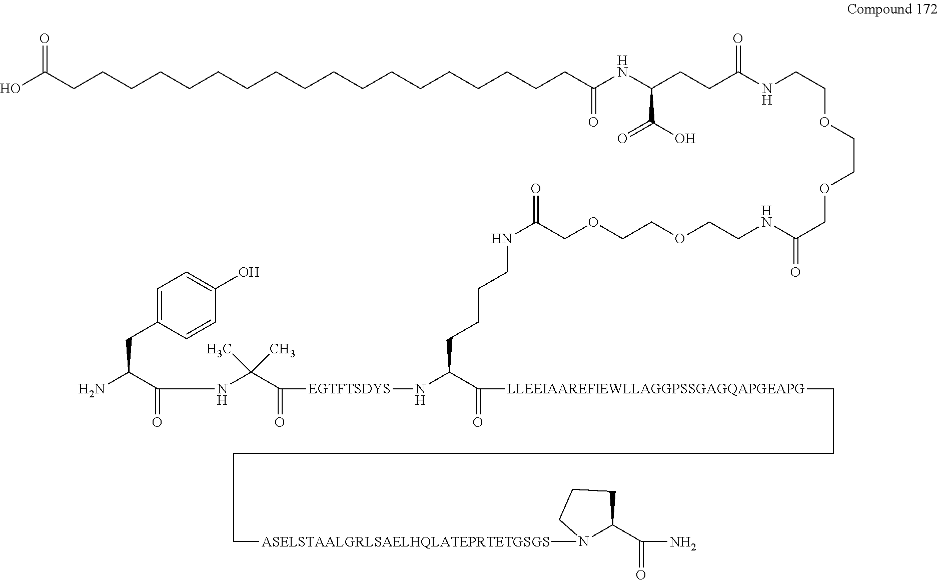

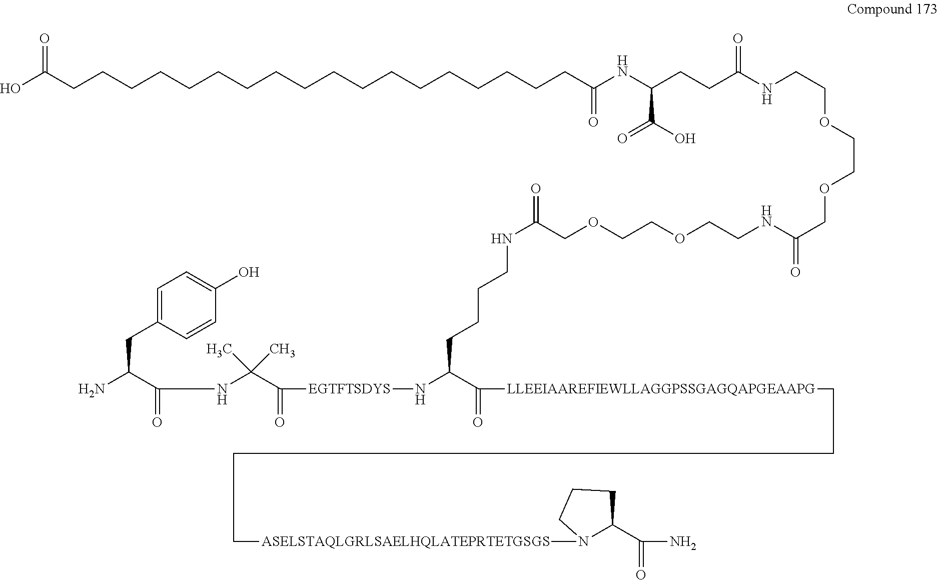

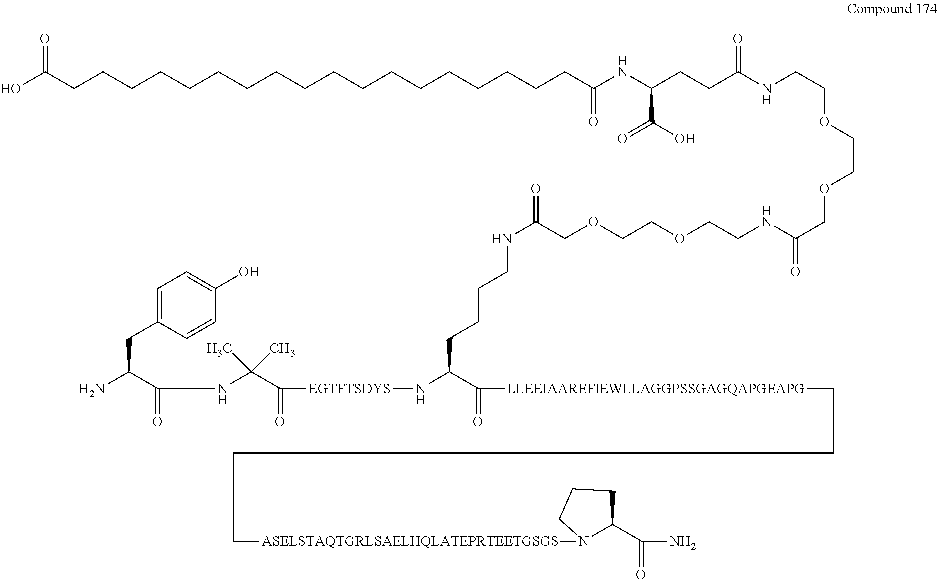

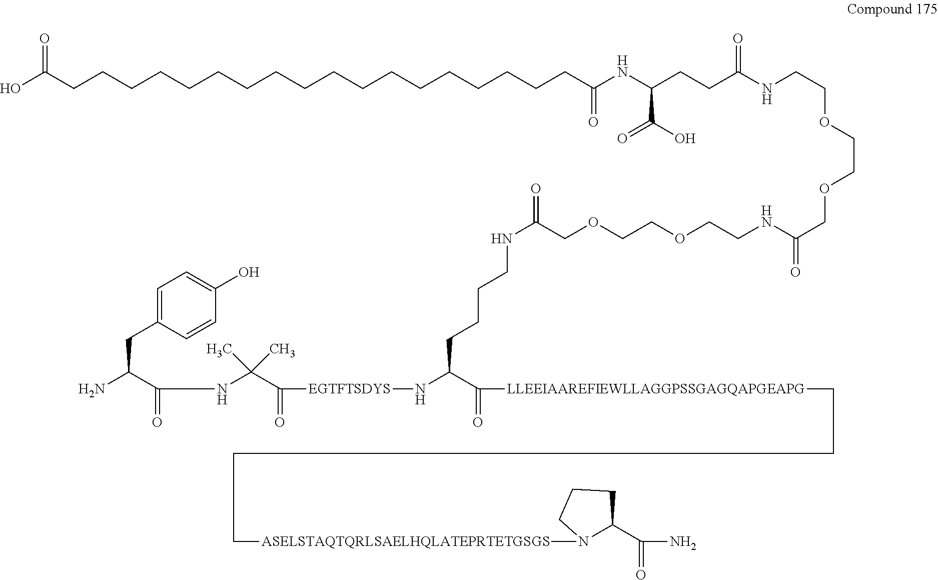

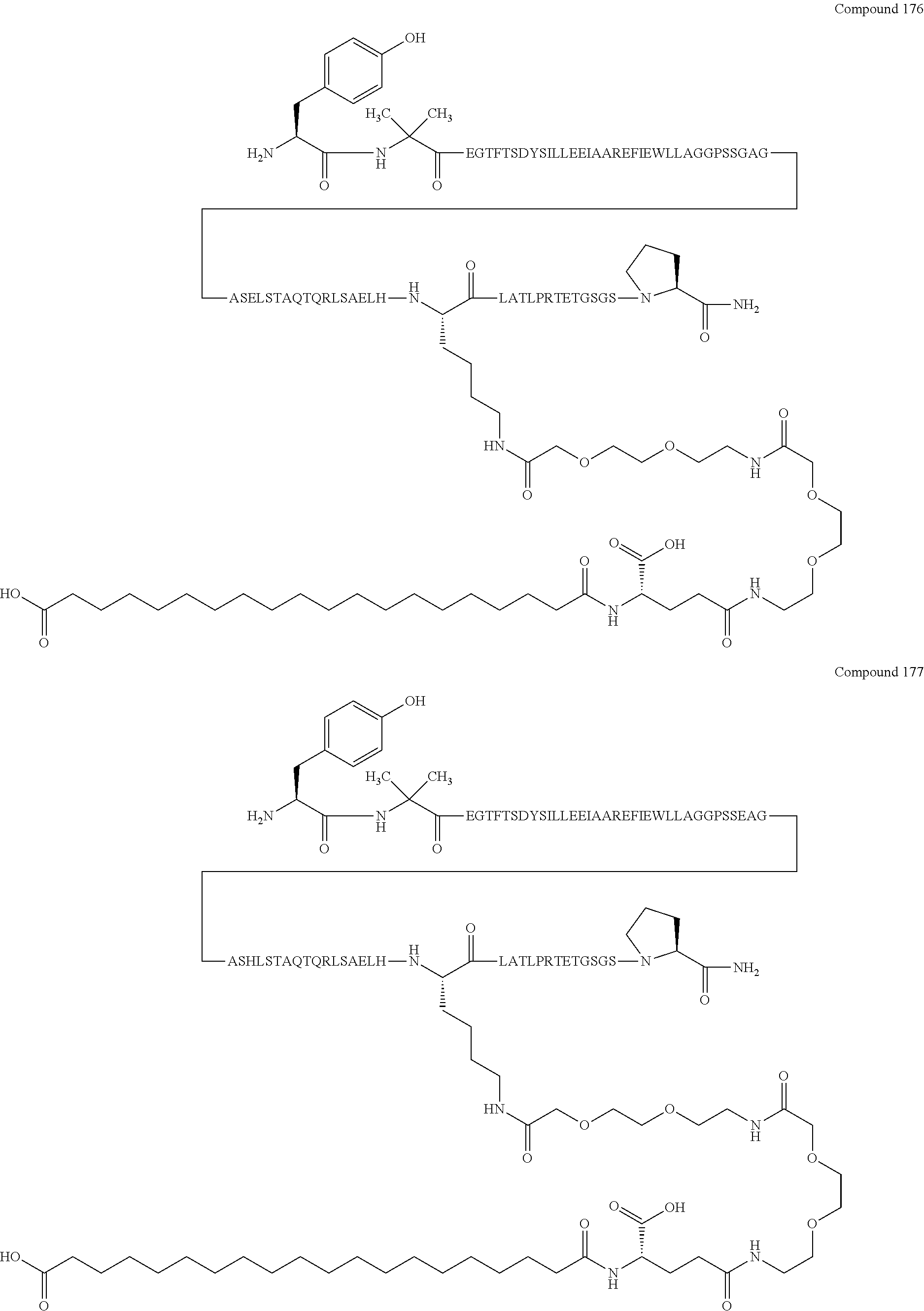

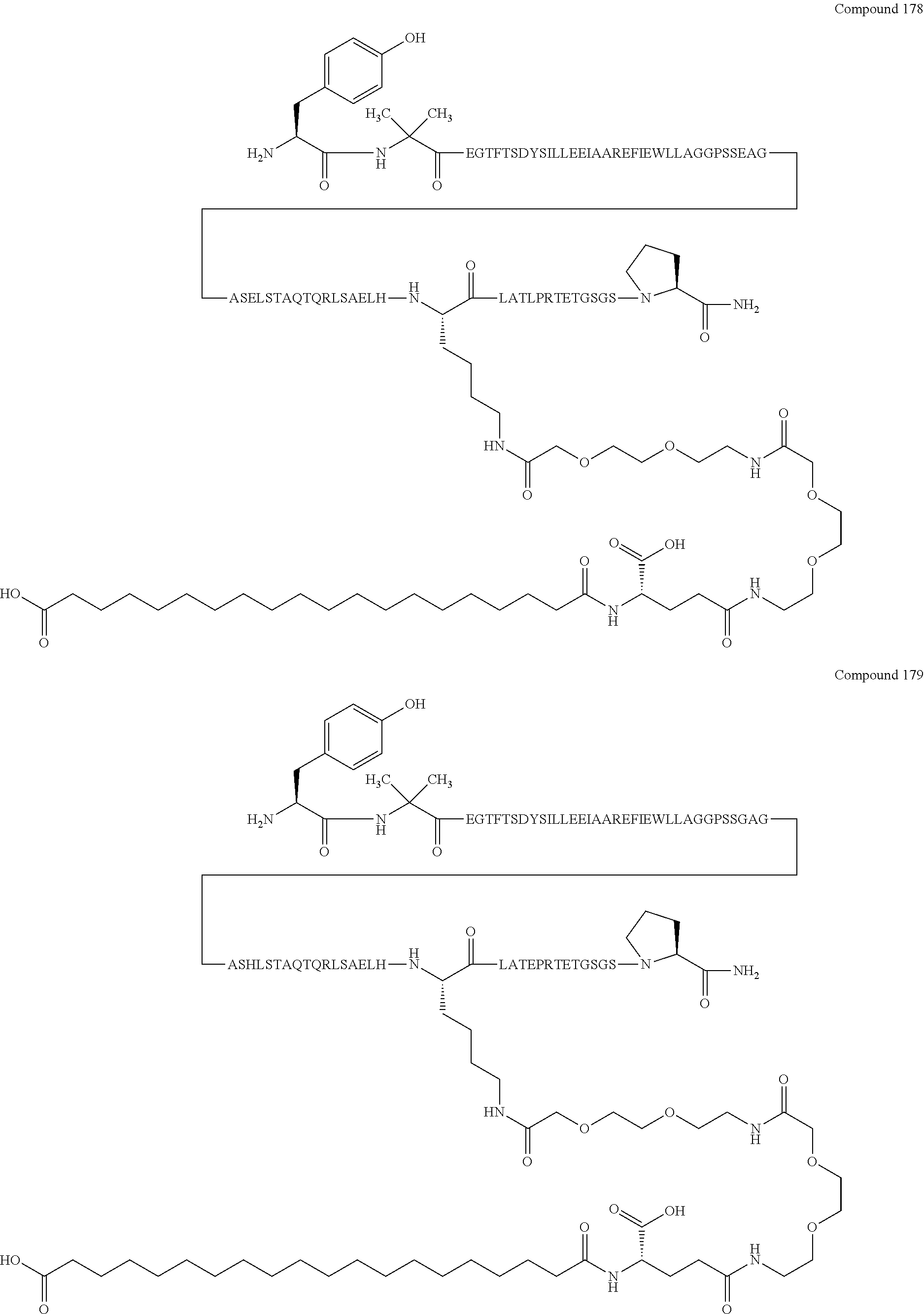

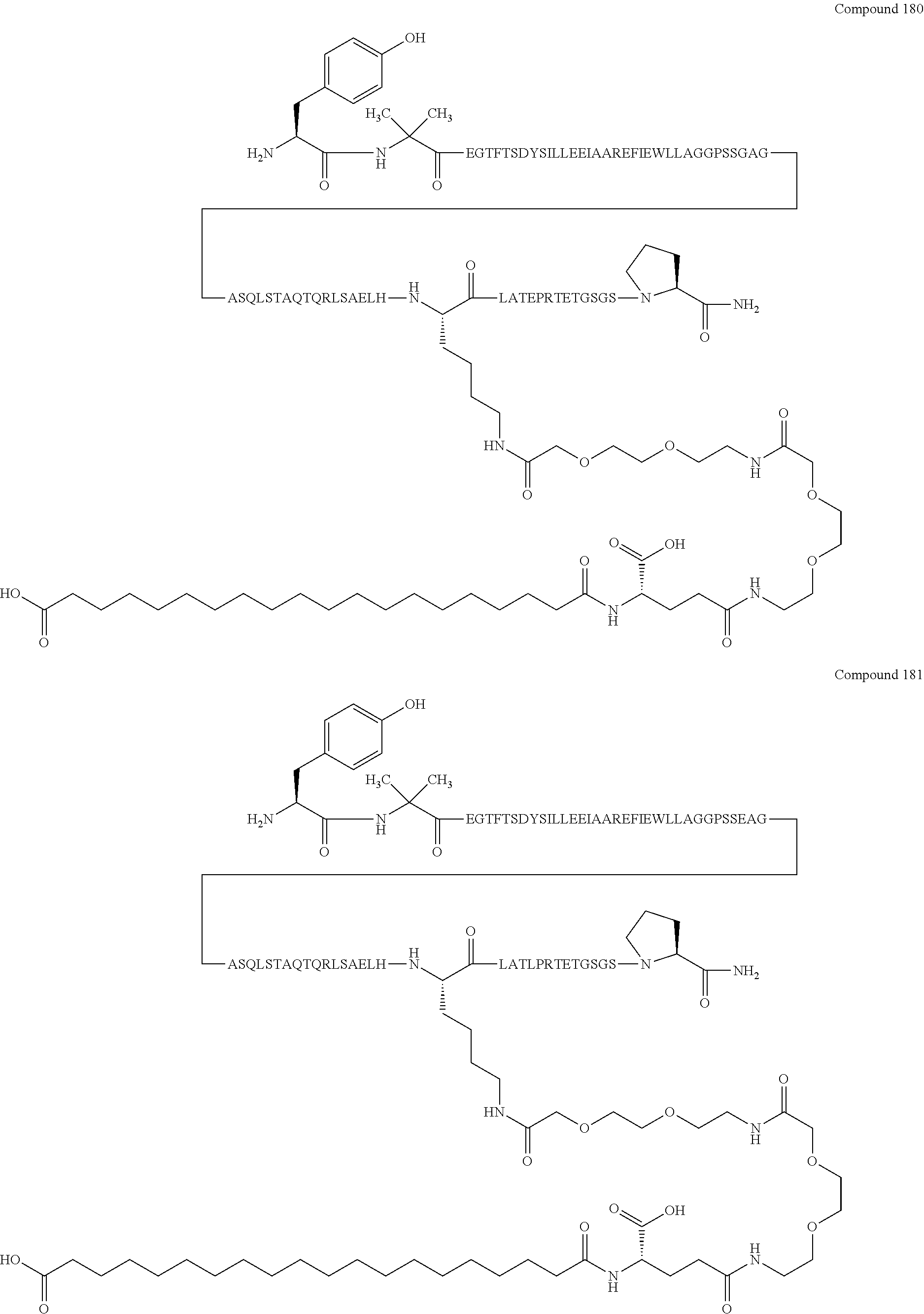

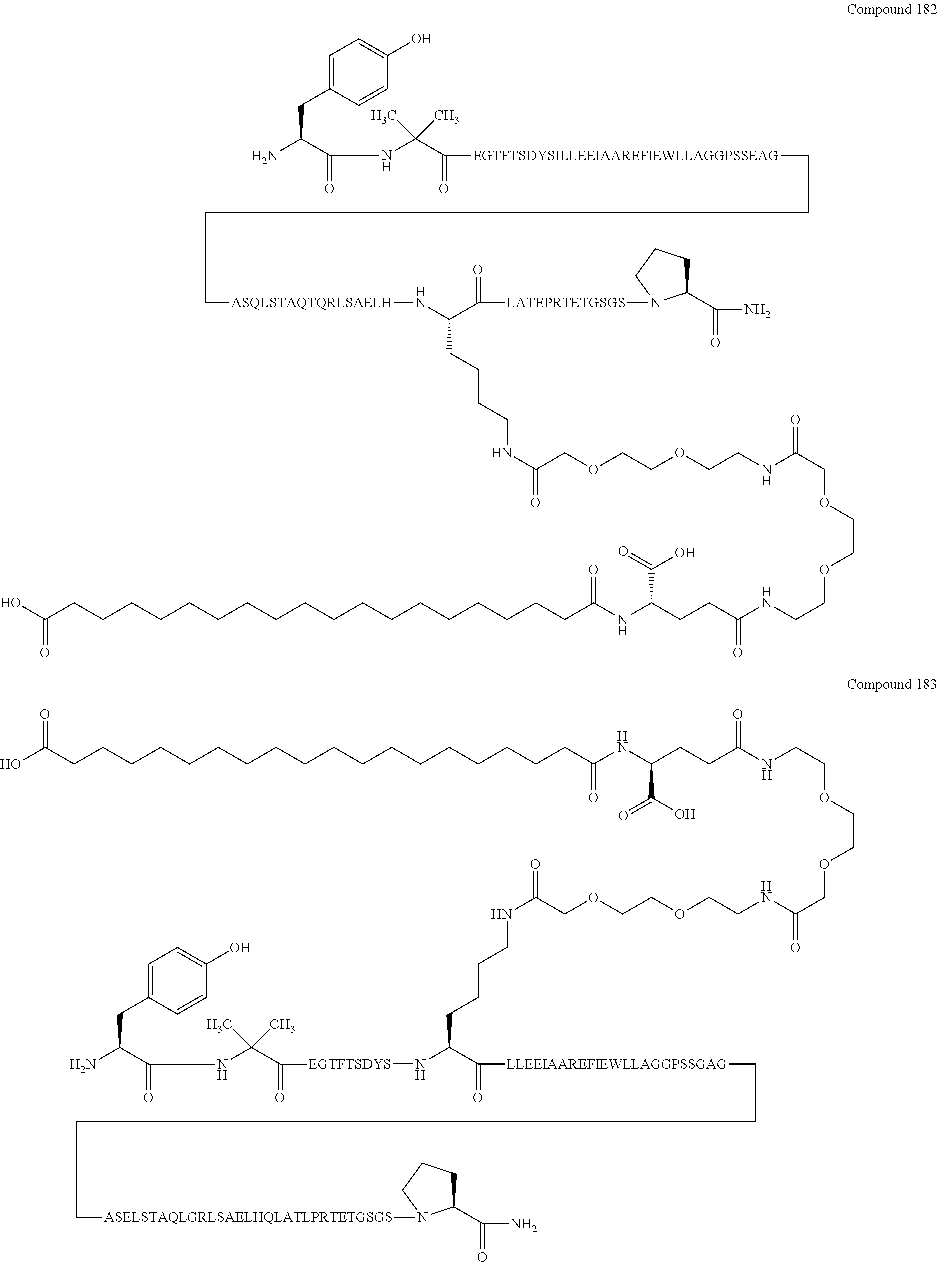

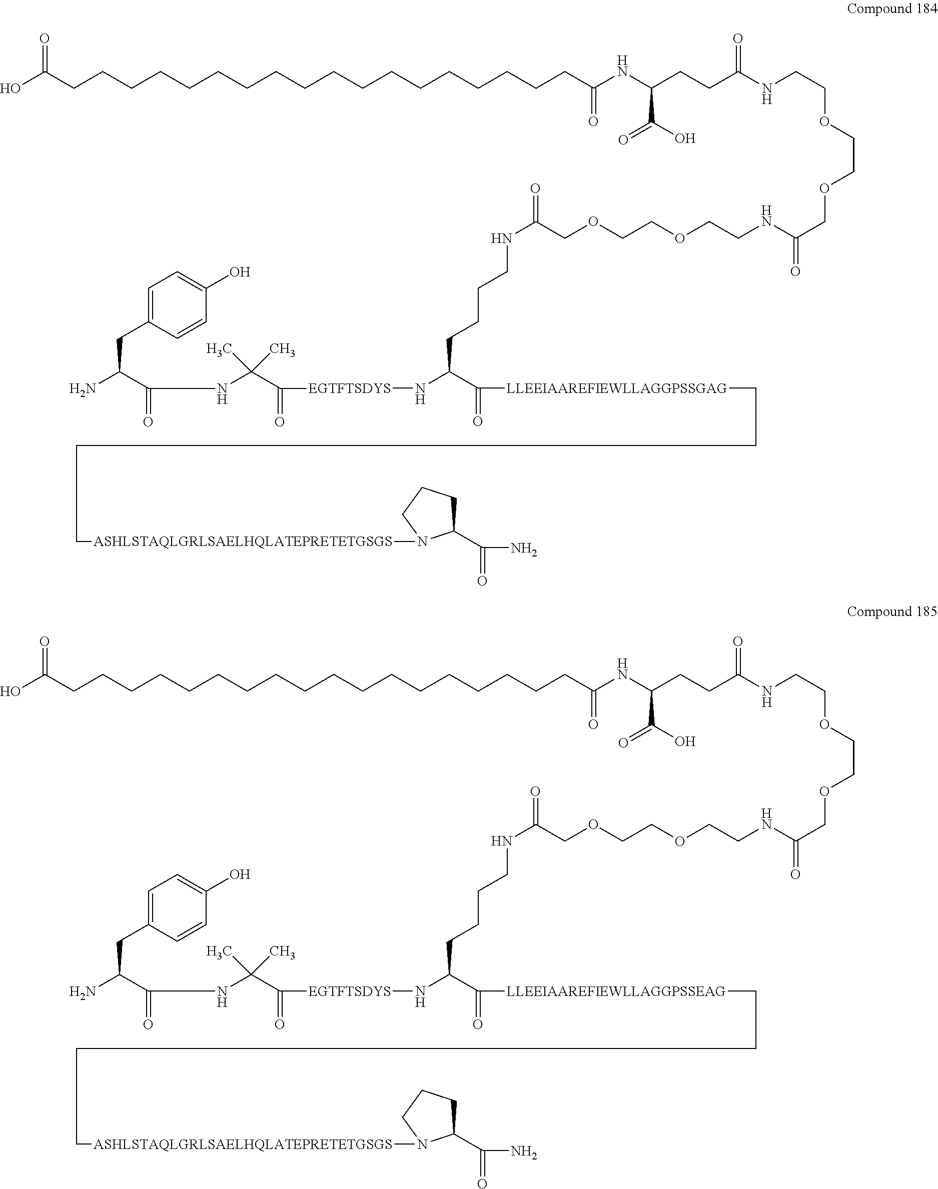

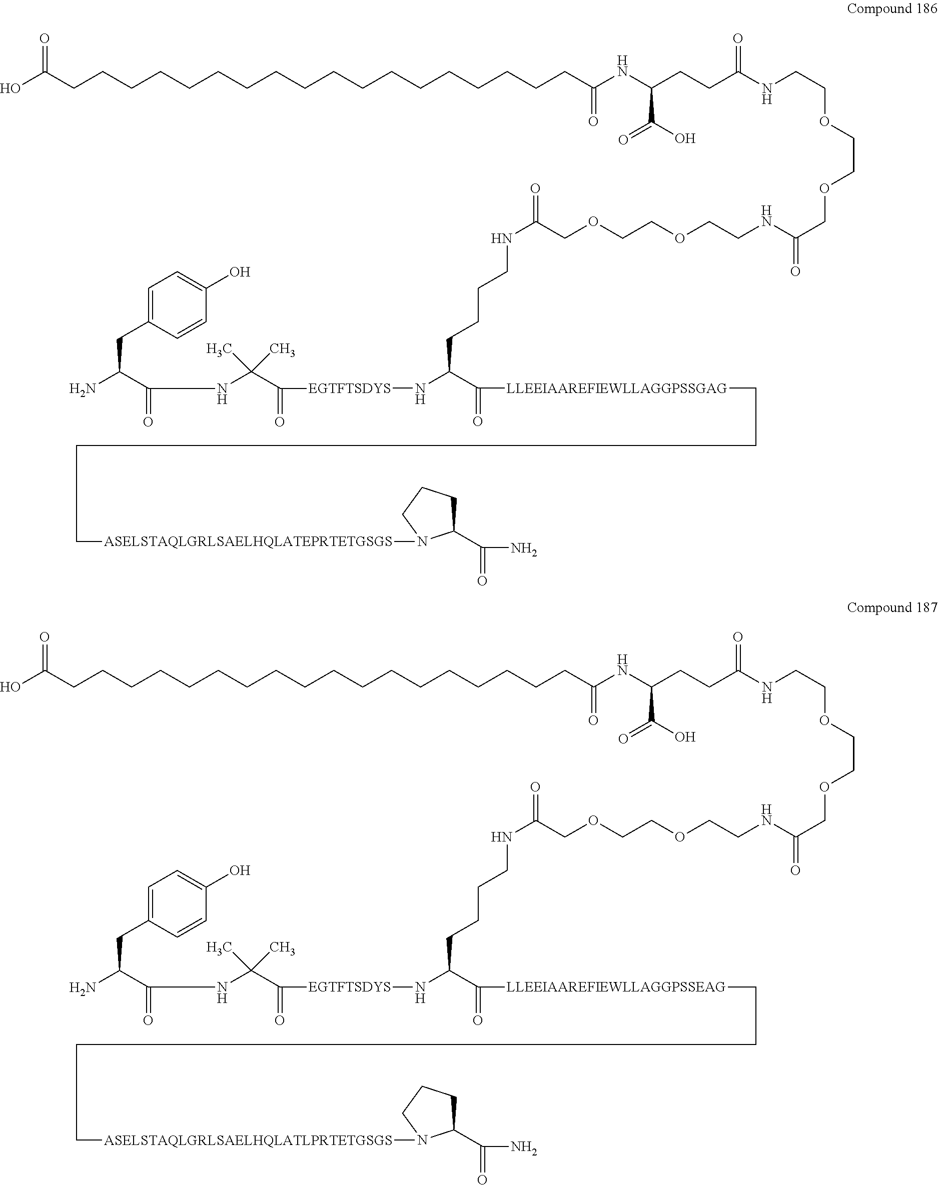

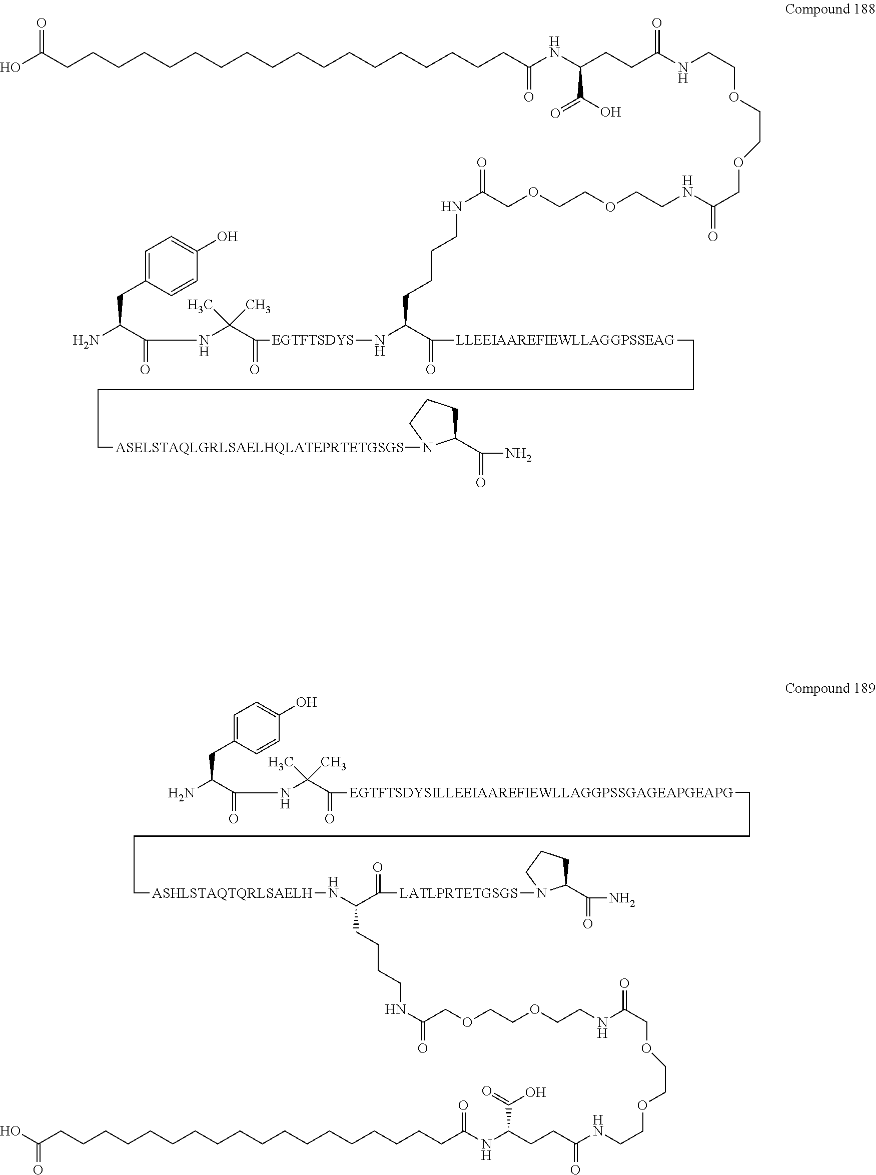

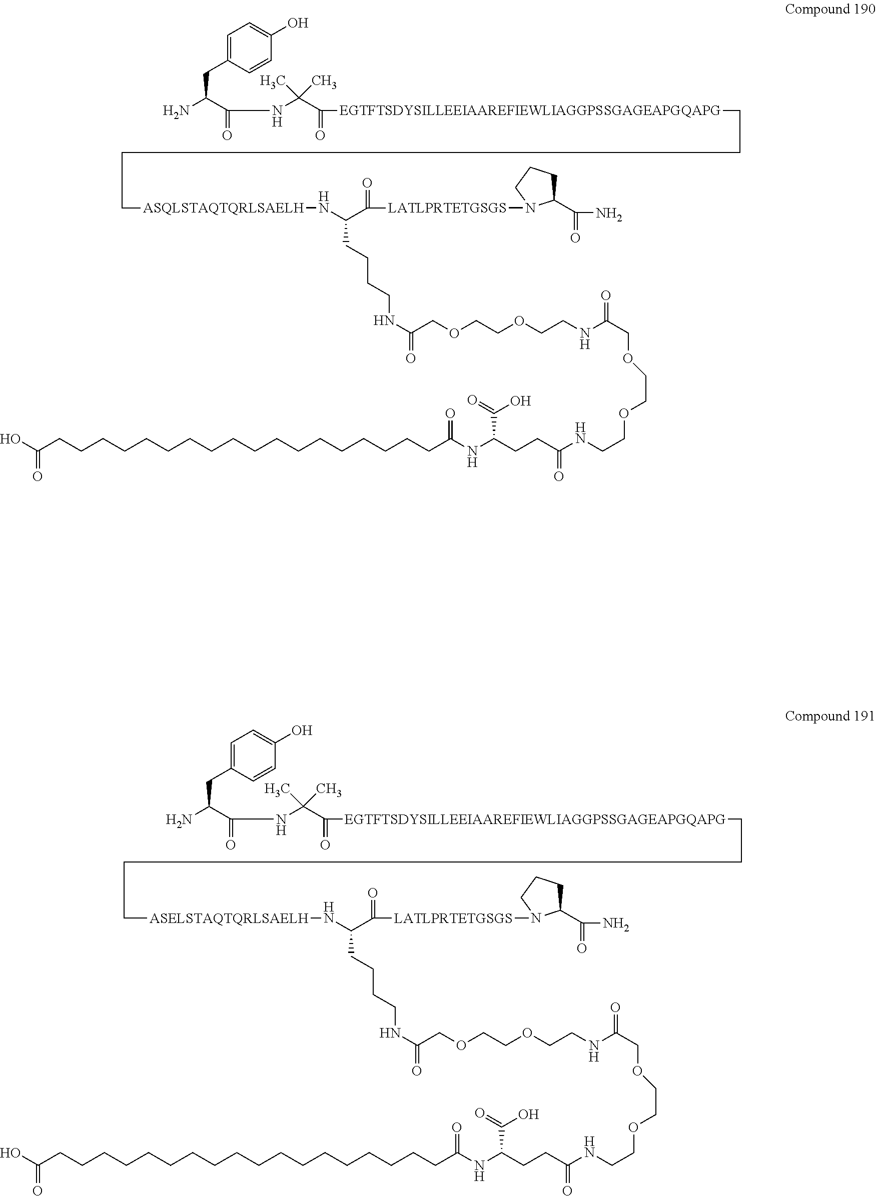

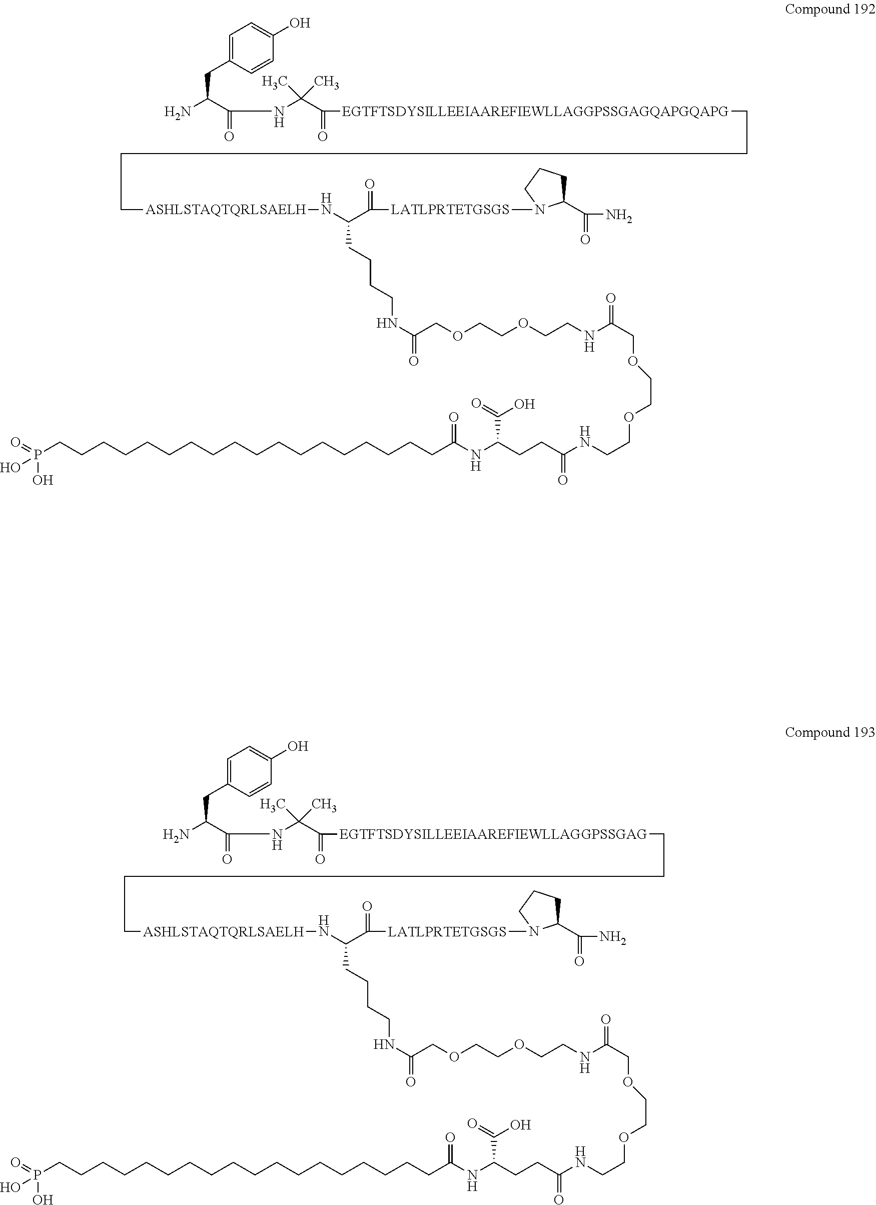

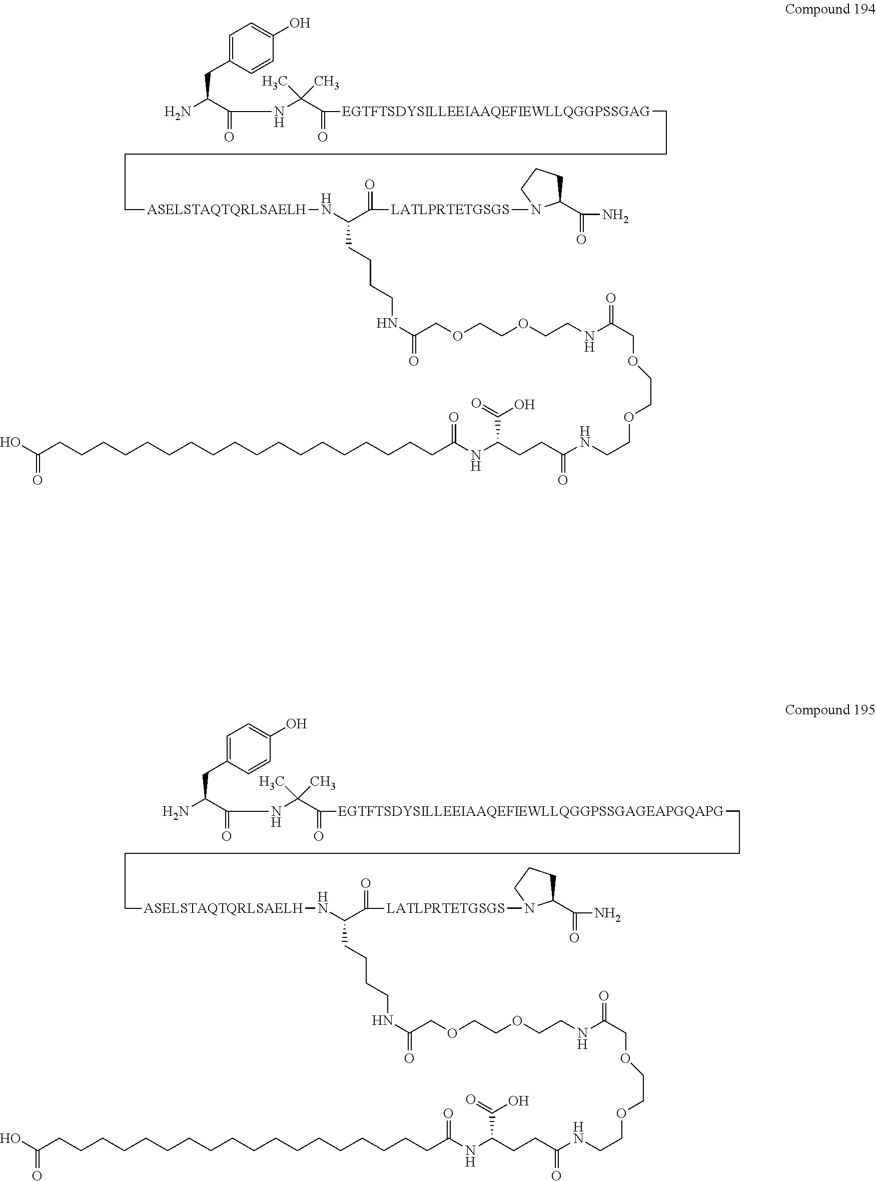

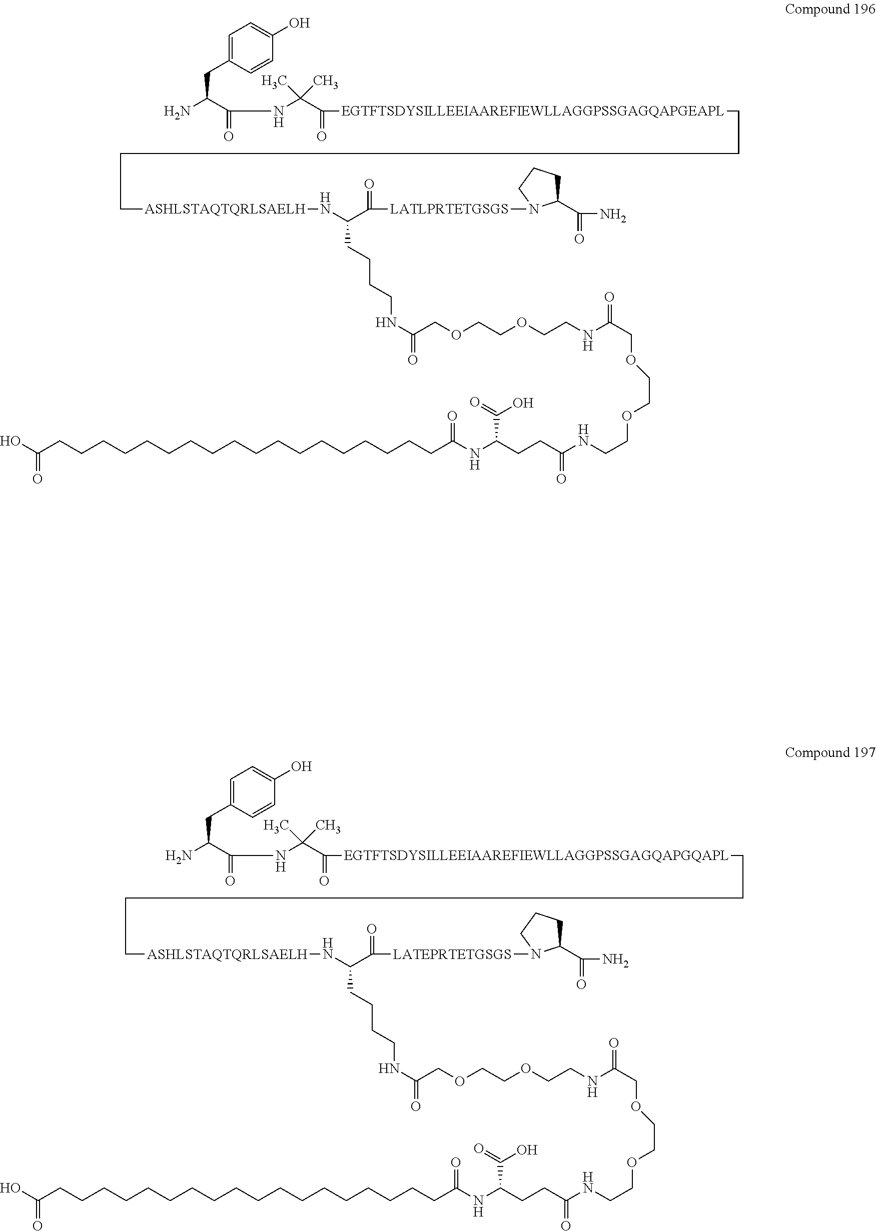

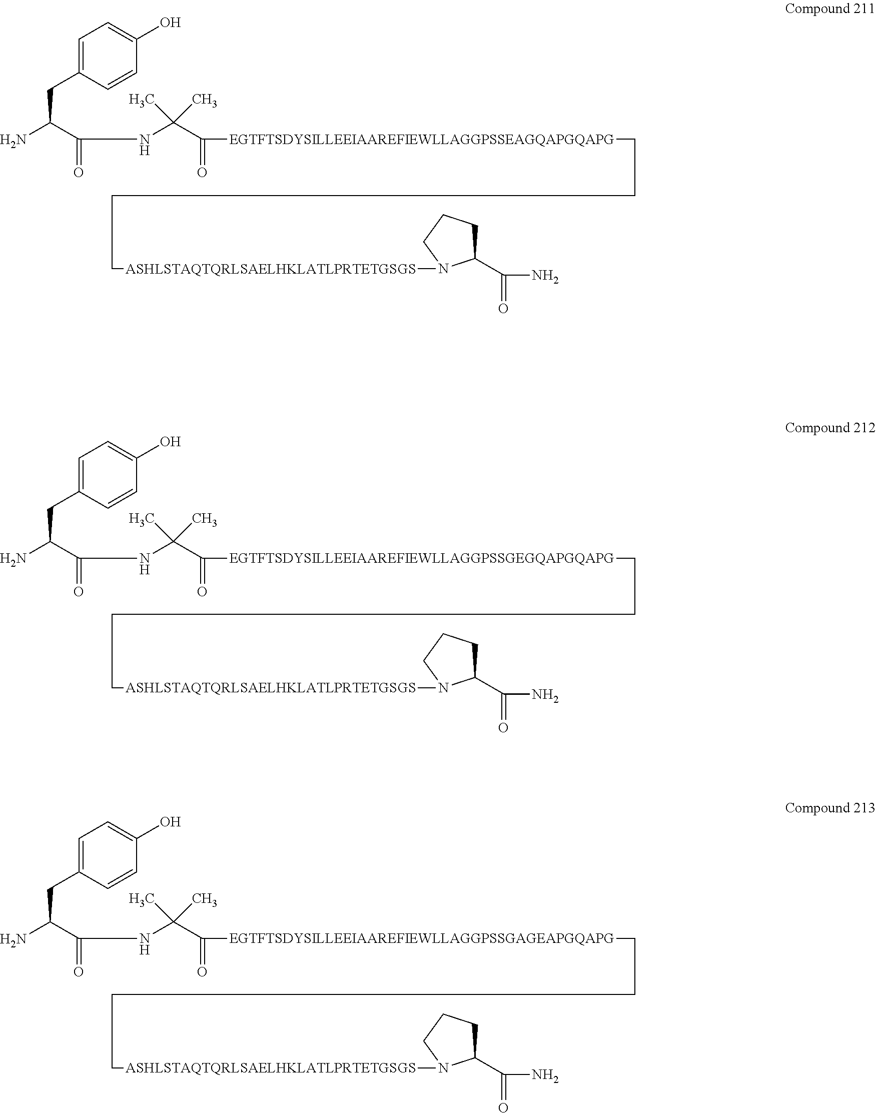

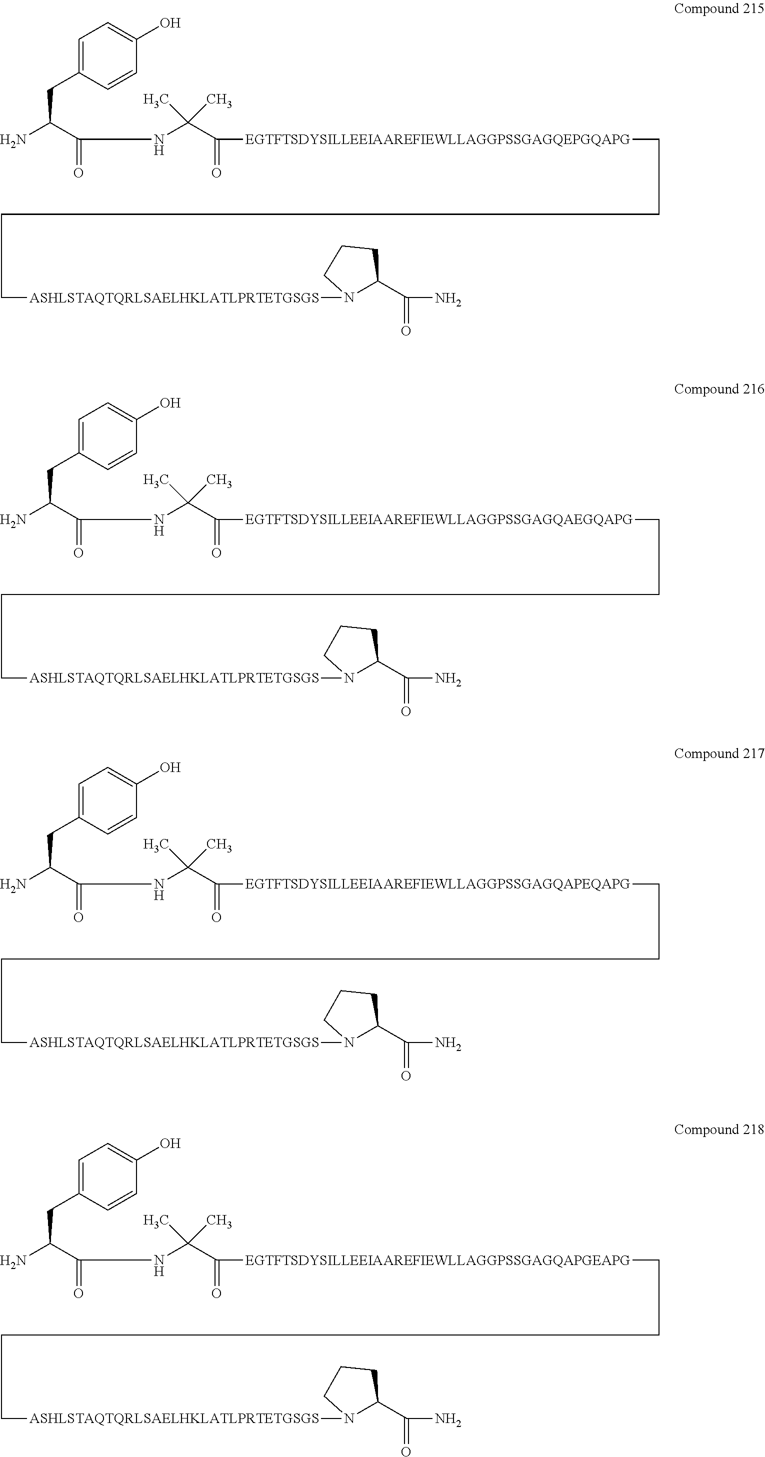

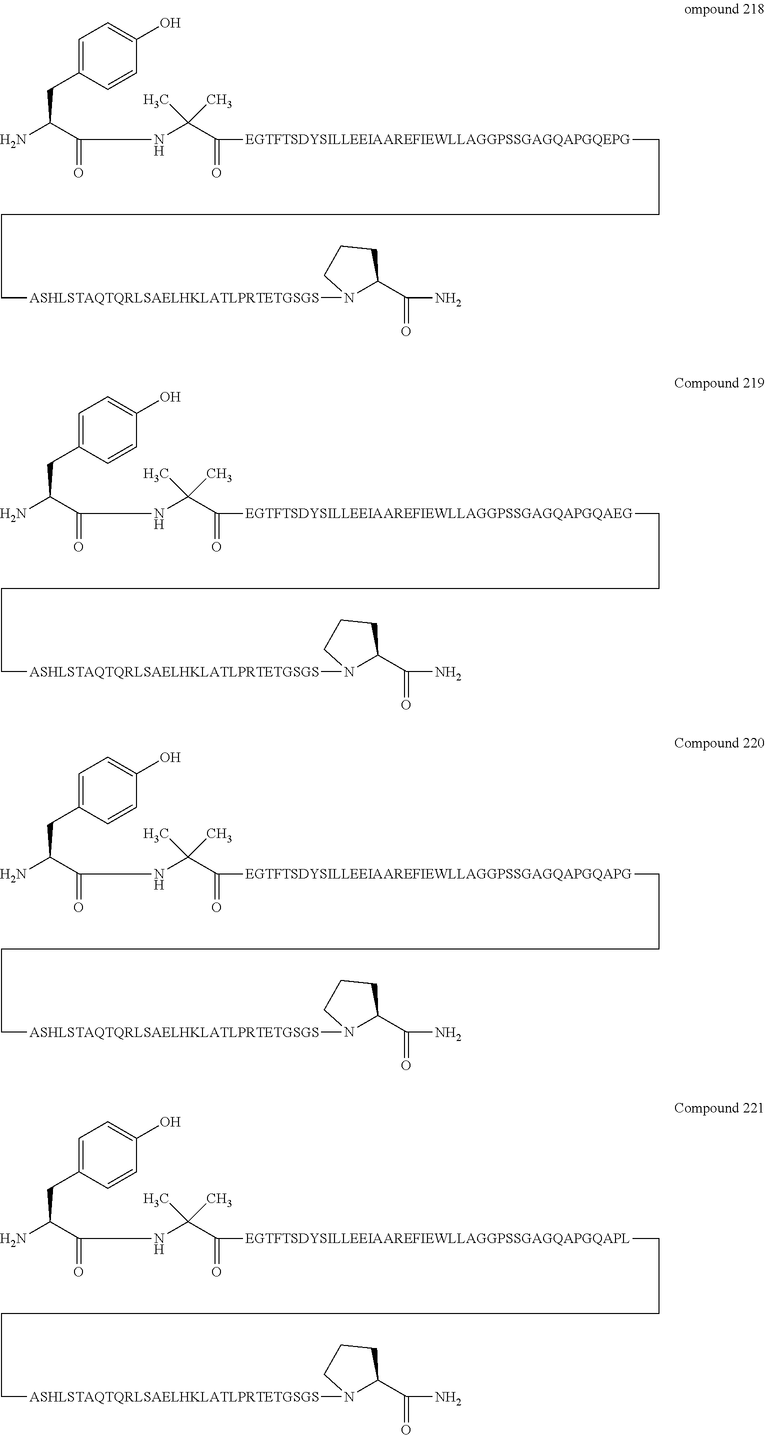

TABLE 9
Structure and amino acid sequences of the synthesized GLP-1-/GIP-/amylin-
receptor tri-agonists according to the invention.

| Com- | SEQ | | | | |
|---|---|---|---|---|---|
| pound | ID | | | | |
| no. | NO: | Amino acid sequences | Protraction moiety | | |
| (Table 4) | | | (Table 6) | (Table 5) | (Table 7) |
| 104 | 243 | YX₂EGTFTSDYSILLEEIAAREFIEWL | Chem. 20 | Chem. 6 | Chem. 27 |
| | | LAGGASSGAGEAPGEAPGASHLST | | | |
| | | AQTQRLSAELHKLATLPRTETGSGS | | | |
| | | P | | | |
| 105 | 244 | YX₂EGTFTSDYSKLLEEIAAREFIEW | Chem. 20 | Chem. 6 | Chem. 27 |
| | | LLAGGPSSGAGASELSTAALGRLSA | | | |
| | | ELHQLATLPRTETGSGSP | | | |
| 106 | 245 | YX₂EGTFTSDYSKLLEEIAAREFIEW | Chem. 20 | Chem. 6 | Chem. 27 |
| | | LLAGGQSSGAGASELSTAQLGRLS | | | |
| | | AELHQLATLPRTETGSGSP | | | |
| 107 | 246 | YX₂EGTFTSDYSKLLEEIAAREFIEW | Chem. 20 | Chem. 6 | Chem. 27 |
| | | LIAGAPSSGAGASELSTAALGRLSA | | | |
| | | ELHQLATLPRTETGSGSP | | | |
| 108 | 247 | YX₂EGTFTSDYSKLLEEIAAREFIEW | Chem. 20 | Chem. 6 | Chem. 27 |
| | | LLAGGPSSGAEASELSTAQLGRLS | | | |
| | | AELHQLATLPRTETGSGSP | | | |
| 109 | 248 | YX₂EGTFTSDYSKLLEEIAAREFIEW | Chem. 20 | Chem. 6 | Chem. 27 |
| | | LIAGGPSEGAGASELSTAQLGRLSA | | | |
| | | ELHQLATLPRTETGSGSP | | | |
| 110 | 249 | YX₂EGTFTSDYSKLLEEIAAREFIEW | Chem. 20 | Chem. 6 | Chem. 27 |
| | | LIAGGGSSGAGASELSTAALGRLSA | | | |
| | | ELHELATLPRTETGSGSP | | | |
| 111 | 250 | YX₂EGTFTSDYSKLLEEIAAREFIEW | Chem. 20 | Chem. 6 | Chem. 27 |
| | | LIAGAPSSGAGASELSTAALGRLSA | | | |
| | | ELHELATLPRTETGSGSP | | | |
| 112 | 251 | YX₂EGTFTSDYSKLLEEIAAREFIEW | Chem. 20 | Chem. 6 | Chem. 27 |
| | | LLAGGGSSGAGASELSTAALGRLS | | | |
| | | AELHELATLPRTETGSGSP | | | |
| 113 | 252 | YX₂EGTFTSDYSKLLEEIAAREFIEW | Chem. 20 | Chem. 6 | Chem. 27 |
| | | LLAGGPSSGAGASELSTAALGRLS | | | |
| | | AELHELATLPRTETGSGSP | | | |
| 114 | 243 | YX₂EGTFTSDYSILLEEIAAREFIEWL | Chem. 20 | Chem. 5 | Chem. 28 |
| | | LAGGASSGAGEAPGEAPGASHLST | | | |
| | | AQTQRLSAELHKLATLPRTETGSGS | | | |
| | | P | | | |
| 115 | 244 | YX₂EGTFTSDYSKLLEEIAAREFIEW | Chem. 20 | Chem. 5 | Chem. 28 |
| | | LLAGGPSSGAGASELSTAALGRLS | | | |
| | | AELHQLATLPRTETGSGSP | | | |
| 116 | 246 | YX₂EGTFTSDYSKLLEEIAAREFIEW | Chem. 20 | Chem. 5 | Chem. 28 |
| | | LIAGAPSSGAGASELSTAALGRLSA | | | |
| | | ELHQLATLPRTETGSGSP | | | |
| 117 | 252 | YX₂EGTFTSDYSKLLEEIAAREFIEW | Chem. 20 | Chem. 5 | Chem. 28 |
| | | LLAGGPSSGAGASELSTAALGRLS | | | |
| | | AELHELATLPRTETGSGSP | | | |
| 118 | 250 | YX₂EGTFTSDYSKLLEEIAAREFIEW | Chem. 20 | Chem. 5 | Chem. 28 |
| | | LIAGAPSSGAGASELSTAALGRLSA | | | |
| | | ELHELATLPRTETGSGSP | | | |
| 119 | 251 | YX₂EGTFTSDYSKLLEEIAAREFIEW | Chem. 20 | Chem. 5 | Chem. 28 |
| | | LLAGGGSSGAGASELSTAALGRLS | | | |
| | | AELHELATLPRTETGSGSP | | | |
| 120 | 170 | YX₂EGTFTSDYSILLEEIAAREFIEWL | Chem. 20 | Chem. 6 | Chem. 27 |
| | | LAGGPSSGAGQAPGQAPGASHLS | | | |
| | | TAQTQRLSAELHKLATLPRTETGSG | | | |
| | | SP | | | |
| 121 | 171 | YX₂EGTFTSDYSILLEEIAAREFIEWL | Chem. 20 | Chem. 6 | Chem. 27 |
| | | IAGGPSSGAGQAPGQAPGASHLST | | | |
| | | AQTQRLSAELHKLATLPRTETGSGS | | | |
| | | P | | | |
| 122 | 172 | YX₂EGTFTSDYSKLLEEIAAREFIEW | Chem. 20 | Chem. 6 | Chem. 27 |
| | | LLAGGPSSGAGQAPGQAPGASHLS | | | |
| | | TAQLGRLSAELHELATLPRTETGSG | | | |
| | | SP | | | |
| 123 | 173 | YX₂EGTFTSDYSKLLEEIAAREFIEW | Chem. 20 | Chem. 6 | Chem. 27 |
| | | LLAGGPSSGAGQAPGQAPGASHLS | | | |
| | | TAQLGRLSAELHQLATLPRTETGSG | | | |
| | | SP | | | |
| 124 | 174 | YX₂EGTFTSDYSKLLEEIAAREFIEW | Chem. 20 | Chem. 6 | Chem. 27 |
| | | LLAGGPSSGAGQAPGQAPGASHLS | | | |
| | | TAQTQRLSAELHELATLPRTETGSG | | | |
| | | SP | | | |
| 125 | 175 | YX₂EGTFTSDYSILLEEIAAREFIEWL | Chem. 20 | Chem. 6 | Chem. 27 |
| | | LAGGPSSGAGQAPGQAPLASHLST | | | |
| | | AQTQRLSAELHKLATLPRTETGSGS | | | |
| | | P | | | |
| 126 | 176 | YX₂EGTFTSDYSKLLEEIAAREFIEW | Chem. 20 | Chem. 6 | Chem. 27 |
| | | LLAGGPSSGAGQAPGQAPGASHLS | | | |
| | | TAALGRLSAELHELATLPRTETGSG | | | |
| | | SP | | | |
| 127 | 177 | YX₂EGTFTSDYSKLLEEIAAREFIEW | Chem. 20 | Chem. 6 | Chem. 27 |
| | | LIAGGPSSGAGQAPGQAPGASHLS | | | |
| | | TAALGRLSAELHELATLPRTETGSG | | | |
| | | SP | | | |
| 128 | 178 | YX₂EGTFTSDYSILLEEIAAREFIEWL | Chem. 20 | Chem. 6 | Chem. 27 |
| | | LAGGPSSGAGQAPGQAPLASHLST | | | |
| | | AQTARLSAELHKLATLPRTETGSGS | | | |
| | | P | | | |
| 129 | 179 | YX₂EGTFTSDYSKLLEEIAAREFIEW | Chem. 20 | Chem. 6 | Chem. 27 |
| | | LLAGGPSSGAGQAPGQAPLASHLS | | | |
| | | TAQLGRLSAELHQLATLPRTETGSG | | | |
| | | SP | | | |
| 130 | 180 | YX₂EGTFTSDYSKLLEEIAAREFIEW | Chem. 20 | Chem. 6 | Chem. 27 |
| | | LLAGGPSSGAGASHLSTAQLGRLS | | | |
| | | AELHQLATLPRTETGSGSP | | | |
| 131 | 181 | YX₂EGTFTSDYSILLEEIAAREFIEWL | Chem. 20 | Chem. 6 | Chem. 27 |
| | | LAGGPSSGAGASHLSTAQTQRLSA | | | |
| | | ELHKLATLPRTETGSGSP | | | |
| 132 | 182 | YX₂EGTFTSDYSILLEEIAAREFIEWL | Chem. 20 | Chem. 6 | Chem. 27 |
| | | LAGGPSSGAGQAPGQAPLASQLST | | | |
| | | AQTQRLSAELHKLATLPRTETGSGS | | | |
| | | P | | | |
| 133 | 183 | YX₂EGTFTSDYSILLEEIAAREFIEWL | Chem. 20 | Chem. 6 | Chem. 27 |
| | | LAGGPSSGAGQAPGQAPLASELST | | | |
| | | AQTQRLSAELHKLATLPRTETGSGS | | | |
| | | P | | | |
| 134 | 184 | YX₂EGTFTSDYSILLEEIAAREFIEWL | Chem. 20 | Chem. 6 | Chem. 27 |
| | | LAGGPSSGEASHLSTAQTQRLSAE | | | |
| | | LHKLATLPRTETGSGSP | | | |
| 135 | 185 | YX₂EGTFTSDYSILLEEIAAREFIEWL | Chem. 20 | Chem. 6 | Chem. 27 |
| | | LAGGPSSGAGQAPGQAPAASHLST | | | |
| | | AQTQRLSAELHKLATLPRTETGSGS | | | |
| | | P | | | |
| 136 | 186 | YX₂EGTFTSDYSILLEEIAAREFIEWL | Chem. 20 | Chem. 6 | Chem. 27 |
| | | LAGGPSSGAGQAPGQAPEASHLST | | | |
| | | AQTQRLSAELHKLATLPRTETGSGS | | | |
| | | P | | | |
| 137 | 187 | YX₂EGTFTSDYSILLEEIAAREFIEWL | Chem. 20 | Chem. 6 | Chem. 27 |
| | | LAGGPSSGAGQAPGQAPPASHLST | | | |
| | | AQTQRLSAELHKLATLPRTETGSGS | | | |
| | | P | | | |
| 138 | 188 | YX₂EGTFTSDYSILLEEIAAREFIEWL | Chem. 20 | Chem. 6 | Chem. 27 |
| | | LAGGPSSGAGQAPGQAPSASHLST | | | |
| | | AQTQRLSAELHKLATLPRTETGSGS | | | |
| | | P | | | |
| 139 | 189 | YX₂EGTFTSDYSILLEEIAAREFIEWL | Chem. 20 | Chem. 6 | Chem. 27 |
| | | LAGGPSSGAGQAPGQAPVASHLST | | | |
| | | AQTQRLSAELHKLATLPRTETGSGS | | | |
| | | P | | | |
| 140 | 190 | YX₂EGTFTSDYSILLEEIAAREFIEWL | Chem. 20 | Chem. 6 | Chem. 27 |
| | | LAGGPSSGAGGGGASHLSTAQTQ | | | |
| | | RLSAELHKLATLPRTETGSGSP | | | |
| 141 | 191 | YX₂EGTFTSDYSILLEEIAAREFIEWL | Chem. 20 | Chem. 6 | Chem. 27 |
| | | LAGGPSSGAGGGGAGELSTAQTQ | | | |
| | | RLSAELHKLATLPRTETGSGSP | | | |
| 142 | 175 | YX₂EGTFTSDYSILLEEIAAREFIEWL | Chem. 19 | Chem. 6 | Chem. 27 |
| | | LAGGPSSGAGQAPGQAPLASHLST | | | |
| | | AQTQRLSAELHKLATLPRTETGSGS | | | |
| | | P | | | |
| 143 | 192 | YX₂EGTFTSDYSILLEEIAAREFIEWL | Chem. 20 | Chem. 6 | Chem. 27 |
| | | IAGGPSSGAGQAPGQAPLASHLST | | | |
| | | AQTQRLSAELHKLATLPRTETGSGS | | | |
| | | P | | | |
| 144 | 193 | YX₂EGTFTSDYSILLEEIAAREFIEWL | Chem. 20 | Chem. 6 | Chem. 27 |
| | | LAGGPSSGAGQAPGQAPGASELST | | | |
| | | AQTQRLSAELHKLATLPRTETGSGS | | | |
| | | P | | | |
| 145 | 194 | YX₂EGTFTSDYSILLEEIAAREFIEWL | Chem. 20 | Chem. 6 | Chem. 27 |
| | | LAGGPSSGAGQAPGQAPGASELST | | | |
| | | AQTARLSAELHKLATLPRTETGSGS | | | |
| | | P | | | |
| 146 | 195 | YX₂EGTFTSDYSILLEEIAAREFIEWL | Chem. 20 | Chem. 6 | Chem. 27 |
| | | LAGGPSSEAGQAPGQAPGASHLST | | | |
| | | AQTQRLSAELHKLATLPRTETGSGS | | | |
| | | P | | | |
| 147 | 196 | YX₂EGTFTSDYSILLEEIAAREFIEWL | Chem. 20 | Chem. 6 | Chem. 27 |
| | | LAGGPSSGEGQAPGQAPGASHLS | | | |
| | | TAQTQRLSAELHKLATLPRTETGSG | | | |
| | | SP | | | |
| 148 | 197 | YX₂EGTFTSDYSILLEEIAAREFIEWL | Chem. 20 | Chem. 6 | Chem. 27 |
| | | LAGGPSSGAGEAPGQAPGASHLST | | | |
| | | AQTQRLSAELHKLATLPRTETGSGS | | | |
| | | P | | | |
| 149 | 198 | YX₂EGTFTSDYSILLEEIAAREFIEWL | Chem. 20 | Chem. 6 | Chem. 27 |
| | | LAGGPSSGAGQEPGQAPGASHLS | | | |
| | | TAQTQRLSAELHKLATLPRTETGSG | | | |
| | | SP | | | |
| 150 | 199 | YX₂EGTFTSDYSILLEEIAAREFIEWL | Chem. 20 | Chem. 6 | Chem. 27 |
| | | LAGGPSSGAGQAEGQAPGASHLS | | | |
| | | TAQTQRLSAELHKLATLPRTETGSG | | | |
| | | SP | | | |
| 151 | 200 | YX₂EGTFTSDYSILLEEIAAREFIEWL | Chem. 20 | Chem. 6 | Chem. 27 |
| | | LAGGPSSGAGQAPEQAPGASHLST | | | |
| | | AQTQRLSAELHKLATLPRTETGSGS | | | |
| | | P | | | |
| 152 | 201 | YX₂EGTFTSDYSILLEEIAAREFIEWL | Chem. 20 | Chem. 6 | Chem. 27 |
| | | LAGGPSSGAGQAPGEAPGASHLST | | | |
| | | AQTQRLSAELHKLATLPRTETGSGS | | | |
| | | P | | | |
| 153 | 202 | YX₂EGTFTSDYSILLEEIAAREFIEWL | Chem. 20 | Chem. 6 | Chem. 27 |
| | | LAGGPSSGAGQAPGQEPGASHLS | | | |
| | | TAQTQRLSAELHKLATLPRTETGSG | | | |
| | | SP | | | |
| 154 | 203 | YX₂EGTFTSDYSILLEEIAAREFIEWL | Chem. 20 | Chem. 6 | Chem. 27 |
| | | LAGGPSSGAGQAPGQAEGASHLS | | | |
| | | TAQTQRLSAELHKLATLPRTETGSG | | | |
| | | SP | | | |
| 155 | 204 | YX₂EGTFTSDYSILLEEIAAREFIEWL | Chem. 20 | Chem. 6 | Chem. 27 |
| | | IAGGPSSGAGQEPGQEPGASHLST | | | |
| | | AQTQRLSAELHKLATLPRTETGSGS | | | |
| | | P | | | |
| 156 | 205 | YX₂EGTFTSDYSILLEEIAAREFIAWL | Chem. 20 | Chem. 6 | Chem. 27 |
| | | IAGGPSSGAGQEPGQEPGASHLST | | | |
| | | AQTQRLSAELHKLATLPRTETGSGS | | | |
| | | P | | | |
| 157 | 206 | YX₂EGTFTSDYSILLEEIAAREFIQWL | Chem. 20 | Chem. 6 | Chem. 27 |
| | | IAGGPSSGAGQEPGQEPGASHLST | | | |
| | | AQTQRLSAELHKLATLPRTETGSGS | | | |
| | | P | | | |
| 158 | 207 | YX₂EGTFTSDYSILLEEIAAREFIEWL | Chem. 20 | Chem. 6 | Chem. 27 |
| | | IAGGPSSGAGASHLSTAQTQRLSA | | | |
| | | ELHKLATLPRTETGSGSP | | | |
| 159 | 208 | YX₂EGTFTSDYSILLEEIAAREFIEWL | Chem. 20 | Chem. 6 | Chem. 27 |
| | | LAGGPSSGAGQAPGQAPASHLSTA | | | |
| | | QTQRLSAELHKLATLPRTETGSGSP | | | |
| 160 | 170 | YX₂EGTFTSDYSILLEEIAAREFIEWL | Chem. 33 | Chem. 6 | Chem. 36 |
| | | LAGGPSSGAGQAPGQAPGASHLS | | | |
| | | TAQTQRLSAELHKLATLPRTETGSG | | | |
| | | SP | | | |
| 161 | 170 | YX₂EGTFTSDYSILLEEIAAREFIEWL | Chem. 34 | Chem. 6 | Chem. 37 |
| | | LAGGPSSGAGQAPGQAPGASHLS | | | |
| | | TAQTQRLSAELHKLATLPRTETGSG | | | |
| | | SP | | | |
| 162 | 209 | YX₂EGTFTSDYSILLEEIAAREFIEWL | Chem. 20 | Chem. 6 | Chem. 27 |
| | | LAGGPSSGAGQAPGQAPGASHLS | | | |
| | | TAQTQRLSAELHKLATEPRTETGS | | | |
| | | GSP | | | |
| 163 | 210 | YX₂EGTFTSDYSILLEEIAAREFIEWL | Chem. 20 | Chem. 6 | Chem. 27 |
| | | LAGGPSSGAGQAPGQAPGASELST | | | |
| | | AQTQRLSAELHKLATEPRTETGSG | | | |
| | | SP | | | |
| 164 | 211 | YX₂EGTFTSDYSILLEEIAAREFIEWL | Chem. 20 | Chem. 6 | Chem. 27 |
| | | LAGGPSSGAGQAPGEAPGASHLST | | | |
| | | AQTQRLSAELHKLATEPRTETGSG | | | |
| | | SP | | | |
| 165 | 212 | YX₂EGTFTSDYSILLEEIAAREFIEWL | Chem. 20 | Chem. 6 | Chem. 27 |
| | | LAGGPSSEAGQAPGQAPGASHLST | | | |
| | | AQTQRLSAELHKLATEPRTETGSG | | | |
| | | SP | | | |
| 166 | 213 | YX₂EGTFTSDYSILLEEIAAREFIEWL | Chem. 20 | Chem. 6 | Chem. 27 |
| | | LAGGPSSGAGQAPGEAPGASELST | | | |
| | | AQTQRLSAELHKLATLPRTETGSGS | | | |
| | | P | | | |
| 167 | 214 | YX₂EGTFTSDYSILLEEIAAREFIEWL | Chem. 20 | Chem. 6 | Chem. 27 |
| | | LAGGPSSEAGQAPGQAPGASELST | | | |
| | | AQTQRLSAELHKLATLPRTETGSGS | | | |
| | | P | | | |
| 168 | 215 | YX₂EGTFTSDYSILLEEIAAREFIEWL | Chem. 20 | Chem. 6 | Chem. 27 |
| | | LAGGPSSEAGQAPGEAPGASHLST | | | |
| | | AQTQRLSAELHKLATLPRTETGSGS | | | |
| | | P | | | |
| 169 | 216 | YX₂EGTFTSDYSKLLEEIAAREFIEW | Chem. 20 | Chem. 6 | Chem. 27 |
| | | LLAGGPSSGAGQAPGQAPGASELS | | | |
| | | TAALGRLSAELHELATLPRTETGSG | | | |
| | | SP | | | |
| 170 | 217 | YX₂EGTFTSDYSKLLEEIAAREFIEW | Chem. 20 | Chem. 6 | Chem. 27 |
| | | LLAGGPSSGAGQAPGQAPGASELS | | | |
| | | TAALGRLSAELHQLATLPRTETGSG | | | |
| | | SP | | | |
| 171 | 218 | YX₂EGTFTSDYSKLLEEIAAREFIEW | Chem. 20 | Chem. 6 | Chem. 27 |
| | | LLAGGPSSGAGQAPGEAPGASELS | | | |
| | | TAALGRLSAELHQLATLPRTETGSG | | | |
| | | SP | | | |
| 172 | 219 | YX₂EGTFTSDYSKLLEEIAAREFIEW | Chem. 20 | Chem. 6 | Chem. 27 |
| | | LLAGGPSSGAGQAPGEAPGASELS | | | |
| | | TAALGRLSAELHQLATEPRTETGSG | | | |
| | | SP | | | |
| 173 | 220 | YX₂EGTFTSDYSKLLEEIAAREFIEW | Chem. 20 | Chem. 6 | Chem. 27 |
| | | LLAGGPSSGAGQAPGEAPGASELS | | | |
| | | TAQLGRLSAELHQLATEPRTETGS | | | |
| | | GSP | | | |
| 174 | 221 | YX₂EGTFTSDYSKLLEEIAAREFIEW | Chem. 20 | Chem. 6 | Chem. 27 |
| | | LLAGGPSSGAGQAPGEAPGASELS | | | |
| | | TAQTGRLSAELHQLATEPRTETGS | | | |
| | | GSP | | | |
| 175 | 222 | YX₂EGTFTSDYSKLLEEIAAREFIEW | Chem. 20 | Chem. 6 | Chem. 27 |
| | | LLAGGPSSGAGQAPGEAPGASELS | | | |
| | | TAQTQRLSAELHQLATEPRTETGS | | | |
| | | GSP | | | |
| 176 | 223 | YX₂EGTFTSDYSILLEEIAAREFIEWL | Chem. 20 | Chem. 6 | Chem. 27 |
| | | LAGGPSSGAGASELSTAQTQRLSA | | | |
| | | ELHKLATLPRTETGSGSP | | | |
| 177 | 224 | YX₂EGTFTSDYSILLEEIAAREFIEWL | Chem. 20 | Chem. 6 | Chem. 27 |
| | | LAGGPSSEAGASHLSTAQTQRLSA | | | |
| | | ELHKLATLPRTETGSGSP | | | |
| 178 | 225 | YX₂EGTFTSDYSILLEEIAAREFIEWL | Chem. 20 | Chem. 6 | Chem. 27 |
| | | LAGGPSSEAGASELSTAQTQRLSA | | | |
| | | ELHKLATLPRTETGSGSP | | | |
| 179 | 226 | YX₂EGTFTSDYSILLEEIAAREFIEWL | Chem. 20 | Chem. 6 | Chem. 27 |
| | | LAGGPSSGAGASHLSTAQTQRLSA | | | |
| | | ELHKLATEPRTETGSGSP | | | |
| 180 | 227 | YX₂EGTFTSDYSILLEEIAAREFIEWL | Chem. 20 | Chem. 6 | Chem. 27 |
| | | LAGGPSSGAGASQLSTAQTQRLSA | | | |
| | | ELHKLATEPRTETGSGSP | | | |
| 181 | 228 | YX₂EGTFTSDYSILLEEIAAREFIEWL | Chem. 20 | Chem. 6 | Chem. 27 |
| | | LAGGPSSEAGASQLSTAQTQRLSA | | | |
| | | ELHKLATLPRTETGSGSP | | | |
| 182 | 229 | YX₂EGTFTSDYSILLEEIAAREFIEWL | Chem. 20 | Chem. 6 | Chem. 27 |
| | | LAGGPSSEAGASQLSTAQTQRLSA | | | |
| | | ELHKLATEPRTETGSGSP | | | |
| 183 | 230 | YX₂EGTFTSDYSKLLEEIAAREFIEW | Chem. 20 | Chem. 6 | Chem. 27 |
| | | LLAGGPSSGAGASELSTAQLGRLS | | | |
| | | AELHQLATLPRTETGSGSP | | | |
| 184 | 231 | YX₂EGTFTSDYSKLLEEIAAREFIEW | Chem. 20 | Chem. 6 | Chem. 27 |
| | | LLAGGPSSGAGASHLSTAQLGRLS | | | |
| | | AELHQLATEPRTETGSGSP | | | |
| 185 | 232 | YX₂EGTFTSDYSKLLEEIAAREFIEW | Chem. 20 | Chem. 6 | Chem. 27 |
| | | LLAGGPSSEAGASHLSTAQLGRLS | | | |
| | | AELHQLATEPRTETGSGSP | | | |
| 186 | 233 | YX₂EGTFTSDYSKLLEEIAAREFIEW | Chem. 20 | Chem. 6 | Chem. 27 |
| | | LLAGGPSSGAGASELSTAQLGRLS | | | |
| | | AELHQLATEPRTETGSGSP | | | |
| 187 | 234 | YX₂EGTFTSDYSKLLEEIAAREFIEW | Chem. 20 | Chem. 6 | Chem. 27 |
| | | LLAGGPSSEAGASELSTAQLGRLS | | | |
| | | AELHQLATLPRTETGSGSP | | | |
| 188 | 235 | YX₂EGTFTSDYSKLLEEIAAREFIEW | Chem. 20 | Chem. 6 | Chem. 27 |
| | | LLAGGPSSEAGASELSTAQLGRLS | | | |
| | | AELHQLATEPRTETGSGSP | | | |
| 189 | 236 | YX₂EGTFTSDYSILLEEIAAREFIEWL | Chem. 20 | Chem. 6 | Chem. 27 |
| | | LAGGPSSGAGEAPGEAPGASHLST | | | |
| | | AQTQRLSAELHKLATLPRTETGSGS | | | |
| | | P | | | |
| 190 | 237 | YX₂EGTFTSDYSILLEEIAAREFIEWL | Chem. 20 | Chem. 6 | Chem. 27 |
| | | IAGGPSSGAGEAPGQAPGASQLST | | | |
| | | AQTQRLSAELHKLATLPRTETGSGS | | | |
| | | P | | | |
| 191 | 238 | YX₂EGTFTSDYSILLEEIAAREFIEWL | Chem. 20 | Chem. 6 | Chem. 27 |
| | | IAGGPSSGAGEAPGQAPGASELST | | | |
| | | AQTQRLSAELHKLATLPRTETGSGS | | | |
| | | P | | | |
| 192 | 170 | YX₂EGTFTSDYSILLEEIAAREFIEWL | Chem. 20 | Chem. 32 | Chem. 35 |
| | | LAGGPSSGAGQAPGQAPGASHLS | | | |
| | | TAQTQRLSAELHKLATLPRTETGSG | | | |
| | | SP | | | |
| 193 | 181 | YX₂EGTFTSDYSILLEEIAAREFIEWL | Chem. 20 | Chem. 32 | Chem. 35 |
| | | LAGGPSSGAGASHLSTAQTQRLSA | | | |
| | | ELHKLATLPRTETGSGSP | | | |
| 194 | 239 | YX₂EGTFTSDYSILLEEIAAQEFIEWL | Chem. 20 | Chem. 6 | Chem. 27 |
| | | LQGGPSSGAGASELSTAQTQRLSA | | | |
| | | ELHKLATLPRTETGSGSP | | | |
| 195 | 240 | YX₂EGTFTSDYSILLEEIAAQEFIEWL | Chem. 20 | Chem. 6 | Chem. 27 |
| | | LQGGPSSGAGEAPGQAPGASELST | | | |
| | | AQTQRLSAELHKLATLPRTETGSGS | | | |
| | | P | | | |
| 196 | 241 | YX₂EGTFTSDYSILLEEIAAREFIEWL | Chem. 20 | Chem. 6 | Chem. 27 |
| | | LAGGPSSGAGQAPGEAPLASHLST | | | |
| | | AQTQRLSAELHKLATLPRTETGSGS | | | |
| | | P | | | |
| 197 | 242 | YX₂EGTFTSDYSILLEEIAAREFIEWL | Chem. 20 | Chem. 6 | Chem. 27 |
| | | LAGGPSSGAGQAPGQAPLASHLST | | | |
| | | AQTQRLSAELHKLATEPRTETGSG | | | |
| | | SP | | | |

wherein X₂ represents always Aib.

### Example 3: LCMS Characterization of the Synthesized Compounds

### LCMS Characterization Method

LCMS analysis was performed on a set up consisting of Waters Acquity UPLC H Class system and Waters Xevo G2-XS QTof. Eluents: A: Milli-Q water; B: MeCN; C: 2% formic acid+0.1% TFA in Milli-Q water.

The analysis was performed at RT (column temperature 60° C.) by injecting an appropriate volume of the sample onto the column. The sample was eluted with a linear gradient of 5-95% B in A, and constant 5% C.

The UPLC conditions, detector settings, and mass spectrometer settings were: Column: Waters Acquity BEH Shield, C-18, 1.7 μm, 2.1 mm×50 mm. Gradient: Linear 5%-95% B, and constant 5% C during 4.0 min at 0.4 ml/min. Total run-time: 7.0 min. Detection: MS sensitivity mode, ionisation method: ES. Scan: 50-5000 amu.

The monoisotopic mass was recorded for the synthesized compounds and their found and calculated values are depicted in Table 10.

TABLE 10
Measured and calculated MS species
for all synthesized compounds

| | Species | m/z found | m/z calc |
|---|---|---|---|
| Reference | [M + 5H]5+ | 1385.469 | 1385.485 |
| compound 1 | [M + 6H]6+ | 1154.892 | 1154.739 |
| Reference | [M + 5H]5+ | 1796.108 | 1795.913 |
| compound 2 | [M + 6H]6+ | 1496.760 | 1496.762 |
| Reference | [M + 5H]5+ | 1675.279 | 1675.266 |
| compound 3 | [M + 6H]6+ | 1396.379 | 1396.223 |
| Reference | [M + 3H]3+ | 1517.136 | 1517.156 |
| compound 4 | [M + 4H]4+ | 1138.116 | 1138.119 |
| Reference | [M + 3H]3+ | 1604.490 | 1604.516 |
| compound 5 | [M + 4H]4+ | 1203.626 | 1203.639 |
| Reference | [M + 3H]3+ | 1469.738 | 1469.758 |
| compound 6 | [M + 4H]4+ | 1102.568 | 1102.570 |
| Reference | [M + 3H]3+ | 1371.374 | 1371.379 |
| compound 7 | [M + 4H]4+ | 1028.789 | 1028.786 |
| Compound 104 | [M + 5H]5+ | 1718.092 | 1718.081 |
| | [M + 6H]6+ | 1431.917 | 1431.902 |
| Compound 105 | [M + 5H]5+ | 1559.819 | 1559.809 |
| | [M + 6H]6+ | 1300.038 | 1300.009 |
| Compound 106 | [M + 5H]5+ | 1577.425 | 1577.415 |
| | [M + 6H]6+ | 1314.854 | 1314.680 |
| Compound 107 | [M + 5H]5+ | 1562.617 | 1562.612 |
| | [M + 6H]6+ | 1302.357 | 1302.345 |
| Compound 108 | [M + 5H]5+ | 1585.825 | 1585.618 |
| | [M + 6H]6+ | 1321.869 | 1321.516 |
| Compound 109 | [M + 5H]5+ | 1579.637 | 1579.616 |
| | [M + 6H]6+ | 1316.695 | 1316.514 |
| Compound 110 | [M + 5H]5+ | 1552.005 | 1552.000 |
| | [M + 6H]6+ | 1293.523 | 1293.501 |
| Compound 111 | [M + 5H]5+ | 1563.022 | 1562.809 |
| | [M + 6H]6+ | 1302.682 | 1302.509 |
| Compound 112 | [M + 5H]5+ | 1552.214 | 1552.000 |
| | [M + 6H]6+ | 1293.509 | 1293.501 |
| Compound 113 | [M + 5H]5+ | 1560.013 | 1560.006 |
| | [M + 6H]6+ | 1300.186 | 1300.173 |
| Compound 114 | [M + 5H]5+ | 1712.498 | 1712.474 |
| | [M + 6H]6+ | 1427.414 | 1427.230 |
| Compound 115 | [M + 5H]5+ | 1554.215 | 1554.203 |
| | [M + 6H]6+ | 1295.349 | 1295.337 |
| Compound 116 | [M + 5H]5+ | 1557.218 | 1557.006 |
| | [M + 6H]6+ | 1297.855 | 1297.673 |
| Compound 117 | [M + 5H]5+ | 1554.408 | 1554.400 |
| | [M + 6H]6+ | 1295.674 | 1295.501 |
| Compound 118 | [M + 5H]5+ | 1557.218 | 1557.203 |
| | [M + 6H]6+ | 1297.855 | 1297.837 |
| Compound 119 | [M + 5H]5+ | 1546.607 | 1546.394 |
| | [M + 6H]6+ | 1289.008 | 1288.829 |
| Compound 120 | [M + 5H]5+ | 1723.077 | 1722.890 |
| | [M + 6H]6+ | 1435.903 | 1435.910 |
| Compound 121 | [M + 5H]5+ | 1723.077 | 1722.890 |
| | [M + 6H]6+ | 1435.903 | 1435.910 |
| Compound 122 | [M + 5H]5+ | 1714.270 | 1714.282 |
| | [M + 6H]6+ | 1428.731 | 1428.736 |
| Compound 123 | [M + 5H]5+ | 1714.287 | 1714.085 |
| | [M + 6H]6+ | 1428.731 | 1428.572 |
| Compound 124 | [M + 5H]5+ | 1726.068 | 1726.082 |
| | [M + 6H]6+ | 1438.557 | 1438.569 |
| Compound 125 | [M + 5H]5+ | 1734.292 | 1734.103 |
| | [M + 6H]6+ | 1445.255 | 1445.253 |
| Compound 126 | [M + 5H]5+ | 1702.867 | 1702.877 |
| | [M + 6H]6+ | 1419.232 | 1419.232 |
| Compound 127 | [M + 5H]5+ | 1702.867 | 1702.877 |
| | [M + 6H]6+ | 1419.232 | 1419.232 |
| Compound 128 | [M + 5H]5+ | 1722.686 | 1722.698 |
| | [M + 6H]6+ | 1435.748 | 1435.750 |
| Compound 129 | [M + 5H]5+ | 1725.286 | 1725.297 |
| | [M + 6H]6+ | 1437.905 | 1437.916 |
| Compound 130 | [M + 5H]5+ | 1572.809 | 1572.817 |
| | [M + 6H]6+ | 1310.841 | 1310.849 |
| Compound 131 | [M + 5H]5+ | 1581.620 | 1581.622 |
| | [M + 6H]6+ | 1318.171 | 1318.186 |
| Compound 132 | [M + 5H]5+ | 1732.298 | 1732.302 |
| | [M + 6H]6+ | 1443.747 | 1443.753 |
| Compound 133 | [M + 5H]5+ | 1732.485 | 1732.499 |
| | [M + 6H]6+ | 1443.918 | 1443.917 |
| Compound 134 | [M + 5H]5+ | 1581.815 | 1581.819 |
| | [M + 6H]6+ | 1318.513 | 1318.350 |
| Compound 135 | [M + 5H]5+ | 1725.677 | 1725.693 |
| | [M + 6H]6+ | 1438.401 | 1438.246 |
| Compound 136 | [M + 5H]5+ | 1737.481 | 1737.294 |
| | [M + 6H]6+ | 1447.902 | 1447.913 |
| Compound 137 | [M + 5H]5+ | 1730.884 | 1730.896 |
| | [M + 6H]6+ | 1442.580 | 1442.581 |
| Compound 138 | [M + 5H]5+ | 1729.080 | 1728.892 |
| | [M + 6H]6+ | 1440.902 | 1440.911 |
| Compound 139 | [M + 5H]5+ | 1731.293 | 1731.299 |
| | [M + 6H]6+ | 1442.907 | 1442.917 |
| Compound 140 | [M + 5H]5+ | 1615.824 | 1615.835 |
| | [M + 6H]6+ | 1346.693 | 1346.697 |
| Compound 141 | [M + 5H]5+ | 1608.428 | 1608.230 |
| | [M + 6H]6+ | 1340.347 | 1340.359 |
| Compound 142 | [M + 5H]5+ | 1705.080 | 1705.088 |
| | [M + 6H]6+ | 1421.068 | 1421.074 |
| Compound 143 | [M + 5H]5+ | 1734.087 | 1734.103 |
| | [M + 6H]6+ | 1445.240 | 1445.253 |
| Compound 144 | [M + 5H]5+ | 1721.276 | 1721.287 |
| | [M + 6H]6+ | 1434.570 | 1434.574 |
| Compound 145 | [M + 5H]5+ | 1709.867 | 1709.882 |
| | [M + 6H]6+ | 1425.067 | 1425.070 |
| Compound 146 | [M + 5H]5+ | 1737.293 | 1737.294 |
| | [M + 6H]6+ | 1448.073 | 1447.913 |
| Compound 147 | [M + 5H]5+ | 1734.479 | 1734.491 |
| | [M + 6H]6+ | 1445.567 | 1445.577 |
| Compound 148 | [M + 5H]5+ | 1723.077 | 1723.087 |
| | [M + 6H]6+ | 1436.229 | 1436.074 |
| Compound 149 | [M + 5H]5+ | 1734.479 | 1734.491 |
| | [M + 6H]6+ | 1445.567 | 1445.577 |
| Compound 150 | [M + 5H]5+ | 1729.284 | 1729.288 |
| | [M + 6H]6+ | 1441.399 | 1441.241 |
| Compound 151 | [M + 5H]5+ | 1737.481 | 1737.294 |
| | [M + 6H]6+ | 1447.902 | 1447.913 |
| Compound 152 | [M + 5H]5+ | 1723.077 | 1723.087 |
| | [M + 6H]6+ | 1436.074 | 1436.074 |
| Compound 153 | [M + 5H]5+ | 1734.479 | 1734.491 |
| | [M + 6H]6+ | 1445.567 | 1445.577 |
| Compound 154 | [M + 5H]5+ | 1729.284 | 1729.288 |
| | [M + 6H]6+ | 1441.244 | 1441.241 |
| Compound 155 | [M + 5H]5+ | 1746.278 | 1746.092 |
| | [M + 6H]6+ | 1455.231 | 1455.245 |
| Compound 156 | [M + 5H]5+ | 1734.479 | 1734.491 |
| | [M + 6H]6+ | 1445.567 | 1445.577 |
| Compound 157 | [M + 5H]5+ | 1745.885 | 1745.895 |
| | [M + 6H]6+ | 1455.075 | 1455.081 |
| Compound 158 | [M + 5H]5+ | 1581.620 | 1581.622 |
| | [M + 6H]6+ | 1318.350 | 1318.186 |
| Compound 159 | [M + 5H]5+ | 1711.475 | 1711.486 |
| | [M + 6H]6+ | 1426.396 | 1426.406 |
| Compound 160 | [M + 5H]5+ | 1774.701 | 1774.507 |
| | [M + 6H]6+ | 1478.920 | 1478.924 |
| Compound 161 | [M + 5H]5+ | 1774.494 | 1774.507 |
| | [M + 6H]6+ | 1478.920 | 1478.924 |
| Compound 162 | [M + 5H]5+ | 1726.068 | 1726.082 |
| | [M + 6H]6+ | 1438.557 | 1438.569 |
| Compound 163 | [M + 5H]5+ | 1724.470 | 1724.478 |
| | [M + 6H]6+ | 1437.222 | 1437.233 |
| Compound 164 | [M + 5H]5+ | 1726.272 | 1726.279 |
| | [M + 6H]6+ | 1438.728 | 1438.733 |
| Compound 165 | [M + 5H]5+ | 1740.673 | 1740.486 |
| | [M + 6H]6+ | 1450.567 | 1450.573 |
| Compound 166 | [M + 5H]5+ | 1721.480 | 1721.484 |
| | [M + 6H]6+ | 1434.895 | 1434.738 |
| Compound 167 | [M + 5H]5+ | 1735.673 | 1735.691 |
| | [M + 6H]6+ | 1446.563 | 1446.577 |
| Compound 168 | [M + 5H]5+ | 1737.481 | 1737.491 |
| | [M + 6H]6+ | 1448.073 | 1448.077 |
| Compound 169 | [M + 5H]5+ | 1701.263 | 1701.274 |
| | [M + 6H]6+ | 1417.891 | 1417.896 |
| Compound 170 | [M + 5H]5+ | 1701.263 | 1701.077 |
| | [M + 6H]6+ | 1417.721 | 1417.732 |
| Compound 171 | [M + 5H]5+ | 1701.263 | 1701.274 |
| | [M + 6H]6+ | 1417.891 | 1417.896 |
| Compound 172 | [M + 5H]5+ | 1704.455 | 1704.466 |
| | [M + 6H]6+ | 1420.543 | 1420.556 |
| Compound 173 | [M + 5H]5+ | 1715.864 | 1715.870 |
| | [M + 6H]6+ | 1430.046 | 1430.060 |
| Compound 174 | [M + 5H]5+ | 1713.457 | 1713.463 |
| | [M + 6H]6+ | 1428.051 | 1428.054 |
| Compound 175 | [M + 5H]5+ | 1727.854 | 1727.670 |
| | [M + 6H]6+ | 1439.892 | 1439.893 |
| Compound 176 | [M + 5H]5+ | 1580.009 | 1580.019 |
| | [M + 6H]6+ | 1317.013 | 1316.850 |
| Compound 177 | [M + 5H]5+ | 1596.026 | 1596.026 |
| | [M + 6H]6+ | 1330.176 | 1330.190 |
| Compound 178 | [M + 5H]5+ | 1594.424 | 1594.423 |
| | [M + 6H]6+ | 1328.848 | 1328.854 |
| Compound 179 | [M + 5H]5+ | 1584.812 | 1584.814 |
| | [M + 6H]6+ | 1320.833 | 1320.846 |
| Compound 180 | [M + 5H]5+ | 1583.004 | 1583.014 |
| | [M + 6H]6+ | 1319.509 | 1319.346 |
| Compound 181 | [M + 5H]5+ | 1594.228 | 1594.226 |
| | [M + 6H]6+ | 1328.684 | 1328.690 |
| Compound 182 | [M + 5H]5+ | 1597.416 | 1597.418 |
| | [M + 6H]6+ | 1331.341 | 1331.350 |
| Compound 183 | [M + 5H]5+ | 1571.203 | 1571.214 |
| | [M + 6H]6+ | 1309.507 | 1309.513 |
| Compound 184 | [M + 5H]5+ | 1576.009 | 1576.009 |
| | [M + 6H]6+ | 1313.495 | 1313.508 |
| Compound 185 | [M + 5H]5+ | 1590.406 | 1590.413 |
| | [M + 6H]6+ | 1325.508 | 1325.512 |
| Compound 186 | [M + 5H]5+ | 1574.401 | 1574.405 |
| | [M + 6H]6+ | 1312.160 | 1312.172 |
| Compound 187 | [M + 5H]5+ | 1585.610 | 1585.618 |
| | [M + 6H]6+ | 1321.502 | 1321.516 |
| Compound 188 | [M + 5H]5+ | 1588.806 | 1588.810 |
| | [M + 6H]6+ | 1324.167 | 1324.176 |
| Compound 189 | [M + 5H]5+ | 1723.485 | 1723.284 |
| | [M + 6H]6+ | 1436.229 | 1436.238 |
| Compound 190 | [M + 5H]5+ | 1721.276 | 1721.287 |
| | [M + 6H]6+ | 1434.570 | 1434.574 |
| Compound 191 | [M + 5H]5+ | 1721.480 | 1721.484 |
| | [M + 6H]6+ | 1434.895 | 1434.738 |
| Compound 192 | [M + 5H]5+ | 1730.271 | 1730.085 |
| | [M + 6H]6+ | 1441.896 | 1441.906 |
| Compound 193 | [M + 5H]5+ | 1588.806 | 1588.817 |
| | [M + 6H]6+ | 1324.182 | 1324.182 |
| Compound 194 | [M + 5H]5+ | 1585.806 | 1585.815 |
| | [M + 6H]6+ | 1321.845 | 1321.680 |
| Compound 195 | [M + 5H]5+ | 1727.276 | 1727.279 |
| | [M + 6H]6+ | 1439.566 | 1439.567 |
| Compound 196 | [M + 5H]5+ | 1734.292 | 1734.299 |
| | [M + 6H]6+ | 1445.411 | 1445.417 |
| Compound 197 | [M + 5H]5+ | 1737.481 | 1737.294 |
| | [M + 6H]6+ | 1447.902 | 1447.913 |
| Compound 211 | [M + 5H]5+ | 1588.594 | 1588.603 |
| | [M + 6H]6+ | 1324.167 | 1324.004 |
| Compound 212 | [M + 5H]5+ | 1585.790 | 1585.800 |
| | [M + 6H]6+ | 1321.666 | 1321.668 |
| Compound 213 | [M + 5H]5+ | 1574.384 | 1574.395 |
| | [M + 6H]6+ | 1312.160 | 1312.164 |
| Compound 214 | [M + 5H]5+ | 1585.790 | 1585.800 |
| | [M + 6H]6+ | 1321.830 | 1321.668 |
| Compound 215 | [M + 5H]5+ | 1580.594 | 1580.597 |
| | [M + 6H]6+ | 1317.325 | 1317.332 |
| Compound 216 | [M + 5H]5+ | 1588.594 | 1588.603 |
| | [M + 6H]6+ | 1324.167 | 1324.004 |
| Compound 217 | [M + 5H]5+ | 1574.384 | 1574.395 |
| | [M + 6H]6+ | 1312.160 | 1312.164 |
| Compound 218 | [M + 5H]5+ | 1585.790 | 1585.800 |
| | [M + 6H]6+ | 1321.666 | 1321.668 |
| Compound 219 | [M + 5H]5+ | 1580.594 | 1580.597 |
| | [M + 6H]6+ | 1317.325 | 1317.332 |
| Compound 220 | [M + 5H]5+ | 1574.189 | 1574.199 |
| | [M + 6H]6+ | 1311.997 | 1312.000 |
| Compound 221 | [M + 5H]5+ | 1585.610 | 1585.411 |
| | [M + 6H]6+ | 1321.339 | 1321.344 |

### Example 4: Human GLP-1-, GlP-, and Amylin-Receptors In Vitro Potency Assay (High Throughput Assay)

GLP-1 receptor Assay

To determine the ability of compounds to activate or agonize the GLP-1 receptor, in vitro potency assay on Baby Hamster Kidney (BHK) cells expressing the human GLP-1 receptor (hGLP-1R) was performed as described below. To assess how the activation of the receptors is potentially influenced by the presence of human serum albumin (HSA), the in vitro assay was performed in the absence of HSA and presence of 1% (w/v) HSA. Unless stated otherwise the reference to the “GLP-1 receptor assay as described in Example 4” throughout the specification shall refer to the described herein assay procedure (hGLP-1R assay) in the absence of HSA.

### Assay Principle

Activation of the human GLP-1 receptor leads to increased intra-cellular concentrations of cyclic AMP (cAMP) and the consequent transcription activation from promoters containing multiple copies of the cAMP response element (CRE). It is thus possible to measure GLP-1 receptor activity using a CRE-luciferase reporter gene introduced into Baby Hamster Kidney (BHK) cells co-expressing the human GLP-1 receptor.

### Cells and Assay Reagents

Cell stocks were prepared by culturing of a cell line stably expressing the human GLP-1 receptor and containing the CRE responsive luciferase (CRE-Luc) reporter gene (BHK 467-12A KZ-10 prepared according to methods known to the person skilled in the art) in growth medium consisting of DMEM (Gibco, 61965-026) supplemented with 10% FBS (Gibco, 10100-147), 1% Penicillin-Streptomycin (Gibco, 15140-122), 1 mM Na-Pyruvate (Gibco, 11360-039), 0.5 mg/mL G418 (Gibco, 10131-027) and 240 nM Methotrexate (Pfizer, 15936). Cells at approximately 80-90% confluence were washed once in PBS (Gibco 14190-094) and loosened from the cell flasks with Versene (Gibco, 15040-033). After centrifugation, the cell pellet was resuspended and diluted to approximately 1.5×10⁶ cells/mL in Recovery™ Cell Culture Freezing Medium (Gibco, 12648-010). Cells were aliquoted and stored at −180° C. until use.

The assay buffer consisted of DMEM without phenol red (Gibco, 11880-028) supplemented with 1× GlutaMAX (Gibco, 35050-038), 10 mM HEPES (Gibco, 15630-056), 1% (w/v) ovalbumin (Sigma, A5503) and 0.1% (v/v) Pluronic F-68 (Gibco, 24040-032) either with HSA or without HSA (Sigma, A9511).

Procedure (hGLP-1R Assay)

To perform the assay, serial dilutions (7-fold dilutions, 7 concentrations per compound and one well containing only assay buffer) of reference compounds and GLP-1-/GlP-/amylin-receptor tri-agonists were performed in assay buffer in a 96-well plate. Serial dilutions were transferred to a 384-well assay plate (Revvity, 6007688) and mixed with an equal volume (10 μL) of assay buffer without or with 3% (w/v) HSA (Sigma, A9511). Frozen stocks of hGLP-1R BHK Cre-Luc cells were thawed in a 37° C. water bath, washed once in PBS (Gibco 14190-094), diluted to 1.5×10⁵ (1.5E+5) cells/mL in assay buffer (without HSA) and added (10 μL) to each well of the 384-well assay plate. After a short centrifugation, the assay plates were incubated for 3 hours at 37° C. in 5% CO₂ and let to equilibrate at room temperature for 10 minutes before the addition of 30 μL steadylite Plus™ Revvity, 6066759) per well. Plates were sealed and incubated at room temperature with gentle shaking for 30 minutes while protected from light. Luminescence was detected on a luminescence plate reader e.g. a Synergy 2 (BioTek). The EC₅₀-values [pM] were calculated by non-linear curve fitting applying a four-parameter logistic model (Hill slope=1) using GraphPad Prism (GraphPad Software, Boston, MA, USA) or by means of TIBCO Enterprise Runtime for R (TIBCO Software, Palo Alto, CA, USA).

### GlP Receptor Assay

To determine the ability of compounds to activate or agonize the GlP receptor, in vitro potency assay on Baby Hamster Kidney (BHK) cells expressing the human GlP receptor (hGlPR) was performed as described below. To assess how the activation of the receptors is potentially influenced by the presence of human serum albumin (HSA), the in vitro assay was performed in the absence of HSA and presence of 1% (w/v) HSA. Unless stated otherwise the reference to the “GlP receptor assay as described in Example 4” throughout the specification shall refer to the described herein assay procedure (hGlPR assay) in the absence of HSA.

### Assay Principle

Activation of the human GlP receptor leads to increased intra-cellular concentrations of cyclic AMP (cAMP) and the consequent transcription activation from promoters containing multiple copies of the cAMP response element (CRE). It is thus possible to measure GlP receptor activity using a CRE-luciferase reporter gene introduced into Baby Hamster Kidney (BHK) cells co-expressing the human GlP receptor.

### Cells and Assay Reagents

Cell stocks were prepared by culturing of a cell line stably expressing the human GlP receptor and containing the CRE responsive luciferase (CRE-Luc) reporter gene (hGlPR BHK Cre-Luc2p clone #5 prepared according to methods known to the person skilled in the art) at 5% CO₂ and 37° C. in growth medium consisting of DMEM (Gibco, 61965-026) supplemented with 10% fetal calf serum (Gibco, 10100-147), 0.5 mg/ml G418 (Gibco, 10131-027), 1% Penicillin-Streptomycin (Gibco, 15140-122) and 0.3 mg/ml Hygromycin B (ThermoFisher, 10687010). Cells at about 80-90% confluency were washed once with PBS (Gibco 14190-094) and detached from the cell flasks with Versene (Gibco, 15040-066). After centrifugation, the cells were counted, resuspended and diluted to approximately 1.5-3.0×10⁶ (1.5E+6 to 3.0E+6) cells/mL in Recovery Cell Culture Freezing Medium (Gibco, 12648-010) and stored at −180° C. in suitable aliquots until use.

The assay buffer consisted of DMEM without phenol red (Gibco, 11880-028) supplemented with 1× GlutaMAX (Gibco, 35050-038), 10 mM HEPES (Gibco, 15630-056), 1% (w/v) ovalbumin (Sigma, A5503) and 0.1% (v/v) Pluronic F-68 (Gibco, 24040-032) either with HSA or without HSA (Sigma, A9511).

Procedure (hGlPR Assay)

To perform the assay, serial dilutions (7-fold dilutions, 7 concentrations per compound and one well containing only assay buffer) of reference compounds and GLP-1-/GlP-/amylin-receptor tri-agonists were prepared in assay buffer in a 96-well plate. Serial dilutions were transferred to a 384-well assay plate (Revvity, 6007688) and mixed with an equal volume (10 μL) of assay buffer without or with 3% HSA (Sigma, A9511). Frozen stocks of hGlPR BHK Cre-Luc cells were thawed in a 37° C. water bath, washed once in PBS (Gibco 14190-094), diluted to 1.5×10⁵ (1.5E+5) cells/mL in assay buffer (without HSA) and added (10 μL) to each well of the 384-well assay plate. After a short centrifugation, the assay plates were incubated for 3 hours at 37° C. in 5% CO₂ and let to equilibrate at room temperature for 10 minutes before the addition of 30 μL steadylite Plus™ (Revvity, 6066759) per well. Plates were sealed and incubated at room temperature with gentle shaking for 30 minutes while protected from light. Luminescence was detected on a luminescence plate reader e.g. a Synergy 2 (BioTek). The EC₅₀-values [pM] were calculated by non-linear curve fitting applying a four-parameter logistic model (Hill slope=1) using GraphPad Prism (GraphPad Software, Boston, MA, USA) or by means of TIBCO Enterprise Runtime for R (TIBCO Software, Palo Alto, CA, USA).

### Amylin Receptor Assay

To determine the ability of compounds to activate or agonize the amylin receptor, in vitro potency assay on Baby Hamster Kidney (BHK) cells expressing the human amylin receptor (hAMYR3) was performed as described below. To assess how the activation of the receptors is potentially influenced by the presence of human serum albumin (HSA), the in vitro assay was performed in the absence of HSA and presence of 1% (w/v) HSA. Unless stated otherwise the reference to the “amylin receptor assay as described in Example 4” throughout the specification shall refer to the described herein assay procedure (hAMYR3 assay) in the absence of HSA.

### Assay Principle

Activation of the human amylin 3 receptor leads to increased intra-cellular concentrations of cAMP and the consequent transcription activation from promoters containing multiple copies of the cAMP response element (CRE). It is thus possible to measure hAMYR3 activity using a CRE-luciferase reporter gene introduced into Baby Hamster Kidney (BHK) cells co-expressing the hAMYR3.

### Cells and Assay Reagents

A BHK cell line was engineered to stably express the human calcitonin receptor(a) and to contain a CRE-responsive luciferase (CRE-Luc) reporter gene according to methods known to the person skilled in the art (Hollex-1 cell line, obtained from Zymogenetics described in U.S. Pat. No. 5,622,839). The cell line was further transfected with human receptor modifying protein 3 (hRAMP3) using standard methods. Association of hRAMP3 with the human calcitonin receptor give rice to the human amylin-3-(a) receptor (hAMYR3)

Cells stocks were prepared by culturing of the hAMYR3 BHK Cre-Luc cell line in growth medium consisting of DMEM (Gibco, 31966-021) supplemented with 10% FBS (Gibco, 10100-147), 1% Penicillin-Streptomycin (Gibco, 15140-122), 0.5 mg/mL Geneticin (Gibco, 10131-027), 0.4 mg/mL Hygromycin (ThermoFisher, 10687010) and 250 nM Methotrexate (Sigma, A6770). Cells at approximately 80-90% confluence were washed once with PBS (Gibco 14190-094) and loosened from the cell flasks with Versene (Gibco, 15040-033) or TrypLE™ (Gibco, 12605-010). After centrifugation, the cell pellet was resuspended and diluted to approximately 2.5-4.0×10⁶ (2.5E+6 to 4.0E+6) cells/mL in Recovery™ Cell Culture Freezing Medium (Gibco, 12648-010). Cells were aliquoted and stored at −180° C. until use.

The assay buffer consisted of DMEM without phenol red (Gibco, 11880-028) supplemented with 1× GlutaMAX (Gibco, 35050-038), 10 mM HEPES (Gibco, 15630-056) and 1% (w/v) ovalbumin (Sigma, A5503) either with or without 0.1% (v/v) Pluronic F-68 (Gibco, 24040-032) and either with or without HSA (Sigma, A9511).

Procedure (hAMYR3)

To perform the assay, hAMYR3 BHK Cre-Luc cells were thawed, washed once in PBS (Gibco 14190-094) and seeded in 40 μL growth medium in a white 384-well culture plate (Revvity, 6007688) at a cell density of 4.0×10³ (4.0E+3) cells/well the day before the experiment. The plate was incubated over night at 37° C. in 5% CO₂. On the day of the assay, serial dilutions (7-fold dilutions, 7 concentrations per compound and one well containing only assay buffer) of reference compounds and GLP-1-/GlP-/amylin-receptor tri-agonists were prepared in assay buffer in 96-well plates. Serial dilutions were then mixed in a new 96-well plate with equal volume (1:1:1 ratio) of assay buffer and either assay buffer without or with 3% HSA (Sigma, A9511). Twenty microliters of the solution mix were transferred to the cells which previously were washed once with PBS (Gibco 14190-094). After a short centrifugation, the assay plates were incubated for 3 hours at 37° C. in 5% CO₂ and let to equilibrate at room temperature for 10 minutes before the addition of 30 μL steadylite Plus™ (Revvity, 6066759) per well. Plates were sealed and incubated at room temperature with gentle shaking for 30 minutes while protected from light. Luminescence was detected on a luminescence plate reader e.g., a Synergy 2 (BioTek). The EC₅₀-values [pM] were calculated by non-linear curve fitting applying a four-parameter logistic model (Hill slope=1.5, shared bottom response within each plate) using GraphPad Prism (GraphPad Software, Boston, MA, USA) or by means of TIBCO Enterprise Runtime for R (TIBCO Software, Palo Alto, CA, USA).

### Results

TABLE 11
In vitro activity data for reference compounds on hGLP-
1R, hGIPR, and hAMYR3 measured in the absence of HSA

| | hGLP-1R | hGIPR | hAMYR3 |
|---|---|---|---|
| Reference | Potency, | Potency | Potency, |
| compound | no HSA, | no HSA, | no HSA, |
| no. | EC₅₀ [pM] | EC₅₀ [pM] | EC₅₀ [pM] |
| 1 | 1680 | 221 | 6.55 |
| 2 | 627 | 21.2 | 52.0 |
| 3 | 238 | 6.79 | 13.8 |
| 4 | no | 2.27 | nd |
| 5 | 72.0 | 4.32 | nd |
| 6 | nd | nd | 2.37 |
| 7 | 4.62 | nd | nd |

The results in Table 11 shows that the reference compounds 4 to 7 are agonists or co-agonists on one or two of the GLP-1 receptor, GlP receptor, and amylin receptor (hAMYR3).

According to Table 11, the activity data for reference compound 2 shows that linking the C-terminus of a potent GLP-1/GlP co-agonist (tirzepatide), via a peptide linker, to the N-terminus of a potent amylin receptor agonist (cagrilintide) does not result in a compound that is equally potent on these three receptors and that can necessarily function as a GLP-1-/GlP-/amylin-receptor tr-agonist (i.e., a compound according to the invention). A comparison of the reference compound 2 with the reference compound 5 (tirzepatide) and reference compound 6 (cagrilintide) illustrates this point. The reference compound 2 significantly loses potency on the GLP-1 receptor and show further some loss of potency on the amylin receptor, when compared to the original compounds, reference compounds 5 (tirzepatide) and 6 (cagrilintide).

The reference compounds 1 to 3 show a functional activation of all three receptors but have all an impaired potency on one or more of the GLP-1-, GlP-, and amylin-receptors compared to the GLP-1/GlP-/amylin receptor tri-agonists of the present invention. Reference compounds 1-3 are therefore not potent and balanced at all three receptors.

TABLE 12
In vitro activity data for the synthesized
compound of the invention on hGLP-1R, hGIPR,
and hAMYR3 measured in the absence of HSA

| | hGLP-1R | hGIPR | hAMYR3 |
|---|---|---|---|
| | Potency, | Potency, | Potency, |
| Compound | no HSA, | no HSA, | no HSA, |
| no. | EC₅₀ [pM] | EC₅₀ [pM] | EC₅₀ [pM] |
| 104 | 24.6 | 4.14 | 8.05 |
| 105 | 15.4 | 2.21 | 8.25 |
| 106 | 20.7 | 2.98 | 6.77 |
| 107 | 9.14 | 2.52 | 10.4 |
| 108 | 19.8 | 2.48 | 13.1 |
| 109 | 10.2 | 3.14 | 12.2 |
| 110 | 6.36 | 3.57 | 11.1 |
| 111 | 9.5 | 3.0 | 15.4 |
| 112 | 19.5 | 2.8 | 16.2 |
| 113 | 15.2 | 2.19 | 12.5 |
| 114 | 23.5 | 5.8 | 7.8 |
| 115 | 26.1 | 4.3 | 7.4 |
| 116 | 13.0 | 3.5 | 8.22 |
| 117 | 23.3 | 2.3 | 10.7 |
| 118 | 11.1 | 3.8 | 15.5 |
| 119 | 35.3 | 6.1 | 10.5 |
| 120 | 25.2 | 2.96 | 7.54 |
| 121 | 9.5 | 2.95 | 7.63 |
| 122 | 21.0 | 3.73 | 7.68 |
| 123 | 19.9 | 3.69 | 7.89 |
| 124 | 21.3 | 3.4 | 15.2 |
| 125 | 23.8 | 3.06 | 6.26 |
| 126 | 21.7 | 3.38 | 11.0 |
| 127 | 5.28 | 3.15 | 12.4 |
| 128 | 43.9 | 6.31 | 7.5 |
| 129 | 25.0 | 3.66 | 5.46 |
| 130 | 21.2 | 4.05 | 7.24 |
| 131 | 29.6 | 4.24 | 5.44 |
| 132 | 29.8 | 3.69 | 5.29 |
| 133 | 21.4 | 3.67 | 8.78 |
| 134 | 27.9 | 3.88 | 7.33 |
| 135 | 33.6 | 4.86 | 8.3 |
| 136 | 34.3 | 4.64 | 10.2 |
| 137 | 28.3 | 3.71 | 5.1 |
| 138 | 19.7 | 2.3 | 5.5 |
| 139 | 19.0 | 2.1 | 7.1 |
| 140 | 28.0 | 3.3 | 10.2 |
| 141 | 24.2 | 2.53 | 7.64 |
| 142 | 26.9 | 3.9 | 6.43 |
| 143 | 28.8 | 3.89 | 6.16 |
| 144 | 20.8 | 2.72 | 8.07 |
| 145 | 25.0 | 4.15 | 10.2 |
| 146 | 34.0 | 2.93 | 6.83 |
| 147 | 33.6 | 3.06 | 7.12 |
| 148 | 25.7 | 2.35 | 6.56 |
| 149 | 30.8 | 3.14 | 8.86 |
| 150 | 23.3 | 2.15 | 7.77 |
| 151 | 24.1 | 2.37 | 9.48 |
| 152 | 20.8 | 2.3 | 8.7 |
| 153 | 21.2 | 2.25 | 7.54 |
| 154 | 33.8 | 3.42 | 8.11 |
| 155 | 10.0 | 4.56 | 11.1 |
| 156 | 9.03 | 6.59 | 11.8 |
| 157 | 8.48 | 5.6 | 9.6 |
| 158 | 11.2 | 3.6 | 11.8 |
| 159 | 26.2 | 3.16 | 7.42 |
| 160 | 17.6 | 2.2 | 8.2 |
| 161 | 19.3 | 2.6 | 9.7 |
| 162 | 15.1 | 1.79 | 10.4 |
| 163 | 15.7 | 1.63 | 20.4 |
| 164 | 14.4 | 1.93 | 10.5 |
| 165 | 24.3 | 2.19 | 13.8 |
| 166 | 15.0 | 1.76 | 9.12 |
| 167 | 23.0 | 2.0 | 9.89 |
| 168 | 25.8 | 2.31 | 6.59 |
| 169 | 13.1 | 1.9 | 15.0 |
| 170 | 15.2 | 1.8 | 6.9 |
| 171 | 10.7 | 2.1 | 11.0 |
| 172 | 12.8 | 2.1 | 20.8 |
| 173 | 11.8 | 1.8 | 22.2 |
| 174 | 14.0 | 2.1 | 72.0 |
| 175 | 14.3 | 2.3 | 34.3 |
| 176 | 26.8 | 3.08 | 14.0 |
| 177 | 35.9 | 4.13 | 8.37 |
| 178 | 27.0 | 3.59 | 12.9 |
| 179 | 18.9 | 3.3 | 11.1 |
| 180 | 13.3 | 2.2 | 9.93 |
| 181 | 22.2 | 2.76 | 7.75 |
| 182 | 21.0 | 3.2 | 17.2 |
| 183 | 14.7 | 2.17 | 7.08 |
| 184 | 13.3 | 1.72 | 7.57 |
| 185 | 15.2 | 2.2 | 11.2 |
| 186 | 14.1 | 2.14 | 16.5 |
| 187 | 31.5 | 4.73 | 17.2 |
| 188 | 20.2 | 3.52 | 31.9 |
| 189 | 26.2 | 2.52 | 7.31 |
| 190 | 6.0 | 2.1 | 10.6 |
| 191 | 7.9 | 2.7 | 16.0 |
| 192 | 35.0 | 3.68 | 8.17 |
| 193 | 49.0 | 3.74 | 8.67 |
| 194 | 303 | 2.89 | 11.0 |
| 195 | 244 | 3.58 | 14.0 |
| 196 | 42.5 | 4.56 | 12.8 |
| 197 | 18.3 | 3.92 | 8.16 |
| 211 | 15.2 | 1.83 | 3.8 |
| 212 | 12.7 | 1.92 | 3.6 |
| 213 | 11.1 | 1.9 | 3.6 |
| 214 | 12.2 | 1.64 | 3.8 |
| 215 | 11.6 | 1.7 | 3.8 |
| 216 | 9.9 | 1.7 | 4.5 |
| 217 | 9.0 | 1.7 | 5.9 |
| 218 | 8.8 | 1.6 | 5.4 |
| 219 | 2.7 | 0.95 | 5.0 |
| 220 | 12.5 | 2.22 | 7.05 |
| 221 | 17.0 | 2.39 | 6.76 |

The results in Table 12 show that the compounds of the present invention display potent functional activation of all three receptors, namely the human GLP-1 receptor, human GlP receptor, and human amylin receptor (hAMYR3).

Most of the GLP-1-/GlP-/amylin-receptor tri-agonists of the present invention agonize the different receptors with EC₅₀ values comparable to the GlP, GLP-1 and amylin receptor agonists and GLP-1/GlP co-agonists disclosed herein as reference compounds 4 to 7. Furthermore, the GLP-1-/GlP-/amylin-receptor tri-agonists of the present invention, as shown in Table 12, display a potent and balanced functional activation of all three receptors, and are, in contrast to the reference compounds 1, 2 and 3, balanced GLP-1-/GlP-/amylin-receptor tri-agonists.

TABLE 13
In vitro activity data for reference compounds on hGLP-
1R, hGIPR, and hAMYR3 measured in the presence of 1% HSA

| | hGLP-1R | hGIPR | hAMYR3 |
|---|---|---|---|
| Reference | Potency, | Potency, | Potency, |
| compound | 1% HSA, | 1% HSA, | 1% HSA, |
| number | EC₅₀ [pM] | EC₅₀ [pM] | EC₅₀ [pM] |
| 1 | 851 | 47.0 | 5.88 |
| 2 | 1920 | 36.5 | 225 |
| 3 | 1730 | 163 | 88.4 |
| 4 | nd | 70.4 | nd |
| 5 | 781 | 56.5 | nd |
| 6 | nd | nd | 82.2 |
| 7 | 117 | nd | nd |

TABLE 14
In vitro activity data for the synthesized compounds
of the invention on hGLP-1R, hGIPR, and hAMYR3
measured in the presence of 1% HSA

| | hGLP-1R | hGIPR | hAMYR3 |
|---|---|---|---|
| | Potency, | Potency, | Potency, |
| Compound | 1% HSA, | 1% HSA, | 1% HSA, |
| No. | EC₅₀ [pM] | EC₅₀ [pM] | EC₅₀ [pM] |
| 104 | 79.2 | 12.7 | 797 |
| 105 | 701 | 69.2 | 223 |
| 106 | 787 | 75.2 | 134 |
| 107 | 386 | 70.2 | 196 |
| 108 | 903 | 81.4 | 451 |
| 109 | 388 | 103 | 239 |
| 110 | 390 | 94.6 | 398 |
| 111 | 464 | 96.3 | 361 |
| 113 | 700 | 90.3 | 335 |
| 120 | 122 | 18.2 | 740 |
| 121 | 47.4 | 16.0 | 472 |
| 122 | 1120 | 116 | 147 |
| 123 | 1190 | 89.5 | 90.3 |
| 124 | 1120 | 152 | 229 |
| 125 | 89.3 | 12.6 | 555 |
| 126 | 865 | 102 | 130 |
| 127 | 277 | 98.4 | 88.0 |
| 128 | 165 | 17.0 | 429 |
| 129 | 998 | 72.6 | 62.6 |
| 130 | 1140 | 99.1 | 220 |
| 131 | 225 | 27.2 | 949 |
| 132 | 64.9 | 12.7 | 568 |
| 133 | 105 | 12.7 | 917 |
| 134 | 220 | 34.7 | 1200 |
| 135 | 123 | 18.6 | 568 |
| 136 | 121 | 20.5 | 629 |
| 137 | 123 | 13.0 | 704 |
| 138 | 56.2 | 8.0 | 555 |
| 139 | 59.7 | 6.0 | 489 |
| 140 | 200 | 19.6 | 878 |
| 141 | 119 | 11.7 | 771 |
| 142 | 102 | 9.99 | 453 |
| 143 | 93.5 | 12.7 | 483 |
| 144 | 76.1 | 8.05 | 779 |
| 145 | 111 | 15.2 | 613 |
| 146 | 92.5 | 12.9 | 842 |
| 147 | 83.0 | 11.0 | 837 |
| 148 | 72.7 | 7.98 | 728 |
| 149 | 88.4 | 16.3 | 1060 |
| 150 | 68.7 | 9.08 | 797 |
| 151 | 75.5 | 10.3 | 822 |
| 152 | 62.4 | 10.5 | 719 |
| 153 | 66.5 | 10.5 | 913 |
| 154 | 80.6 | 14.2 | 1140 |
| 155 | 55.6 | 14.0 | 652 |
| 156 | 91.8 | 26.5 | 876 |
| 157 | 77.4 | 16.3 | 636 |
| 158 | 112 | 19.6 | 638 |
| 159 | 103 | 14.1 | 716 |
| 160 | 55.9 | 8.7 | 715 |
| 161 | 54.2 | 7.7 | 1150 |
| 162 | 61.2 | 6.0 | 988 |
| 163 | 68.1 | 7.43 | 2230 |
| 164 | 60.3 | 7.59 | 1180 |
| 165 | 85.0 | 9.11 | 598 |
| 166 | 54.8 | 6.19 | 1300 |
| 167 | 66.5 | 5.85 | 1340 |
| 168 | 63.0 | 6.42 | 915 |
| 169 | 517 | 74.1 | 184 |
| 170 | 685 | 36.8 | 127 |
| 171 | 468 | 39.0 | 110 |
| 172 | 578 | 85.2 | 353 |
| 173 | 750 | 63.8 | 290 |
| 174 | 602 | 93.2 | 1650 |
| 175 | 731 | 82.3 | 407 |
| 176 | 125 | 20.4 | 706 |
| 177 | 181 | 18.7 | 779 |
| 178 | 173 | 17.0 | 1480 |
| 179 | 119 | 20.9 | 1020 |
| 180 | 80.5 | 11.9 | 1100 |
| 181 | 98 | 19.7 | 897 |
| 182 | 139 | 18.6 | 2740 |
| 183 | 692 | 73.6 | 144 |
| 184 | 718 | 61.6 | 123 |
| 185 | 954 | 72.3 | 192 |
| 186 | 830 | 85.5 | 208 |
| 187 | 1110 | 123 | 476 |
| 188 | 1150 | 154 | 1040 |
| 189 | 78.9 | 9.53 | 762 |
| 190 | 21.8 | 3.9 | 386 |
| 191 | 26 | 6.4 | 815 |
| 192 | 55.7 | 8.22 | 291 |
| 193 | 110 | 12.0 | 275 |
| 194 | 763 | 15.7 | 1660 |
| 195 | 528 | 9.52 | 1690 |
| 196 | 201 | 21.6 | 641 |
| 197 | 148 | 18.4 | 882 |

Absolute EC₅₀ values generated in the presence of 1% HSA may vary greatly depending on the HSA batch used. Thus, the absolute data cannot in itself be used, but will need to be compared to a reference compound run using the same HSA batch.

### Example 5: Experimental Protocol for Efficacy Testing on Appetite Using an Ad Libitum Fed Rat Model

Male Sprague Dawley (SD) rats from Taconic, Denmark were used for the acute food intake experiments, wherein the principles of laboratory animal care were followed.

The rats weighed 250-350 g at start of experiment and had not been on an obesogenic diet. The rats arrived at least seven days before the start of the experiment to allow acclimatization to experimental settings. Immediately upon arrival, rats were changed to a reversed light cycle (dark from 11 am-11 pm) and were transferred to an automated food intake measuring system (HM2system, MBRose; Faaborg, Denmark) with three rats housed per cage. Rats had ad libitum access to a 10% fat diet (Research Diets D12450B) and water, and were housed at room temperature (˜22° C.). To enable recording of individual food intake, rats were ID chipped during the acclimatization period. Since rats are normally active and consume most of their daily calories during the dark period, rats were dosed in the morning right before lights were turned off. Such a set-up results in the lowest data variation and highest test sensitivity. Each dose of tri-agonist was tested in a group of 5-8 rats. A vehicle group of 5-8 rats was included in each set of testing. Each cage contained animals from three different treatment groups (to eliminate potential cage effects (e.g. cage malfunction) on primary readout: food intake). The rats were dosed once subcutaneously (s.c.) with the compounds of interest or vehicle according to body weight (10, 30, or 100 nmol/kg) in vehicle using a NovoPen® (Novo Nordisk, Bagsværd, Denmark). Injection volume was 0.5 m/kg.

The compounds of the invention were formulated in the following vehicle: 8 mM phosphate; 250 mM glycerol; 0.007% polysorbate 20, pH 7.4.

After dosing, the rats were returned to their home cages, where they continued to have ad libitum access to the 10% fat diet and water. Food consumption was recorded continuously by the HM2 system from 24h leading up to test substance administration (baseline) and 0-72 h after test compound administration. Data recorded and obtained by the HM2 system were stored in a HMBase SQL database (Firebird® relational database management system) and were processed by HM2Lab software (MBRose; Faaborg, Denmark) installed in an embedded computer. The feeding system is a highly sensitive system with a load resolution of 0.001 g. In addition to recording food intake, the system records number of feeding events, which is defined as a 0.001 g reduction of food within 5s (Detailed information's of the system can be found in Rathod, Y. D., and Di Fulvio, M. (2021). The feeding microstructure of male and female mice. PLoS One 16, e0246569.) At the end of the experimental session, the animals were euthanized.

Table 15 shows acute food intake in normal weight (lean) rats based on above-described protocol for efficacy testing on appetite. The results allowed assessment of in vivo effect on food intake and provided an indication of the compounds' duration of action. Data are expressed as average percent inhibition relative to average food intake in vehicle group at each study day (day 1 [0-24 hours], day 2 [24-48 hours] and day 3 [48-72 hours]) and food intake was performed in rats up to 72 h. Food intake at each study day (e.g. day 1) means cumulative food intake during, i.e. over the course of this day (i.e. the 24 hours period).

TABLE 15
Results for acute food intake in normal weight
rats after a single dose of tri-agonist

Food intake at three different
doses (relative to vehicle in [%])

| | | | |
|---|---|---|---|
| Compound | Day 1 | Day 2 | Day 3 |

| | |
|---|---|
| No. | 10 nmol/kg/30 nmol/kg/100 nmol/kg |

| | | | |
|---|---|---|---|
| 104 | −1/−19/— | −21/−46/— | 0/−20/— |
| 105 | −13/—/−72 | −15/—/−100 | −12/—/−77 |
| 107 | −22/−28/−76 | −26/−44/−96 | −14/−11/−48 |
| 113 | −12/−14/−43 | −23/−29/−89 | −17/−9/−48 |
| 120 | −42 | −57 | −13 |
| 121 | −51 | −85 | −52 |
| 122 | −44 | −44 | −11 |
| 124 | −24 | −35 | 4 |
| 125 | −53 | −58 | −10 |
| 126 | −23 | −37 | 6 |
| 129 | −68 | −67 | −32 |
| 130 | −61 | −58 | 1 |
| 131 | −27 | −56 | −8 |
| 132 | −36 | −67 | −16 |
| 133 | −27 | −35 | −8 |
| 134 | −10 | −25 | 0 |
| 136 | −12 | −19 | 27 |
| 137 | −24 | −49 | 4 |
| 144 | −38 | −6 | −11 |
| 145 | −31 | −52 | −14 |
| 146 | −55 | −66 | −23 |
| 147 | −32 | −62 | −2 |
| 148 | −34 | −38 | −5 |
| 152 | −26 | −30 | 10 |
| 160 | −30 | −32 | −24 |
| 168 | −34 | −31 | −25 |
| 176 | −12 | 2 | 7 |
| 183 | −42 | −42 | −23 |
| 184 | −45 | −36 | −20 |
| 189 | −41 | −25 | −24 |

Where only one value is listed, food intake is measured at a dose of 10 nmol/kg. “−X” means that food intake is reduced by “X %” when compared to vehicle.

Following dosing of the GLP-1-/GlP-/amylin-receptor tri-agonists of the present invention to rats, it was observed that many of them induced profound food intake inhibition, compared to vehicle treatment, as can be deduced from the data presented in Table 15. In general, the compounds in Table 15 showed similar or improved reduction in food intake compared to reference compound 1 as disclosed in WO 2023/288313 at a much lower dose than the disclosed dosage level of 50 nmol/kg of reference compound 1.

### Example 6: Pharmacokinetic Study in Minipigs and Rats

The purpose of this study is to determine the half-life (t_{1/2}) in vivo of the inventive GLP-1-/GlP-/amylin-receptor tri-agonists after intravenous (i.v.) administration to minipigs or rats, i.e., the residence time in the body and thereby their time of action. This is done in a pharmacokinetic (PK) study, where the terminal half-life (t_{1/2}) of the compound or derivative in question is determined. By terminal half-life is meant the time it takes to halve a certain plasma concentration in the terminal elimination phase and reflects the duration of drug activity in the body.

### Minipig Study

Female Göttingen minipigs, obtained from Ellegaard Göttingen Minipigs (Dalmose, Denmark), approximately 8-12 months of age and weighing approximately 20-30 kg were used in the studies. The minipigs were housed individually (pigs with permanent catheters) in pens with straw as bedding and fed restrictedly once daily with Altromin 9023 minipig diet (Altromin Spezialfutter GmbH & Co. KG).

After three weeks of acclimatisation two permanent central venous catheters were implanted in vena cava caudalis in each animal. The animals were allowed at least 10 days recovery after the surgery and were then used for repeated pharmacokinetic studies with a suitable wash-out period between successive dosing.

The compounds of the invention were formulated (40 nmol/ml) in the following vehicle: 8 mM phosphate; 250 mM glycerol; 0.007% polysorbate 20, pH 7.4.

Intravenous injections (the volume corresponding to 0.05 ml/kg and dose of 2 nmol/kg/derivative) of the derivatives were given through one catheter, and blood was sampled at predefined time points for up till 14 days post dosing (preferably from the other catheter).

Blood samples (for example 1.3 ml) were collected in EDTA (1.3 ml tube containing K₃EDTA to yield 1.6 mg K₃EDTA/ml blood) coated tubes and then centrifuged at 4° C. and 2000×g for 10 minutes.

### Sampling and Analysis

Plasma was within 30 min after centrifugation pipetted into Micronic tubes stored on dry ice and were afterwards kept at −20° C. until analysed for plasma concentration of the compounds using LCMS.

Plasma concentrations of the peptides of the invention were assayed by plasma protein precipitation and analysed by liquid chromatography mass spectrometry (LC-MS). Calibrators were prepared by spiking blank plasma from minipigs with the compounds in the typical range from 0.05 to 200 nM. LLoQ was typically in the range of 0.2-2 nM. Calibrators, plasma blanks or study samples were prepared for LC-MS by protein precipitation by adding 4 volumes of ethanol containing 20 nM of internal standard (structurally similar analogue with different mass) to one volume of sample followed by centrifugation at 6200 rpm at 4° C. for 10 minutes. The supernatant was diluted with 1 volumes of Milli-Q water containing 1% Formic acid before injection on the LC-MS system. Individual plasma concentration-time profiles were analysed by a non-compartmental pharmacokinetic method (NCA) in Phoenix v. 6.4 (Pharsight Inc., Mountain View, CA, USA), and the resulting terminal half-lives (harmonic mean) determined. The LC-MS analysis was carried out using a TurboFlow HPLC system from Thermo Fisher Scientific (Bremen, Germany) coupled to either a Q Exactive Orbitrap or Altis Triple Quadrupole (TQ) Mass Spectrometer. The LC mobile phases consisted of A: MQ water with 5% organic solvent (50% methanol/50% acetonitrile) and 1% formic acid and B: MQ water with 95% organic solvent (50% methanol/50% acetonitrile) and 1% formic acid. A TurboFlow Cyclone 0.5×100 mm column from Thermo Fischer Scientific (Bremen, Germany) was used for extraction, before analytical elution on a XBridge Peptide BEH C18 300 Å, 3.5 μm, 2.1×50 mm column for the analysis both operated at 60° C. Typically 40-45% B and 75-80% B were used for loading and elution on the TurboFlow column, respectively, followed by a linear gradient elution typically from around 45% B to 85% B over 2.33 minutes on the analytical column. The Orbitrap mass spectrometer were operating in positive ionization mode with a spray voltage of 4.0 kV using Parallel Reaction Monitoring scan mode using 5 m/z isolation windows on the most abundant charge state of the compounds with a resolution of 35K on the Orbitrap MS. The TQ mass spectrometer were operating in positive ionization mode with a spray voltage of 4.0 kV using Single Reaction Monitoring scan mode using Q1 and Q3 resolutions of 1.2 (FWHM).

For all compounds, individual optimal fragmentation collision energies were found and used. The data was processed using the Quan Browser in the Xcalibur software from Thermo Fisher Scientific (Bremen, Germany) by fitted the data to linear calibration curves (weighed 1/x²), used for calculating the concentration in the plasma samples. Quality control samples were included. The deviation between nominal and calculated concentration in the calibrators and quality control samples were below 15%.

### Rat Study

Male Sprague-Dawley rats (250-320 g) were obtained from Charles River, Germany or Janvier Labs France. Animals were housed two per cage with free access to rodent diet (Altromin 1324) and water throughout the study.

Following a 12-day acclimation period, tri-agonist compounds formulated in 8 mM phosphate; 250 mM glycerol, pH 7.4 were administered by i.v. injection (50 nmol/0.5 ml/kg), and blood was sampled at various pre-defined time points up to 72h post dosing.

### Sampling and Analysis

Blood samples (˜120 μl/sample) were collected in K₃EDTA coated vials and centrifuged at 2000×g for 5 minutes at 4° C. Plasma samples (50-60 μl/sample) were transferred to micronic tubes, snap frozen and stored at −20° C. until analysed for plasma concentration of the compounds using LC-MS. Plasma concentrations of the peptides of the invention were assayed by plasma protein precipitation and analysed by liquid chromatography mass spectrometry (LC-MS). Calibrators were prepared by spiking blank plasma from rat with the compounds in the typical range from 0.5 to 2000 nM. LLOQ was typically in the range of 0.5-1 nM. Calibrators, plasma blanks or study samples were prepared for LC-MS by protein precipitation by adding 6 volumes of methanol containing 20 nM of internal standard (structurally similar analogue with different mass) to one volume of sample followed by centrifugation at 6400 rpm at 4° C. for 10 minutes. The supernatant was diluted with 1 volumes of Milli-Q water containing 1% Formic acid before injection on the LC-MS system. The LC-MS analysis was carried out using a TurboFlow or HPLC system from Thermo Fisher Scientific (Bremen, Germany) coupled to either a Q Exactive Plus or Q Exactive HF Orbitrap Mass Spectrometer. The LC mobile phases consisted of A: MQ water with 5% organic solvent (50% methanol/50% acetonitrile) and 1% formic acid and B: MQ water with 95% organic solvent (50% methanol/50% acetonitrile) and 1% formic acid and for HPLC system mobile phase A consists of 99.9% milli-Q water and 0.1% formic acid; mobile phase B consists of 99.9% acetonitrile and 0.1% formic acid. TurboFlow Cyclone 0.5×100 mm column from Thermo Fischer Scientific (Bremen, Germany) was used for extraction, before analytical elution on a XBridge Peptide BEH C18 300 Å, 3.5 μm, 2.1×50 mm column for the analysis both operated at 60° C. The analytical column Accucore 150-C4 (2.1×100 mm, 2.6 uM from Thermo Fischer Scientific) was used on the HPLC system. Typically, 20% B and 75% B were used for loading and elution on the TurboFlow column, respectively, followed by a linear gradient elution typically from around 49% B to 89% B over 4 minutes on the analytical column. HPLC gradient was in range from 20 to 80% B. Q Exactive Plus or Q Exactive HF Orbitrap mass spectrometers were operating in positive ionization mode with a spray voltage of 4.0 kV using with the single ion monitoring (SIM) scan mode using 5 m/z isolation windows on the most abundant charge state of the compounds with a resolution of 35K and 60K on the mass spectrometers respectively. The data was processed using the Quan Browser in the Xcalibur software from Thermo Fisher Scientific (Bremen, Germany) by fitted the data to linear calibration curves (weighed 1/x2), used for calculating the concentration in the plasma samples. Quality control samples were included. The deviation between nominal and calculated concentration in the calibrators and quality control samples were below 15%.

### Results

TABLE 16
Terminal half-life as measured after i.v.
administration to rats or minipigs

| | Terminal half-life as | Terminal half-life as |
|---|---|---|
| | measured after i.v. | measured after i.v. |
| Compound | administration to rats | administration to minipigs |
| no. | t_{1/2} [hours] | t_{1/2} [hours] |
| 104 | 26.8 | |
| 105 | 16.9 | |
| 107 | 14 | |
| 113 | 16 | |
| 120 | | 95 |
| 125 | | 99.8 |
| 129 | | 112 |
| 130 | | 182 |
| 144 | | 135 |
| 176 | | 132 |
| 183 | 17.5 | 182 |
| 189 | | 131 |

As shown in Table 16, the tested GLP-1-/GlP-/amylin-receptor tri-agonists of the present invention have very long half-lives (t_{1/2}) of at least 14 hours in rats or 95 hours in minipigs. It is contemplated that, based on these half-lives in rats and minipigs, half-lives in humans will be sufficient for at least once-weekly administration via liquid subcutaneous injection or at least once daily administration via oral tablet.

### Example 7: Chemical Stability Assessment in Formulation

The assay was performed to investigate the extend of chemical degradation in vitro over time upon incubation at 370° C. for a period of 2 weeks.

Peptide solutions were prepared by dissolving freeze-dried powder in 8 mM phosphate buffer pH 7.4 to a target of 1 mg/mL. The pH of peptide solutions was adjusted to 7.4 with 0.02 M HCl or 0.02 M NaOH. Samples were filled in Agilent HPLC vials with fixed insert. Vials were capped to prevent evaporation. The HPLC vials were incubated at 370° C. and samples were withdrawn at different time points over a period of 2 weeks, flash frozen at −80° C., and stored at −20° C. until analysis.

Sample analysis was carried out using UPLC coupled to UV detection at 215 nm and MS (UPLC-UV-MS). One μL of sample was injected into a Waters Acquity UPLC with a flow-through-needle injection system and on to a Waters Acquity CSH C18 column (1*150 mm), with a particle size of 1.7 μm and held at 55° C. A flow-rate of 100 μL/min was delivered with a Binary solvent manager pump having 0.1% formic acid in water as solvent A and 0.1% formic acid in acetonitrile as solvent B. Gradient elution was carried out using 20% B from 0 to 2 min followed by 20 to 50% B from 2 to 20 min in a total run time of 30 min. The identity of the peptide was confirmed by MS and the peak purity, area %, from the UV signal at 215 nm was plotted against time and the slope from linear regression was used to calculate the purity loss per week (Table 17).

TABLE 17
Chemical stability assessment of peptides in
8 mM phosphate buffer pH 7.4 (1 mg/mL) at 37°
C. Data is given as purity loss in % per week.

| Compound | Purity loss per week |
|---|---|
| number | at 37° C. [in %] |
| 104 | 1.7 |
| 105 | 1.4 |
| 106 | 0.3 |
| 107 | 1.0 |
| 108 | 1.1 |
| 109 | 0.6 |
| 110 | 0.9 |
| 113 | 0.3 |
| 120 | 1.2 |
| 121 | 1.5 |
| 122 | 1.3 |
| 123 | 1.0 |
| 124 | 1.5 |
| 125 | 0.5 |
| 126 | 1.2 |
| 127 | 1.7 |
| 129 | 1.7 |
| 130 | 1.6 |
| 132 | 1.3 |
| 133 | 1.4 |
| 134 | 1.4 |
| 135 | 0.7 |
| 137 | 0.8 |
| 138 | 1.2 |
| 139 | 1.3 |
| 140 | 0.8 |
| 141 | 1.4 |
| 144 | 1.2 |
| 145 | 0.8 |
| 146 | 1.4 |
| 147 | 1.0 |
| 148 | 1.4 |
| 149 | 1.1 |
| 150 | 1.6 |
| 151 | 1.3 |
| 152 | 1.3 |
| 153 | 1.4 |
| 154 | 1.4 |
| 159 | 0.5 |
| 160 | 1.2 |
| 161 | 0.9 |
| 162 | 1.0 |
| 163 | 1.4 |
| 164 | 1.2 |
| 165 | 1.0 |
| 166 | 0.8 |
| 167 | 0.9 |
| 168 | 0.7 |
| 169 | 1.5 |
| 170 | 0.6 |
| 171 | 1.0 |
| 172 | 0.9 |
| 173 | 1.3 |
| 174 | 1.6 |
| 175 | 1.5 |
| 176 | 0.9 |
| 177 | 0.3 |
| 178 | 1.1 |
| 179 | 0.3 |
| 180 | 0.7 |
| 181 | 1.3 |
| 182 | 0.7 |
| 183 | 1.4 |
| 184 | 1.2 |
| 185 | 1.4 |
| 186 | 1.0 |
| 188 | 1.3 |
| 189 | 0.5 |
| 190 | 1.4 |
| 191 | 0.9 |
| 192 | 0.5 |
| 193 | 0.4 |
| 194 | 0.4 |
| 195 | 0.5 |

All GLP-1-/GlP-/amylin-receptor tri-agonists tested in this assay show an acceptable chemical stability with an acceptable rate of degradation (less than 5% of purity loss per week) in an aqueous buffer (at 37° C.). Most of the tested GLP-1-/GlP-/amylin-receptor tri-agonists show a good chemical stability having less than 2.0% purity loss per week, or even more an excellent chemical stability (less than 1.5% purity loss per week). Accordingly, the GLP-1-/GlP-/amylin-receptor tri-agonists of the present invention are considered to be chemically stable in solution.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A GLP-1-/GlP-/amylin-receptor tri-agonist comprising peptide Z1-L1-Z2;
wherein Z1 comprises
(SEQ ID NO: 168)
YX₂EGTFTSDYSX₁₂LLEEIAAX₂₀EFIX₂₄WLX₂₇X₂₈GX₃₀X₃₁SX₃₃
X₃₄;
wherein
X₂ represents Aib,
X₁₂ represents Lys (K) or lie (1),
X₂₀ represents Arg (R),
X₂₄ represents Glu (E),
X₂₇ represents Leu (L) or lie (1),
X₂₈ represents Ala (A),
X₃₀ represents Gly (G), or Ala (A)
X₃₁ represents (P), Gly (G), or Ala (A),
X₃₃ represents Ser (S),
X₃₄ represents Gly (G);
wherein L1 is a peptide linker; and
wherein Z2 comprises AX₅₂X₅₃LSTAX₅₈X₅₉X₆₀RLSAELHX₆₈LATX₇₂PRTETGSGSP (SEQ ID NO: 165);
wherein
X₅₂ represents Ser (S),
X₅₃ represents Glu (E) or His (H),
X₅₈ represents Ala (A) or Gln (Q),
X₅₉ represents Leu (L) or Thr (T),
X₆₀ represents Gly (G) or Gln (Q),
X₆₈ represents Glu (E) or Lys (K), or Gln (Q),
X₇₂ represents Leu (L);
or a pharmaceutically acceptable salt thereof.

2. The GLP-1-/GlP-/amylin-receptor tri-agonist according to claim 1, wherein the peptide linker L1 comprises X₄₁X₄₂X₄₃X₄₄X₄₅X₄₆X₄₇X₄₈X₄₉X₅₀,
wherein
X₄₁ represents Ala (A), Gly (G) or Glu (E),
X₄₂ represents Glu (E), Gly (G) or is absent,
X₄₃ represents Glu (E), Gly (G), Gln (Q) or absent,
X₄₄ represents Ala (A), Glu (E), Gly (G) or absent,
X₄s represents Glu (E), Gly (G), Pro (P) or is absent,
X₄₆ represents Glu (E), Gly (G) or is absent,
X₄₇ represents Gln (Q), Glu (E) or is absent,
Xₐ represents Ala (A), Glu (E) or is absent,
X₄₉ represents Glu (E), Pro (P) or is absent,
X₅₀ represents Ala (A), Glu (E), Gly (G), Leu (L), Pro (P), Ser (S), Val (V) or is absent.

3. The GLP-1-/GlP-/amylin-receptor tri-agonist according to claim 2, wherein the peptide linker L1 is AG.

4. The GLP-1-/GlP-/amylin-receptor tri-agonist according to claim 1, wherein the peptide Z1-L1-Z2 comprises the amino acid sequence selected from the group consisting of:
(SEQ ID NO: 230)
YX₂EGTFTSDYSKLLEEIAAREFIEWLLAGGPSSGAGASELSTAQLGRLS
AELHQLATLPRTETGSGSP,
(SEQ ID NO: 243)
YX₂EGTFTSDYSILLEEIAAREFIEWLLAGGASSGAGEAPGEAPGASHLS
TAQTQRLSAELHKLATLPRTETGSGSP,
(SEQ ID NO: 244)
YX₂EGTFTSDYSKLLEEIAAREFIEWLLAGGPSSGAGASELSTAALGRLS
AELHQLATLPRTETGSGSP,
(SEQ ID NO: 246)
YX₂EGTFTSDYSKLLEEIAAREFIEWLIAGAPSSGAGASELSTAALGRLS
AELHQLATLPRTETGSGSP,
(SEQ ID NO: 250)
YX₂EGTFTSDYSKLLEEIAAREFIEWLIAGAPSSGAGASELSTAALGRLS
AELHELATLPRTETGSGSP,
(SEQ ID NO: 251)
YX₂EGTFTSDYSKLLEEIAAREFIEWLLAGGGSSGAGASELSTAALGRLS
AELHELATLPRTETGSGSP,
and
(SEQ ID NO: 252)
YX₂EGTFTSDYSKLLEEIAAREFIEWLLAGGPSSGAGASELSTAALGRL
SAELHELATLPRTETGSGSP;
wherein X₂ is Aib.

5. The GLP-1-/GlP-/amylin-receptor tri-agonist according to claim 1, further comprising a protraction moiety.

6. The GLP-1-/GlP-/amylin-receptor tri-agonist according to claim 5, wherein the protraction moiety comprises a C₁₂-C₂₀ diacid.

7. The GLP-1-/GlP-/amylin-receptor tri-agonist according to claim 5, wherein the protraction moiety further comprises a linker.

8. The GLP-1-/GlP-/amylin-receptor tri-agonist according to 5, wherein the protraction moiety is 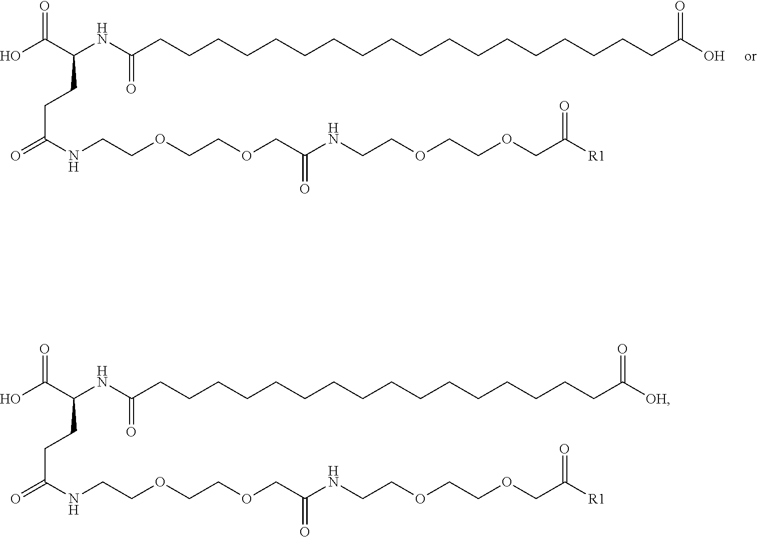
wherein R1 is the point of attachment of the protraction moiety to the epsilon amino group of the lysine in the peptide Z1-L1-Z2.

9. The GLP-1-/GlP-/amylin-receptor tri-agonist according to claim 1, wherein the GLP-1-/GlP-/amylin-receptor tri-agonist GLP-1 is

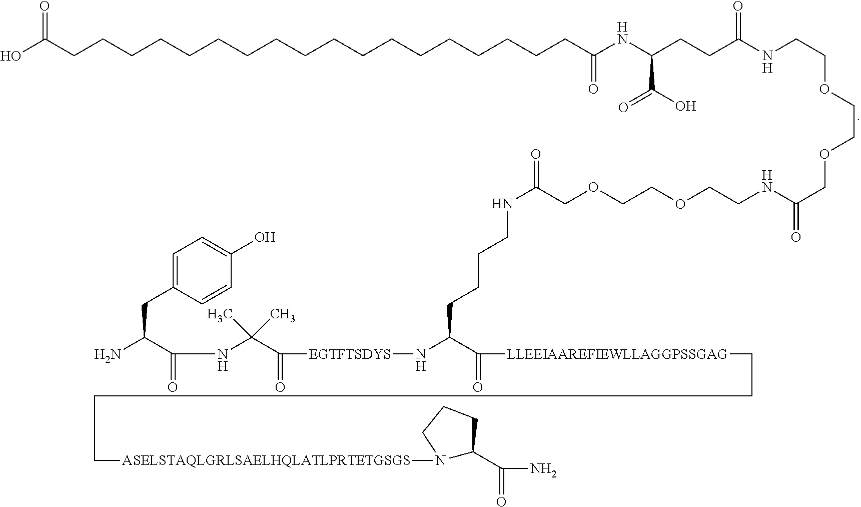

10. The GLP-1-/GlP-/amylin-receptor tri-agonist according to claim 1, wherein the GLP-1-/GlP-/amylin-receptor tri-agonist GLP-1 is

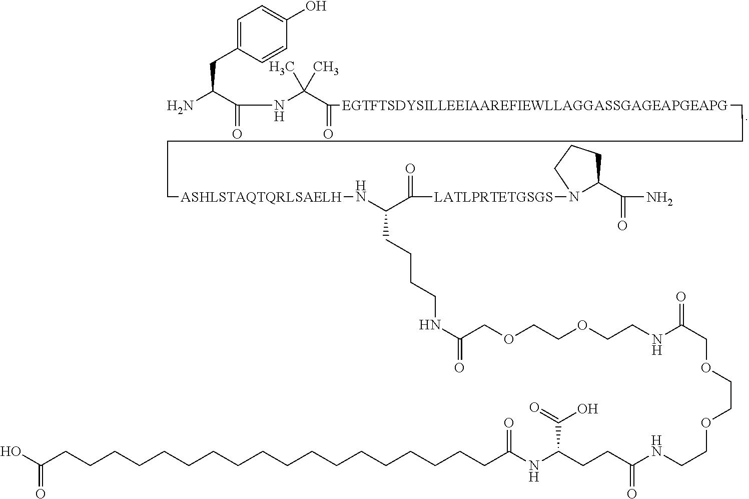

11. The GLP-1-/GlP-/amylin-receptor tri-agonist according to claim 1, wherein the GLP-1-/GlP-/amylin-receptor tri-agonist GLP-1 is

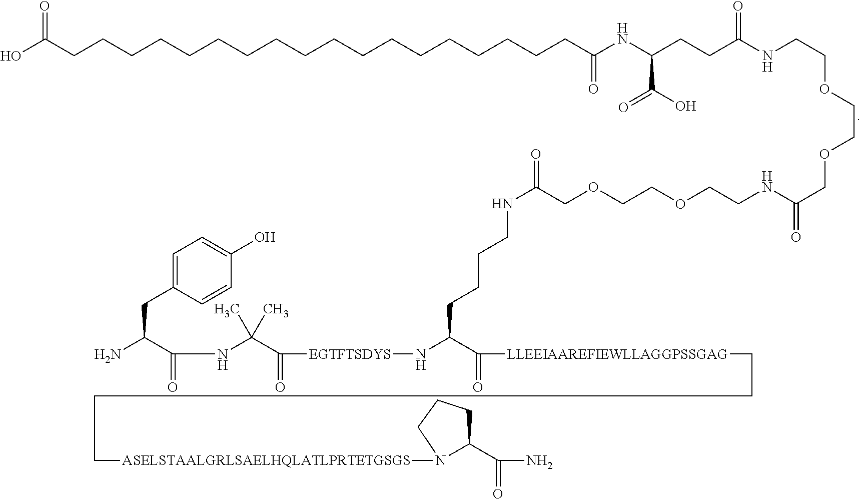

12. The GLP-1-/GlP-/amylin-receptor tri-agonist according to claim 1, wherein the GLP-1-/GlP-/amylin-receptor tri-agonist GLP-1 is

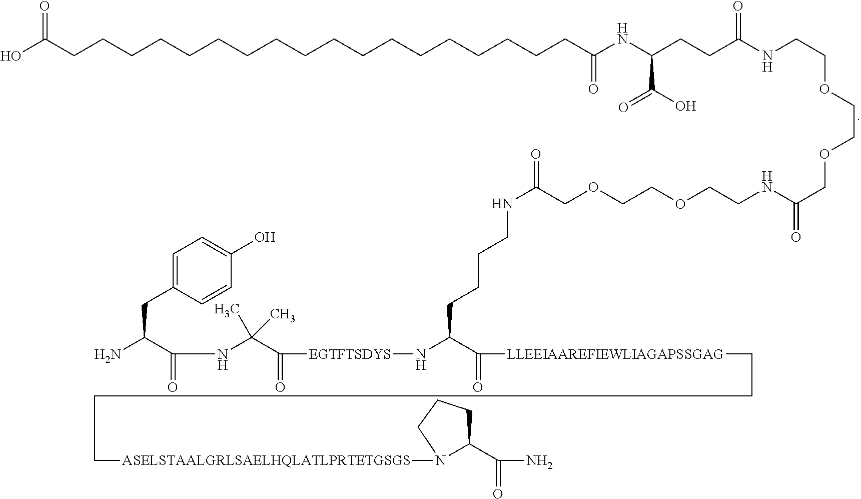

13. The GLP-1-/GlP-/amylin-receptor tri-agonist according to claim 1, wherein the GLP-1-/GlP-/amylin-receptor tri-agonist GLP-1 is

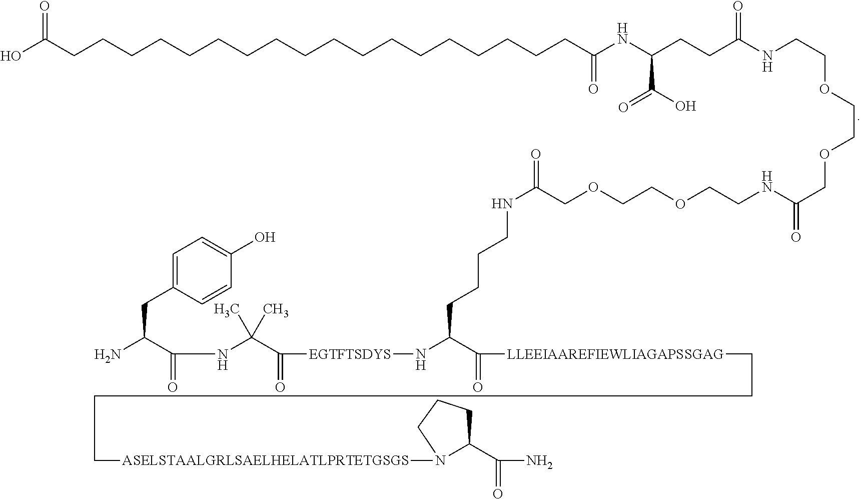

14. The GLP-1-/GlP-/amylin-receptor tri-agonist according to claim 1, wherein the GLP-1-/GlP-/amylin-receptor tri-agonist GLP-1 is

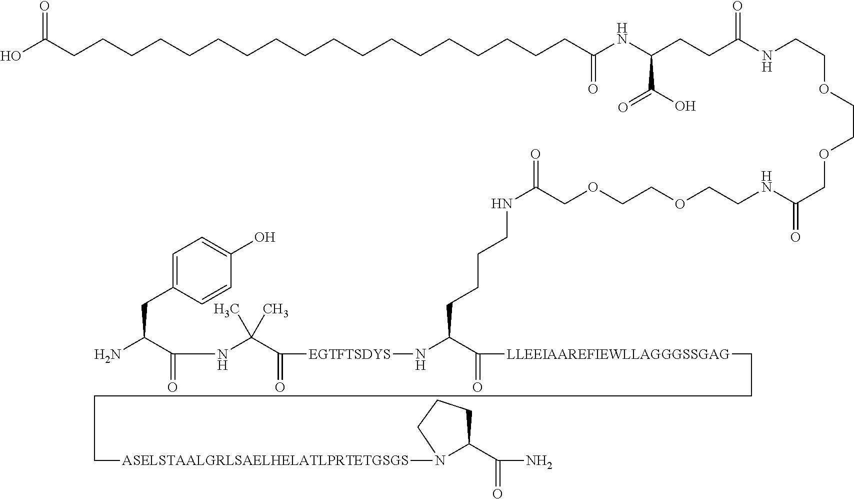

15. The GLP-1-/GlP-/amylin-receptor tri-agonist according to claim 1, wherein the GLP-1-/GlP-/amylin-receptor tri-agonist GLP-1 is

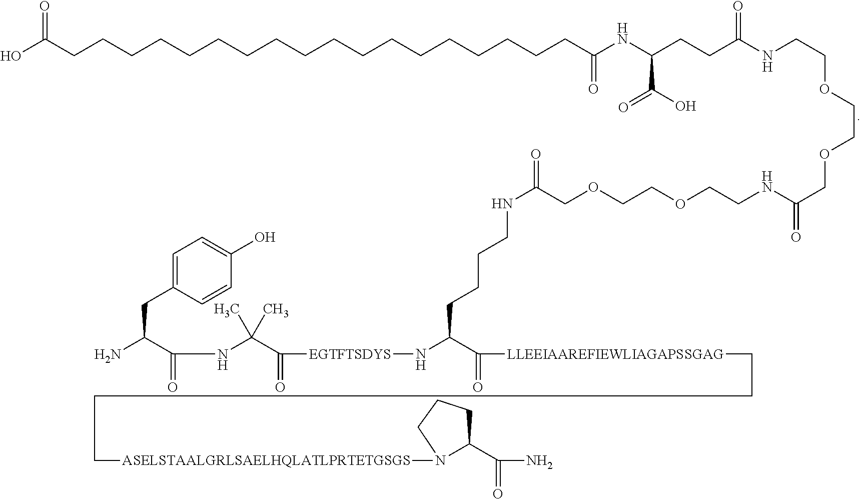

16. A pharmaceutical composition comprising a GLP-1-/GlP-/amylin-receptor tri-agonist according to claim 1 and one or more pharmaceutically acceptable excipients.

17. A method of treating obesity, comprising administering the GLP-1-/GlP-/amylin-receptor tri-agonist according to claim 1 to a subject in need thereof.

18. The method according to claim 17, wherein the peptide linker L1 comprises X₄₁X₄₂X₄₃X₄₄X₄₅X₄₆X₄₇X₄₈X₄₉X₅₀,
wherein
X₄₁ represents Ala (A), Gly (G) or Glu (E),
X₄₂ represents Glu (E), Gly (G) or is absent,
X₄₃ represents Glu (E), Gly (G), Gln (Q) or absent,
X₄₄ represents Ala (A), Glu (E), Gly (G) or absent,
X₄₅ represents Glu (E), Gly (G), Pro (P) or is absent,
X₄₆ represents Glu (E), Gly (G) or is absent,
X₄₇ represents Gln (Q), Glu (E) or is absent,
X₄₈ represents Ala (A), Glu (E) or is absent,
X₄₉ represents Glu (E), Pro (P) or is absent,
X₅₀ represents Ala (A), Glu (E), Gly (G), Leu (L), Pro (P), Ser (S), Val (V) or is absent.

19. The method according to claim 18, wherein the peptide linker L1 is AG.

20. The method according to claim 17, wherein the peptide Z1-L1-Z2 comprises the amino acid sequence selected from the group consisting of:
(SEQ ID NO: 230)
YX₂EGTFTSDYSKLLEEIAAREFIEWLLAGGPSSGAGASELSTAQLGRLS
AELHQLATLPRTETGSGSP,
(SEQ ID NO: 243)
YX₂EGTFTSDYSILLEEIAAREFIEWLLAGGASSGAGEAPGEAPGASHLS
TAQTQRLSAELHKLATLPRTETGSGSP,
(SEQ ID NO: 244)
YX₂EGTFTSDYSKLLEEIAAREFIEWLLAGGPSSGAGASELSTAALGRLS
AELHQLATLPRTETGSGSP,
(SEQ ID NO: 246)
YX₂EGTFTSDYSKLLEEIAAREFIEWLIAGAPSSGAGASELSTAALGRLS
AELHQLATLPRTETGSGSP,
(SEQ ID NO: 250)
YX₂EGTFTSDYSKLLEEIAAREFIEWLIAGAPSSGAGASELSTAALGRLS
AELHELATLPRTETGSGSP,
(SEQ ID NO: 251)
YX₂EGTFTSDYSKLLEEIAAREFIEWLLAGGGSSGAGASELSTAALGRLS
AELHELATLPRTETGSGSP,
and
(SEQ ID NO: 252)
YX₂EGTFTSDYSKLLEEIAAREFIEWLLAGGPSSGAGASELSTAALGRL
SAELHELATLPRTETGSGSP;
wherein X₂ is Aib.

21. The method according to claim 17, further comprising a protraction moiety.

22. The method according to claim 17, wherein the protraction moiety comprises a C₁₂-C₂₀ diacid.

23. The method according to claim 17, wherein the protraction moiety further comprises a linker.

24. The method according to claim 17, wherein the GLP-1-/GlP-1/amylin-receptor tri-agonist is

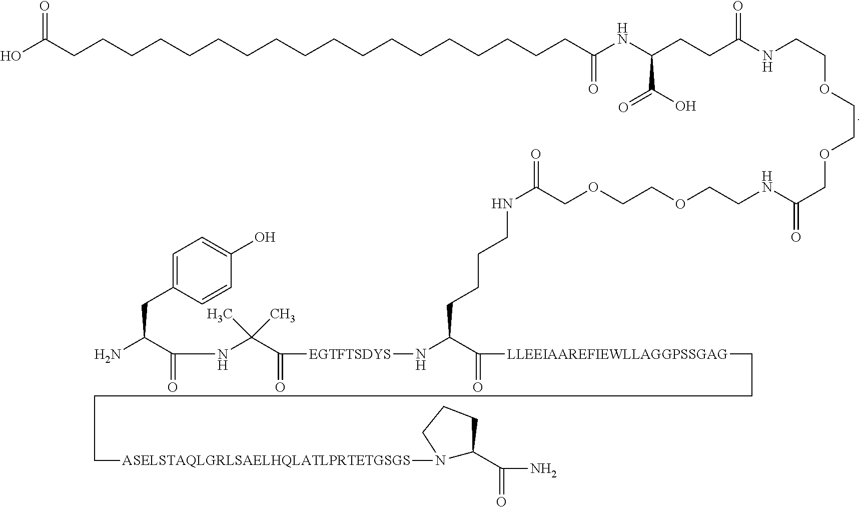

25. The method according to claim 17, wherein the GLP-1-/GlP-/amylin-receptor tri-agonist GLP-1 is

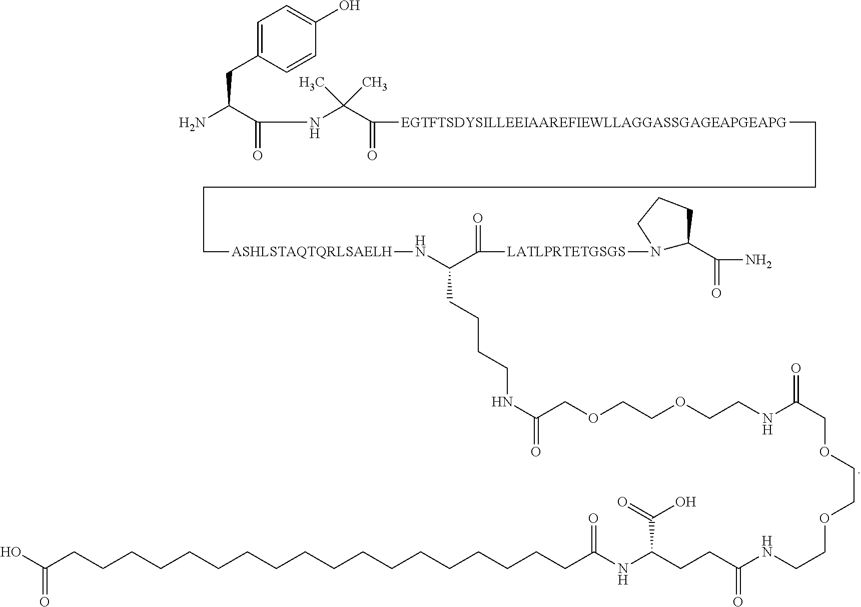

26. The method according to claim 17, wherein the GLP-1-/GlP-/amylin-receptor tri-agonist GLP-1 is

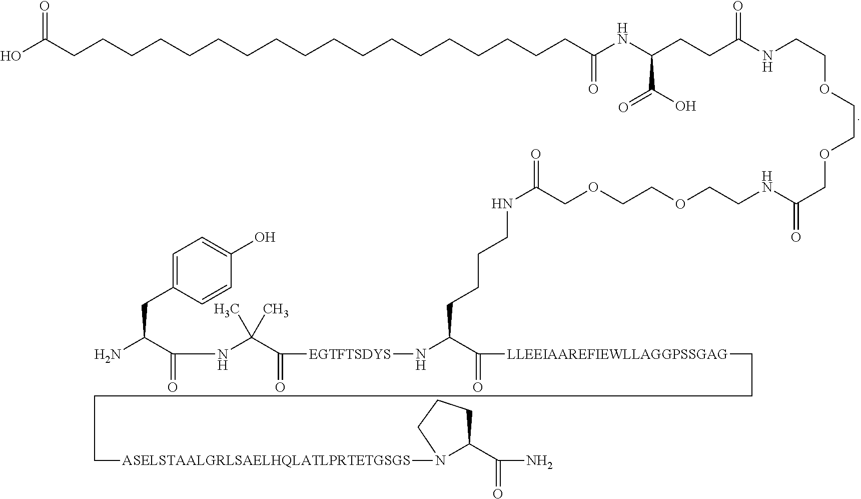

27. The method according to claim 17, wherein the GLP-1-/GlP-/amylin-receptor tri-agonist GLP-1 is

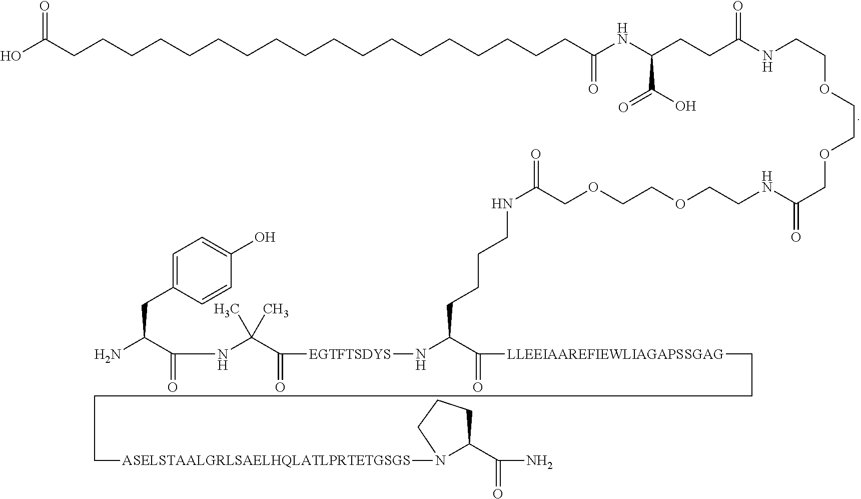

28. The method according to claim 17, wherein the GLP-1-/GlP-/amylin-receptor tri-agonist GLP-1 is

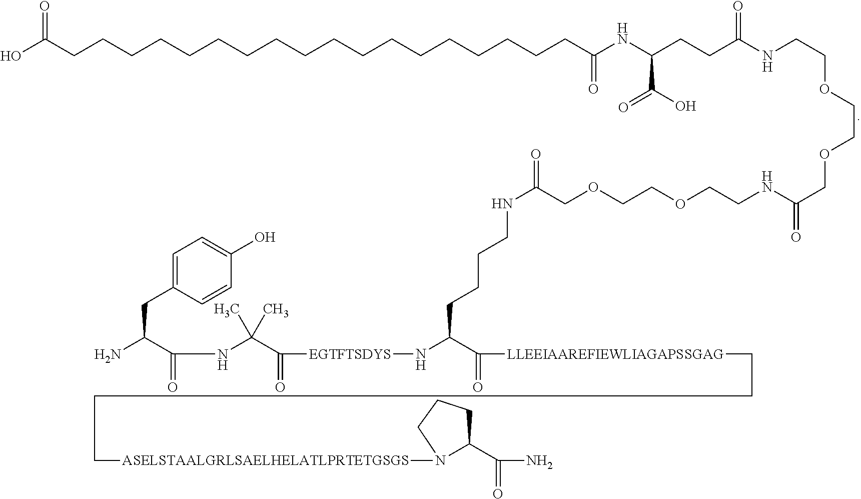

29. The method according to claim 17, wherein the GLP-1-/GlP-/amylin-receptor tri-agonist GLP-1 is

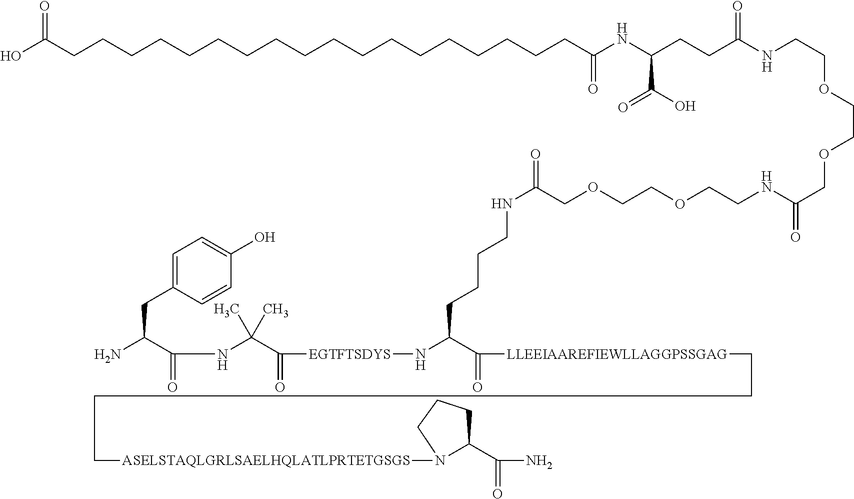

30. The method according to claim 17, wherein the GLP-1-/GlP-/amylin-receptor tri-agonist GLP-1 is [chemical structure]


SEQUENCE LISTING (252 sequences)

SEQ ID NO: 1 - protein, 34 aa, synthetic construct
  SITE 2: note = Aib
YXEGTFTSDYSILLEEQAAREFIEWLLAGGPSKG

SEQ ID NO: 2 - protein, 32 aa, synthetic construct
  MOD_RES 32: note = last position: C-terminal amide
ASELSTAALGRLSAELHELATLPRTETGSGSP

SEQ ID NO: 3 - skipped

SEQ ID NO: 4 - protein, 32 aa, synthetic construct
  VARIANT 3: note = Q, E, H, K
  VARIANT 8: note = A, Q
  VARIANT 9: note = Q, L, T
  VARIANT 10: note = A, G, Q
  VARIANT 15: note = E, K
  VARIANT 18: note = R, Q, E, G, H, K, T, Y
  VARIANT 20: note = A, K
  VARIANT 21: note = D, T
  VARIANT 24: note = R, K
  VARIANT 31: note = A, S
ASXLSTAXXXRLSAXLHXLXXLPXTETGSGXP

SEQ ID NO: 5 - protein, 80 aa, synthetic construct
  VARIANT 2: note = Aib
  VARIANT 12: note = I, K
  VARIANT 20: note = R, Q
  VARIANT 24: note = A, E, Q
  VARIANT 28: note = A, Q
  VARIANT 33: note = E, S
  VARIANT 34: note = G, E
  VARIANT 35: note = A, E, G
  VARIANT 36: note = Q, E, G, L, P or absent
  VARIANT 37: note = A, Q, E, G, P or absent
  VARIANT 38: note = A, Q, E, G, P or absent
  VARIANT 39: note = E, G, P, S, T or absent
  VARIANT 40: note = E, G, L, Q or absent
  VARIANT 41: note = A, Q, E, G, F or absent
  VARIANT 42: note = A, Q, E, G, T, P, V or absent,
  VARIANT 43: note = E, N, P, T or absent,
  VARIANT 44: note = A, Q, E, G, L, P, S, V or absent
  VARIANT 45: note = A or absent
  VARIANT 46: note = Q or absent
  VARIANT 47: note = T or absent
  VARIANT 48: note = L or absent
  VARIANT 50: note = G, S
  VARIANT 51: note = Q, E, H
  VARIANT 56: note = A, Q
  VARIANT 57: note = L, T
  VARIANT 58: note = A, G, Q
  VARIANT 66: note = Q, E, K
  VARIANT 70: note = L, E
  VARIANT 30: note = G, A
  VARIANT 31: note = G, Q, A, P
YXEGTFTSDYSXLLEEIAAXEFIXWLJXGXXSXXXXXXXXXXXXXXXXAXXLSTAXXXRLSAELHXLATXPRTETGSGSP

SEQ ID NO: 6-9 - skipped (4 entries)

SEQ ID NO: 10 - protein, 76 aa, synthetic construct
  SITE 2: note = Aib
  VARIANT 12: note = I, K
  VARIANT 20: note = R, Q
  VARIANT 24: note = A, E, Q
  VARIANT 28: note = A, Q
  VARIANT 30: note = G, A
  VARIANT 31: note = G, Q, A, P
  VARIANT 33: note = E, S
  VARIANT 34: note = G, E
  VARIANT 35: note = A, E, G
  VARIANT 36: note = E, G, or absent
  VARIANT 37: note = Q, E, G, or absent
  VARIANT 38: note = A, E, G, or absent
  VARIANT 39: note = E, G, P, or absent
  VARIANT 40: note = E, G, or absent
  VARIANT 41: note = Q, E or absent
  VARIANT 42: note = A, E, or absent
  VARIANT 43: note = E, P or absent
  VARIANT 44: note = A, E, G, L, P, V or absent,
  VARIANT 46: note = G, S
  VARIANT 47: note = Q, E, H
  VARIANT 52: note = A, Q
  VARIANT 53: note = L, T
  VARIANT 54: note = A, G, Q
  VARIANT 62: note = Q, E, K
  VARIANT 66: note = L, E
YXEGTFTSDYSXLLEEIAAXEFIXWLJXGXXSXXXXXXXXXXXXAXXLSTAXXXRLSAELHXLATXPRTETGSGSP

SEQ ID NO: 11 - protein, 63 aa, synthetic construct
  SITE 1: note = Position 1 = D-tyrosine
  DISULFID 33..38: note = Intrachain Disulfide bond: C33-C38
  MOD_RES 63: note = last position: C-terminal amide
YAEGTFTSDYSIALDKIAQQAFVQWLLAGGPSCNTATCVLGRLSQELHRLQTYPRTNTGSNTY

SEQ ID NO: 12 - protein, 80 aa, synthetic construct
  SITE 2: note = AIB
  SITE 13: note = AIB
  DISULFID 44..50: note = Intrachain Disulfide bond: C45-C50
  MOD_RES 80: note = last position: C-terminal amide
YXEGTFTSDYSIXLDKIAQKAFVQWLIAGGPSSGAPPPSGGGGKCNTATCATQRLAEFLRHSSNNFGPILPPTNVGSNTP

SEQ ID NO: 13 - protein, 76 aa, synthetic construct
  SITE 2: note = AIB
  SITE 13: note = AIB
  MOD_RES 76: note = last position: C-terminal amide
YXEGTFTSDYSIXLDKIAQKAFVQWLIAGGPSSGAPPPSGGGGEASELSTAALGRLSAELHELATLPRTETGSGSP

SEQ ID NO: 14 - protein, 31 aa, synthetic construct
  MOD_RES 1: note = Position 1 = N-acetyl
  SITE 1: note = Position 1 = D-tyrosine
  SITE 2: note = AIB
  SITE 24: note = Nle
  MOD_RES 31: note = last position: C-terminal amide
YXEGTFISDYSIAXDKIRQQDFVKWLLAQKP

SEQ ID NO: 15 - protein, 39 aa, synthetic construct
  SITE 2: note = AIB
  SITE 13: note = AIB
  MOD_RES 39: note = last position: C-terminal amide
YXEGTFTSDYSIXLDKIAQKAFVQWLIAGGPSSGAPPPS

SEQ ID NO: 16 - protein, 37 aa, synthetic construct
  DISULFID 2..7: note = Intrachain Disulfide bond: C2-C7
  MOD_RES 37: note = last position: C-terminal amide
KCNTATCATQRLAEFLRHSSNNFGPILPPTNVGSNTP

SEQ ID NO: 17 - protein, 31 aa, synthetic construct
  SITE 2: note = AIB
HXEGTFTSDVSSYLEGQAAKEFIAWLVRGRG

SEQ ID NO: 18-19 - skipped (2 entries)

SEQ ID NO: 20 - protein, 34 aa, synthetic construct
  SITE 2: note = AIB
YXEGTFTSDYSILLEEIAAREFIEWLLAGGPSSG

SEQ ID NO: 21 - protein, 34 aa, synthetic construct
  SITE 2: note = Aib
YXEGTFTSDYSILLEEIAAREFIEWLIAGGPSSG

SEQ ID NO: 22 - protein, 34 aa, synthetic construct
  SITE 2: note = Aib
YXEGTFTSDYSKLLEEIAAREFIEWLLAGGPSSG

SEQ ID NO: 23 - protein, 34 aa, synthetic construct
  SITE 2: note = Aib
YXEGTFTSDYSKLLEEIAAREFIEWLIAGGPSSG

SEQ ID NO: 24 - protein, 34 aa, synthetic construct
  SITE 2: note = Aib
YXEGTFTSDYSILLEEIAAREFIEWLLAGGPSSE

SEQ ID NO: 25 - protein, 34 aa, synthetic construct
  SITE 2: note = Aib
YXEGTFTSDYSILLEEIAAREFIAWLIAGGPSSG

SEQ ID NO: 26 - protein, 34 aa, synthetic construct
  SITE 2: note = Aib
YXEGTFTSDYSILLEEIAAREFIQWLIAGGPSSG

SEQ ID NO: 27 - protein, 34 aa, synthetic construct
  SITE 2: note = Aib
YXEGTFTSDYSKLLEEIAAREFIEWLLAGGPSSE

SEQ ID NO: 28 - protein, 34 aa, synthetic construct
  SITE 2: note = Aib
YXEGTFTSDYSILLEEIAAQEFIEWLLQGGPSSG

SEQ ID NO: 29 - protein, 34 aa, synthetic construct
  SITE 2: note = Aib
YXEGTFTSDYSILLEEIAAREFIEWLLAGGASSG

SEQ ID NO: 30 - protein, 34 aa, synthetic construct
  SITE 2: note = Aib
YXEGTFTSDYSKLLEEIAAREFIEWLLAGGQSSG

SEQ ID NO: 31 - protein, 34 aa, synthetic construct
  SITE 2: note = Aib
YXEGTFTSDYSKLLEEIAAREFIEWLIAGAPSSG

SEQ ID NO: 32 - protein, 34 aa, synthetic construct
  SITE 2: note = Aib
YXEGTFTSDYSKLLEEIAAREFIEWLIAGGPSEG

SEQ ID NO: 33 - protein, 34 aa, synthetic construct
  SITE 2: note = Aib
YXEGTFTSDYSKLLEEIAAREFIEWLIAGGGSSG

SEQ ID NO: 34 - protein, 34 aa, synthetic construct
  SITE 2: note = Aib
YXEGTFTSDYSKLLEEIAAREFIEWLLAGGGSSG

SEQ ID NO: 35-39 - skipped (5 entries)

SEQ ID NO: 40 - protein, 32 aa, synthetic construct
  MOD_RES 32: note = last position: C-terminal amide
ASHLSTAQTQRLSAELHKLATLPRTETGSGSP

SEQ ID NO: 41 - protein, 32 aa, synthetic construct
  MOD_RES 32: note = last position: C-terminal amide
ASHLSTAQLGRLSAELHELATLPRTETGSGSP

SEQ ID NO: 42 - protein, 32 aa, synthetic construct
  MOD_RES 32: note = last position: C-terminal amide
ASHLSTAQLGRLSAELHQLATLPRTETGSGSP

SEQ ID NO: 43 - protein, 32 aa, synthetic construct
  MOD_RES 32: note = last position: C-terminal amide
ASHLSTAQTQRLSAELHELATLPRTETGSGSP

SEQ ID NO: 44 - protein, 32 aa, synthetic construct
  MOD_RES 32: note = last position: C-terminal amide
ASHLSTAALGRLSAELHELATLPRTETGSGSP

SEQ ID NO: 45 - protein, 32 aa, synthetic construct
  MOD_RES 32: note = last position: C-terminal amide
ASHLSTAQTARLSAELHKLATLPRTETGSGSP

SEQ ID NO: 46 - protein, 32 aa, synthetic construct
  MOD_RES 32: note = last position: C-terminal amide
ASQLSTAQTQRLSAELHKLATLPRTETGSGSP

SEQ ID NO: 47 - protein, 32 aa, synthetic construct
  MOD_RES 32: note = last position: C-terminal amide
ASELSTAQTQRLSAELHKLATLPRTETGSGSP

SEQ ID NO: 48 - protein, 32 aa, synthetic construct
  MOD_RES 32: note = last position: C-terminal amide
AGELSTAQTQRLSAELHKLATLPRTETGSGSP

SEQ ID NO: 49 - protein, 32 aa, synthetic construct
  MOD_RES 32: note = last position: C-terminal amide
ASELSTAQTARLSAELHKLATLPRTETGSGSP

SEQ ID NO: 50 - protein, 32 aa, synthetic construct
  MOD_RES 32: note = last position: C-terminal amide
ASHLSTAQTQRLSAELHKLATEPRTETGSGSP

SEQ ID NO: 51 - protein, 32 aa, synthetic construct
  MOD_RES 32: note = last position: C-terminal amide
ASELSTAQTQRLSAELHKLATEPRTETGSGSP

SEQ ID NO: 52 - skipped

SEQ ID NO: 53 - protein, 32 aa, synthetic construct
  MOD_RES 32: note = last position: C-terminal amide
ASELSTAALGRLSAELHQLATLPRTETGSGSP

SEQ ID NO: 54 - protein, 32 aa, synthetic construct
  MOD_RES 32: note = last position: C-terminal amide
ASELSTAALGRLSAELHQLATEPRTETGSGSP

SEQ ID NO: 55 - protein, 32 aa, synthetic construct
  MOD_RES 32: note = last position: C-terminal amide
ASELSTAQLGRLSAELHQLATEPRTETGSGSP

SEQ ID NO: 56 - protein, 32 aa, synthetic construct
  MOD_RES 32: note = last position: C-terminal amide
ASELSTAQTGRLSAELHQLATEPRTETGSGSP

SEQ ID NO: 57 - protein, 32 aa, synthetic construct
  MOD_RES 32: note = last position: C-terminal amide
ASELSTAQTQRLSAELHQLATEPRTETGSGSP

SEQ ID NO: 58 - protein, 32 aa, synthetic construct
  MOD_RES 32: note = last position: C-terminal amide
ASQLSTAQTQRLSAELHKLATEPRTETGSGSP

SEQ ID NO: 59 - protein, 32 aa, synthetic construct
  MOD_RES 32: note = last position: C-terminal amide
ASELSTAQLGRLSAELHQLATLPRTETGSGSP

SEQ ID NO: 60 - protein, 32 aa, synthetic construct
  MOD_RES 32: note = last position: C-terminal amide
ASHLSTAQLGRLSAELHQLATEPRTETGSGSP

SEQ ID NO: 61 - protein, 32 aa, synthetic construct
  VARIANT 3: note = E,H
  VARIANT 8: note = A,Q
  VARIANT 9: note = L,T
  VARIANT 10: note = G,Q
  VARIANT 18: note = Q,E,K
ASXLSTAXXXRLSAELHXLATLPRTETGSGSP

SEQ ID NO: 62 - protein, 34 aa, synthetic construct
  SITE 2: note = Aib
  VARIANT 12: note = I,K
  VARIANT 24: note = A,E,Q
  VARIANT 28: note = A, Q
  VARIANT 31: note = G,Q,A,P
  VARIANT 33: note = E,S
  VARIANT 34: note = G,E
  VARIANT 30: note = G,A
YXEGTFTSDYSXLLEEIAAREFIXWLJXGXXSXX

SEQ ID NO: 63-124 - skipped (62 entries)

SEQ ID NO: 125 - protein, 4 aa, synthetic construct
APPE

SEQ ID NO: 126 - protein, 4 aa, synthetic construct
GGGE

SEQ ID NO: 127 - protein, 6 aa, synthetic construct
AGQAPG

SEQ ID NO: 128 - protein, 8 aa, synthetic construct
APPPSGGG

SEQ ID NO: 129 - protein, 9 aa, synthetic construct
APPPSGGGE

SEQ ID NO: 130 - protein, 9 aa, synthetic construct
APPPSGGGG

SEQ ID NO: 131 - protein, 10 aa, synthetic construct
ALAQTLAQTL

SEQ ID NO: 132 - protein, 10 aa, synthetic construct
ALAQTLFVNQ

SEQ ID NO: 133 - protein, 10 aa, synthetic construct
ALAQTLGTNE

SEQ ID NO: 134 - protein, 10 aa, synthetic construct
ALQAPGQAPG

SEQ ID NO: 135 - protein, 10 aa, synthetic construct
ALQAPGQAPL

SEQ ID NO: 136 - protein, 10 aa, synthetic construct
AGQAPGQAPG

SEQ ID NO: 137 - protein, 10 aa, synthetic construct
AGQAPGQAPL

SEQ ID NO: 138 - protein, 10 aa, synthetic construct
GGGEGGGEGE

SEQ ID NO: 139 - protein, 10 aa, synthetic construct
GQAPGQAPGE

SEQ ID NO: 140 - protein, 10 aa, synthetic construct
GQEPGQEPGE

SEQ ID NO: 141 - protein, 14 aa, synthetic construct
APPPSLAQTLAQTL

SEQ ID NO: 142 - protein, 5 aa, synthetic construct
AGGGG

SEQ ID NO: 143 - protein, 10 aa, synthetic construct
AGEAPGQAPG

SEQ ID NO: 144 - protein, 10 aa, synthetic construct
AGEAPGEAPG

SEQ ID NO: 145 - protein, 10 aa, synthetic construct
AGQAPGQAPA

SEQ ID NO: 146 - protein, 10 aa, synthetic construct
AGQAPGQAPE

SEQ ID NO: 147 - protein, 10 aa, synthetic construct
AGQAPGQAPP

SEQ ID NO: 148 - protein, 10 aa, synthetic construct
AGQAPGQAPS

SEQ ID NO: 149 - protein, 10 aa, synthetic construct
AGQAPGQAPV

SEQ ID NO: 150 - protein, 10 aa, synthetic construct
EGQAPGQAPG

SEQ ID NO: 151 - protein, 10 aa, synthetic construct
AGQEPGQAPG

SEQ ID NO: 152 - protein, 10 aa, synthetic construct
AGQAEGQAPG

SEQ ID NO: 153 - protein, 10 aa, synthetic construct
AGQAPEQAPG

SEQ ID NO: 154 - protein, 10 aa, synthetic construct
AGQAPGEAPG

SEQ ID NO: 155 - protein, 10 aa, synthetic construct
AGQAPGQEPG

SEQ ID NO: 156 - protein, 10 aa, synthetic construct
AGQAPGQAEG

SEQ ID NO: 157 - protein, 10 aa, synthetic construct
AGQEPGQEPG

SEQ ID NO: 158 - protein, 9 aa, synthetic construct
AGQAPGQAP

SEQ ID NO: 159 - protein, 10 aa, synthetic construct
AGQAPGEAPL

SEQ ID NO: 160-161 - skipped (2 entries)

SEQ ID NO: 162 - protein, 34 aa, synthetic construct
  SITE 2: note = Aib
  VARIANT 12: note = I, K
  VARIANT 20: note = R, Q
  VARIANT 24: note = A, E, Q
  VARIANT 28: note = A, Q
  VARIANT 34: note = G, E
YXEGTFTSDYSXLLEEIAAXEFIXWLJXGGPSSX

SEQ ID NO: 163 - protein, 34 aa, synthetic construct
  SITE 2: note = Aib
  VARIANT 12: note = I,K
YXEGTFTSDYSXLLEEIAAREFIEWLLAGGPSSG

SEQ ID NO: 164 - skipped

SEQ ID NO: 165 - protein, 32 aa, synthetic construct
  VARIANT 2: note = G,S
  VARIANT 3: note = Q, E, H
  VARIANT 8: note = A, Q
  VARIANT 9: note = L, T
  VARIANT 10: note = A, G, Q
  VARIANT 18: note = Q, E, K
  VARIANT 22: note = L, E
AXXLSTAXXXRLSAELHXLATXPRTETGSGSP

SEQ ID NO: 166 - protein, 32 aa, synthetic construct
  VARIANT 3: note = E, H
ASXLSTAQTQRLSAELHKLATLPRTETGSGSP

SEQ ID NO: 167 - protein, 34 aa, synthetic construct
  SITE 2: note = Aib
  VARIANT 12: note = I,K
  VARIANT 34: note = G,E
YXEGTFTSDYSXLLEEIAAREFIEWLJAGGPSSX

SEQ ID NO: 168 - protein, 34 aa, synthetic construct
  SITE 2: note = Aib
  VARIANT 12: note = I,K
  VARIANT 20: note = R,Q
  VARIANT 24: note = A,E,Q
  VARIANT 28: note = A,Q
  VARIANT 30: note = G,A
  VARIANT 31: note = G,Q,A,P
  VARIANT 33: note = E,S
  VARIANT 34: note = G,E
YXEGTFTSDYSXLLEEIAAXEFIXWLJXGXXSXX

SEQ ID NO: 169 - protein, 34 aa, synthetic construct
  SITE 2: note = Aib
  VARIANT 12: note = I,K
  VARIANT 30: note = G,A
  VARIANT 31: note = A,G,P
YXEGTFTSDYSXLLEEIAAREFIEWLJAGXXSSG

SEQ ID NO: 170 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPGASHLSTAQTQRLSAELHKLATLPRTETGSGSP

SEQ ID NO: 171 - protein, 76 aa, synthetic construct
  SITE 2: note = Aib
  MOD_RES 76: note = last position: C-terminal amide
YXEGTFTSDYSILLEEIAAREFIEWLIAGGPSSGAGQAPGQAPGASHLSTAQTQRLSAELHKLATLPRTETGSGSP

SEQ ID NO: 172 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPGASHLSTAQLGRLSAELHELATLPRTETGSGSP

SEQ ID NO: 173 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPGASHLSTAQLGRLSAELHQLATLPRTETGSGSP

SEQ ID NO: 174 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPGASHLSTAQTQRLSAELHELATLPRTETGSGSP

SEQ ID NO: 175 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPLASHLSTAQTQRLSAELHKLATLPRTETGSGSP

SEQ ID NO: 176 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPGASHLSTAALGRLSAELHELATLPRTETGSGSP

SEQ ID NO: 177 - protein, 76 aa, synthetic construct
  SITE 2: note = Aib
  MOD_RES 76: note = last position: C-terminal amide
YXEGTFTSDYSKLLEEIAAREFIEWLIAGGPSSGAGQAPGQAPGASHLSTAALGRLSAELHELATLPRTETGSGSP

SEQ ID NO: 178 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPLASHLSTAQTARLSAELHKLATLPRTETGSGSP

SEQ ID NO: 179 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPLASHLSTAQLGRLSAELHQLATLPRTETGSGSP

SEQ ID NO: 180 - protein, 68 aa, synthetic construct
  SITE 2: note = Aib
  MOD_RES 68: note = last position: C-terminal amide
YXEGTFTSDYSKLLEEIAAREFIEWLLAGGPSSGAGASHLSTAQLGRLSAELHQLATLPRTETGSGSP

SEQ ID NO: 181 - protein, 68 aa, synthetic construct
  SITE 2: note = Aib
  MOD_RES 68: note = last position: C-terminal amide
YXEGTFTSDYSILLEEIAAREFIEWLLAGGPSSGAGASHLSTAQTQRLSAELHKLATLPRTETGSGSP

SEQ ID NO: 182 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPLASQLSTAQTQRLSAELHKLATLPRTETGSGSP

SEQ ID NO: 183 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPLASELSTAQTQRLSAELHKLATLPRTETGSGSP

SEQ ID NO: 184 - protein, 67 aa, synthetic construct
  SITE 2: note = Aib
  MOD_RES 67: note = last position: C-terminal amide
YXEGTFTSDYSILLEEIAAREFIEWLLAGGPSSGEASHLSTAQTQRLSAELHKLATLPRTETGSGSP

SEQ ID NO: 185 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPAASHLSTAQTQRLSAELHKLATLPRTETGSGSP

SEQ ID NO: 186 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPEASHLSTAQTQRLSAELHKLATLPRTETGSGSP

SEQ ID NO: 187 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPPASHLSTAQTQRLSAELHKLATLPRTETGSGSP

SEQ ID NO: 188 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPSASHLSTAQTQRLSAELHKLATLPRTETGSGSP

SEQ ID NO: 189 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPVASHLSTAQTQRLSAELHKLATLPRTETGSGSP

SEQ ID NO: 190 - protein, 71 aa, synthetic construct
  SITE 2: note = Aib
  MOD_RES 71: note = last position: C-terminal amide
YXEGTFTSDYSILLEEIAAREFIEWLLAGGPSSGAGGGGASHLSTAQTQRLSAELHKLATLPRTETGSGSP

SEQ ID NO: 191 - protein, 71 aa, synthetic construct
  SITE 2: note = Aib
  MOD_RES 71: note = last position: C-terminal amide
YXEGTFTSDYSILLEEIAAREFIEWLLAGGPSSGAGGGGAGELSTAQTQRLSAELHKLATLPRTETGSGSP

SEQ ID NO: 192 - protein, 76 aa, synthetic construct
  SITE 2: note = Aib
  MOD_RES 76: note = last position: C-terminal amide
YXEGTFTSDYSILLEEIAAREFIEWLIAGGPSSGAGQAPGQAPLASHLSTAQTQRLSAELHKLATLPRTETGSGSP

SEQ ID NO: 193 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPGASELSTAQTQRLSAELHKLATLPRTETGSGSP

SEQ ID NO: 194 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPGASELSTAQTARLSAELHKLATLPRTETGSGSP

SEQ ID NO: 195 - protein, 76 aa, synthetic construct
  SITE 2: note = Aib
  MOD_RES 76: note = last position: C-terminal amide
YXEGTFTSDYSILLEEIAAREFIEWLLAGGPSSEAGQAPGQAPGASHLSTAQTQRLSAELHKLATLPRTETGSGSP

SEQ ID NO: 196 - protein, 76 aa, synthetic construct
  SITE 2: note = Aib
  MOD_RES 76: note = last position: C-terminal amide
YXEGTFTSDYSILLEEIAAREFIEWLLAGGPSSGEGQAPGQAPGASHLSTAQTQRLSAELHKLATLPRTETGSGSP

SEQ ID NO: 197 - protein, 76 aa, synthetic construct
  SITE 2: note = Aib
  MOD_RES 76: note = last position: C-terminal amide
YXEGTFTSDYSILLEEIAAREFIEWLLAGGPSSGAGEAPGQAPGASHLSTAQTQRLSAELHKLATLPRTETGSGSP

SEQ ID NO: 198 - protein, 76 aa, synthetic construct
  SITE 2: note = Aib
  MOD_RES 76: note = last position: C-terminal amide
YXEGTFTSDYSILLEEIAAREFIEWLLAGGPSSGAGQEPGQAPGASHLSTAQTQRLSAELHKLATLPRTETGSGSP

SEQ ID NO: 199 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAEGQAPGASHLSTAQTQRLSAELHKLATLPRTETGSGSP

SEQ ID NO: 200 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPEQAPGASHLSTAQTQRLSAELHKLATLPRTETGSGSP

SEQ ID NO: 201 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGEAPGASHLSTAQTQRLSAELHKLATLPRTETGSGSP

SEQ ID NO: 202 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQEPGASHLSTAQTQRLSAELHKLATLPRTETGSGSP

SEQ ID NO: 203 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAEGASHLSTAQTQRLSAELHKLATLPRTETGSGSP

SEQ ID NO: 204 - protein, 76 aa, synthetic construct
  SITE 2: note = Aib
  MOD_RES 76: note = last position: C-terminal amide
YXEGTFTSDYSILLEEIAAREFIEWLIAGGPSSGAGQEPGQEPGASHLSTAQTQRLSAELHKLATLPRTETGSGSP

SEQ ID NO: 205 - protein, 76 aa, synthetic construct
  SITE 2: note = Aib
  MOD_RES 76: note = last position: C-terminal amide
YXEGTFTSDYSILLEEIAAREFIAWLIAGGPSSGAGQEPGQEPGASHLSTAQTQRLSAELHKLATLPRTETGSGSP

SEQ ID NO: 206 - protein, 76 aa, synthetic construct
  SITE 2: note = Aib
  MOD_RES 76: note = last position: C-terminal amide
YXEGTFTSDYSILLEEIAAREFIQWLIAGGPSSGAGQEPGQEPGASHLSTAQTQRLSAELHKLATLPRTETGSGSP

SEQ ID NO: 207 - protein, 68 aa, synthetic construct
  SITE 2: note = Aib
  MOD_RES 68: note = last position: C-terminal amide
YXEGTFTSDYSILLEEIAAREFIEWLIAGGPSSGAGASHLSTAQTQRLSAELHKLATLPRTETGSGSP

SEQ ID NO: 208 - protein, 75 aa, synthetic construct
  SITE 2: note = Aib
  MOD_RES 75: note = last position: C-terminal amide
YXEGTFTSDYSILLEEIAAREFIEWLLAGGPSSGAGQAPGQAPASHLSTAQTQRLSAELHKLATLPRTETGSGSP

SEQ ID NO: 209 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPGASHLSTAQTQRLSAELHKLATEPRTETGSGSP

SEQ ID NO: 210 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPGASELSTAQTQRLSAELHKLATEPRTETGSGSP

SEQ ID NO: 211 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGEAPGASHLSTAQTQRLSAELHKLATEPRTETGSGSP

SEQ ID NO: 212 - protein, 76 aa, synthetic construct
  SITE 2: note = Aib
  MOD_RES 76: note = last position: C-terminal amide
YXEGTFTSDYSILLEEIAAREFIEWLLAGGPSSEAGQAPGQAPGASHLSTAQTQRLSAELHKLATEPRTETGSGSP

SEQ ID NO: 213 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGEAPGASELSTAQTQRLSAELHKLATLPRTETGSGSP

SEQ ID NO: 214 - protein, 76 aa, synthetic construct
  SITE 2: note = Aib
  MOD_RES 76: note = last position: C-terminal amide
YXEGTFTSDYSILLEEIAAREFIEWLLAGGPSSEAGQAPGQAPGASELSTAQTQRLSAELHKLATLPRTETGSGSP

SEQ ID NO: 215 - protein, 76 aa, synthetic construct
  SITE 2: note = Aib
  MOD_RES 76: note = last position: C-terminal amide
YXEGTFTSDYSILLEEIAAREFIEWLLAGGPSSEAGQAPGEAPGASHLSTAQTQRLSAELHKLATLPRTETGSGSP

SEQ ID NO: 216 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPGASELSTAALGRLSAELHELATLPRTETGSGSP

SEQ ID NO: 217 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGQAPGASELSTAALGRLSAELHQLATLPRTETGSGSP

SEQ ID NO: 218 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGEAPGASELSTAALGRLSAELHQLATLPRTETGSGSP

SEQ ID NO: 219 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGEAPGASELSTAALGRLSAELHQLATEPRTETGSGSP

SEQ ID NO: 220 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGEAPGASELSTAQLGRLSAELHQLATEPRTETGSGSP

SEQ ID NO: 221 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGEAPGASELSTAQTGRLSAELHQLATEPRTETGSGSP

SEQ ID NO: 222 - protein, 76 aa, synthetic construct
  SITE 2: note = Aib
  MOD_RES 76: note = last position: C-terminal amide
YXEGTFTSDYSKLLEEIAAREFIEWLLAGGPSSGAGQAPGEAPGASELSTAQTQRLSAELHQLATEPRTETGSGSP

SEQ ID NO: 223 - protein, 68 aa, synthetic construct
  SITE 2: note = Aib
  MOD_RES 68: note = last position: C-terminal amide
YXEGTFTSDYSILLEEIAAREFIEWLLAGGPSSGAGASELSTAQTQRLSAELHKLATLPRTETGSGSP

SEQ ID NO: 224 - protein, 68 aa, synthetic construct
  SITE 2: note = Aib
  MOD_RES 68: note = last position: C-terminal amide
YXEGTFTSDYSILLEEIAAREFIEWLLAGGPSSEAGASHLSTAQTQRLSAELHKLATLPRTETGSGSP

SEQ ID NO: 225 - protein, 68 aa, synthetic construct
  SITE 2: note = Aib
  MOD_RES 68: note = last position: C-terminal amide
YXEGTFTSDYSILLEEIAAREFIEWLLAGGPSSEAGASELSTAQTQRLSAELHKLATLPRTETGSGSP

SEQ ID NO: 226 - protein, 68 aa, synthetic construct
  SITE 2: note = Aib
  MOD_RES 68: note = last position: C-terminal amide
YXEGTFTSDYSILLEEIAAREFIEWLLAGGPSSGAGASHLSTAQTQRLSAELHKLATEPRTETGSGSP

SEQ ID NO: 227 - protein, 68 aa, synthetic construct
  SITE 2: note = Aib
  MOD_RES 68: note = last position: C-terminal amide
YXEGTFTSDYSILLEEIAAREFIEWLLAGGPSSGAGASQLSTAQTQRLSAELHKLATEPRTETGSGSP

SEQ ID NO: 228 - protein, 68 aa, synthetic construct
  SITE 2: note = Aib
  MOD_RES 68: note = last position: C-terminal amide
YXEGTFTSDYSILLEEIAAREFIEWLLAGGPSSEAGASQLSTAQTQRLSAELHKLATLPRTETGSGSP

SEQ ID NO: 229 - protein, 68 aa, synthetic construct
  SITE 2: note = Aib
  MOD_RES 68: note = last position: C-terminal amide
YXEGTFTSDYSILLEEIAAREFIEWLLAGGPSSEAGASQLSTAQTQRLSAELHKLATEPRTETGSGSP

SEQ ID NO: 230 - protein, 68 aa, synthetic construct
  SITE 2: note = Aib
  MOD_RES 68: note = last position: C-terminal amide
YXEGTFTSDYSKLLEEIAAREFIEWLLAGGPSSGAGASELSTAQLGRLSAELHQLATLPRTETGSGSP

SEQ ID NO: 231 - protein, 68 aa, synthetic construct
  SITE 2: note = Aib
  MOD_RES 68: note = last position: C-terminal amide
YXEGTFTSDYSKLLEEIAAREFIEWLLAGGPSSGAGASHLSTAQLGRLSAELHQLATEPRTETGSGSP

SEQ ID NO: 232 - protein, 68 aa, synthetic construct
  SITE 2: note = Aib
  MOD_RES 68: note = last position: C-terminal amide
YXEGTFTSDYSKLLEEIAAREFIEWLLAGGPSSEAGASHLSTAQLGRLSAELHQLATEPRTETGSGSP

SEQ ID NO: 233 - protein, 68 aa, synthetic construct
  SITE 2: note = Aib
  MOD_RES 68: note = last position: C-terminal amide
YXEGTFTSDYSKLLEEIAAREFIEWLLAGGPSSGAGASELSTAQLGRLSAELHQLATEPRTETGSGSP

SEQ ID NO: 234 - protein, 68 aa, synthetic construct
  SITE 2: note = Aib
  MOD_RES 68: note = last position: C-terminal amide
YXEGTFTSDYSKLLEEIAAREFIEWLLAGGPSSEAGASELSTAQLGRLSAELHQLATLPRTETGSGSP

SEQ ID NO: 235 - protein, 68 aa, synthetic construct
  SITE 2: note = Aib
  MOD_RES 68: note = last position: C-terminal amide
YXEGTFTSDYSKLLEEIAAREFIEWLLAGGPSSEAGASELSTAQLGRLSAELHQLATEPRTETGSGSP

SEQ ID NO: 236 - protein, 76 aa, synthetic construct
  SITE 2: note = Aib
  MOD_RES 76: note = last position: C-terminal amide
YXEGTFTSDYSILLEEIAAREFIEWLLAGGPSSGAGEAPGEAPGASHLSTAQTQRLSAELHKLATLPRTETGSGSP

SEQ ID NO: 237 - protein, 76 aa, synthetic construct
  SITE 2: note = Aib
  MOD_RES 76: note = last position: C-terminal amide
YXEGTFTSDYSILLEEIAAREFIEWLIAGGPSSGAGEAPGQAPGASQLSTAQTQRLSAELHKLATLPRTETGSGSP

SEQ ID NO: 238 - protein, 76 aa, synthetic construct
  SITE 2: note = Aib
  MOD_RES 76: note = last position: C-terminal amide
YXEGTFTSDYSILLEEIAAREFIEWLIAGGPSSGAGEAPGQAPGASELSTAQTQRLSAELHKLATLPRTETGSGSP

SEQ ID NO: 239 - protein, 68 aa, synthetic construct
  SITE 2: note = Aib
  MOD_RES 68: note = last position: C-terminal amide
YXEGTFTSDYSILLEEIAAQEFIEWLLQGGPSSGAGASELSTAQTQRLSAELHKLATLPRTETGSGSP

SEQ ID NO: 240 - protein, 76 aa, synthetic construct
  SITE 2: note = Aib
  MOD_RES 76: note = last position: C-terminal amide
YXEGTFTSDYSILLEEIAAQEFIEWLLQGGPSSGAGEAPGQAPGASELSTAQTQRLSAELHKLATLPRTETGSGSP

SEQ ID NO: 241 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGEAPLASHLSTAQTQRLSAELHKLATLPRTETGSGSP

SEQ ID NO: 242 - protein, 76 aa, synthetic construct
  SITE 2: note = Aib
  MOD_RES 76: note = last position: C-terminal amide
YXEGTFTSDYSILLEEIAAREFIEWLLAGGPSSGAGQAPGQAPLASHLSTAQTQRLSAELHKLATEPRTETGSGSP

SEQ ID NO: 243 - protein, 76 aa, synthetic construct
  SITE 2: note = Aib
  MOD_RES 76: note = last position: C-terminal amide
YXEGTFTSDYSILLEEIAAREFIEWLLAGGASSGAGEAPGEAPGASHLSTAQTQRLSAELHKLATLPRTETGSGSP

SEQ ID NO: 244 - protein, 68 aa, synthetic construct
  SITE 2: note = Aib
  MOD_RES 68: note = last position: C-terminal amide
YXEGTFTSDYSKLLEEIAAREFIEWLLAGGPSSGAGASELSTAALGRLSAELHQLATLPRTETGSGSP

SEQ ID NO: 245 - protein, 68 aa, synthetic construct
  SITE 2: note = Aib
  MOD_RES 68: note = last position: C-terminal amide
YXEGTFTSDYSKLLEEIAAREFIEWLLAGGQSSGAGASELSTAQLGRLSAELHQLATLPRTETGSGSP

SEQ ID NO: 246 - protein, 68 aa, synthetic construct
  SITE 2: note = Aib
  MOD_RES 68: note = last position: C-terminal amide
YXEGTFTSDYSKLLEEIAAREFIEWLIAGAPSSGAGASELSTAALGRLSAELHQLATLPRTETGSGSP

SEQ ID NO: 247 - protein, 68 aa, synthetic construct
  SITE 2: note = Aib
  MOD_RES 68: note = last position: C-terminal amide
YXEGTFTSDYSKLLEEIAAREFIEWLLAGGPSSGAEASELSTAQLGRLSAELHQLATLPRTETGSGSP

SEQ ID NO: 248 - protein, 68 aa, synthetic construct
  SITE 2: note = Aib
  MOD_RES 68: note = last position: C-terminal amide
YXEGTFTSDYSKLLEEIAAREFIEWLIAGGPSEGAGASELSTAQLGRLSAELHQLATLPRTETGSGSP

SEQ ID NO: 249 - protein, 68 aa, synthetic construct
  SITE 2: note = Aib
  MOD_RES 68: note = last position: C-terminal amide
YXEGTFTSDYSKLLEEIAAREFIEWLIAGGGSSGAGASELSTAALGRLSAELHELATLPRTETGSGSP

SEQ ID NO: 250 - protein, 68 aa, synthetic construct
  SITE 2: note = Aib
  MOD_RES 68: note = last position: C-terminal amide
YXEGTFTSDYSKLLEEIAAREFIEWLIAGAPSSGAGASELSTAALGRLSAELHELATLPRTETGSGSP

SEQ ID NO: 251 - protein, 68 aa, synthetic construct
  SITE 2: note = Aib
  MOD_RES 68: note = last position: C-terminal amide
YXEGTFTSDYSKLLEEIAAREFIEWLLAGGGSSGAGASELSTAALGRLSAELHELATLPRTETGSGSP

SEQ ID NO: 252 - protein, 68 aa, synthetic construct
  SITE 2: note = Aib
  MOD_RES 68: note = last position: C-terminal amide
YXEGTFTSDYSKLLEEIAAREFIEWLLAGGPSSGAGASELSTAALGRLSAELHELATLPRTETGSGSP